(19) United States
(12) Patent Application Publication
(10) Pub. No.: US 2025/0152729 A1
(43) Pub. Date: May 15, 2025

(54) IMMUNOTHERAPY FOR CANCER

(71) Applicants: Cancer Research Technology Limited, Greater London (GB); Medimmune Limited, Cambridge (GB)

(72) Inventors: Alexandra Addyman, London (GB); Georgina Anderson, Cambridge (GB); Mark Austin, Cambridge (GB); Michelle Barnard, Cambridge (GB); Denice Tsz Yau Chan, Cambridge (GB); Michael Chapman, Cambridge (GB); Agata Diamandakis, Cambridge (GB); Maria Groves, Cambridge (GB); William Hawthorne, Cambridge (GB); Stuart Haynes, Cambridge (GB); Lesley Jenkinson, Cambridge (GB); Jean-Martin Lapointe, Cambridge (GB); Kirsty-Jane Martin, Cambridge (GB); Louise Slater, Cambridge (GB); Tristan Vaughan, Cambridge (GB)

(73) Assignees: Cancer Research Technology Limited, Greater London, LN (GB); Medimmune Limited, Cambridge, CB (GB)

(21) Appl. No.: 18/718,334

(22) PCT Filed: Dec. 12, 2022
(86) PCT No.: PCT/GB2022/053174

§ 371 Date: Jun. 10, 2024

(30) Foreign Application Priority Data
Dec. 11, 2021 (GB) GB 2117928.8

(51) Int. Cl. A61K 47/68; C07K 14/725; C07K 16/28

(52) U.S. Cl. CPC A61K 47/6849; A61K 47/68031; A61K 47/68033; A61K 47/6889; C07K 14/7051; C07K 16/2803; C07K 2317/622

(57) ABSTRACT

An antibody-drug conjugate (ADC) comprising an anti-human SEMA4A humanised or human antibody, a linker and a cytotoxin.

### BACKGROUND TO THE INVENTION

Cancer immunotherapy, the induction of the immune system to attack tumour cells, has a long history, and has seen a recent resurgence of interest. This has been driven both by the success of immune checkpoint blockade and of cancer-directed immune therapies. The latter, exploiting the targeting of a cell surface protein on the cancer cell, is exemplified by monoclonal antibody therapies (e.g., rituximab, trastuzumab), introduced at the end of the twentieth century, and chimeric antigen receptor-T (CAR-T) cells, introduced at the start of the twenty-first. However, resistance to single-agent immunotherapy is frequent and is commonly driven by antigen escape, i.e., the downregulation of the antigenic target by the cancer cell. Antigen escape may be driven by intra-tumour heterogeneity, wherein subclones expressing low or absent levels of target protein gain a competitive growth advantage during immunotherapy. To overcome this resistance, novel immunotherapies should be targeted against antigens essential for cancer cell survival and combination treatments may be used. Strategies which combine multiple monoclonal antibodies are particularly attractive, in part because of low toxicity, and in part because immunophenotyping of cancer cells can reveal susceptible and resistant sub-populations and lead to rational therapeutic decisions. There is thus an urgent need to identify novel target proteins on the surface of cancer cells.

A complete understanding of the surface proteome of cancer cells would be particularly valuable to advance treatment. However, this knowledge has proven elusive with available technologies. Correlation between RNA and protein expression is modest, making transcriptomic approaches inefficient. Comprehensive identification of cell surface proteins by whole-cell proteomic approaches is limited by their low abundance, hydrophobicity, and lack of protease cleavage sites, as well as inaccurate quantitation. To overcome these limitations, various technologies have been employed to isolate and identify cancer cell surface proteins. However, these approaches have mainly focused on cancer cell lines instead of primary cells, have failed to capture the entirety of cell surface proteins, and/or are not quantitative. Consequently, these types of proteomic studies have failed to yield relevant cancer immunotherapy targets.

WO2021195536 describes analysis of the expression of cell surface candidate targets in multiple myeloma (MM). Surface proteins of 7 different MM cell lines were biotinylated and subjected to spectrometry analysis thereby identifying 4761 proteins, an integrated database was used to generate cell surface molecule annotation, which was combined with exclusion based on expression levels to identify 326 surface proteins for further analysis by STRING. A heatmap revealed protein annotation of 94 selected targets in several normal tissues and organs of the whole body. Molecules with high expression in any normal tissue except haematopoietic tissues and molecules with annotation in less than 2 out of 3 proteomic databases were excluded. A Venn diagram overlap of targets with a potential biological relevance and therapeutic relevance revealed 67 common targets, of which 24 were selected based on expression profile in primary patients and were used for validation in patient samples including: CCR1, CD28, CD320, FCRL3, IFNGR1, IL12RB1, IL27RA, IL2RG, IL6R, ITGA4, KCNN4, LAX1, LILRB1, LILRB4, LRRC8A, LRRC8D, PLXNA3, PLXNC1, S1PR4, SELPLG, SEMA4A, SLAMF6, TLR1 and BCMA.

Anderson et al. (2022) describe plasma membrane profiling of primary human myeloma cells to identify cell surface proteins of a primary cancer. A novel approach to prioritize immunotherapy targets was used to identify a cell surface protein not previously implicated in myeloma, semaphorin-4A (SEMA4A). It was shown that expression of SEMA4A is essential for normal myeloma cell growth in vitro, indicating that myeloma cells cannot down regulate the protein to avoid detection and also shown that SEMA4A would not be identified as a myeloma therapeutic target by standard CRISPR/Cas9 knockout screens because of exon skipping. Targeting of SEMA4A using a murine antibody-drug conjugate was demonstrated in vitro and in vivo.

MM is a cancer of plasma cells and causes fatigue, bone pain, pathological fractures, immunosuppression, and renal failure. Despite the introduction of novel therapies in recent years, including monoclonal antibody therapy and CAR-T cell therapy, resistance to treatment is inevitable. MM therefore remains invariably fatal and there is an urgent need for novel therapies.

### Statement of Invention—

The invention provides:
1. An antibody-drug conjugate (ADC) comprising an anti-human-SEMA4A human or humanised antibody or an antigen-binding fragment thereof, a linker and a cytotoxin.
2. An ADC according to clause 1, wherein the antibody comprises a V_{H} comprising HCDR1, HCDR2 and HCDR3 and a V_{L} comprising LCDR1, LCDR2 and LCDR3, wherein the CDRs are selected from the CDRs of:
   (a) 5E3 (SEQ ID NOS.: 3, 4, 5, 12, 13, 14);
   (b) 5Hg1-5Lg1 (SEQ ID NOS.: 21, 22, 23, 30, 31, 32);
   (c) 5Hg1-5Lg2 (SEQ ID NOS.: 39, 40, 41, 48, 49, 50);
   (d) 5Hg2-5Lg1 (SEQ ID NOS.: 57, 58, 59, 66, 67, 68);
   (e) 5Hg2-5Lg2 (SEQ ID NOS.: 75, 76, 77, 84, 85, 86);
   (f) 5Hg2-5Lg3 (SEQ ID NOS.: 93, 94, 95, 102, 103, 104);
   (g) 5Hg2-5Lg5 (SEQ ID NOS.: 111, 112, 113, 120, 121, 122);
   (h) 5Hg2-5Lg6 (SEQ ID NOS.: 129, 130, 131, 138, 139, 140);
   (i) 5Hg4-5Lg1 (SEQ ID NOS.: 147, 148, 149, 156, 157, 158);
   (j) 5Hg4-5Lg2 (SEQ ID NOS.: 165, 166, 167, 174, 175, 176);
   (k) 5Hg4-5Lg4 (SEQ ID NOS.: 183, 184, 185, 192, 193, 194);
   (l) 5Hg4-5Lg5 (SEQ ID NOS.: 201, 202, 203, 210, 211, 212);
   (m) 5Hg5-5Lg2 (SEQ ID NOS.: 219, 220, 221, 228, 229, 230);
   (n) C0120903 (SEQ ID NOS.: 237, 238, 239, 246, 247, 248);
   (o) C0120904 (SEQ ID NOS.: 255, 256, 257, 264, 265, 266);
   (p) C0120905 (SEQ ID NOS.: 273, 274, 275, 282, 283, 284);
   (q) C0120906 (SEQ ID NOS.: 291, 292, 293, 300, 301, 302);
   (r) C0120910 (SEQ ID NOS.: 309, 310, 311, 318, 319, 320);
   (s) C0120913 (SEQ ID NOS.: 327, 328, 329, 336, 337, 338);
   (t) C0120914 (SEQ ID NOS.: 345, 346, 347, 354, 355, 356);
   (u) C0120917 (SEQ ID NOS.: 363, 364, 365, 372, 373, 374);
   (v) C0120918 (SEQ ID NOS.: 381, 382, 383, 390, 391, 392);
   (w) C0120919 (SEQ ID NOS.: 399, 400, 401, 408, 409, 410);
   (x) C0120920 (SEQ ID NOS.: 417, 418, 419, 426, 427, 428);
   (y) C0120921 (SEQ ID NOS.: 435, 436, 437, 444, 445, 446);
   (z) C0120922 (SEQ ID NOS.: 453, 453, 455, 462, 463, 464);
   (aa) C0120923 (SEQ ID NOS.: 471, 472, 473, 480, 481, 482);
   (bb) C0120924 (SEQ ID NOS.: 489, 490, 491, 498, 499, 500);
   (cc) C0120925 (SEQ ID NOS.: 507, 508, 509, 516, 517, 518);
   (dd) C0120926 (SEQ ID NOS.: 525, 526, 527, 534, 535, 536);
   (ee) C0120927 (SEQ ID NOS.: 543, 544, 545, 552, 553, 554);
   (ff) C0120928 (SEQ ID NOS.: 561, 562, 563, 570, 571, 572);
   (gg) C0120929 (SEQ ID NOS.: 579, 580, 581, 588, 589, 590) and
   (hh) C0120930 (SEQ ID NOS.: 597, 598, 599, 606, 607, 608)
wherein the CDRs are defined according by Kabat nomenclature.
3. An ADC according to clause 1 or clause 2, wherein the antibody comprises a V_{H} and a V_{L} selected from the V_{H} and V_{L} of:
   a. 5Hg1-5Lg1 (SEQ ID NOS.: 20 and 29);
   b. 5Hg1-5Lg2 (SEQ ID NOS.: 38 and 47);
   c. 5Hg2-5Lg1 (SEQ ID NOS.: 56 and 65);
   d. 5Hg2-5Lg2 (SEQ ID NOS.: 74 and 83);
   e. 5Hg2-5Lg3 (SEQ ID NOS.: 92 and 101);
   f. 5Hg2-5Lg5 (SEQ ID NOS.: 110 and 119);
   g. 5Hg2-5Lg6 (SEQ ID NOS.: 128 and 137);
   h. 5Hg4-5Lg1 (SEQ ID NOS.: 146 and 155);
   i. 5Hg4-5Lg2 (SEQ ID NOS.: 164 and 173);
   j. 5Hg4-5Lg4 (SEQ ID NOS.: 182 and 191);
   k. 5Hg4-5Lg5 (SEQ ID NOS.: 200 and 209);
   l. 5Hg5-5Lg2 (SEQ ID NOS.: 218 and 227);
   m. C0120903 (SEQ ID NOS.: 236 and 245);
   n. C0120904 (SEQ ID NOS.: 254 and 263);
   o. C0120905 (SEQ ID NOS.: 272 and 281);
   p. C0120906 (SEQ ID NOS.: 290 and 299);
   q. C0120910 (SEQ ID NOS.: 308 and 317);
   r. C0120913 (SEQ ID NOS.: 326 and 335);
   s. C0120914 (SEQ ID NOS.: 344 and 353);
   t. C0120917 (SEQ ID NOS.: 362 and 371);
   u. C0120918 (SEQ ID NOS.: 380 and 389);
   v. C0120919 (SEQ ID NOS.: 398 and 407);
   w. C0120920 (SEQ ID NOS.: 416 and 425);
   x. C0120921 (SEQ ID NOS.: 434 and 443);
   y. C0120922 (SEQ ID NOS.: 452 and 461);
   z. C0120923 (SEQ ID NOS.: 470 and 479);
   aa. C0120924 (SEQ ID NOS.: 488 and 497);
   bb. C0120925 (SEQ ID NOS.: 506 and 515);
   cc. C0120926 (SEQ ID NOS.: 524 and 533);
   dd. C0120927 (SEQ ID NOS.: 542 and 551);
   ee. C0120928 (SEQ ID NOS.: 560 and 569);
   ff. C0120929 (SEQ ID NOS.: 578 and 587); and
   gg. C0120930 (SEQ ID NOS.: 596 and 605);
wherein the sequences are defined by Kabat nomenclature.
4. An ADC according to any one of the preceding clauses wherein the cytotoxin is a microtubule inhibitor (e.g., a maytansinoid or an auristatin);
5. An ADC according to any one of the preceding clauses wherein the cytotoxin is selected from monomethyl auristatin F (MMAF), monomethyl auristatin E (MMAE, vedotin) and mertansine (DM1, e.g., as emtansine with an SMCC linker).
6. An ADC according to any one of the preceding clauses wherein the linker is a non-cleavable linker or is a cleavable linker.
7. An ADC according to any one of clauses 1 to 6 wherein the cytotoxin is monomethyl auristatin E (MMAE) and the linker is a mc-vcPAB linker (malemide-based linker, cysteine linked) or the cytotoxin is mertansine (DM1) and the linker is a SMCC linker (NHS-ester based, lysine).
8. An ADC according to any one of the preceding clauses wherein the ADC bound to SEMA4A is capable of being internalised into a cell expressing human SEMA4A on its surface.
9. An ADC according to any one of the preceding clauses wherein the ADC bound to SEMA4A is capable of being internalised into a cell expressing human SEMA4A on its surface and internalisation of the ADC into the cell results in cell death.
10. A human or humanised anti-human SEMA4A antibody comprising a V_{H} comprising HCDR1, HCDR2 and HCDR3 and a V_{L} comprising LCDR1, LCDR2 and LCDR3, wherein the CDRs are selected from the CDRs of:
   (a) 5E3 (SEQ ID NOS.: 3, 4, 5, 12, 13, 14);
   (b) 5Hg1-5Lg1 (SEQ ID NOS.: 21, 22, 23, 30, 31, 32);
   (c) 5Hg1-5Lg2 (SEQ ID NOS.: 39, 40, 41, 48, 49, 50);
   (d) 5Hg2-5Lg1 (SEQ ID NOS.: 57, 58, 59, 66, 67, 68);
   (e) 5Hg2-5Lg2 (SEQ ID NOS.: 75, 76, 77, 84, 85, 86);
   (f) 5Hg2-5Lg3 (SEQ ID NOS.: 93, 94, 95, 102, 103, 104);
   (g) 5Hg2-5Lg5 (SEQ ID NOS.: 111, 112, 113, 120, 121, 122);
   (h) 5Hg2-5Lg6 (SEQ ID NOS.: 129, 130, 131, 138, 139, 140);
   (i) 5Hg4-5Lg1 (SEQ ID NOS.: 147, 148, 149, 156, 157, 158);
   (j) 5Hg4-5Lg2 (SEQ ID NOS.: 165, 166, 167, 174, 175, 176);
   (k) 5Hg4-5Lg4 (SEQ ID NOS.: 183, 184, 185, 192, 193, 194);
   (l) 5Hg4-5Lg5 (SEQ ID NOS.: 201, 202, 203, 210, 211, 212);
   (m) 5Hg5-5Lg2 (SEQ ID NOS.: 219, 220, 221, 228, 229, 230);
   (n) C0120903 (SEQ ID NOS.: 237, 238, 239, 246, 247, 248);
   (o) C0120904 (SEQ ID NOS.: 255, 256, 257, 264, 265, 266);
   (p) C0120905 (SEQ ID NOS.: 273, 274, 275, 282, 283, 284);
   (q) C0120906 (SEQ ID NOS.: 291, 292, 293, 300, 301, 302);
   (r) C0120910 (SEQ ID NOS.: 309, 310, 311, 318, 319, 320);
   (s) C0120913 (SEQ ID NOS.: 327, 328, 329, 336, 337, 338);
   (t) C0120914 (SEQ ID NOS.: 345, 346, 347, 354, 355, 356);
   (u) C0120917 (SEQ ID NOS.: 363, 364, 365, 372, 373, 374);
   (v) C0120918 (SEQ ID NOS.: 381, 382, 383, 390, 391, 392);
   (w) C0120919 (SEQ ID NOS.: 399, 400, 401, 408, 409, 410);
   (x) C0120920 (SEQ ID NOS.: 417, 418, 419, 426, 427, 428);
   (y) C0120921 (SEQ ID NOS.: 435, 436, 437, 444, 445, 446);
   (z) C0120922 (SEQ ID NOS.: 453, 454, 455, 462, 463, 464);
   (aa) C0120923 (SEQ ID NOS.: 471, 472, 473, 480, 481, 482);
   (bb) C0120924 (SEQ ID NOS.: 489, 490, 491, 498, 499, 500);
   (cc) C0120925 (SEQ ID NOS.: 507, 508, 509, 516, 517, 518);
   (dd) C0120926 (SEQ ID NOS.: 525, 526, 527, 534, 535, 536);
   (ee) C0120927 (SEQ ID NOS.: 543, 544, 545, 552, 553, 554);
   (ff) C0120928 (SEQ ID NOS.: 561, 562, 563, 570, 571, 572);
   (gg) C0120929 (SEQ ID NOS.: 579, 580, 581, 588, 589, 590) and
   (hh) C0120930 (SEQ ID NOS: 597, 598, 599, 606, 607, 608)
wherein the sequence of the CDRs is defined by Kabat nomenclature.
11. An antibody according to clause 10 wherein the antibody comprises a V_{H} and a V_{L} selected from the V_{H} and V_{L} of:
   (a) 5Hg1-5Lg1 (SEQ ID NOS.: 20 and 29);
   (b) 5Hg1-5Lg2 (SEQ ID NOS.: 38 and 47);
   (c) 5Hg2-5Lg1 (SEQ ID NOS.: 56 and 65);
   (d) 5Hg2-5Lg2 (SEQ ID NOS.: 74 and 83);
   (e) 5Hg2-5Lg3 (SEQ ID NOS.: 92 and 101);
   (f) 5Hg2-5Lg5 (SEQ ID NOS.: 110 and 119);
   (g) 5Hg2-5Lg6 (SEQ ID NOS.: 128 and 137);
   (h) 5Hg4-5Lg1 (SEQ ID NOS.: 146 and 155);
   (i) 5Hg4-5Lg2 (SEQ ID NOS.: 164 and 173);
   (j) 5Hg4-5Lg4 (SEQ ID NOS.: 182 and 191);
   (k) 5Hg4-5Lg5 (SEQ ID NOS.: 200 and 209);
   (l) 5Hg5-5Lg2 (SEQ ID NOS.: 218 and 227);
   (m) C0120903 (SEQ ID NOS.: 236 and 245);
   (n) C0120904 (SEQ ID NOS.: 254 and 263);
   (o) C0120905 (SEQ ID NOS.: 272 and 281);
   (p) C0120906 (SEQ ID NOS.: 290 and 299);
   (q) C0120910 (SEQ ID NOS.: 308 and 317);
   (r) C0120913 (SEQ ID NOS.: 326 and 335);
   (s) C0120914 (SEQ ID NOS.: 344 and 353);
   (t) C0120917 (SEQ ID NOS.: 362 and 371);
   (u) C0120918 (SEQ ID NOS.: 380 and 389);
   (v) C0120919 (SEQ ID NOS.: 398 and 407);
   (w) C0120920 (SEQ ID NOS.: 416 and 425);
   (x) C0120921 (SEQ ID NOS.: 434 and 443);
   (y) C0120922 (SEQ ID NOS.: 452 and 461);
   (z) C0120923 (SEQ ID NOS.: 470 and 479);
   (aa) C0120924 (SEQ ID NOS.: 488 and 497);
   (bb) C0120925 (SEQ ID NOS.: 506 and 515);
   (cc) C0120926 (SEQ ID NOS.: 524 and 533);
   (dd) C0120927 (SEQ ID NOS.: 542 and 551);
   (ee) C0120928 (SEQ ID NOS.: 560 and 569);
   (ff) C0120929 (SEQ ID NOS.: 578 and 587) and
   (gg) C0120930 (SEQ ID NOS.: 596 and 605)
wherein the V_{H} and V_{L} sequences are defined by Kabat nomenclature.
12. A chimeric antigen receptor (CAR) comprising an antigen-binding domain of the monoclonal antibody of any one of clauses 10 or 11 linked to a T-cell activation moiety.
13. A CAR of clause 12, wherein the antigen-binding domain comprises a single chain Fv (scFv) fragment of a monoclonal antibody of any one of clauses 10 or 11.
14. A composition comprising an ADC according to any one of clauses 1 to 9, or an anti-human SEMA4A antibody according to any one of clauses 10 or 11, or a CAR of any one of clauses 12 and 13 and a diluent.
15. An ADC according to any one of clauses 1 to 9, an antibody according to any one of clauses 10 or 11, a CAR according to any one of clauses 12 or 13 or a composition according to clause 14:
   a. for use as a medicament;
   b. for use as a medicament for the treatment of cancer;
   c. for use in the treatment of haematological cancer;
   d. for use in the treatment of a haematological cancer selected from multiple myeloma (MM), non-Hodgkin's lymphoma (NHL), acute myeloid leukaemia (AML), diffuse large B-cell lymphoma (DLBCL) and follicular lymphoma (FL);
   e. for use in the manufacture of a medicament for the therapeutic treatment of a cancer;
   f. for use in the manufacture of a medicament for the therapeutic treatment of a haematological cancer;
   g. for use in the manufacture of a medicament for the therapeutic treatment of a haematological cancer selected from MM, NHL, AML, DLBCL and FL, or,
   h. for inducing cell death in cells expressing SEMA4A at the cell surface.
16. A method of treatment of a cancer, such as a haematological cancer, e.g., a haematological cancer selected from MM, NHL, AML, DLBCL and FL, comprising administration of an ADC according to any one of clauses 1 to 9, an antibody according to any one of clauses 10 or 11, a CAR according to any one of clauses 12 or 13 or a composition according to clause 14, to a subject.
17. A method for manufacture of an ADC according to any one of clauses 1 to 9 comprising conjugation of an antibody according to clause 10 or clause 11 to a cytotoxin via a linker.
18. An isolated recombinant DNA or RNA sequence comprising a sequence encoding an isolated antibody or antigen-binding fragment thereof, according to any one of clauses 10 or 11.
19. An isolated recombinant DNA sequence of clause 18 which is a vector.
20. An isolated recombinant DNA sequence of clause 18 or clause 19 which is an expression vector.
21. An isolated recombinant DNA sequence of any one of clauses 18 to 20 encoding an antibody or antigen-binding fragment thereof, according to any one of clauses 10 or 11 under control of a promoter.
22. A host cell comprising a DNA or RNA sequence according to any one of clauses 18 to 21.
23. A host cell of clause 22 capable of expressing an isolated antibody or antigen-binding fragment thereof, of any one of clauses 10 or 11.
24. A method of making an isolated antibody or antigen-binding fragment thereof, of clause 10 or 11 comprising culturing a host cell according to clause 22 or 23 in conditions suitable for expression of the isolated antibody or antigen-binding fragment thereof.

The present invention provides an antibody-drug conjugate (ADC) comprising a human or humanised antibody, or an antigen-binding fragment thereof, directed against human Semaphorin4A (SEMA4A) conjugated to a cytotoxin. The term “antibody-drug conjugate”, as used herein, refers to a compound comprising an antibody, such as a humanised or human monoclonal antibody (mAb) or an antigen-binding fragment thereof attached to a cytotoxic agent (generally a small molecule drug with a high systemic toxicity) via a chemical linker. In some embodiments, an ADC may comprise a small molecule cytotoxin that has been chemically modified to contain a linker. The linker is then used to conjugate the cytotoxin to the antibody, or antigen-binding fragment thereof. In some embodiments, upon binding to the target antigen on the surface of the cell, the cytotoxin can be released by cleavage of the linker or proteolysis, the cytotoxin can then bind to its target and induce cell death.

The ADCs described herein may comprise a whole antibody or an antibody fragment. A whole antibody typically consists of four polypeptides: two identical copies of a heavy (H) chain polypeptide and two identical copies of a light (L) chain polypeptide. Each of the heavy chains contains one N-terminal variable (V_{H}) region and three C-terminal constant (CH1, CH2 and CH3) regions, and each light chain contains one N-terminal variable (V_{L}) region and one C-terminal constant (CL) region. The variable regions of each pair of light and heavy chains form the antigen binding site of an antibody. The V_{H} and V_{L} regions have the same general structure, with each region comprising four framework regions, whose sequences are relatively conserved. The framework regions are connected by three complementarity determining regions (CDRs). The three CDRs, known as CDR1, CDR2, and CDR3, form the “hypervariable region” of each variable domain, which is responsible for antigen binding.

The ADC may comprise an antigen-binding fragment of a humanised or human antibody. The terms “antibody fragment,” “antigen-binding fragment,” “functional fragment of an antibody,” and “antigen-binding portion” are used interchangeably herein and refer to one or more fragments or portions of an antibody that retain the ability to specifically bind to an antigen. The antibody fragment may comprise, for example, one or more CDRs, the variable region (or portions thereof), or combinations thereof, optionally in further combination with the constant region (or portions thereof). Examples of antibody fragments include, but are not limited to, (i) a Fab fragment, which is a monovalent fragment consisting of the V_{L}, V_{H}, CL, and CH1 domains; (ii) a F(ab′)2 fragment, which is a bivalent fragment comprising two Fab fragments linked by a disulfide bridge at the hinge region; (iii) a Fv fragment consisting of the V_{L} and V_{H} domains of a single arm of an antibody; (iv) a single chain Fv (scFv), which is a monovalent molecule consisting of the two domains of the Fv fragment (i.e., V_{L} and V_{H}) joined by a synthetic linker which enables the two domains to be synthesized as a single polypeptide chain and (v) a diabody, which is a dimer of polypeptide chains, wherein each polypeptide chain comprises a V_{H} connected to a V_{L} by a peptide linker that is too short to allow pairing between the V_{H} and V_{L} on the same polypeptide chain, thereby driving the pairing between the complementary domains on different V_{H}-V_{L} polypeptide chains to generate a dimeric molecule having two functional antigen binding sites. Antibody fragments are known in the art and are described in more detail in, e.g., U.S. Patent Application Publication 2009/0093024 A1.

An ADC of the invention comprising a humanised antibody of the invention or comprising a human antibody of the invention is capable of binding specifically to recombinant human SEMA4A (UniProt ID Q9H3S1), and may also bind to recombinant cynomolgus SEMA4A (UniProt ID G7NV79) and/or to recombinant mouse SEMA4A (UniProt ID Q62178).

In one embodiment, the ADC comprises a variable region of a humanised or fully human anti-human-SEMA4A antibody. In this respect, the ADC may comprise a light chain variable region, a heavy chain variable region, or both a light chain variable region and a heavy chain variable region of an anti-human-SEMA4A monoclonal antibody. Preferably, the ADC comprises a light chain variable region and a heavy chain variable region of an anti-human-SEMA4A antibody. The terms “cytotoxin” and “cytotoxic agent” refer to any molecule that inhibits or prevents the function of cells and/or causes destruction of cells (cell death), and/or exerts anti-proliferative effects. It will be appreciated that a cytotoxin or cytotoxic agent of an ADC also is referred to in the art as the “payload” or “warhead” of the ADC. A number of classes of cytotoxic agents are known in the art to have potential utility in ADC molecules and can be used in the ADC described herein. An exemplary class of cytotoxic agents includes anti-microtubule agents such as a tubulysin, a maytansinoid, an auristatin, or derivatives thereof. More specifically, the cytotoxic agent may be, for example MMAF, MMAE or DM1, DM4.

In one embodiment, the cytotoxic agent may be an anti-microtubule agent. The terms “anti-microtubule agent” and “microtubule-targeting agent,” are synonymous and refer to an agent that inhibits cell division by interfering with microtubules. Tubulysins are members of a class of natural products isolated from myxobacterial species (Sasse et al., 2000) which act as mitotic poisons that inhibit tubulin polymerization and lead to cell cycle arrest and apoptosis (Steinmetz et al., 2004; Khalil et al., 2006; Kaur et al., 2006). Examples of tubulysins are disclosed in, for example, International Patent Application Publication Nos. WO 2015/157594, WO 2004/005326, WO 2012/019123, WO 2009/134279, WO 2009/055562, WO 2004/005327; U.S. Pat. Nos. 7,776,841, 7,754,885, and 7,816,377; and U.S. Patent Application Publications 2010/0240701, 2011/0021568, and 2011/0263650.

Maytansinoids inhibit polymerization of the microtubule protein tubulin, thereby preventing formation of microtubules (see, e.g., U.S. Pat. No. 6,441,163 and Remillard et al., 1975). Maytansinoids have been shown to inhibit tumour cell growth in vitro using cell culture models, and in vivo using laboratory animal systems. Moreover, the cytotoxicity of maytansinoids is 1,000-fold greater than conventional chemotherapeutic agents, such as, for example, methotrexate, daunorubicin, and vincristine (see, e.g., U.S. Pat. No. 5,208,020). Maytansinoids include maytansine, maytansinol, C-3 esters of maytansinol, and other maytansinol analogues and derivatives (see, e.g., U.S. Pat. Nos. 5,208,020 and 6,441,163). C-3 esters of maytansinol can be naturally occurring or synthetically derived. Moreover, both naturally occurring and synthetic C-3 maytansinol esters can be classified as a C-3 ester with simple carboxylic acids, or a C-3 ester with derivatives of N-methyl-L-alanine, the latter being more cytotoxic than the former. Synthetic maytansinoid analogues also are known in the art and described in, for example, Kupchan et al., 1978. Methods for generating maytansinol and analogues and derivatives thereof are described in, for example, U.S. Pat. No. 4,151,042. Examples of maytansinoids that may be used in connection with the ADC described herein include, but are not limited to, N2′-deacetyl-N2′-(3-mercapto-1-oxopropyl)-maytansine (DM1) and N2′-deacetyl-N2′-(4-mercapto-4-methyl-1-oxopentyl)-maytansine (DM4).

Auristatins represent a class of highly potent anti-mitotic agents that have shown substantial preclinical activity at well-tolerated doses (Law et al., 2006; Ma et al., 2006; Tse et al., 2006; Oflazoglu et al., 2008, Oflazoglu et al., 2008). Auristatin ADCs are currently being evaluated in preclinical and clinical trials. Examples of auristatins that may be used in connection with the ADC described herein include, but are not limited to, monomethyl auristatin E (MMAE) and the related molecule monomethyl auristatin F (MMAF) (see, e.g., Doronina et al., 2003; Doronina et al., 2006).

The SEMA4A humanised or human monoclonal antibody, or antigen-binding fragment thereof, may be conjugated to a cytotoxin using any suitable method known in the art, including site-specific or non-site specific conjugation methods. Conventional conjugation strategies for antibodies typically rely on stochastically, that is randomly (i.e., non-specifically) conjugating the payload to the antibody, antigen-binding fragment thereof, through lysines or cysteines. Accordingly, in some aspects the antibody or antigen-binding fragment thereof is randomly conjugated to a cytotoxic agent, for example, by partial reduction of the antibody or antibody fragment, followed by reaction with a desired agent with or without a linker moiety attached. For example, the antibody or antigen-binding fragment thereof, may be reduced using dithiothreitol (DTT) or a similar reducing agent. The cytotoxic agent, with or without a linker moiety attached thereto, can then be added at a molar excess to the reduced antibody or antibody fragment in the presence of dimethyl sulfoxide (DMSO). After conjugation, excess free cysteine may be added to quench unreacted agent. The reaction mixture may then be purified and buffer-exchanged into phosphate buffered saline (PBS).

In other embodiments, the cytotoxic agent may be conjugated to the SEMA4A monoclonal antibody using site-specific conjugation methods at specific reactive amino acid residues, yielding homogeneous ADC preparations with uniform stoichiometry. Site-specific conjugation may be through a cysteine residue or a non-natural amino acid. In one embodiment, the cytotoxic agent may be conjugated to the antibody, or antigen binding fragment thereof, through at least one cysteine residue. In particular, for example, a cytotoxic agent may be chemically conjugated to the side chain of an amino acid at a specific Kabat position (Kabat et al., 1991) in the Fc region of the SEMA4A monoclonal antibody. In this regard, the cytotoxic agent may be conjugated to the SEMA4A monoclonal antibody through a cysteine residue at any suitable position in the Fc region of the antibody. Alternatively, the cytotoxic agent may be conjugated to the SEMA4A monoclonal antibody or antigen binding fragment thereof through a thiol-maleimide linkage, such as, for example, via a sulfhydryl reactive group at the hinge and heavy-light chains.

The SEMA4A humanised or human monoclonal ADC described herein comprises at least one cytotoxin molecule conjugated thereto; however, the SEMA4A humanised or human monoclonal antibody may comprise any suitable number of cytotoxin molecules conjugated thereto (e.g., 1, 2, 3, 4, or more cytotoxin molecules) to achieve a desired therapeutic effect. Accordingly, an ADC of the invention may have a drug-antibody ratio (DAR) of, for example, 1, 2, 3, 4, 5, 6, 7, or 8. DAR is the average drug (cytotoxin) to antibody ratio for a given preparation of ADC. DAR is a measure of drug loading for an ADC.

In some embodiments, the invention provides a humanised or human monoclonal antibody, or an antigen-binding fragment thereof, directed against human SEMA4A described above independent of an ADC.

Humanised and human antibodies of the invention and ADC or CAR comprising such antibodies are capable of binding specifically to recombinant human SEMA4A (UniProt ID Q9H3S1, SEQ ID NO: 613) and may also bind recombinant cynomolgus SEMA4A (UniProt ID G7NV79, SEQ ID NO: 614) and/or recombinant mouse SEMA4A (UniProt ID Q62178, SEQ ID NO: 615).

The humanised or human antibody, or an antigen-binding fragment thereof, directed against human SEMA4A may comprise any suitable binding affinity to human SEMA4A or an epitope thereof. The term “affinity” refers to the equilibrium constant for the reversible binding of two agents and is expressed as the dissociation constant (K_{D}). The affinity of an antibody or antigen-binding fragment thereof for an antigen or epitope of interest can be measured using any method known in the art. Such methods include, for example, fluorescence activated cell sorting (FACS), surface plasmon resonance (e.g., Biacore, ProteOn), biolayer interferometry (BLI, e.g., Octet), kinetics exclusion assay (e.g., KinExA), separable beads (e.g., magnetic beads), antigen panning, and/or enzyme-linked immunosorbent assay (ELISA) (Janeway et al., 2001). It is known in the art that the binding affinity of a particular antibody will vary depending on the method that is used to analyze the binding affinity.

Affinity of a binding agent to a ligand, such as affinity of an antibody for an epitope, can be, for example, from about 1 nM to about 100 nM. In one embodiment, the monoclonal antibody or an antigen-binding fragment thereof may bind to human SEMA4A with a K_{D} less than or equal to 500, 400, 300, 200 or 100 nanomolar (e.g., 100 nM, about 90 nM, about 80 nM, about 70 nM, about 60 nM, about 50 nM, about 40 nM, about 30 nM, about 20 nM, or about 10 nM, or a range defined by any two of the foregoing values). In another embodiment, the monoclonal antibody may bind to human SEMA4A with a K_{D} less than or equal to 10 nanomolar (e.g., about 9 nM, about 8.5 nM, about 8 nM, about 7.5 nM, about 7 nM, about 6.5 nM, about 6 nM, about 5.5 nM, about 5 nM, about 4.5 nM, about 4 nM, about 3.5 nM, about 3 nM, about 2.5 nM, about 2 nM, about 1.5 nM, about 1 nM, or a range defined by any two of the foregoing values).

An antigen-binding portion or fragment of a humanised or human antibody of the invention can be of any size so long as the portion binds to human SEMA4A.

An antibody or antigen-binding fragment thereof of the invention may be produced by recombinant means. A “recombinant antibody” is an antibody which has been produced by a recombinantly engineered host cell. An antibody or antigen-binding fragment thereof in accordance with the invention is optionally isolated or purified. The term “antibody” or “antibody molecule” describes an immunoglobulin whether natural or partly or wholly synthetically produced. An antigen-binding protein of the invention may be an antibody, preferably a monoclonal antibody, and may be human or non-human, chimeric or humanised. The antibody molecule is preferably a monoclonal antibody molecule. Examples of antibodies are the immunoglobulin isotypes, such as immunoglobulin G, and their isotypic subclasses, such as IgG1, IgG2, IgG3 and IgG4, as well as fragments thereof. The four human subclasses (IgG1, IgG2, IgG3 and IgG4) each contain a different heavy chain; but they are highly homologous and differ mainly in the hinge region and the extent to which they activate the host immune system. IgG1 and IgG4 contain two inter-chain disulphide bonds in the hinge region, IgG2 has 4 and IgG3 has 11 inter-chain disulphide bonds.

The terms “antibody” and “antibody molecule”, as used herein, include antibody fragments, such as Fab and scFv fragments, provided that said fragments comprise a CDR-based antigen binding site for an epitope of human SEMA4A. Examples of antibody fragments include but are not limited to Fv, Fab, F(ab′), Fab′-SH, F(ab′)2; diabodies; linear antibodies; single-chain antibody molecules (e.g., scFv) and domain antibodies (sdAbs). Unless the context requires otherwise, the terms “antigen-binding protein”, “antibody” or “antibody molecule”, as used herein, is thus equivalent to “antibody or antigen-binding fragment thereof”.

Antibodies are immunoglobulins, which have the same basic structure consisting of two heavy and two light chains forming two Fab arms containing identical domains that are attached by a flexible hinge region to the stem of the antibody, the Fc domain, giving the classical ‘Y’ shape. The Fab domains consist of two variable and two constant domains, with a variable heavy (V_{H}) and constant heavy 1 (CH1) domain on the heavy chain and a variable light (V_{L}) and constant light (CL) domain on the light chain. The two variable domains (V_{H} and V_{L}) form the variable fragment (Fv), which provides the CDR-based antigen specificity of the antibody, with the constant domains (CH1 and V_{L}) acting as a structural framework. Each variable domain contains three hypervariable loops, known as complementarity determining regions (CDRs). On each of the V_{H} and V_{L} domains, the three CDRs (CDR1, CDR2, and CDR3) are flanked by four less-variable framework (FW) regions (FW1, FW2, FW3 and FW4) to give a structure FW1-CDR1-FW2-CDR2-FW3-CDR3-FW4. The CDRs provide a specific antigen recognition site on the surface of the antibody.

Both Kabat and ImMunoGeneTics (IMGT) numbering nomenclature may be used herein. Generally, unless otherwise indicated (explicitly or by context) amino acid residues are numbered herein according to the Kabat numbering scheme (Kabat et al., 1991). For those instances when the IMGT numbering scheme is used, amino acid residues are numbered herein according to the ImMunoGeneTics (IMGT) numbering scheme described in Lefranc et al., 2005.

It is possible to take monoclonal and other antibodies and use techniques of recombinant DNA technology to produce other antibodies or chimeric molecules which generally retain the specificity of the original antibody. Such techniques may involve introducing the CDRs into a different immunoglobulin framework, or grafting variable regions onto a different immunoglobulin constant region. Introduction of the CDRs of one immunoglobulin into another immunoglobulin is described for example in EP-A-184187, GB2188638A or EP-A-239400. Alternatively, a hybridoma or other cell producing an antibody molecule may be subject to genetic mutation or other changes, which may or may not alter the binding specificity of antibodies produced.

Antibody humanisation involves the transfer, or “grafting”, of critical non-human amino acids onto a human antibody framework. Primarily this includes the grafting of amino acids in the complementarity-determining regions (CDRs), but potentially also other framework amino acids critical for the V_{H}-V_{L} interface and for orientation of the CDRs. Humanisation seeks to introduce human content to reduce the risk of immunogenicity, while retaining the original binding activity of the non-human parental antibody. The term “humanised antibody” is intended to refer to antibodies in which CDR sequences derived from the germline of another mammalian species have been grafted onto human framework sequences; optionally additional framework region modifications can be made within the human framework sequences. The term “humanised antibody” includes antibodies in which CDR sequences derived from the germline of another mammalian species have been grafted onto human framework sequences and optimised (for example by affinity maturation), e.g., by modification of one or more amino acid residues in one or more of the CDRs and/or in one or more framework sequence to modulate or improve a biological property of the humanised antibody, e.g., to increase affinity, or to modulate the on rate and/or off rate for binding of the antibody to its target epitope.

Variable domains employed in the invention may be obtained or derived from any germline or rearranged human variable domain, or may be a synthetic variable domain based on consensus or actual sequences of known human variable domains. A repertoire of variable domains may be displayed in a suitable host system, such as the phage display system of WO92/01047, which is herein incorporated by reference in its entirety, or any of a subsequent large body of literature, including Kay, Winter & McCafferty [Kay, B. K., Winter, J., and McCafferty, J. (1996) Phage Display of Peptides and Proteins: A Laboratory Manual, San Diego: Academic Press], so that suitable binding members may be selected. Other suitable host systems include, but are not limited to, yeast display, bacterial display, T7 display, viral display, cell display, ribosome display and covalent display.

As antibodies can be modified in a number of ways, the term “antigen-binding protein” or “antibody” should be construed as covering antibody fragments, derivatives, functional equivalents and homologues of antibodies, including any polypeptide comprising an immunoglobulin binding domain, an aptamer, affimer or bicyclic peptide, whether natural or wholly or partially synthetic.

Chimeric molecules comprising an immunoglobulin binding domain, or equivalent, fused to another polypeptide are therefore included. Cloning and expression of chimeric antibodies are described in EP-A-0120694 and EP-A-0125023.

An example of an antibody fragment comprising both CDR sequences and CH3 domain is a minibody, which comprises a scFv joined to a CH3 domain (Hu et al., 1996).

A domain (single-domain) antibody is a peptide, usually about 110 amino acids long, comprising one variable domain (V_{H}) of a heavy-chain antibody, or of an IgG. A single-domain antibody (sdAb), (e.g., nanobody), is an antibody fragment consisting of a single monomeric variable antibody domain. Like a whole antibody (comprising two heavy and two light chains), it is an antigen-binding protein able to bind selectively to a specific antigen. Domain antibodies have a molecular weight of only 12-15 kDa and are thus much smaller than antibodies composed of two heavy protein chains and two light chains (150-160 kDa), and domain antibodies are even smaller than Fab fragments (˜50 kDa, one light chain and half a heavy chain) and single-chain variable fragments (˜25 kDa, two variable domains, one from a light and one from a heavy chain). Single-domain antibodies have been engineered from heavy-chain antibodies found in camelids; these are termed V_{HH} fragments. Cartilaginous fish also have heavy-chain antibodies (IgNAR, ‘immunoglobulin new antigen receptor’), from which single-domain antibodies called VNAR fragments can be obtained. A domain (single-domain) antibody may be a V_{H} or V_{L}. A domain antibody may be a V_{H} or V_{L} of human or murine origin. Although most single-domain antibodies are heavy chain variable domains, light chain single-domain antibodies (V_{L}) have also been shown to bind specifically to target epitopes.

Protein scaffolds have relatively defined three-dimensional structures and typically contain one or more regions which are amenable to specific or random amino acid sequence variation, to produce antigen-binding regions within the scaffold that are capable of binding to an antigen.

A humanised or human antibody or antigen-binding fragment of the invention binds specifically to human SEMA4A. The term “specific” may refer to the situation in which the antibody molecule will not show any significant binding to molecules other than its specific binding partner(s), here an epitope of human SEMA4A. The term “specific” is also applicable where the antibody is specific for particular epitopes, such as an epitope of human SEMA4A that is carried by a number of antigens in which case the antibody molecule will be able to bind to the various antigens carrying the epitope. The epitope may be present in human SEMA4A expressed on the cell surface or soluble SEMA4A (sSEMA4A) shed from the cell surface or expressed recombinantly. In some embodiments a humanised or human antibody or antigen-binding fragment of the invention binds specifically to human SEMA4A and binds to cynomolgus and/or mouse SEMA4A, accordingly, a humanised or human antibody or antigen-binding fragment of the invention may bind specifically to human SEMA4A and be cross reactive with cynomolgus and/or mouse SEMA4A.

In some embodiments of the invention, the humanised antibodies and antigen-binding fragments thereof are humanised versions of mouse 5E3 mAb (Cat. #148402, BioLegend, Inc., USA), comprising the set of six CDRs of mouse 5E3 (SEQ ID NOS.: 3, 4, 5, 12, 13 and 14) and human framework sequences.

In some aspects of the invention humanised and human antibodies and antigen-binding fragments thereof of the invention bind to an epitope bound by mouse 5E3 mAb (Cat. #148402, BioLegend, Inc., USA) or compete with the mouse mAb 5E3 for binding to an epitope on SEMA4A, preferably human SEMA4A.

Amino acids may be referred to by their one letter or three letter codes, or by their full name. The one and three letter codes, as well as the full names, of each of the twenty standard amino acids are set out below in Table 1.

TABLE 1
Amino acids, one and three-letter codes.

| Amino acid | One letter code | Three letter code |
|---|---|---|
| Alanine | A | Ala |
| Arginine | R | Arg |
| Asparagine | N | Asn |
| Aspartic acid | D | Asp |
| Cysteine | C | Cys |
| Glutamic acid | E | Glu |
| Glutamine | Q | Gln |
| Glycine | G | Gly |
| Histidine | H | His |
| Isoleucine | I | Ile |
| Leucine | L | Leu |
| Lysine | K | Lys |
| Methionine | M | Met |
| Phenylalanine | F | Phe |
| Proline | P | Pro |
| Serine | S | Ser |
| Threonine | T | Thr |
| Tryptophan | W | Tryp |
| Tyrosine | Y | Tyr |
| Valine | V | Val |

In preferred embodiments, the invention provides a humanised or human antibody or an antigen-binding fragment thereof comprising the set of six CDRs HCDR1, HCDR2, HCDR3, LCDR1, LCDR2, and LCDR3 of a clone selected from:
a) 5E3 (SEQ ID NOS.: 3, 4, 5, 12, 13, 14);
b) 5Hg1-5Lg1 (SEQ ID NOS.: 21, 22, 23, 30, 31, 32);
c) 5Hg1-5Lg2 (SEQ ID NOS.: 39, 40, 41, 48, 49, 50);
d) 5Hg2-5Lg1 (SEQ ID NOS.: 57, 58, 59, 66, 67, 68);
e) 5Hg2-5Lg2 (SEQ ID NOS.: 75, 76, 77, 84, 85, 86);
f) 5Hg2-5Lg3 (SEQ ID NOS.: 93, 94, 95, 102, 103, 104);
g) 5Hg2-5Lg5 (SEQ ID NOS.: 111, 112, 113, 120, 121, 122);
h) 5Hg2-5Lg6 (SEQ ID NOS.: 129, 130, 131, 138, 139, 140);
i) 5Hg4-5Lg1 (SEQ ID NOS.: 147, 148, 149, 156, 157, 158);
j) 5Hg4-5Lg2 (SEQ ID NOS.: 165, 166, 167, 174, 175, 176);
k) 5Hg4-5Lg4 (SEQ ID NOS.: 183, 184, 185, 192, 193, 194);
I) 5Hg4-5Lg5 (SEQ ID NOS.: 201, 202, 203, 210, 211, 212);
m) 5Hg5-5Lg2 (SEQ ID NOS.: 219, 220, 221, 228, 229, 230);
n) C0120903 (SEQ ID NOS.: 237, 238, 239, 246, 247, 248);
o) C0120904 (SEQ ID NOS.: 255, 256, 257, 264, 265, 266);
p) C0120905 (SEQ ID NOS.: 273, 274, 275, 282, 283, 284);
q) C0120906 (SEQ ID NOS.: 291, 292, 293, 300, 301, 302);
r) C0120910 (SEQ ID NOS.: 309, 310, 311, 318, 319, 320);
s) C0120913 (SEQ ID NOS.: 327, 328, 329, 336, 337, 338);
t) C0120914 (SEQ ID NOS.: 345, 346, 347, 354, 355, 356);
u) C0120917 (SEQ ID NOS.: 363, 364, 365, 372, 373, 374);
v) C0120918 (SEQ ID NOS.: 381, 382, 383, 390, 391, 392);
w) C0120919 (SEQ ID NOS.: 399, 400, 401, 408, 409, 410);
x) C0120920 (SEQ ID NOS.: 417, 418, 419, 426, 427, 428);
y) C0120921 (SEQ ID NOS.: 435, 436, 437, 444, 445, 446);
z) C0120922 (SEQ ID NOS.: 453, 453, 455, 462, 463, 464);
aa) C0120923 (SEQ ID NOS.: 471, 472, 473, 480, 481, 482);
bb) C0120924 (SEQ ID NOS.: 489, 490, 491, 498, 499, 500);
cc) C0120925 (SEQ ID NOS.: 507, 508, 509, 516, 517, 518);
dd) C0120926 (SEQ ID NOS.: 525, 526, 527, 534, 535, 536);
ee) C0120927 (SEQ ID NOS.: 543, 544, 545, 552, 553, 554);
ff) C0120928 (SEQ ID NOS.: 561, 562, 563, 570, 571, 572);
gg) C0120929 (SEQ ID NOS.: 579, 580, 581, 588, 589, 590) and
hh) C0120930 (SEQ ID NOS.: 597, 598, 599, 606, 607, 608);

wherein the CDRs are defined according to Kabat.

For each clone, the CDRs are spaced by framework regions FW1, FW2, FW3 and FW4, to give a structure in the format FW1-CDR1-FW2-CDR2-FW3-CDR3-FW4.

The invention provides a human or humanised antibody or an antigen-binding fragment thereof comprising a V_{H} and/or V_{L} sequence of a clone selected from
a) 5Hg1-5Lg1 (SEQ ID NOS.: 20 and 29);
b) 5Hg1-5Lg2 (SEQ ID NOS.: 38 and 47);
c) 5Hg2-5Lg1 (SEQ ID NOS.: 56 and 65);
d) 5Hg2-5Lg2 (SEQ ID NOS.: 74 and 83);
e) 5Hg2-5Lg3 (SEQ ID NOS.: 92 and 101);
f) 5Hg2-5Lg5 (SEQ ID NOS.: 110 and 119);
g) 5Hg2-5Lg6 (SEQ ID NOS.: 128 and 137);
h) 5Hg4-5Lg1 (SEQ ID NOS.: 146 and 155);
i) 5Hg4-5Lg2 (SEQ ID NOS.: 164 and 173);
j) 5Hg4-5Lg4 (SEQ ID NOS.: 182 and 191);
k) 5Hg4-5Lg5 (SEQ ID NOS.: 200 and 209);
I) 5Hg5-5Lg2 (SEQ ID NOS.: 218 and 227);
m) C0120903 (SEQ ID NOS.: 236 and 245);
n) C0120904 (SEQ ID NOS.: 254 and 263);
o) C0120905 (SEQ ID NOS.: 272 and 281);
p) C0120906 (SEQ ID NOS.: 290 and 299);
q) C0120910 (SEQ ID NOS.: 308 and 317);
r) C0120913 (SEQ ID NOS.: 326 and 335);
s) C0120914 (SEQ ID NOS.: 344 and 353);
t) C0120917 (SEQ ID NOS.: 362 and 371);
u) C0120918 (SEQ ID NOS.: 380 and 389);
v) C0120919 (SEQ ID NOS.: 398 and 407);
w) C0120920 (SEQ ID NOS.: 416 and 425);
x) C0120921 (SEQ ID NOS.: 434 and 443);
y) C0120922 (SEQ ID NOS.: 452 and 461);
z) C0120923 (SEQ ID NOS.: 470 and 479);
aa) C0120924 (SEQ ID NOS.: 488 and 497);
bb) C0120925 (SEQ ID NOS.: 506 and 515);
cc) C0120926 (SEQ ID NOS.: 524 and 533);
dd) C0120927 (SEQ ID NOS.: 542 and 551);
ee) C0120928 (SEQ ID NOS.: 560 and 569);
ff) C0120929 (SEQ ID NOS.: 578 and 587); and
gg) C0120930 (SEQ ID NOS.: 596 and 605);

wherein the sequences are defined according to Kabat.

In some instances antibodies were selected for germlining. The amino acid sequences of the V_{H} and V_{L} domains of the antibodies were compared to human germline V, D and J regions accessible via IMGT (ImMunoGeneTics; www.imgt.org) or ImmuneDiscover databases and the closest germline was identified by sequence similarity. The germlining process consisted of reverting framework residues in the V_{H} and V_{L} domains to the closest germline sequence to identically match human antibodies.

An antibody or an antigen-binding fragment thereof of the invention may comprise one or more, e.g., 1, 2, 3, 4, 5, 6, 7, 8, 9 or 10 further amino acid modifications in the V_{H} and/or V_{L} sequences, provided that functional properties of the antibody are retained.

A modification may be an amino acid substitution, deletion or insertion; preferably, the modification is a substitution. In preferred embodiments in which one or more amino acids are substituted with another amino acid, the substitutions may be conservative substitutions, for example according to Table 2. In some embodiments, amino acids in the same category in the middle column are substituted for one another, i.e., a non-polar amino acid is substituted with another non-polar amino acid, for example. In some embodiments, amino acids in the same line in the rightmost column are substituted for one another.

TABLE 2
Amino acids

| | | |
|---|---|---|
| Aliphatic | Non-polar | G A P |
| | | I L V |
| | Polar - uncharged | C S T M |
| | | N Q |
| | Polar - charged | D E |
| | | K R |
| Aromatic | | H F W Y |

In some embodiments, substitution(s) may be functionally conservative. That is, in some embodiments the substitution may not affect (or may not substantially affect) one or more functional properties (e.g., binding affinity) of the antibody molecule comprising the substitution as compared to the equivalent unsubstituted antibody molecule.

In a preferred embodiment, an antibody or an antigen-binding fragment thereof of the invention may comprise a V_{H} and/or V_{L} domain sequence with one or more amino acid sequence alterations (addition, deletion, substitution and/or insertion of an amino acid residue), preferably 20 alterations or fewer, 15 alterations or fewer, 10 alterations or fewer, 5 alterations or fewer, 4 alterations or fewer, 3 alterations or fewer, 2 alterations or fewer, or 1 alteration compared with the V_{H} and/or V_{L} sequences of the invention set forth herein.

In preferred embodiments, the invention provides a humanised or human antibody or an antigen-binding fragment thereof comprising a V_{H} and/or V_{L} domain with an amino acid sequence which has at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the V_{H} and/or V_{L} amino acid sequence of a clone selected from:
a) 5E3 (SEQ ID NOS.: 2 and 11);
b) 5Hg1-5Lg1 (SEQ ID NOS.: 20 and 29);
c) 5Hg1-5Lg2 (SEQ ID NOS.: 38 and 47);
d) 5Hg2-5Lg1 (SEQ ID NOS.: 56 and 65);
e) 5Hg2-5Lg2 (SEQ ID NOS.: 74 and 83);
f) 5Hg2-5Lg3 (SEQ ID NOS.: 92 and 101);
g) 5Hg2-5Lg5 (SEQ ID NOS.: 110 and 119);
h) 5Hg2-5Lg6 (SEQ ID NOS.: 128 and 137);
i) 5Hg4-5Lg1 (SEQ ID NOS.: 146 and 155);
j) 5Hg4-5Lg2 (SEQ ID NOS.: 164 and 173);
k) 5Hg4-5Lg4 (SEQ ID NOS.: 182 and 191);
I) 5Hg4-5Lg5 (SEQ ID NOS.: 200 and 209);
m) 5Hg5-5Lg2 (SEQ ID NOS.: 218 and 227);
n) C0120903 (SEQ ID NOS.: 236 and 245);
o) C0120904 (SEQ ID NOS.: 254 and 263);
p) C0120905 (SEQ ID NOS.: 272 and 281);
q) C0120906 (SEQ ID NOS.: 290 and 299);
r) C0120910 (SEQ ID NOS.: 308 and 317);
s) C0120913 (SEQ ID NOS.: 326 and 335);
t) C0120914 (SEQ ID NOS.: 344 and 353);
u) C0120917 (SEQ ID NOS.: 362 and 371);
v) C0120918 (SEQ ID NOS.: 380 and 389);
w) C0120919 (SEQ ID NOS.: 398 and 407);
x) C0120920 (SEQ ID NOS.: 416 and 425);
y) C0120921 (SEQ ID NOS.: 434 and 443);
z) C0120922 (SEQ ID NOS.: 452 and 461);
aa) C0120923 (SEQ ID NOS.: 470 and 479);
bb) C0120924 (SEQ ID NOS.: 488 and 497);
cc) C0120925 (SEQ ID NOS.: 506 and 515);
dd) C0120926 (SEQ ID NOS.: 524 and 533);
ee) C0120927 (SEQ ID NOS.: 542 and 551);
ff) C0120928 (SEQ ID NOS.: 560 and 569);
gg) C0120929 (SEQ ID NOS.: 578 and 587); and
hh) C0120930 (SEQ ID NOS.: 596 and 605);

when defined by Kabat nomenclature.

In a preferred embodiment of the invention, a humanised or human antibody or an antigen-binding fragment thereof of the invention comprises a V_{H} domain amino acid sequence comprising the set of HCDRs: HCDR1, HCDR2, and HCDR3, and/or a V_{L} domain amino acid sequence comprising the set of LCDRs: LCDR1, LCDR2, and LCDR3 of a clone selected from:
(a) 5E3 (SEQ ID NOS.: 3, 4, 5, 12, 13, 14);
(b) 5Hg1-5Lg1 (SEQ ID NOS.: 21, 22, 23, 30, 31, 32);
(c) 5Hg1-5Lg2 (SEQ ID NOS.: 39, 40, 41, 48, 49, 50);
(d) 5Hg2-5Lg1 (SEQ ID NOS.: 57, 58, 59, 66, 67, 68);
(e) 5Hg2-5Lg2 (SEQ ID NOS.: 75, 76, 77, 84, 85, 86);
(f) 5Hg2-5Lg3 (SEQ ID NOS.: 93, 94, 95, 102, 103, 104);
(g) 5Hg2-5Lg5 (SEQ ID NOS.: 111, 112, 113, 120, 121, 122);
(h) 5Hg2-5Lg6 (SEQ ID NOS.: 129, 130, 131, 138, 139, 140);
(i) 5Hg4-5Lg1 (SEQ ID NOS.: 147, 148, 149, 156, 157, 158);
(j) 5Hg4-5Lg2 (SEQ ID NOS.: 165, 166, 167, 174, 175, 176);
(k) 5Hg4-5Lg4 (SEQ ID NOS.: 183, 184, 185, 192, 193, 194);
(l) 5Hg4-5Lg5 (SEQ ID NOS.: 201, 202, 203, 210, 211, 212);
(m) 5Hg5-5Lg2 (SEQ ID NOS.: 219, 220, 221, 228, 229, 230);
(n) C0120903 (SEQ ID NOS.: 237, 238, 239, 246, 247, 248);
(o) C0120904 (SEQ ID NOS.: 255, 256, 257, 264, 265, 266);
(p) C0120905 (SEQ ID NOS.: 273, 274, 275, 282, 283, 284);
(q) C0120906 (SEQ ID NOS.: 291, 292, 293, 300, 301, 302);
(r) C0120910 (SEQ ID NOS.: 309, 310, 311, 318, 319, 320);
(s) C0120913 (SEQ ID NOS.: 327, 328, 329, 336, 337, 338);
(t) C0120914 (SEQ ID NOS.: 345, 346, 347, 354, 355, 356);
(u) C0120917 (SEQ ID NOS.: 363, 364, 365, 372, 373, 374);
(v) C0120918 (SEQ ID NOS.: 381, 382, 383, 390, 391, 392);
(w) C0120919 (SEQ ID NOS.: 399, 400, 401, 408, 409, 410);
(x) C0120920 (SEQ ID NOS.: 417, 418, 419, 426, 427, 428);
(y) C0120921 (SEQ ID NOS.: 435, 436, 437, 444, 445, 446);
(z) C0120922 (SEQ ID NOS.: 453, 453, 455, 462, 463, 464);
(aa) C0120923 (SEQ ID NOS.: 471, 472, 473, 480, 481, 482);
(bb) C0120924 (SEQ ID NOS.: 489, 490, 491, 498, 499, 500);
(cc) C0120925 (SEQ ID NOS.: 507, 508, 509, 516, 517, 518);
(dd) C0120926 (SEQ ID NOS.: 525, 526, 527, 534, 535, 536);
(ee) C0120927 (SEQ ID NOS.: 543, 544, 545, 552, 553, 554);
(ff) C0120928 (SEQ ID NOS.: 561, 562, 563, 570, 571, 572);
(gg) C0120929 (SEQ ID NOS.: 579, 580, 581, 588, 589, 590) and
(hh) C0120930 (SEQ ID NOS.: 597, 598, 599, 606, 607, 608);

wherein the CDRs are defined according to Kabat, and the V_{H} and/or V_{L} domain has an amino acid sequence with at least 70%, at least 75%, at least 80%, at least 81%, at least 82%, at least 83%, at least 84%, at least 85%, at least 86%, at least 87%, at least 88%, at least 89%, at least 90%, at least 91%, at least 92%, at least 93%, at least 94%, at least 95%, at least 96%, at least 97%, at least 98%, or at least 99% sequence identity to the sequence of a clone selected from:
a. 5E3 (SEQ ID NOS.: 2 and 11);
b. 5Hg1-5Lg1 (SEQ ID NOS.: 20 and 29);
c. 5Hg1-5Lg2 (SEQ ID NOS.: 38 and 47);
d. 5Hg2-5Lg1 (SEQ ID NOS.: 56 and 65);
e. 5Hg2-5Lg2 (SEQ ID NOS.: 74 and 83);
f. 5Hg2-5Lg3 (SEQ ID NOS.: 92 and 101);
g. 5Hg2-5Lg5 (SEQ ID NOS.: 110 and 119);
h. 5Hg2-5Lg6 (SEQ ID NOS.: 128 and 137);
i. 5Hg4-5Lg1 (SEQ ID NOS.: 146 and 155);
j. 5Hg4-5Lg2 (SEQ ID NOS.: 164 and 173);
k. 5Hg4-5Lg4 (SEQ ID NOS.: 182 and 191);
l. 5Hg4-5Lg5 (SEQ ID NOS.: 200 and 209);
m. 5Hg5-5Lg2 (SEQ ID NOS.: 218 and 227);
n. C0120903 (SEQ ID NOS.: 236 and 245);
o. C0120904 (SEQ ID NOS.: 254 and 263);
p. C0120905 (SEQ ID NOS.: 272 and 281);
q. C0120906 (SEQ ID NOS.: 290 and 299);
r. C0120910 (SEQ ID NOS.: 308 and 317);
s. C0120913 (SEQ ID NOS.: 326 and 335);
t. C0120914 (SEQ ID NOS.: 344 and 353);
u. C0120917 (SEQ ID NOS.: 362 and 371);
v. C0120918 (SEQ ID NOS.: 380 and 389);
w. C0120919 (SEQ ID NOS.: 398 and 407);
x. C0120920 (SEQ ID NOS.: 416 and 425);
y. C0120921 (SEQ ID NOS.: 434 and 443);
z. C0120922 (SEQ ID NOS.: 452 and 461);
aa. C0120923 (SEQ ID NOS.: 470 and 479);
bb. C0120924 (SEQ ID NOS.: 488 and 497);
cc. C0120925 (SEQ ID NOS.: 506 and 515);
dd. C0120926 (SEQ ID NOS.: 524 and 533);
ee. C0120927 (SEQ ID NOS.: 542 and 551);
ff. C0120928 (SEQ ID NOS.: 560 and 569);
gg. C0120929 (SEQ ID NOS.: 578 and 587); and
hh. C0120930 (SEQ ID NOS.: 596 and 605);

when defined by Kabat nomenclature.

Sequence identity is commonly defined with reference to the algorithm GAP (Wisconsin GCG package, Accelerys Inc, San Diego USA). GAP uses the Needleman and Wunsch algorithm to align two complete sequences, maximising the number of matches and minimising the number of gaps. Generally, default parameters are used, with a gap creation penalty equaling 12 and a gap extension penalty equaling 4. Use of GAP may be preferred but other algorithms may be used, e.g., BLAST (which uses the method of Altschul et al., 1990), FASTA (which uses the method of Pearson and Lipman 1988), or the Smith-Waterman algorithm (Smith and Waterman 1981), or the TBLASTN program, of Altschul et al., (1990) supra, generally employing default parameters. In particular, the psi-Blast algorithm may be used (Altschul et al., 1997). Sequence alignments may also be performed using CLUSTAL (W) algorithm.

The antibody may comprise a CH2 domain. The CH2 domain is preferably located at the N-terminus of the CH3 domain, as in the case in a human IgG molecule. The CH2 domain of the antibody is preferably the CH2 domain of human IgG1, IgG2, IgG3, or IgG4, more preferably the CH2 domain of human IgG1. The sequences of human IgG domains are known in the art. The antibody may comprise an immunoglobulin hinge region, or part thereof, at the N-terminus of the CH2 domain. The immunoglobulin hinge region allows the two CH2-CH3 domain sequences to associate and form a dimer. Preferably, the hinge region, or part thereof, is a human IgG1, IgG2, IgG3 or IgG4 hinge region, or part thereof. More preferably, the hinge region, or part thereof, is an IgG1 hinge region, or part thereof.

The sequence of the CH3 domain is not particularly limited. Preferably, the CH3 domain is a human immunoglobulin G domain, such as a human IgG1, IgG2, IgG3, or IgG4 CH3 domain, most preferably a human IgG1 CH3 domain.

An antibody of the invention may comprise a human IgG1, IgG2, IgG3, or IgG4 constant region. The sequences of human IgG1, IgG2, IgG3, or IgG4 CH3 domains are known in the art. An antibody of the invention may comprise a human IgG constant region, e.g., a human IgG1 constant region. An antibody of the invention may comprise a human IgG Fc that has been modified to permit conjugation with a linker and cytotoxin.

An antibody of the invention may comprise a human IgG heavy chain, such as a human IgG1 heavy chain, engineered to contain one or more, e.g., 2, site-specific engineered cysteines that enable conjugation of cytotoxin to the antibody via a linker in a controlled manner as described in Dimasi et al., 2017; Li et al., 2016 and Gallagher et al., 2019 (e.g., a human IgG1 heavy chain with 239iCys and S442C (EU numbering) in the CH2 and CH3 Fc domain, respectively to generate a drug to antibody ratio of 4 (human IgG1 Alya). An antibody of the invention may comprise a human lambda light chain constant domains or human kappa light chain constant domains.

An antibody of the invention may comprise a human IgG Fc with effector function.

Fc receptors (FcRs) are key immune regulatory receptors connecting the antibody mediated (humoral) immune response to cellular effector functions. Receptors for all classes of immunoglobulins have been identified, including FcγR (IgG), FcεRI (IgE), FcαRI (IgA), FcμR (IgM) and FcδR (IgD). There are three classes of receptors for human IgG found on leukocytes: CD64 (FcγRI), CD32 (FcγRIIa, FcγRIIb and FcγRIIc) and CD16 (FcγRIIIa and FcγRIIIb). FcγRI is classed as a high affinity receptor (nanomolar range affinity) while FcγRII and FcγRIII are low to intermediate affinity (micromolar range affinity).

In antibody-dependent cell-mediated cytotoxicity (ADCC), FcγRs on the surface of effector cells (natural killer cells, macrophages, monocytes and eosinophils) bind to the Fc region of an IgG which itself is bound to a target cell. Upon binding a signalling pathway is triggered which results in the secretion of various substances, such as lytic enzymes, perforin, granzymes and tumour necrosis factor, which mediate in the destruction of the target cell. The level of ADCC effector function varies depending on the specific IgG subtype. Although the level of variation is dependent on the allotype and specific FcγR, in simple terms ADCC effector function is high for human IgG1 and IgG3, and low for IgG2 and IgG4. See Table 3 below for IgG subtype variation in effector functions, ranked in decreasing potency.

TABLE 3
IgG subtype variation in effector functions, ranked in decreasing potency.

| Effector Function | Species | IgG Subtype Potency |
|---|---|---|
| ADCC | Human | IgG1 ≥ IgG3 >> IgG4 > IgG2 |
| | Mouse | IgG2b > IgG2a > IgG1 >> IgG3 |
| C1q Binding | Human | IgG3 > IgG1 >> IgG2 > IgG4 |
| | Mouse | IgG2a ≥ IgG2b > IgG3 > IgG1 |

FcγRs bind to IgG asymmetrically across the hinge and upper CH2 region. Knowledge of the binding site has resulted in engineering efforts to modulate IgG effector functions.

Antibodies of the invention may have an Fc with effector function, enhanced effector function or with reduced effector function.

The potency of antibodies can be increased by enhancement of the ability to mediate cellular cytotoxicity functions, such as ADCC and antibody-dependent cell-mediated phagocytosis (ADCP). A number of mutations within the Fc domain have been identified that either directly or indirectly enhance binding of Fc receptors and significantly enhance cellular cytotoxicity: the mutations S239D/A330L/1332E (“3M”), F243L or G236A. Alternatively enhancement of effector function can be achieved by modifying the glycosylation of the Fc domain, FcγRs interact with the carbohydrates on the CH2 domain and the glycan composition has a substantial effect on effector function activity. Afucosylated (non-fucosylated) antibodies, exhibit greatly enhanced ADCC activity through increased binding to FcγRIIIa.

Activation of ADCC and CDC may be desirable for some therapeutic antibodies, however, in some embodiments, an antibody that does not activate effector functions is preferred.

Due to their lack of effector functions, IgG4 antibodies are the preferred IgG subclass for receptor blocking without cell depletion. However IgG4 molecules can exchange half-molecules in a dynamic process termed Fab-arm exchange. This phenomenon can occur between therapeutic antibodies and endogenous IgG4. The S228P mutation has been shown to prevent this recombination process allowing the design of IgG4 antibodies with a reduced propensity for Fab-arm exchange.

Fc engineering approaches have been used to determine the key interaction sites for the IgG1 Fc domain with Fcγ receptors and C1q and then mutate these positions to reduce or abolish binding. Through alanine scanning the binding site of C1q to a region covering the hinge and upper CH2 of the Fc domain was identified. The CH2 domain of an antibody or fragment of the invention may comprise one or more mutations to decrease or abrogate binding of the CH2 domain to one or more FcγRs, such as FcγRI, FcγRIIa, FcγRIIb, FcγRIII and/or to complement. CH2 domains of human IgG domains normally bind to FcγRs and complement, decreased binding to FcγRs is expected to decrease antibody-dependent cell-mediated cytotoxicity (ADCC) and decreased binding to complement is expected to decrease the complement-dependent cytotoxicity (CDC) activity of the antibody molecule. Mutations to decrease or abrogate binding of the CH2 domain to one or more FcγRs and/or complement are known in the art. An antibody molecule of the invention may comprise an Fc with modifications K322A/L234A/L235A or L234F/L235E/P331S (“TM”), which almost completely abolish FcγR and C1q binding. An antibody molecule of the invention may comprise a CH2 domain, wherein the CH2 domain comprises alanine residues at EU positions 234 and 235 (positions 1.3 and 1.2 by IMGT numbering) (“LALA mutation”). Furthermore, complement activation and ADCC can be decreased by mutation of P329 (position according to EU numbering), e.g., to either P329A or P329G. The antibody molecule of the invention may comprise a CH2 domain, wherein the CH2 domain comprises alanine residues at EU positions 234 and 235 (positions 1.3 and 1.2 by IMGT numbering) and an alanine (LALA-PA) or glycine (LALA-PG) at EU position 329 (position 114 by IMGT numbering). Additionally or alternatively an antibody molecule of the invention may comprise an alanine, glutamine or glycine at EU position 297 (position 84.4 by IMGT numbering).

Modification of glycosylation on asparagine 297 of the Fc domain, which is known to be required for optimal FcγR interaction may confer a loss of binding to FcγRs; a loss of binding to FcγRs has been observed in N297 point mutations. An antibody molecule of the invention may comprise an Fc with an N297A, N297G or N297Q mutation. An antibody molecule of the invention with an aglycosyl Fc domain may be obtained by enzymatic deglycosylation, by recombinant expression in the presence of a glycosylation inhibitor, or following the expression of Fc domains in bacteria.

IgG naturally persists for a prolonged period in the serum due to FcRn-mediated recycling, giving the IgG a typical half-life of approximately 21 days. Half-life can be extended by engineering the pH-dependent interaction of the Fc domain with FcRn to increase affinity at pH 6.0 while retaining minimal binding at pH 7.4. The T250Q/M428L variant, conferred an approximately 2-fold increase in IgG half-life (assessed in rhesus monkeys), while the M252Y/S254T/T256E variant (“YTE”), gave an approximately 4-fold increase in IgG half-life (assessed in cynomolgus monkeys). Extending half-life may allow the possibility of decreasing administration frequency, while maintaining or improving efficacy.

Immunoglobulins are known to have a modular architecture comprising discrete domains, which can be combined in a multitude of different ways to create multispecific, e.g., bispecific, trispecific, or tetraspecific antibody formats. Exemplary multispecific antibody formats are described in Spiess et al., 2015; Kontermann 2012, for example. The antibodies of the invention may be employed in such multispecific formats.

The invention provides a humanised or human antibody or antigen-binding fragment thereof, capable of competing with an antibody of the invention described herein (e.g., comprising a set of HCDR and LCDRs of 5E3 (SEQ ID NOS: 3, 4, 5, 12, 13, 14) when defined by Kabat nomenclature) and/or a humanised variant of the V_{H} and V_{L} amino acid sequences of Clone 5E3 (SEQ ID NOs: 2 and 11), for binding to an isolated recombinant human SEMA4A (SEQ ID NO: 613) peptide comprising an epitope, when assessed in a competition assay.

The invention provides a humanised or human antibody or antigen-binding fragment thereof, capable of competing for binding to an isolated recombinant human SEMA4A (SEQ ID NO: 613) peptide comprising an epitope, with a clone selected from:
a. 5E3 (SEQ ID NOS: 2 and 11)
b. 5Hg1-5Lg1 (SEQ ID NOS.: 20 and 29);
c. 5Hg1-5Lg2 (SEQ ID NOS.: 38 and 47);
d. 5Hg2-5Lg1 (SEQ ID NOS.: 56 and 65);
e. 5Hg2-5Lg2 (SEQ ID NOS.: 74 and 83);
f. 5Hg2-5Lg3 (SEQ ID NOS.: 92 and 101);
g. 5Hg2-5Lg5 (SEQ ID NOS.: 110 and 119);
h. 5Hg2-5Lg6 (SEQ ID NOS.: 128 and 137);
i. 5Hg4-5Lg1 (SEQ ID NOS.: 146 and 155);
j. 5Hg4-5Lg2 (SEQ ID NOS.: 164 and 173);
k. 5Hg4-5Lg4 (SEQ ID NOS.: 182 and 191);
l. 5Hg4-5Lg5 (SEQ ID NOS.: 200 and 209);
m. 5Hg5-5Lg2 (SEQ ID NOS.: 218 and 227);
n. C0120903 (SEQ ID NOS.: 236 and 245);
o. C0120904 (SEQ ID NOS.: 254 and 263);
p. C0120905 (SEQ ID NOS.: 272 and 281);
q. C0120906 (SEQ ID NOS.: 290 and 299);
r. C0120910 (SEQ ID NOS.: 308 and 317);
s. C0120913 (SEQ ID NOS.: 326 and 335);
t. C0120914 (SEQ ID NOS.: 344 and 353);
u. C0120917 (SEQ ID NOS.: 362 and 371);
v. C0120918 (SEQ ID NOS.: 380 and 389);
w. C0120919 (SEQ ID NOS.: 398 and 407);
x. C0120920 (SEQ ID NOS.: 416 and 425);
y. C0120921 (SEQ ID NOS.: 434 and 443);
z. C0120922 (SEQ ID NOS.: 452 and 461);
aa. C0120923 (SEQ ID NOS.: 470 and 479);
bb. C0120924 (SEQ ID NOS.: 488 and 497);
cc. C0120925 (SEQ ID NOS.: 506 and 515);
dd. C0120926 (SEQ ID NOS.: 524 and 533);
ee. C0120927 (SEQ ID NOS.: 542 and 551);
ff. C0120928 (SEQ ID NOS.: 560 and 569);
gg. C0120929 (SEQ ID NOS.: 578 and 587); and
hh. C0120930 (SEQ ID NOS.: 596 and 605);

wherein the sequences are defined according to Kabat nomenclature and competition for binding is assessed in a competition assay.

Competition assays may be selected from cell-based and cell-free binding assays including an immunoassay such as ELISA, homogeneous time resolved fluorescence (HTRF), flow cytometry, fluorescent microvolume assay technology (FMAT) assay, Mirrorball, high content imaging based fluorescent immunoassays, radioligand binding assays, bio-layer interferometry (BLI), surface plasmon resonance (SPR) and thermal shift assays.

An antibody that binds to the same epitope as, or an epitope overlapping with, a reference antibody refers to an antibody that blocks binding of the reference antibody to its binding partner (e.g., an antigen or “target”) in a competition assay by 50% or more, and/or conversely, the reference antibody blocks binding of the antibody to its binding partner in a competition assay by 50% or more. Such antibodies are said to compete for binding to an epitope of interest. An antibody may compete by binding the same epitope as, or an epitope overlapping with, the epitope of a reference antibody.

In embodiments of the invention anti-human-SEMA4A humanised or human antibodies described herein can be used to generate an antibody single-chain variable fragment which can then be used to prepare a chimeric antigen receptor (CAR). The antibody single-chain variable fragment is a chimeric protein made up of the light (V_{L}) and heavy (V_{H}) chains of immunoglobulins, connected by a short linker peptide. In some embodiments the linker between the V_{L} and V_{H} regions consists of hydrophilic residues comprising glycine and serine to confer flexibility and glutamate and lysine to confer solubility. In some aspects, the antibody single-chain variable fragment can be covalently linked to an intracellular immune cell signaling domain typically through a transmembrane domain to create a CAR. The immune cell signaling domain can be a T-cell, NK cell, macrophage, and/or a myeloid cell domain.

An anti-human-SEMA4A humanised or human antibody single-chain variable fragment may be covalently linked to an intracellular T-cell signaling or activation domain, for example via a transmembrane domain to create a CAR. When CARs are expressed in T-cells this provides T cells with the ability to target SEMA4A, in particular human SEMA4A. The invention thus provides CAR T-cells comprising a CAR comprising an anti-human-SEMA4A antibody single-chain variable fragment of an antibody described herein, that binds specifically to SEMA4A, in particular human SEMA4A, for use to treat haematological cancers, for example in MM, NHL, AML, DLBCL or FL patients; methods of treating a patient with a haematological cancer, e.g., MM, NHL, AML, DLBCL or FL, may comprise administering such CAR T-cells to a patient in need of therapy.

The transmembrane domain of a CAR may comprise a hydrophobic alpha helix that spans the cell membrane that anchors the CAR to the plasma membrane, bridging the extracellular antigen recognition domains (i.e., humanised or human antibody single-chain variable fragment) with the intracellular signaling region. The CAR may further comprise a hinge region between the antigen recognition domains and the transmembrane domain. The hinge may serve to enhance the flexibility of the scFv and reduce spatial constraints between the CAR and its target antigen, SEMA4A. The hinge sequence may be based on membrane-proximal regions from immune molecules such as IgG, CD8, and CD28. A CAR of the present disclosure may comprise a CD3-zeta cytoplasmic domain as a CAR endodomain component. T cells require co-stimulatory molecules in addition to CD3 signaling to persist after activation. The endodomain of a CAR may include one or more chimeric domains from co-stimulatory proteins. Signaling domains from a wide variety of co stimulatory molecules have been successfully tested, and may be selected from CD28, CD27, CD134 (OX40), and CD137. The endodomains of CAR receptors may comprise co-stimulatory domains to augment T cell activity, co-stimulatory domains may be selected from those of CD28 or 4-1BB, CD28-4-1BB or CD28-OX40, and cytokines, such as IL-2, IL-5, and IL-12.

The invention also provides a nucleic acid or set of nucleic acids encoding an antibody or antigen-binding fragment of the invention, as well as a vector comprising such a nucleic acid or set of nucleic acids.

Where the nucleic acid encodes the V_{H} and V_{L} domain, or heavy and light chain, of an antibody molecule of the invention, the two domains or chains may be encoded on the same or on separate nucleic acid molecules.

An isolated nucleic acid molecule may be used to express an antibody molecule of the invention. The nucleic acid will generally be provided in the form of a recombinant vector for expression. Another aspect of the invention thus provides a vector comprising a nucleic acid as described above. Suitable vectors can be chosen or constructed, containing appropriate regulatory sequences, including promoter sequences, terminator fragments, polyadenylation sequences, enhancer sequences, marker genes and other sequences as appropriate. Preferably, the vector contains appropriate regulatory sequences to drive the expression of the nucleic acid in a host cell. Vectors may be plasmids, viral e.g., phage, or phagemid, as appropriate.

A nucleic acid molecule or vector as described herein may be introduced into a host cell. Techniques for the introduction of nucleic acid or vectors into host cells are well established in the art and any suitable technique may be employed. A range of host cells suitable for the production of recombinant antibody molecules are known in the art, and include bacterial, yeast, insect or mammalian host cells. A preferred host cell is a mammalian cell, such as a CHO, NS0, or HEK cell, for example a HEK293 cell.

A recombinant host cell comprising a nucleic acid or the vector of the invention is also provided. Such a recombinant host cell may be used to produce an antigen-binding protein (e.g., antibody) of the invention. Thus, also provided is a method of producing an antigen-binding protein, e.g., antibody, of the invention, the method comprising culturing the recombinant host cell under conditions suitable for production of the antigen-binding protein, e.g., antibody. The method may further comprise a step of isolating and/or purifying the antigen-binding protein, e.g., antibody.

Thus the invention provides a method of producing an antigen-binding protein, e.g., antibody, of the invention comprising expressing a nucleic acid encoding the antigen-binding protein, e.g., antibody, in a host cell and optionally isolating and/or purifying the antigen-binding protein, e.g., antibody, thus produced. Methods for culturing host cells are well-known in the art. Techniques for the purification of recombinant antigen-binding proteins, e.g., antibodies, are well-known in the art and include, for example high-pressure liquid chromatograph (HPLC), fast protein liquid chromatograph (FPLC) or affinity chromatography, e.g., using Protein A or Protein L. In some embodiments, purification may be performed using an affinity tag on an antigen-binding protein, e.g., antibody. The method may also comprise formulating the antigen-binding protein, e.g., antibody, into a pharmaceutical composition, optionally with a pharmaceutically acceptable excipient or other substance as described below.

Antigen-binding proteins, e.g., antibodies, of the invention are expected to find application in therapeutic applications, in particular therapeutic applications in humans, for example in the treatment of a haematological cancers, such as MM, NHL, AML, DLBCL or FL.

Also provided is a composition, such as a pharmaceutical composition, comprising an ADC, antigen-binding protein, e.g., antibody, or CAR according to the invention and an excipient, such as a pharmaceutically acceptable diluent.

The invention further provides an ADC, antigen-binding protein, e.g., antibody, or CAR of the invention, for use in a method of treatment. Also provided is a method of treating a patient, wherein the method comprises administering to the patient a therapeutically-effective amount of an ADC, antigen-binding protein, e.g., antibody, or CAR according to the invention. Further provided is the use of an ADC, antigen-binding protein, e.g., antibody, or CAR according to the invention for use in the manufacture of a medicament. A patient, as referred to herein, is preferably a human patient.

The invention also provides an ADC, antigen-binding protein, e.g., antibody, or CAR of the invention, for use in a method of treating a haematological cancer, such as MM, NHL, AML, DLBCL or FL, wherein the method comprises administering to the patient a therapeutically-effective amount of an ADC, antigen-binding protein, e.g., antibody, or CAR according to the invention. Further provided is the use of an ADC, antigen-binding protein, e.g., antibody, or CAR according to the invention for use in the manufacture of a medicament for the treatment haematological cancer, such as multiple myeloma MM, NHL, AML, DLBCL or FL, in a patient. In another aspect, the invention relates to an ADC, antigen-binding protein, e.g., antibody, or CAR of the invention for use in: a) treating, b) delaying progression of, c) prolonging the survival of, and/or (d) providing relief of symptoms in a patient suffering from a haematological cancer, such as MM, NHL, AML, DLBCL or FL.

The ADC, antigen-binding protein, e.g., antibody, or CAR as described herein may thus be for use for therapeutic applications, in particular for the treatment of a haematological cancer, such as MM, NHL, AML, DLBCL or FL.

An ADC, antigen-binding protein, e.g., antibody, or CAR as described herein may be used in a method of treatment of the human or animal body. Related aspects of the invention provide;
(i) an ADC, antigen-binding protein, e.g., antibody, or CAR described herein for use as a medicament,
(ii) an ADC, antigen-binding protein, e.g., antibody, or CAR described herein for use in a method of treatment of a disease or disorder,
(iii) the use of an ADC, antigen-binding protein, e.g., antibody, or CAR described herein in the manufacture of a medicament for use in the treatment of a disease or disorder; and,
(iv) a method of treating a disease or disorder in an individual, wherein the method comprises administering to the individual a therapeutically effective amount of an ADC, antigen-binding protein, e.g., antibody, or CAR as described herein.

The individual may be a patient, preferably a human patient.

Treatment may be any treatment or therapy in which some desired therapeutic effect is achieved, for example, the inhibition or delay of the progress of the condition, and includes a reduction in the rate of progress, a halt in the rate of progress, amelioration of the condition, cure or remission (whether partial or total) of the condition, preventing, ameliorating, delaying, abating or arresting one or more symptoms and/or signs of the condition or prolonging survival of an individual or patient beyond that expected in the absence of treatment.

Treatment as a prophylactic measure (i.e., prophylaxis) is also included. For example, an individual susceptible to or at risk of the recurrence of a haematological cancer such as MM, NHL, AML, DLBCL or FL may be treated as described herein. Such treatment may prevent or delay the reoccurrence of the disease in the individual.

Whilst an ADC, antigen-binding protein, e.g., antibody, or CAR may be administered alone, ADC, antigen-binding proteins, e.g., antibodies, or CAR will usually be administered in the form of a pharmaceutical composition, which may comprise at least one component in addition to the ADC, antigen-binding protein, e.g., antibody, or CAR. Another aspect of the invention therefore provides a pharmaceutical composition comprising an ADC, antigen-binding protein, e.g., antibody, or CAR as described herein. A method comprising formulating an ADC, antigen-binding protein, e.g., antibody, or CAR into a pharmaceutical composition is also provided.

Pharmaceutical compositions may comprise, in addition to the ADC, antigen-binding protein, e.g., antibody, or CAR a pharmaceutically acceptable excipient, carrier, buffer, stabilizer or other materials well known to those skilled in the art. The term “pharmaceutically acceptable” as used herein pertains to compounds, materials, compositions, and/or dosage forms which are, within the scope of sound medical judgement, suitable for use in contact with the tissues of a subject (e.g., human) without excessive toxicity, irritation, allergic response, or other problem or complication, commensurate with a reasonable benefit/risk ratio. Each carrier, excipient, etc. must also be “acceptable” in the sense of being compatible with the other ingredients of the formulation. The precise nature of the carrier or other material will depend on the route of administration, which may be by infusion, injection or any other suitable route, as discussed below.

For parenteral, for example subcutaneous or intravenous administration, e.g., by injection, the pharmaceutical composition comprising the ADC, antigen-binding protein, e.g., antibody, or CAR may be in the form of a parenterally acceptable aqueous solution which is pyrogen-free and has suitable pH, isotonicity and stability. In some embodiments, an ADC, antigen-binding protein, e.g., antibody, or CAR of the invention may be provided in a lyophilised form for reconstitution prior to administration. For example, lyophilised antigen-binding proteins, e.g., antibodies may be reconstituted in sterile water or saline prior to administration to an individual.

Administration may be in a “therapeutically effective amount”, this being sufficient to show benefit to an individual. The actual amount administered, and rate and time-course of administration, will depend on the nature and severity of what is being treated, the particular individual being treated, the clinical condition of the individual, the cause of the disorder, the site of delivery of the composition, the type of ADC, antigen-binding protein, e.g., antibody, or CAR, the method of administration, the scheduling of administration and other factors. Prescription of treatment may depend on the severity of the symptoms and/or progression of a disease being treated. A therapeutically effective amount or suitable dose of an ADC, antigen-binding protein, e.g., antibody, or CAR can be determined by comparing in vitro activity and in vivo activity in an animal model. Methods for extrapolation of effective dosages in mice and other test animals to humans are known. The precise dose will depend upon a number of factors, including whether the size and location of the area to be treated, and the precise nature of the ADC, antigen-binding protein, e.g., antibody, or CAR.

Haematological malignancies are a diverse group of haematological cancers that affect the blood, bone marrow and lymphatic systems. The main categories are lymphoma, leukaemia, myeloma, myelodysplastic syndromes and myeloproliferative neoplasms. In a preferred embodiment, an ADC, antigen-binding protein, e.g., antibody, or CAR as described herein may be for use in a method of treating a haematological cancer, such as MM, NHL, AML, DLBCL or FL.

### LIST OF FIGURES

FIG. 1. Primary screening of 70 un-purified humanised IgGs in a Fab-ZAP cell kill assay in NCI-H929 cells identified 18 clones that showed a cell kill of ≥30%. All 70 un-purified clones were screened at a final concentration of 1.56%. 12 out of 18 hit clones (•) were re-expressed and purified for further characterisation. 10 out of the 12 clones selected for progression also showed inhibition in the 5E3 epitope competition assay (see FIG. 2).

FIG. 2. Primary screening of 320 un-purified humanised IgGs in an epitope competition assay in MM.1S cells identified 17 clones that showed an inhibition of ≥ 30%. All 320 un-purified clones were screened at a final concentration of 25%, 10 out of 17 hit clones (•) were re-expressed and purified for further characterisation. Two clones that showed <30% inhibition were also re-expressed and purified for further characterisation: 5Hg2/5Lg6 and 5Hg4/5Lg4. All 12 clones selected for further characterisation were also identified as hits in the Fab-ZAP cell kill assay.

FIG. 3. Primary screening of un-purified fully human phage display IgGs in a Fab-ZAP cell kill assay in NCI-H929 cells identified a panel of clones that showed a cell kill of ≥ 15%. All un-purified clones were screened at a final assay concentration of 3.13%. 10 clones (•) were re-expressed and purified for further characterisation.

FIG. 4. Primary screening of un-purified fully human phage display IgGs in a binding assay in MM.1S cells identified clones that bound with a fluorescence intensity >1 million FLU. All un-purified clones were tested at a final assay concentration of 1.56%. Binding assay data was used in conjunction with Fab-ZAP assay data (FIG. 3) to select a panel of 10 clones (•) that were hits in the Fab-ZAP assay and showed good binding (>1 million FLU) to MM.1S cells. C0120910 was progressed as it did show binding when tested at a final assay concentration of 25% (data not shown).

FIG. 5. Measuring binding specificity of humanised leads (A) and fully human phage display leads (B) expressed in the human IgG1 Alya vector. The ability of leads to bind to expi293 cells transiently transfected with human SEMA4A (i), mouse SEMA4A (ii), cyno SEMA4A (iii), human SEMA4B (iv) and mock transfected expi293 cells (v) was measured using the Mirrorball. Binding of leads was compared to mouse 5E3 expressed as human IgG1 Alya. C0021144 IgG was included as a positive control for binding to mock transfected expi293 cells. A commercially available anti SEMA4B polyclonal antibody (R&D Systems, AF5485) was included as a positive control for binding to human SEMA4B transfected expi293 cells. For a description of human IgG1 Alya vector see example 4.1.

FIG. 6. Measuring the ability of humanised leads (A) and fully human leads (B) expressed in the human IgG1 Alya vector to kill NCI-H929 cells in a Fab-ZAP cell kill assay. Cell kill ability of all clones was compared to mouse 5E3 in the human IgG1 Alya vector. A number of humanised clones showed IC50 values comparable to the mouse 5E3 antibody (V_{H} and V_{L} SEQ ID NOS: 2 and 11). Several humanised clones did not demonstrate complete cell kill. For a description of human Alya IgG1 vector see example 4.1.

FIG. 7. Testing humanised leads expressed in the human IgG1 Alya vector in an epitope competition assay. The ability of increasing concentrations of humanised and fully human phage display leads expressed in the human IgG1 Alya vector to inhibit the binding of a fixed concentration of mouse 5E3 to SEMA4A endogenously expressed on MM.1S cells was measured. The fixed concentration of 5E3 was a mouse IgG1 isotype and binding of this was detected using an anti-mouse antibody labelled with Alexa Fluor 647. Mouse 5E3 expressed as a human isotype (human IgG1 Alya) was competed into the assay and test clones were compared against this. All humanised clones showed some level of competition with 5E3.

FIG. 8. Epitope binning. The OctetRED (Pall ForteBio) instrument was used as described in section 4.6 to perform epitope binning, grouping lead anti-human SEMA4A clones into bins based upon binding to recombinant human SEMA4A. In the in-tandem format, the antigen (biotinylated recombinant human SEMA4A) immobilized onto a streptavidin biosensor was presented to two competing analytes, in consecutive steps. Binding to distinct, non-overlapping epitopes was indicated if saturation with the first Fab fragment (mouse 5E3) did not block binding of the second fully human phage display derived IgG. All humanised leads had been shown to compete with 5E3 epitope in section 3.2. FIG. 8A is representative of data with fully human clones that have overlapping or competing epitopes with 5E3. FIGS. 8B-D show data corresponding to fully human clones with non-overlapping or non-competing epitopes with 5E3 (B) and (C) and with each other (D).

FIG. 9. Measuring binding specificity of humanised leads (A) and fully human leads (B) expressed in the human IgG1 Alya vector and conjugated to McMMAF. The ability of leads to bind to expi293 cells transiently transfected with human SEMA4A (i), human SEMA4B (ii) and mock transfected expi293 cells (iii) was measured using the Mirrorball. Binding was compared to mouse 5E3 as human IgG1 expressed in Alya vector (Alya IgG1) and McMMAF coupled. C0021144 IgG was included as a positive control for binding to mock transfected expi293 cells; C0021144 was not conjugated to McMMAF. A commercially available anti SEMA4B polyclonal antibody (R&D Systems, AF5485) was included as a positive control for binding to human SEMA4B transfected expi293 cells; AF5485 was not conjugated to McMMAF. For a description of human IgG1 Alya vector see example 4.1.

FIG. 10. Measuring the ability of humanised leads conjugated to McMMAF expressed in the human IgG1Alya vector to kill NCI-H929 (A), MM1.S (B), KARPAS 25 (C) and K562 (D) cells in a cell kill assay. Cell kill ability of all clones was compared to mouse 5E3 as Alya IgG1 conjugated to McMMAF. Humanised clones showed a range of IC50 values across cell lines, with no cell kill observed in K562 cells, a negative control cell line for SEMA4A expression. For a description of human Alya IgG1 vector see example 4.1. J6MO anti-BCMA monoclonal antibody control (Tai et al., 2014).

FIG. 11. Measuring the ability of fully human leads conjugated to McMMAF expressed in the human Alya IgG1 vector to kill NCI-H929 (A), MM1.S (B) and K562 (C) cells in a cell kill assay. Cell kill ability of all clones was compared to mouse 5E3 as Alya IgG1 conjugated to McMMAF. Fully human clones showed a range of IC50 values, with some being slightly more potent than mouse 5E3 as Alya IgG1 conjugated to McMMAF. As expected, clones did not elicit any cell kill, in K562 cells, a negative control cell line for SEMA4A expression. For a description of human Alya IgG1 vector see example 4.1. J6MO anti-BCMA monoclonal antibody control (Tai et al., 2014).

FIG. 12. Cell internalisation of McMMAF-conjugated humanised and fully human leads expressed in the human Alya IgG1 vector. The internalisation of each lead and control antibody was evaluated at a range of concentrations over a 24 hour period. (A) The percentage of cells scored as positive for Fab-pHast was plotted over time for every concentration of each antibody, in the same manner as is shown here for mouse 5E3 human Alya IgG1. Every time-course was subjected to an Area Under Curve (AUC) analysis, as demonstrated for the 2.5 nM 5E3 (mouse 5E3 human Alya IgG1 McMMAF coupled) data in panel (B). The Internalisation Titration Curves plotted from the AUC analyses for a panel of humanised leads and fully human clones (all in the human Alya IgG1 McMMAF-conjugate format) monoclonal are presented in panel (C). The Mean Stain Integrated Intensity was also extracted for each concentration and time point and plotted as a fold change in signal over baseline, as is shown in panel (D). For a description of human Alya IgG1 vector see example 4.1.

FIG. 13. Measuring binding affinities (KD) of SEMA4A specific human antibodies (Alya IgG1 conjugated to McMMMAF) by flow cytometry. KD values for all clones were compared to mouse 5E3 as human Alya IgG1 and McMMAF coupled. Humanised leads, 5Hg1/5Lg1 and 5Hg2/5Lg2 showed similar K_{D} values to mouse 5E3. The fully human, phage display derived leads had lower affinities than mouse 5E3. For a description of human Alya IgG1 vector see example 4.1.

FIG. 14. Measuring binding specificity of fully human, germlined leads expressed in the human Alya IgG1 vector. The ability of germlined leads to bind to expi293 cells transiently transfected with human SEMA4A (i), mouse SEMA4A (ii), cynomolgus SEMA4A (iii), human SEMA4B (iv) and mock transfected expi293 cells (v) was measured using the Mirrorball. Binding was compared to mouse 5E3 (in human Alya IgG1 not coupled to McMMAF). C0021144 IgG was included as a positive control for binding to mock transfected expi293 cells. A commercially available anti SEMA4B polyclonal antibody (R&D Systems, AF5485) was included as a positive control for binding to human SEMA4B transfected expi293 cells. All 6 fully human germlined clones tested showed strong binding to human, mouse and cynomolgus SEMA4A and no binding to human SEMA4B and mock transfected expi293 cells. For a description of human Alya IgG1 vector see example 4.1.

FIG. 15. Low expression of soluble SEMA4A in the serum in both healthy and myeloma patients. (A) Standard curve for the ELISA using recombinant human SEMA4A (rSEMA4A). The lower limit of detection (LLOD) was determined as the average of the blank sample plus three standard deviations of the blank. The limit of detection was calculated as the lowest concentration tested (1.56 ng/ml) above the LLOD. (B) sSEMA4A in healthy and myeloma patient serum was detected by ELISA and found to be 3.3 ng/ml and 8.1 ng/ml respectively.

FIG. 16. Assessing the impact of soluble SEMA4A on the potency of mouse 5E3 and fully human leads in a cell kill assay using NCI-H929 cells. A concentration of up to 100 ng/ml sSEMA4A had no significant impact on the ability of either mouse 5E3 or the fully human clones, each as Alya IgG1 conjugated with McMMAF, to kill NCI-H929 cells in terms of IC50 values. For a description of human Alya IgG1 vector see example 4.1.

FIG. 17. Comparative sequence alignment for SEMA4A lead panel VH domains, alongside the mouse 5E3 VH sequence. Individual VH CDR sequences are highlighted in bold type, grey background and by black bars. Sequence number as defined by Kabat and Wu (1991).

FIG. 18. Comparative sequence alignment for SEMA4A lead panel V_{L} domains, alongside the mouse 5E3 V_{L} sequence. Individual V_{L} CDR sequences are highlighted in bold type, grey background and by black bars. Sequence number as defined by Kabat and Wu (1991).

### EXAMPLES

### Example 1: Humanisation of 5E3 mAb

### 1.1 Rationale Design of 5E3 Humanised Germline Variants

The anti-human SEMA4A monoclonal mouse IgG1 (A) 5E3 was purchased from BioLegend® (Catalogue number: 148402) and its primary amino acid sequence reverse engineered. Specifically, 5E3 was digested with either trypsin, chymotrypsin, or a combination of trypsin and AspN. Each of the three digests were analysed by LC/MS/MS using an Orbitrap Fusion mass spectrometer (Thermofisher Scientific). A search of the resulting data was run using Peaks software and a database of mouse germline sequences, followed by manual interpretation.

A panel of 10 V_{H} and 7 V_{L} human germline sequences were chosen as frameworks for mouse 5E3 humanisation. Human germline choice was guided by aligning the original non-humanised mouse 5E3 sequence to all human germline sequences listed within the IMGT® database, (www.IMGT.org). Human germlines having the highest amino acid sequence homology to the mouse 5E3 sequence were prioritised for subsequent 5E3 CDR grafting. Specifically, human germlines were engineered to be either ‘3× V_{H} CDR grafts’, ‘3× V_{L} CDR grafts’ or ‘1× V_{H} CDR graft’ (Table 4). Each V_{H} and V_{L} gene sequence was synthesised de novo and cloned into the appropriate IgG expression vector (Persic, et al., 1997) using standard molecular biology techniques.

TABLE 4
Human V_{H} and V_{L} germline sequences chosen as candidate frameworks
for 5E3 humanisation. 10× V_{H} and 7× V_{L} human germlines were chosen
based on their overall similarity to the original mouse 5E3 sequence. All
genes were synthesised de novo (Genewiz).

| | | |
|---|---|---|
| ID | Closest Human Germline | Notes |

5E3 Grafts (Heavy Chain)

| | | |
|---|---|---|
| 5Hg1 | IGHV1-46*01/IGHJ4*01 | 3× CDR graft |
| 5Hg2 | IGHV1-3*01/IGHJ4*01 | 3× CDR graft |
| 5Hg3 | IGHV1-69*11/IGHJ4*01 | 3× CDR graft |
| 5Hg4 | IGHV7-4-1*01/IGHJ4*01 | 3× CDR graft |
| 5Hg5 | IGHV5-10-1*04/IGHJ4*01 | 3× CDR graft |
| 5Hg6 | IGHV4-34*04/IGHJ4*01 | 3× CDR graft |
| 5Hg7 | IGHV3-23*04/IGHJ4*01 | 3× CDR graft |
| 5Hg8 | IGHV1-46*01/IGHJ4*01 | V_{H}CDR3 only graft |
| 5Hg9 | IGHV7-4-1*01/IGHJ4*01 | V_{H}CDR3 only graft |
| 5Hg10 | IGHV5-10-1*04/IGHJ4*01 | V_{H}CDR3 only graft |

5E3 Grafts (Lambda Light Chain)

| | | |
|---|---|---|
| 5Lg1 | IGLV7-46*01/IGLJ3*02 | 3× CDR graft |
| 5Lg2 | IGLV7-46*02/IGLJ3*02 | 3× CDR graft |
| 5Lg3 | IGLV8-61*01/IGLJ3*02 | 3× CDR graft |
| 5Lg4 | IGLV2-18*01/IGLJ3*02 | 3× CDR graft |
| 5Lg5 | IGLV1-40*03/IGLJ3*02 | 3× CDR graft |
| 5Lg6 | IGLV3-27*01/IGLJ3*02 | 3× CDR graft |
| 5Lg7 | IGLV4-60*03/ IGLJ3*02 | 3× CDR graft |

### 1.2 High Throughput Expression of Un-Purified IgG

The panel of humanised 5E3 clones were expressed as huIgG1 in a high throughput manner (Screening in Product Format or SiPF) using Expi293TMF cells following Life Technologies Expi293 Expression System protocol for 96-well microtiter plates (Protocol #CO25793 0912). Purified DNA of each of the 10 humanised 5E3 V_{H} cloned into pEU1.3 and 7 humanised 5E3 V_{L} constructs cloned into either pEU3.4 (kappa) or pEU4.4 (lambda) vectors were combined as a matrix in a 96 well plate generating 70 possible V_{H}×V_{L} combinations. The combined DNA was diluted in OptiMEM and used to transfect Expi293TMF cells in a 96 deepwell block (Merck, cat: AXYPDW20CS) using ExpiFectamine as transfection reagent. Cells were grown 37° C./8% CO₂/80% humidity shaking at 1000 rpm and fed (using enhance 1 and enhance 2) 18 hours post transfection. IgGs were harvested 4 days post transfection and used as crude, un-purified IgGs in subsequent assays.

### Example 2: Isolation of Fully Human Anti-SEMA4A Antibodies from Naïve Libraries by Phage Display

### 2.1 Production of Recombinant Human, Cynomolgus and Mouse SEMA4A

Recombinant human SEMA4A (UniProt ID Q9H3S1, amino acids 33-683), recombinant cynomolgus SEMA4A (UniProt ID G7NV79, amino acids 32-679) and recombinant mouse SEMA4A (UniProt ID Q62178, amino acids 33-682) with C-terminal Avi tag and His10 tag (SEQ ID NOS: 616, 618, 617, respectively) were cloned into pcDNA3.1 vector and transiently expressed in the Expi293 expression system. The proteins were subsequently purified using His Trap HP His tag protein purification columns (40 mM Imidazole wash, 400 mM Imidazole elution). The proteins (human SEMA4A at 75 kDa, cynomolgus SEMA4A at 79 kDa and mouse SEMA4A at 79 kDa) were buffer exchanged into 50 mM Bicine pH 8.3 and biotinylated using BirA biotin ligase (3 mg of biotin ligase per 10 nmol of substrate protein). The proteins were finally buffer exchanged into Dulbecco's phosphate buffered saline (DPBS) with 5% (w/v) Trehalose (pH 7).

TABLE 5
Sequence of SEMA4A AviHis10-tagged proteins produced.

| | |
|---|---|
| Human | MALPALGLDPWSLLGLFLFQLLQLLLPTTTAGGGGQGPMPRVRYYAGDERR |
| SEMA4A | ALSFFHQKGLQDFDTLLLSGDGNTLYVGAREAILALDIQDPGVPRLKNMIPWP |
| AviHis10 | ASDRKKSECAFKKKSNETQCFNFIRVLVSYNVTHLYTCGTFAFSPACTFIELQ |
| SEQ ID | DSYLLPISEDKVMEGKGQSPFDPAHKHTAVLVDGMLYSGTMNNFLGSEPILM |
| NO: 616 | RTLGSQPVLKTDNFLRWLHHDASFVAAIPSTQVVYFFFEETASEFDFFERLHT |
| | SRVARVCKNDVGGEKLLQKKWTTFLKAQLLCTQPGQLPFNVIRHAVLLPADS |
| | PTAPHIYAVFTSQWQVGGTRSSAVCAFSLLDIERVFKGKYKELNKETSRWTT |
| | YRGPETNPRPGSCSVGPSSDKALTFMKDHFLMDEQVVGTPLLVKSGVEYTR |
| | LAVETAQGLDGHSHLVMYLGTTTGSLHKAVVSGDSSAHLVEEIQLFPDPEPV |
| | RNLQLAPTQGAVFVGFSGGVWRVPRANCSVYESCVDCVLARDPHCAWDPE |
| | SRTCCLLSAPNLNSWKQDMERGNPEWACASGPMSRSLRPQSRPQIIKEVLA |
| | VPNSILELPCPHLSALASYYWSHGPAAVPEASSTVYNGSLLLIVQDGVGGLY |
| | QCWATENGFSYPVISYWVDSQDQTLALDPELAGIPREHVKVPLTRVSGGAA |
| | LAAQQSYWPHGTGGGSGGGSGLNDIFEAQKIEWHEHHHHHHHHHH |
| Mouse | MTGGQGPMPRVKYHAGDGHRALSFFQQKGLRDFDTLLLSDDGNTLYVGAR |
| SEMA4A | EAVLALNIQNPGIPRLKNMIPWPASERKKTECAFKKKSNETQCFNFIRVLVSY |
| AviHis10 | NATHLYACGTFAFSPACTFIELQDSLLLPILIDKVMDGKGQSPFDPVHKHTAV |
| SEQ ID | LVDGMLYSGTMNNFLGSEPILMRTLGSQPVLKTDIFLRWLHADASFVAAIPST |
| NO: 617 | QVVYFFFEETASEFDFFEELYISRVAQVCKNDVGGEKLLQKKWTTFLKAQLL |
| | CAQPGQLPFNIIRHAVLLPADSPSVSRIYAVFTSQWQVGGTRSSAVCAFSLT |
| | DIERVFKGKYKELNKETSRWTTYRGSEVSPRPGSCSMGPSSDKALTFMKDH |
| | FLMDEHVVGTPLLVKSGVEYTRLAVESARGLDGSSHVVMYLGTSTGSLHKA |
| | VVPQDSSAYLVEEIQLSPDSEPVRNLQLAPAQGAVFAGFSGGIWRVPRANC |
| | SVYESCVDCVLARDPHCAWDPESRLCSLLSGSTKPWKQDMERGNPEWVC |
| | TRGPMARSPRRQSPPQLIKEVLTVPNSILELPCPHLSALASYHWSHGRAKISE |
| | ASATVYNGSLLLLPQDGVGGLYQCVATENGYSYPVVSYWVDSQDQPLALDP |
| | ELAGVPRERVQVPLTRVGGGASMAAQRSYWPHGTGGGSGGGSGLNDIFEA |
| | QKIEWHEHHHHHHHHHH |
| Cyno | MGGGGQGPMPRVRYYAGDERRALSFFHQKGLQDFDTLLLSGNGNTLYVGA |
| SEMA4A | REAILALDIQDPGVPRLKNMIPWPASDRKKSECAFKKKSNETQCFNFIRVLVS |
| AviHis10 | YNVTHLYTCGTFAFSPACTFIELQDSHLLPILEDKVMEGKGQSPFDPAHKHTA |
| SEQ ID | VLVDGMLYSGTMNNFLGSEPILMRTLGSQPVLKTDNFLRWLHPDASFVAAIP |
| NO: 618 | STQVVYFFFEETASEFDFFERLHTSRVARVCKNDVGGEKLLQRKWTTFLKA |
| | QLLCTKPGQLPFNVIRHAVLLPADSPTAPHIYAVFTSQWQIGGTRSSAVCAFS |
| | LLDIERVFKGKYKELNKETSRWTTYRGPETNPRPGSCSVGPSSDKALTFMK |
| | DHFLMDEQVVGTPLLVKSGVEYTRLAVETAQGLDGRSHLVMYLGTTTGSLH |
| | KAVVSGDSGAHLVEEIQLFPDPEPVRNLQLAPTQGAVFVGFSGGVWRVPRA |
| | NCSVYESCVDCVLAQDPHCAWDPESRTCCFLSAPTLNSWKQDMERGNPE |
| | WACASGPMSRSLRPQSRPQIIKEVLAVPNSFLELPCPHLSALASYYWSHGPA |
| | AVPEASSTVYNGSLLLIVQDGVGGLYQCWATENGFSYPVVSYWVDSQDQSL |
| | ALDPELAGIPREHVEVPLTRVSGGTALAAQRSGTGGGSGGGSGLNDIFEAQ |
| | KIEWHEHHHHHHHHHH |

2.2 Phage Display Selections to Isolate SEMA4A Specific, Fully Human scFv

Soluble phage display selections were performed using five naïve libraries (nFL, DP47, CS, BMV and EG3) cloned into a phagemid vector based on the filamentous phage M13 (Vaughan et al., 1996; Lloyd et al., 2008). Anti-SEMA4A scFv antibodies were isolated from the phage display libraries using a series of selection cycles on recombinant, biotinylated human and mouse SEMA4A protein (avi-SEMA4A-His10, made in house) essentially as previously described (Hawkins et al., 1992; Vaughan et al., 1996). In brief, for the first round of solution-phase selections, biotinylated human SEMA4A in DPBS pH 7 was added (final concentration of 100 nM biotinylated human SEMA4A) to purified phage particles that had been pre-incubated for 1 hour in Marvel-PBS (3% w/v) containing Streptavidin-coupled paramagnetic beads (Dynabeads® M280, Invitrogen Life Sciences, UK). Streptavidin beads were removed prior to addition of antigen. Phage particles that bound to the biotinylated human SEMA4A were captured using new Streptavidin-coupled paramagnetic beads, and weakly-bound phage were removed by a series of wash cycles using PBS-Tween (0.1% v/v). Bound phage particles were eluted from the beads using Trypsin (10 μg/ml final concentration diluted in 0.1 M sodium phosphate buffer; pH 7), infected into E. coli TG1 bacteria and rescued for the next round of selection (Vaughan et al., 1996). Two subsequent rounds of selection were carried out as previously described but with a reduced concentration of biotinylated SEMA4A antigen, specifically 50 nM of biotinylated human or mouse SEMA4A at round 2 and 25 nM of biotinylated human SEMA4A at round 3. Mouse antigen was introduced at round 2 to drive for mouse cross-reactivity.

2.3 Identification of SEMA4A Specific Fully Human scFv (Un-Purified) Using a Direct Binding Assay on Cells (Mirrorball)

In order to determine whether un-purified scFv from periplasmic preparations could bind to cell surface expressed human SEMA4A they were incubated with expi293 cells that were transiently transfected with human SEMA4A (Origene, US; cat: SC323738). Binding of un-purified scFv was detected using two antibodies—a mouse anti c-myc antibody (Bio-Rad, US; cat: MCA2200GA) binding to a myc tag expressed on the scFv followed by an anti-mouse antibody conjugated to Alexa Fluor 647 (Invitrogen, US; cat: A21235).

Selection outputs were screened at a single concentration as un-purified bacterial periplasmic extracts containing scFv, prepared in 200 mM tris buffer pH 7.4, 0.5 mM EDTA and 0.5 M sucrose. 10 μl of un-purified scFv samples were added to a Greiner® 384 well assay plate (Greiner Bio-one, UK; cat: 781906). This was followed by the addition of 10 μl of antibody detection mix containing 8 nM mouse anti c-myc antibody and 8 nM anti-mouse antibody conjugated to Alexa Fluor 647 and 20 μl cell suspension at 75,000 cells/ml.

Non-specific binding wells (negative controls) were defined for each plate by using a negative control un-purified scFv in place of the test scFv sample. Clones binding non-specifically were identified using a concurrent assay with expi293 cells that had been mock transfected (i.e., human SEMA4A DNA was omitted during transfections).

All dilutions were performed in Hanks' balanced salt solution (Sigma, UK; cat: H8264) containing 0.1% bovine serum albumin (BSA) (Sigma, UK; cat: A9576) (assay buffer). Assay plates were incubated at room temperature in the dark for 4 hours prior to reading on a Mirrorball fluorescence cytometer (SPT Labtech, UK) using a 640 nm laser for excitation and measuring emission in the FL-4 channel (650 and 690 nm). Data was analysed as Count x median mean intensity (x FLU).

226 sequence unique hits were identified in the direct binding assay that were reformatted from scFv to human IgG1 as described in section 2.4.

2.4 Reformatting of SEMA4A Binding Antibodies from Fully Human scFv to IgG1 (Un-Purified IgG)

226 un-purified scFv periplasm extracts that showed specific binding to human SEMA4A by Mirrorball assay were subjected to DNA sequencing (Osbourn et al., 1996; Vaughan et al., 1996). The 226 unique scFvs were pooled into a lambda and a kappa pool based on their sequences. In addition, the entire outputs were subjected to the following cloning strategy. Using a 2-step strategy, the scFvs were batch converted into IgGs, firstly replacing the (G₄S)₃ scFv linker with a DNA segment coding for the hinge and constant domains for Heavy Chain (HC) and the promoter and signal sequence for Light Chain (LC) and secondly replacing the scFv backbone with the LC constant domains and the HC signal sequence in the IgG vector (as described in Xiao et al., 2017). The final expression vector is bicistronic with both HC and LC under the control of a CMV promoter. The converted IgGs were subsequently expressed as crude IgGs as described in section 3.2.

### Example 3: Functional Screening of Un-Purified 5E3 Humanised and Fully Human, Phage Display Derived, IgG1s—In Primary Screens

Example 3 describes the primary screening of un-purified IgG1s (section 1.2) from the 5E3 humanisation approach (Example 1) and from the phage display approach (Example 2). Both sets of IgGs were screened for function in a Fab-ZAP cell kill assay. The 5E3 humanisation IgGs were also screened for their ability to compete for binding to the 5E3 epitope in a mouse 5E3 epitope competition assay. The fully human, phage display derived, clones were also screened for their ability to bind to MM.1S multiple myeloma cells that endogenously express human SEMA4A.

3.1 Discovery of mAbs that can Internalise into and Kill NCI-H929 Cells Using the Fab-ZAP Assay

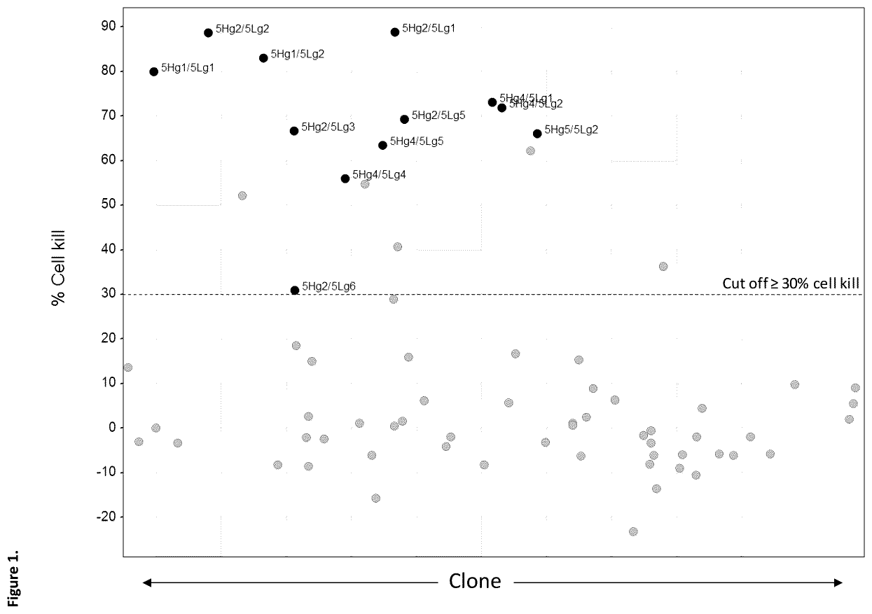

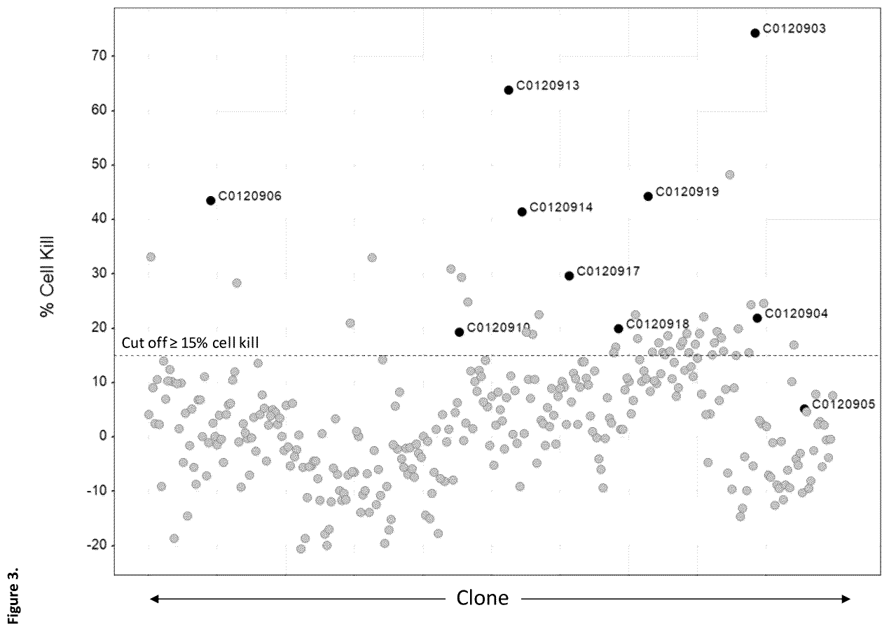

The Fab-ZAP assay assessed the ability of the un-purified IgGs to bind, internalize, and kill NCI-H929 multiple myeloma cells in vitro using a secondary-saporin conjugate, Fab-ZAP (Advanced Targeting Systems, San Diego, CA, IT-51), CellTiter Glo 2.0 (Promega, Madison, WI, G9248) and a Pherastar plate reader (BMG Labtech, Germany). Fab-ZAP is an anti-human Fab that has been conjugated to the warhead saporin. Un-purified humanised 5E3 and fully human, phage display derived, human IgG1s were pre-diluted 8-fold and 4-fold, respectively. The un-purified human IgG1s were contained in Expi293 expression medium (Gibco, UK, A1435101). Following an 8-fold dilution into the assay, final assay concentrations were 1.56 and 3.13%, respectively. 5 μl of pre-diluted un-purified human IgG1 were pre-incubated with 5 μl of 80 nM Fab-ZAP for 30 minutes at room temperature in a 384 well assay plate (Greiner Bio-one, UK, 781091). 2,000 NCI-H929 cells were added to the assay wells in a volume of 30 μl. The assay plate was incubated for 72 hours at 37° C./5% CO₂/95% humidity. Cell kill was assessed by the addition of 20 μl CellTiter Glo 2.0, followed by a 10 minute incubation, before reading the plate for luminescence on the Pherastar. All dilutions were performed in RPMI 1640 (Gibco, UK, A10491-01) supplemented with 10% foetal bovine serum (FBS) (Gibco, UK, 10500-064) and 55 UM β-mercaptoethanol (Gibco, UK, 21985-023). Maximum assay signal was defined by wells containing cells only. Background assay signal was defined by wells containing buffer only i.e., no cells. Data were analysed and graphed using GraphPad Prism software (GraphPad Software, Inc, La Jolla, CA). The results of this experiment are shown in FIG. 1 and FIG. 3.

3.2 Demonstrating mAbs have an Overlapping Epitope to Mouse 5E3 Using an IgG Epitope Competition Assay

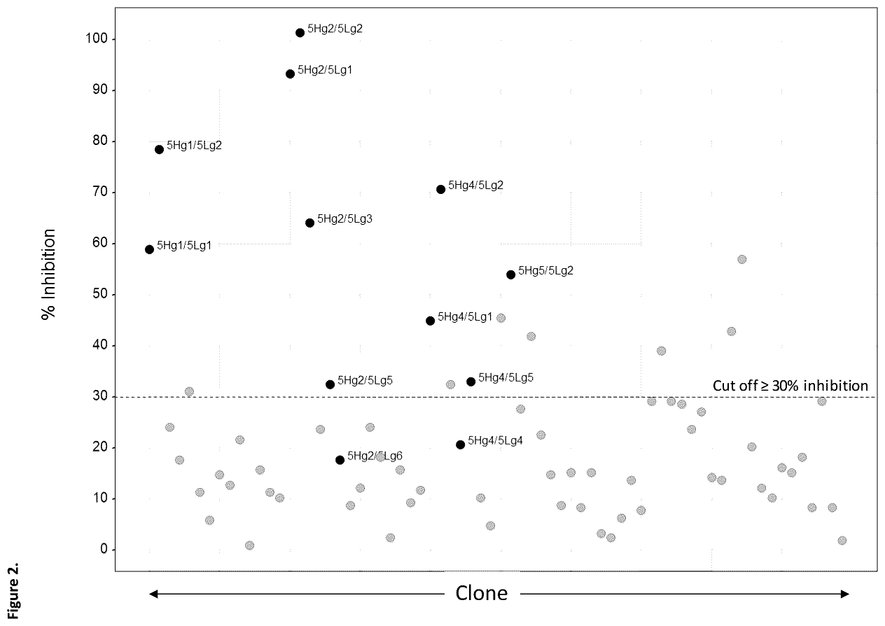

The 5E3 epitope competition assay assessed the ability of the un-purified IgGs expressed in a human IgG1 backbone to compete for binding to the mouse 5E3 epitope on human SEMA4A endogenously expressed by MM.1S cells, a human multiple myeloma cell line. The assay utilised a secondary-Alexa Fluor 647 conjugate, 5E3 expressed in a mouse IgG1 (A) backbone (BioLegend®, San Diego, CA, 148402) and a Mirrorball fluorescence cytometer (SPT Labtech, UK). Binding of 5E3 mouse IgG1 to MM.1S cells was measured via an anti-mouse secondary labelled with Alexa Fluor 647 (Invitrogen, A21235) in the presence of a single concentration of each un-purified human IgG1. Occupation of the 5E3 epitope on SEMA4A by the un-purified IgG resulted in a reduction in the fluorescence signal, as measured by the Mirrorball. 10 μl undiluted un-purified IgG, contained in Expi293 expression medium (Gibco, UK, A1435101), were added to the assay plate (Greiner Bio-one, UK, 781906), followed by 10 ul of 1.2 nM 5E3 mouse IgG1 and 20 μl MM.1S cell suspension containing 1,500 cells that was spiked with 4 nM anti-mouse Alexa Fluor 647. The assay plate was incubated for 4 hours at room temperature, in the dark prior to reading on a Mirrorball fluorescence cytometer (SPT Labtech, UK) using a 640 nm laser for excitation and measuring emission in the FL-4 channel (650-690 nm). Data was analysed as Count x median mean intensity (x FLU). All dilutions were performed in Hanks' balanced salt solution (Sigma, UK; cat: H8264) containing 0.1% (w/V) BSA (Sigma, UK; cat: A9576). Maximum binding signal was determined by analysing the binding of 5E3 mouse IgG1 to MM.1S cells in the absence of competitor un-purified human IgG1. Background binding signal was determined by analysing the fluorescence generated in the absence of 5E3 mouse IgG1. Data were analysed and graphed using GraphPad Prism software (GraphPad Software, Inc, La Jolla, CA). The results of this experiment are shown in FIG. 2.

3.3 Demonstration of Specific Binding of mAbs to the MM1.S Cell Line Using a Mirrorball Binding Assay

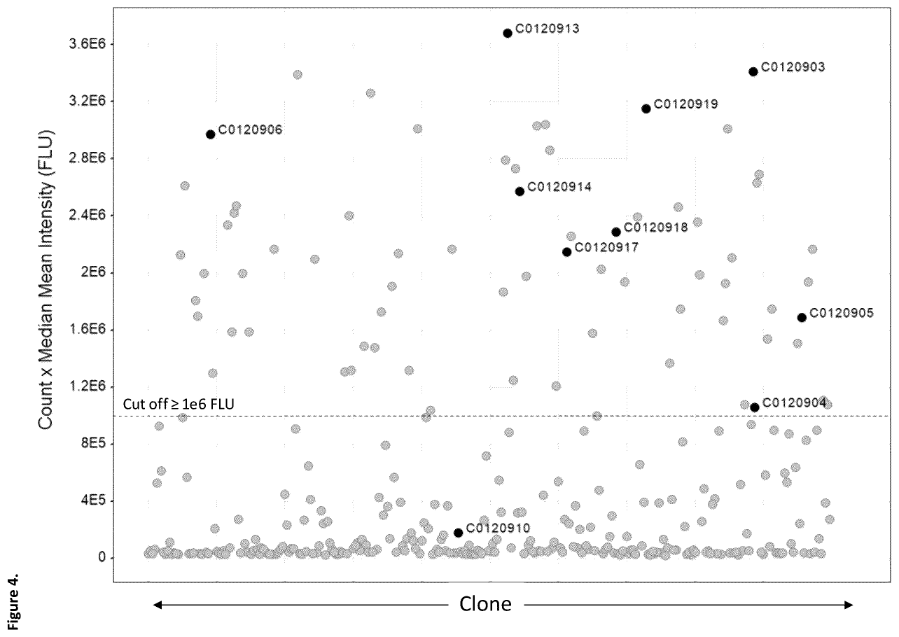

The MM.1S binding assay assessed the ability of the un-purified human IgG1s to bind directly to human SEMA4A that was endogenously expressed by MM.1S cells. Direct binding of a single concentration of un-purified human IgG1s to MM.1S cells was measured via an anti-human secondary labelled with Alexa Fluor 647 (Invitrogen, A21445). 10 μl undiluted un-purified IgG, contained in Expi293 expression medium (Gibco, UK, A1435101), were added to the assay plate (Greiner Bio-one, UK, 781906), followed by 10 μl 8 nM anti-human Alexa Fluor 647 and 20 μl MM.1S cell suspension containing 1,500 cells. The assay plate was incubated for 4 hours at room temperature in the dark prior to reading on a Mirrorball fluorescence cytometer (SPT Labtech, UK) using a 640 nm laser for excitation and measuring emission in the FL-4 channel (650-690 nm). Data was analysed as Count x median mean intensity (x FLU). All dilutions were performed in Hanks' balanced salt solution (Sigma, UK; cat: H8264) containing 0.1% (w/v) BSA (Sigma, UK; cat: A9576). Data were analysed and graphed using GraphPad Prism software (GraphPad Software, Inc, La Jolla, CA). The results of this experiment are shown in FIG. 4.

18 humanised clones were identified as hits in the Fab-ZAP assay (cell kill ≥30%) and 17 humanised clones as hits (inhibition ≥30%) in the mouse 5E3 epitope competition assay. 12 clones that were hits in both assays plus two clones that were Fab-ZAP only hits (5Hg2/5Lg6 and 5Hg4/5Lg4) were re-expressed and purified for further characterisation.

49 fully human, phage display derived clones were identified as hits in the Fab-ZAP assay (cell kill ≥15%). The 49 clones were further triaged based on whether they bound to MM.1S cells endogenously expressing human SEMA4A. This resulted in a panel of 10 clones that were hits in the Fab-ZAP assay and bound to MM.1S cells. These 10 clones were re-formatted from human IgG1 to human Alya IgG1 and expressed and purified for further characterisation as described in section 4.1.

### Example 4: Secondary Screening of Purified Human Alya IgG1

### 4.1 Reformatting of SEMA4A Binding Antibodies to Human Alya IgG1

Specific variable fragments were expressed as a human IgG1-ADC isotype (Alya IgG1) which consists of human IgG1 constant chain with modifications to introduce 2 cysteines into each heavy chain fragment to which drug molecules can be covalently conjugated—this results in a ‘drug-to-antibody’ ratio of four (4DAR). Variable heavy chain (V_{H}) and variable light chain (V_{L}) domains into vectors expressing whole human antibody heavy and light chains respectively. The variable heavy chains were cloned into a mammalian expression vector (pEU1.22, Alya vector) containing the human heavy chain constant domains and regulatory elements to express whole IgG1 heavy chain in mammalian cells. pEU1.22 is engineered to contain two site-specific cysteines that enable controlled drug conjugation (239iCys and S442C (EU numbering) in the CH2 and CH3 Fc domain, respectively). Similarly, the variable light chain domain was cloned into a mammalian expression vector for the expression of the human lambda light chain constant domains (pEU4.4) or human kappa light chain constant domains (pEU3.4) and regulatory elements to express whole IgG light chain in mammalian cells. The vector for expression of heavy chain was originally described by Thompson et al., 2016. Vectors for the expression of light chains were originally described in Persic, et al., 1997.

To obtain clones as IgG, the heavy and light chain IgG expression vectors were transiently transfected into ExpiCHO (ThermoScientific UK; cat. number: A29133) cells where the antibody was expressed and secreted into the medium. Harvested media was filtered prior to purification. The IgGs were purified using Protein A chromatography (HiTrap Fibro PrismA, Cytiva, UK). Culture supernatants were loaded onto an appropriate Protein A column pre-equilibrated in 25 mM Tris pH 7.4, 50 mM NaCl. Bound IgG was eluted from the column using 0.1 M Sodium Citrate pH 3.0, 100 mM NaCl. The IgGs were buffer exchanged into PBS. The purified IgGs were passed through a 0.2 μm filter and the concentration of IgG was determined by absorbance at 280 nm using an extinction coefficient based on the amino acid sequence of the IgG. The purified IgGs were analysed for aggregation or degradation using SEC-HPLC and SDS-PAGE techniques.

### 4.2 Confirming Binding Profile of SEMA4A Specific Human Alya IgG (Purified, Unconjugated) Using a Direct Binding Assay (Mirrorball)

Hits from the primary screening were re-formatted into the human Alya IgG1 vector and re-expressed and purified as described in section 4.1. In addition, a single clone was identified that contained an Asparagine deamidation motif (Asn Gly) within its V_{H}CDR2. This potential sequence liability was de-risked by synthesising de novo two clonal variant V_{H} genes (Asn Ala and Gln Gly) and cloning these sequences into the human Alya IgG1 expression vector. To determine the specificity profile of each purified human Alya IgG1, a dilution series was tested for binding to human SEMA4A, mouse SEMA4A, cyno SEMA4A, human SEMA4B and mock transfected cells (transient transfections set up in expi 293 cells.) The five assays employed methods described in section 2.3, using the appropriate cells and substituting the un-purified scFv periplasmic preparation with the recombinant human Alya IgG1 dilution series. Data was analysed as Count x median mean intensity (x FLU).

A further modification to the assay was the substitution of mouse anti c-myc antibody and anti-mouse antibody conjugated to Alexa Fluor 647 with anti-human Alexa Fluor 647 detection reagent (ThermoFisher Scientific, UK; cat: A21445) as the recombinant human Alya IgG1 had no c-myc tag.

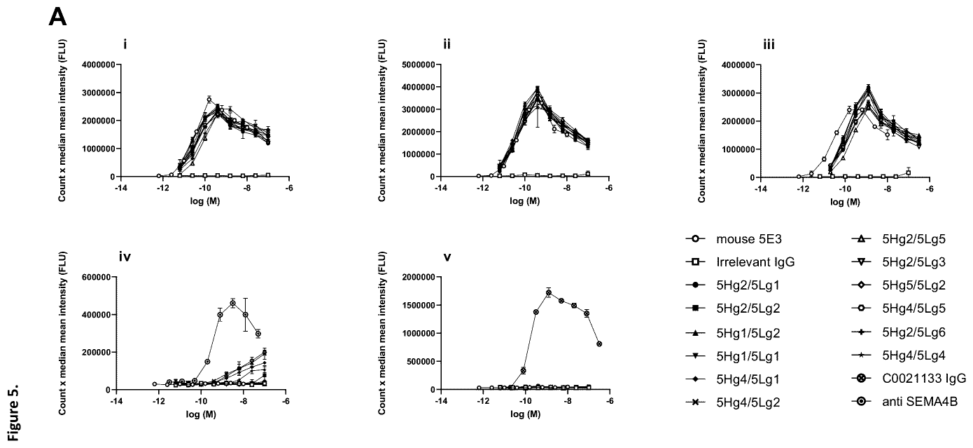

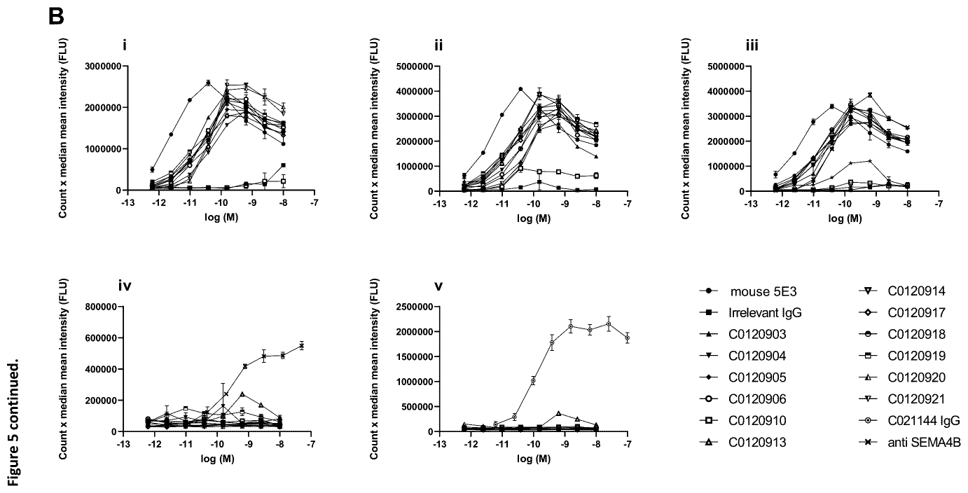

The required binding profile was for binding to human SEMA4A, mouse SEMA4A, cyno SEMA4A but not to human SEMA4B and mock transfected expi293 cells. The results of this experiment are shown in FIG. 5.

### 4.3 Confirming Cell Kill Ability of SEMA4A Specific Human Alya IgG1 (Purified, Unconjugated) Using a Fab-ZAP Cell Kill Assay

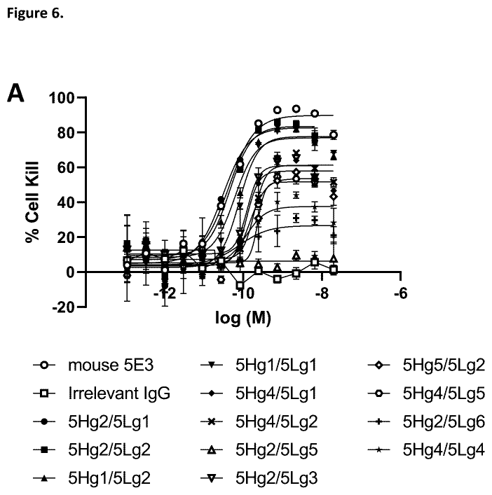

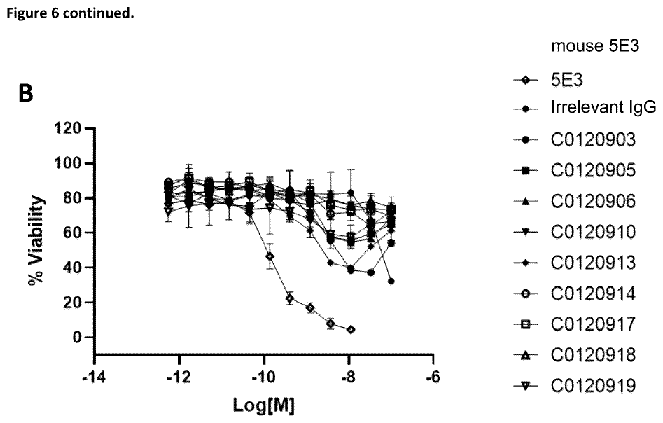

Hits from the primary screening that were re-formatted into the human Alya IgG1 vector and re-expressed and purified as described in section 4.1 were tested in a Fab-ZAP cell kill assay. Fab-ZAP assay methods are described in Example 3.1 and were used to test purified human Alya IgG1s. The purified human Alya IgG1s were diluted to an 8× stock and then serially diluted using 4-fold dilutions to generate a 12 point concentration response curve. 5 μl of the concentration response curve were transferred to the assay plate in duplicate in place of un-purified IgG. The ability of humanised leads and fully human leads, expressed in the human Alya IgG1 vector, to kill NCI-H929 cells in a Fab-ZAP cell kill assay are shown in FIGS. 6A and 6B, respectively. Table 6 shows the cell kill ability of humanised lead clones compared to mouse 5E3 in human IgG1 Alya vector. A number of humanised clones showed IC50 values comparable to 5E3.

TABLE 6
(FIG. 6) Cell kill ability of humanised lead clones expressed in the human
Alya IgG1 vector compared to mouse 5E3 IgG1 Alya in a FabZAP assay.

| | IC₅₀ | Max % |
|---|---|---|
| Clone ID | (pM) | Cell Kill |
| 5E3 | 38 | 89 |
| 5Hg2/5Lg1 | 38 | 78 |
| 5Hg2/5Lg6 | 42 | 22 |
| 5Hg2/5Lg2 | 52 | 83 |
| 5Hg1/5Lg1 | 66 | 76 |
| 5Hg1/5Lg2 | 74 | 74 |
| 5Hg2/5Lg3 | 113 | 58 |
| 5Hg4/5Lg4 | 119 | 33 |
| 5Hg4/5Lg5 | 119 | 50 |
| 5Hg4/5Lg1 | 122 | 56 |
| 5Hg4/5Lg2 | 151 | 58 |
| 5Hg5/5Lg2 | 167 | 51 |
| 5Hg2/5LgS | 675 | 8 |

### 4.4 IgG Competition Assay to Assess Epitope Binding of SEMA4A Specific Alya IgGs (Purified, Unconjugated)

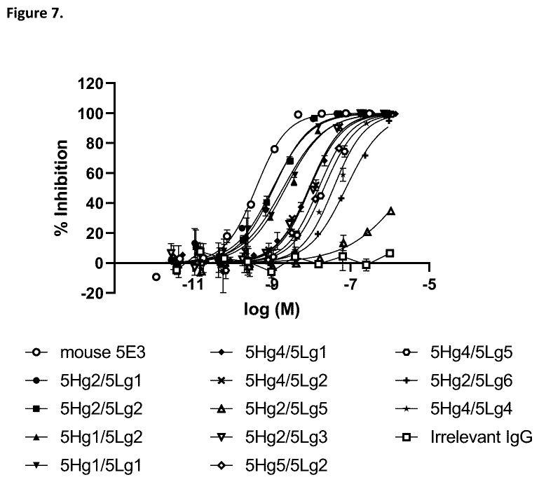

Hits from the primary screening were re-formatted into the human Alya IgG1 vector and re-expressed and purified as described in section 4.1. 5E3 epitope competition assay methods are described in section 3.2 and were used to test purified human Alya IgG1s. The purified human Alya IgG1s were diluted to a 4× stock and then serially diluted using 4-fold dilutions to generate a 10 point concentration response curve. 10 μl of the concentration response curve were transferred to the assay plate in duplicate in place of un-purified IgG. The results of this experiment are shown in FIG. 7 and Table 7.

TABLE 7
(FIG. 7) IC₅₀ values of mouse 5E3 humanised leads expressed in a
human Alya IgG1 vector in an IgG epitope competition assay

| Clone ID | IC₅₀ (nM) |
|---|---|
| 5E3 | 0.3 |
| 5Hg2/5Lg1 | 1.1 |
| 5Hg2/5Lg2 | 1.1 |
| 5Hg1/5Lg2 | 2.1 |
| 5Hg1/5Lg1 | 2.0 |
| 5Hg4/5Lg1 | 9.7 |
| 5Hg4/5Lg2 | 8.4 |
| 5Hg2/5Lg5 | 1919.7 |
| 5Hg2/5Lg3 | 11.3 |
| 5HE5/5Lg2 | 14.9 |
| 5Hg4/5Lg5 | 20.4 |
| 5Hg2/5Lg6 | 71.8 |
| 5Hg4/5Lg4 | 45.5 |
| Irrelevant IgG | Inactive |

### 4.5 Generation of Fab Fragments to Enable Kinetic Profiling of Improved Variants

Equivalent to the conversion of scFv to IgG1—ADC (section 4.1), the scFvs were also converted to Fab fragments, sub-cloning the Variable heavy chain (V_{H}) and variable light chain (V_{L}) domains into vectors expressing part of human antibody heavy and the whole light chains respectively. The variable heavy chains were cloned into a mammalian expression vector (pEU1.3 Fab) that contains the CH1 human heavy chain constant domain, part of the hinge region, and required regulatory elements to express the Fab heavy chain fragment in mammalian cells. Similarly, the variable light chain domain was cloned into a mammalian expression vector for the expression of the human lambda light chain constant domains (pEU4.4) or human kappa light chain constant domains (pEU3.4) and regulatory elements to express whole IgG light chain in mammalian cells.

To obtain clones as Fab, the Fab heavy and light chain IgG expression vectors were transiently transfected into ExpiCHO cells (ThermoScientific UK; cat. number: A29133) where the Fab fragments was expressed and secreted into the medium. Harvested media was filtered prior to purification. The Fab fragments were purified using an affinity matrix recognising the CH1 domain of human IgG antibodies (Capture Select CH1-XL Columns, ThermoFisher Scientific, UK). Culture supernatants were loaded onto an appropriate Capture Select column pre-equilibrated in PBS. Bound Fab fragment was eluted from the column using 50 mM Sodium Acetate buffer pH 4.0-4.5. The Fab fragments were buffer exchanged into PBS. The purified Fab fragments were passed through a 0.2 μm filter and the concentration of the Fab fragments was determined by measuring their absorbance at 280 nm using an extinction coefficient based on the amino acid sequence of the Fab fragment. The purified Fab fragments were analysed for aggregation or degradation using SEC-HPLC and SDS-PAGE techniques.

### 4.6 Determination of Binding Affinities of the 5E3 Humanised and Fully Human Lead Panel to Human SEMA4A and Cyno SEMA4A Using Bio-Layer Interferometry

The OctetRED (Pall ForteBio) instrument was used to assess the kinetic parameters of the interactions between the lead anti-SEMA4A IgGs and recombinantly produced human SEMA4A and cynomolgus SEMA4A. The Octet biosensor uses an optical analytical technique that analyses the interference pattern of white light reflected from two surfaces: a layer of immobilised protein on the sensor tip, and an internal reference layer. Any changes in binding at the biosensor tip result in a shift in interference pattern, which can be measured in real time. Molecules associating with or dissociating from ligands at the biosensor tip shift the interference pattern and generate a response on the Octet system which is recorded by the acquisition software.

Typically, a defined concentration of the analyte species is brought into contact with the coupled ligand and any binding is detected as an increase in signal (association phase). This is followed by a period of buffer rinse, during which dissociation of the analyte species from the surface immobilised ligand can be observed as a decrease in signal (dissociation phase). Repetition of this with a range of analyte concentrations provides data for the analysis of binding kinetics.

An Octet Kinetics buffer (PBS containing 0.01% (v/v) BSA and 0.002% (v/v) Tween20) is typically used as the diluent buffer for the analyte samples and as the flow buffer during the dissociation phase. The experimental data is recorded as shift in interference pattern (nm) over time, which is directly proportional to the optical thickness at the biosensor tip, which in turn is an approximate measure of the mass of analyte bound. The proprietary Octet Data Analysis software package can then be used to process data and fit binding models to the data sets. Returned association (ka, M-1 s-1) and dissociation (kd, s-1) rate constants allow calculation of dissociation (K_{D}, M) affinity constants.

The affinity of binding between the analytes (humanised and phage display derived IgGs as human Alya IgG1s and Fabs) and human SEMA4A and cynomolgus SEMA4A, was estimated using assays in which the biotinylated SEMA4A antigen was captured on a streptavidin sensor tip. A fresh sensor tip was used for each measurement and no regeneration was used. A series of dilutions of the lead Fabs and human Alya IgG1s (3.25-240 nM) were individually placed in contact with the ligand surface for a sufficient amount of time to observe sensorgrams that could be fitted to an appropriate binding model with confidence (typically 5 minutes), followed by an appropriate length of dissociation time (typically 10 minutes). Blank reference (0 nM analyte) data were subtracted from each dataset to reduce the impact of any buffer artefacts or non-specific binding effects. A 1:1 binding model was then fitted simultaneously to the data from each analyte titration using the Octet Evaluation software (Table 8).

TABLE 8
Affinity calculations for humanised and fully human clones as Fab
fragments and human Alya IgG1 to recombinant SEMA4A protein by
Bio-Layer Interferometry.

| | humanSEMA4A | | | | cynoSEMA4A | | | |
|---|---|---|---|---|---|---|---|---|
| | K_{D(observed)} | | | | K_{D(observed)} | | | |
| Sample ID | IgG | | K_{D} Fab | | IgG | | K_{D} Fab | |
| Mouse 5E3 | ≤1 | nM | 9 | nM | ≤1 | nM | 9 | nM |

| | | | | | | | |
|---|---|---|---|---|---|---|---|
| 5Hg1/5Lg1 | ≤2 | nM | 66 | nM | not | 73 | nM |
| Fab | | | | | determined | | |
| 5Hg2/5Lg2 | ≤2 | nM | 41 | nM | not | 48 | nM |
| Fab | | | | | determined | | |

| | | | | | | |
|---|---|---|---|---|---|---|
| C0120903 | 6 | nM | not | 20 | nM | not |
| | | | determined | | | determined |
| C0120905 | 10 | nM | not | 9 | nM | not |
| | | | determined | | | determined |
| C0120906 | 11 | nM | not | 2 | nM | not |
| | | | determined | | | determined |
| C0120914 | 21 | nM | not | 97 | nM | not |
| | | | determined | | | determined |
| C0120917 | 14 | nM | not | 6 | nM | not |
| | | | determined | | | determined |
| C0120918 | 9 | nM | not | 9 | nM | not |
| | | | determined | | | determined |

4.7 Epitope Binning of Antibodies from Humanised and Fully Human Antibody Panels Using Octet

The OctetRED (Pall ForteBio) instrument was used as described in section 4.6 to perform epitope binning, grouping lead anti-human SEMA4A clones into bins based upon binding to recombinant human SEMA4A. This grouping is performed using cross competition assays, in which the competitive binding of analyte pairs to a specific antigen is characterised. In this in-tandem format the antigen (biotinylated recombinant human SEMA4A) immobilized onto a streptavidin biosensor is presented to the two competing analytes in consecutive steps. Binding to distinct non-overlapping epitopes is indicated if saturation with the first Fab fragment (mouse 5E3) does not block binding of the second IgG.

A defined concentration of the analyte species is brought into contact with the coupled ligand and any binding is detected as an increase in signal (association phase). Epitope binning is achieved by saturating this first association (determined during affinity measurements, typically 5 minutes), followed by a second association step where the sensor is dipped into a well containing both the first analyte (mouse 5E3 Fab) as well as a second analyte (naïve SEMA4A IgG) (typically 5 minutes). The first analyte is included to maintain saturation whilst the second analyte attempts to bind. This is followed by an appropriate length of dissociation time (typically 10 minutes). A fresh sensor tip was used for each measurement and no regeneration was used.

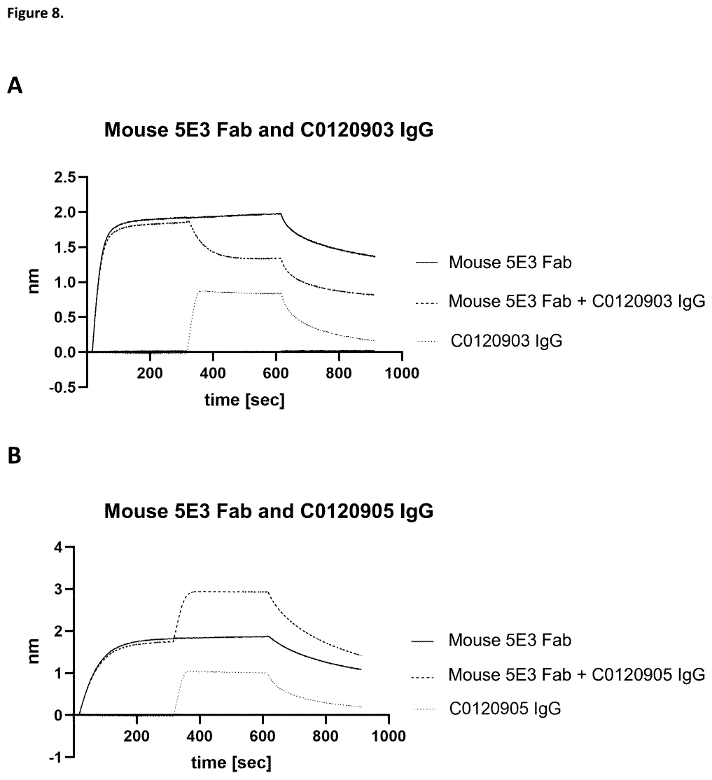

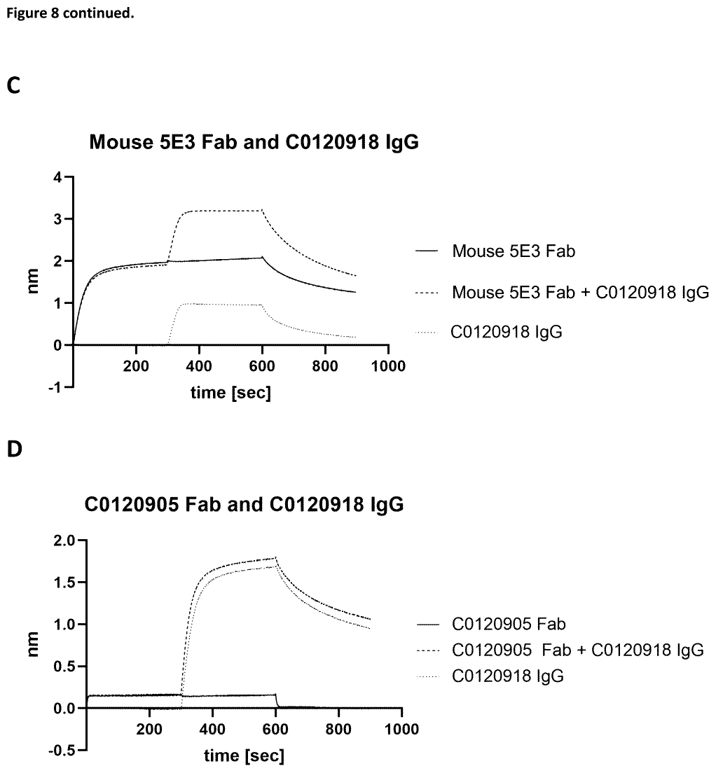

The results of this experiment are shown in FIG. 8. FIG. 8A is representative of data generated from two clones that have overlapping or competing epitopes. FIGS. 8B-D show data corresponding to clones with non-overlapping epitopes.

### Example 5: Conjugation of Linker and Warhead (McMMAF) to Human Alya IgG1

### 5.1 Conjugation of SEMA4A Specific Human Alya IgG1s to McMMAF

Humanised and fully human leads were cloned into the human Alya IgG1 vector and expressed, purified and concentrated to 10 mg/ml in DPBS, pH 7. Each protein was mildly reduced using TCEP (Tris-(2-carboxyethyl)-phosphine Hydrochoride, Sigma cat: 75259) at a 40-fold molar excess of TCEP to antibody for 3 hours at 37° C. Post desalt (Zeba™ Spin Desalting Columns, ThermoFisher Scientific, cat: 89882) into PBS, pH 7.2, 1 mM EDTA, the IgG was oxidated using dhAA (Dehydroascorbic acid, Sigma cat: 261556) at a 20-fold molar excess of dhAA to antibody for 4 hours at RT. The MMAF conjugation was performed over night at 4° C. using McMMAF (MedChemExpress, cat: HY-15578) at a 12-fold molar excess MMAF to Antibody. The reaction was stopped using NAC (N-acetyl-L-cysteine, Sigma-Aldrich, cat: A7250) at a 4-fold molar excess for 15 minutes at RT. Finally, each conjugated SEMA4A IgG (human Alya IgG1) was buffer exchanged into PBS, pH 7.2, 1 mM EDTA, concentrated and purified using Superose 6 Increase 10/300 GL (Sigma-Aldrich, cat: GE29-0915-96) using PBS, pH 7.2, 1 mM EDTA. The IgGs were run on SDS PAGE (NuPAGE 4-12% BisTris gels (12 well), ThermoFisher Scientific, cat: NP0322PK2) at 200V for 60 minutes alongside each unconjugated SEMA4A human Alya IgG1 to confirm conjugation.

### 5.2 Reconfirmation of Binding/Specificity of Lead Panel (Human Alya IgG1s Conjugated to McMMAF) to SEMA4A (Mirrorball)

The human Alya IgG1 clones were conjugated to McMMAF as described in section 5.1. Following the conjugation, it was necessary to retest these clones to reconfirm their binding profile to human SEMA4A, human SEMA4B and mock transfected cells (transient transfections set up in expi 293 cells).

To determine the specificity profile of each purified human Alya IgG1 conjugated to McMMAF, a dilution series was tested for binding to human SEMA4A, human SEMA4B and mock transfected cells (transient transfections set up in expi 293 cells.) The three assays employed methods described in section 2.3, using the appropriate cells and substituting the un-purified scFv periplasmic preparation with the human Alya IgG1 conjugated to McMMAF. Data was analysed as Count x median mean intensity (x FLU).

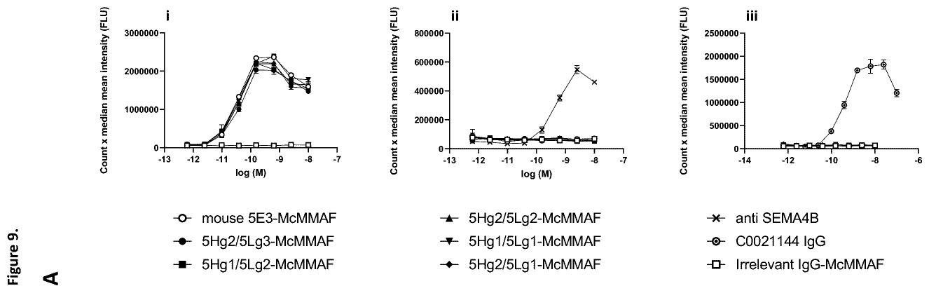

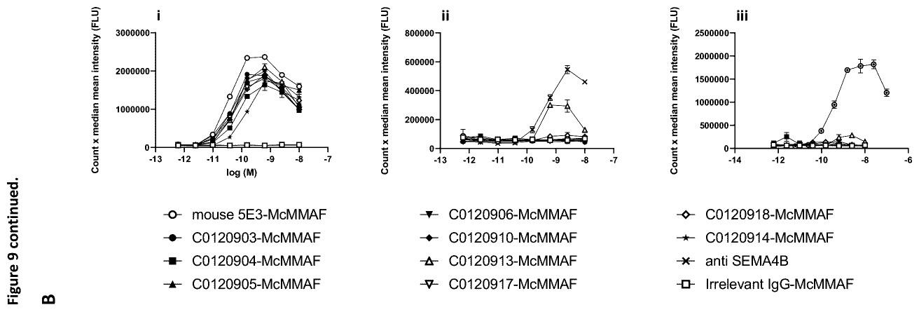

A further modification to the assay was the substitution of mouse anti c-myc antibody and anti-mouse antibody conjugated to Alexa Fluor 647 with anti-human Alexa Fluor 647 detection reagent (ThermoFisher Scientific, UK; cat: A21445) as the recombinant IgG had no c-myc tag. The required binding profile was for binding to human SEMA4A but not to human SEMA4B and mock transfected expi293 cells. The results of this experiment are shown in FIG. 9.

5.3 Cell Kill Assays with SEMA4A Lead Panel (Human Alya IgG1 Conjugated to McMMAF) Benchmarked Against J6MO in MM.1S, NCI-H929 and Karpas-25 Cell Lines

The cell kill assay assessed the ability of SEMA4A specific human Alya IgG1s conjugated to McMMAF to bind, internalize, and kill MM.1S, NCI-H929 and Karpas-25 multiple myeloma cells and K562 leukaemia cells in vitro using CellTiter Glo 2.0 (Promega, Madison, WI, G9248) and a Pherastar plate reader (BMG Labtech, Germany). The capacity of the conjugates to kill all four cell lines was tested and benchmarked relative to an McMMAF-conjugated BCMA-specific Alya IgG1, known as J6MO (Tai et al., 2014). The three cell lines were selected for these assays as high (MM.1S), medium (NCI-H929) and low (Karpas-25) expressors of human SEMA4A. K562 cells do not express human SEMA4A.

In a comparison of SEMA4A and BCMA expression levels the NCI-H929 cells were shown to express equivalent levels of both targets (3E5 antibody binding sites per cell for both SEMA4A and BCMA, determined using Quantum™ Simply Cellular® anti-Mouse IgG beads cat #815, Bangs Laboratories, Inc, Indiana, USA). This cell line was considered to be a suitable line to understand the potency of the SEMA4A panel of mAbs versus J6MO for cell kill and internalisation rates.

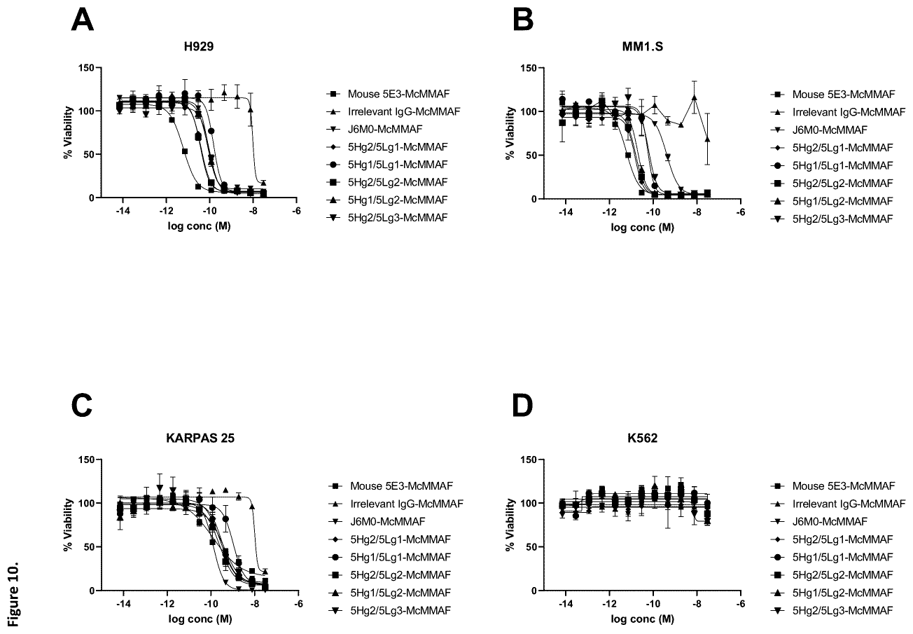

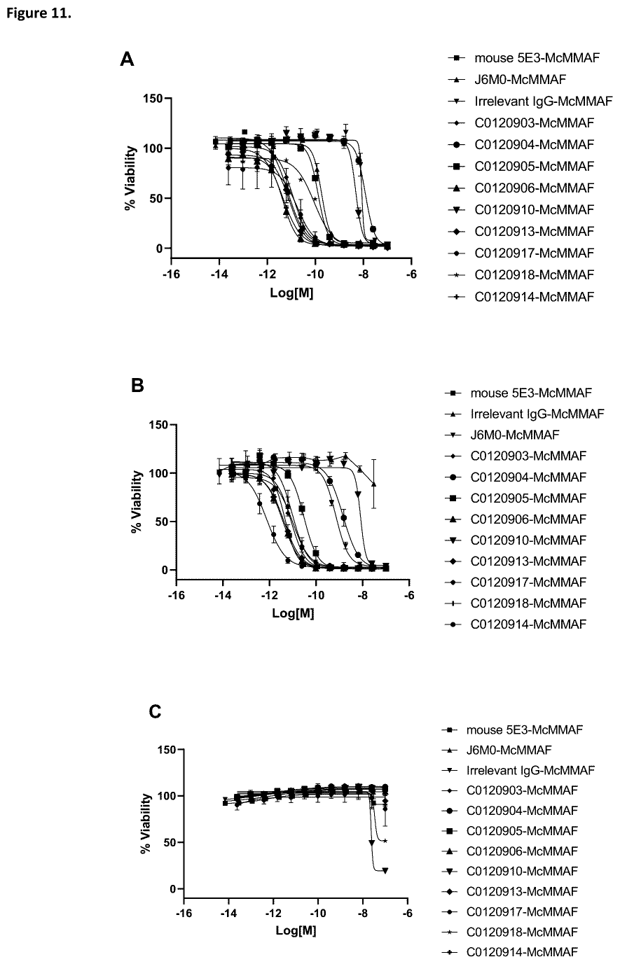

The McMMAF conjugates were diluted to a 4× stock and then serially diluted using 4-fold dilutions to generate a 12-point concentration response curve. 10 μl of the concentration response curves were added to the assay plate (Greiner Bio-one, UK, 781091) followed by 30 μl cell suspension containing 2,000 MM.1S, NCI-H929, Karpas-25 or K562 cells. The assay plate was incubated for 96 hours at 37° C./5% CO₂/95% humidity. Cell kill was assessed by the addition of 20 μl CellTiter Glo 2.0, followed by a 10 minute incubation, before reading the plate for luminescence on the Pherastar. All dilutions were performed in RPMI 1640 (Gibco, UK, A10491-01) supplemented with 10% FBS (Gibco, UK, 10500-064) except for assays testing Karpas-25 cells where assay media was also supplemented with 55 UM β-mercaptoethanol (Gibco, UK, 21985-023). Maximum assay signal was defined by wells containing cells only. Background assay signal was defined by wells containing buffer only i.e., no cells. Data were analysed and graphed using GraphPad Prism software (GraphPad Software, Inc, La Jolla, CA). The results of this experiment are shown in FIG. 10 and Table 9, and in FIG. 11 and Table 10.

TABLE 9
Cell kill ability of 5E3 humanised clones (human Alya IgG1 conjugated
to McMMAF) was compared to mouse 5E3 as a human Alya IgG1
coupled to McMMAF.

| | | | Average | |
|---|---|---|---|---|
| | Average | Average | KARPAS | Average |
| | NCI-H929 | MM1.S | 25 IC₅₀ | K562 IC₅₀ |
| Antibody | IC₅₀ [pM] | IC₅₀ [pM] | [pM] | [pM] |
| Mouse 5E3 | 7.53 | 5.9525 | 125.6 | Inactive |
| Irrelevant IgG | Inactive | Inactive | 11345 | Inactive |
| J6M0 | 74.55 | 423.85 | 104.145 | Inactive |
| 5Hg2/5Lg1 | 33.64 | 11.752 | 227.9 | Inactive |
| 5Hg1/5Lg1 | 100.26 | 35.805 | 624.65 | Inactive |
| 5Hg2/5Lg2 | 34.265 | 10.6705 | 180.55 | Inactive |
| 5Hg1/5Lg2 | 59.37 | 15.76 | 316.75 | Inactive |
| 5Hg2/5Lg3 | 863.045 | 101.85 | 315.8 | Inactive |

TABLE 10
Cell kill ability of fully human, phage display derived clones (human
Alya IgG1 conjugated to McMMAF) was compared to mouse 5E3 as a
human Alya IgG1 coupled to McMMAF. A number of fully human
clones showed a range of IC₅₀ values, some comparable or slightly more
potent than mouse 5E3. As expected, no clone elicited cell kill in the
negative (non-SEMA4A expressing) control K562 cell line.

| | Average NCI-H929 | Average MM1.S | Average K562 |
|---|---|---|---|
| Antibody | IC₅₀ [pM] | IC₅₀ [pM] | IC₅₀ [pM] |
| Mouse 5E3 | 5.1 | 5.8 | Inactive |
| C0120903 | 7.1 | 8.8 | Inactive |
| C0120904 | 18095 | 1491 | Inactive |
| C0120905 | 90.8 | 63.5 | Inactive |
| C0120906 | 3.0 | 4.7 | Inactive |
| C0120910 | 3994 | 7492 | >[1000 pM] |
| C0120913 | 5.3 | 6.7 | Inactive |
| C0120914 | 2.5 | 1.1 | Inactive |
| C0120917 | 18.1 | 10.9 | Inactive |
| C0120918 | 55.8 | 13.2 | >[1000 pM] |
| Irrelevant IgG | 8859 | NA | Inactive |
| J6M0 | 109.8 | 1211 | Inactive |

### 5.4 Internalisation of Humanised and Fully Human Lead Panel (Human Alya IgG1 Conjugated to McMMAF) Benchmarked Against J6MO in the NCI-H929 Cell Line

The humanised and fully human lead panel as human Alya IgG1 clones were conjugated to McMMAF as described in section 5.1. The capacity of these conjugated IgGs to be internalised into a SEMA4A expressing multiple myeloma cell line was tested and benchmarked relative to an McMMAF-conjugated BCMA-specific human Alya IgG1, known as J6MO (Tai et al., 2014). The NCI-H929 cell line, with equivalent expression of BCMA (3.3×10⁵ antibody binding sites per cell) and SEMA4A (3.2×10⁵ antibody binding sites per cell), was selected for this purpose.

Internalisation into NCI-H929 cells was visualised using a Fab-pHast internalisation assay. This assay was set up using known concentrations of McMMAF-conjugated Alya IgGs, Fab-pHast (Advanced Targeting Systems, San Diego, CA, PH-01), Hoechst 33342 (Invitrogen, UK, H3570) and an ImageXpress Micro high content imaging system (Molecular Devices, San Jose, CA).

Fab-pHast is an anti-human secondary conjugated to a pH-sensitive fluorescent label. The neutral pH of tissue culture media quenches Fab-pHast fluorescence. It is only when the antibody-secondary complex is internalised to the acidic compartments of the endocytic pathway that significant Fab-pHast fluorescence can be detected.

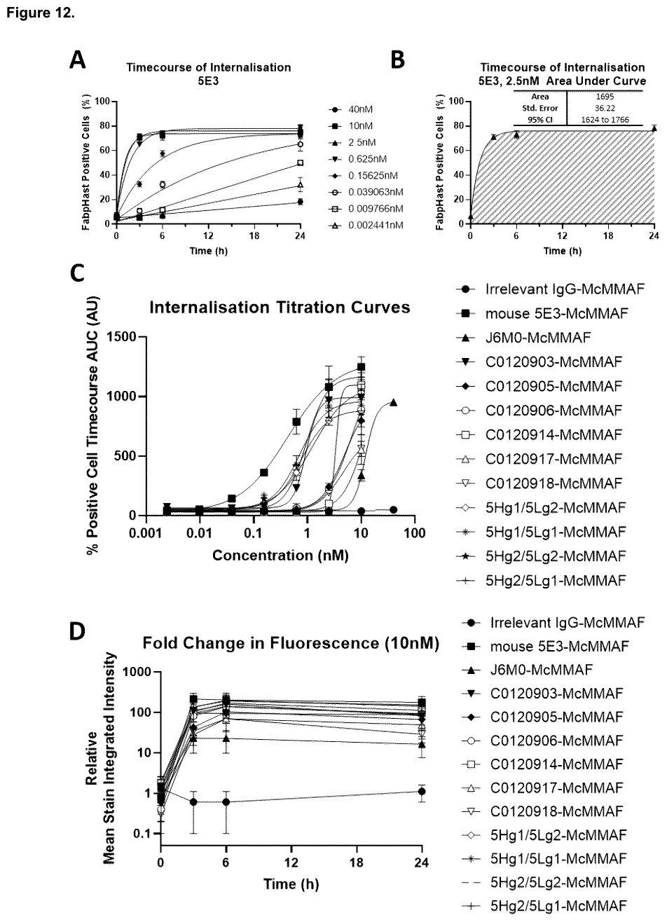

The humanised and fully human lead panel, as McMMAF conjugated human Alya IgG1, were labelled by dilution to a known concentration in 10× Fab-pHast solution. A serial dilution was set up in the same solution to create a titration curve, before incubating the labelling reactions at room temperature for 20 minutes. Labelled antibodies were added at a 1 in 10 ratio to NCI-H929 cells that had previously been seeded in serum free RPMI 1640 (Gibco, UK, A10491-01) on Poly-D-Lysine-coated 96-well tissue culture plates. The treated cells were incubated at 37° C./5% CO₂/95% humidity and imaged on the blue and yellow channels of the ImageXpress Micro at 3, 6 and 24 hours. These images were processed using the MetaXpress 6 software package (Molecular Devices, San Jose, CA) to identify objects in the blue (Hoescht staining of all nuclei) and yellow (Fab-pHast label that has been internalised) channels based on defined thresholds. The software reported both the percentage of Fab-pHast positive cells and their Mean Integrated Intensity (defined as the sum of all Fab-pHast positive pixel intensities divided by the total number of Hoescht positive nuclei). These data were analysed and graphed using GraphPad Prism software (GraphPad Software, Inc, La Jolla, CA). Representative results of this experiment are shown in FIG. 12 with the averaged results of duplicate experiments below in Table 11.

TABLE 11
The internalisation of humanised and fully human lead panel, as human
Alya IgG1 clones conjugated to McMMAF Internalisation was quantified
using a pH sensitive label and compared to the commercially available
SEMA4A antibody mouse 5E3 as well as the BCMA-targeting benchmark
J6M0. All the anti-SEMA4A clones tested accumulate to a higher level in
cells than J6M0, and most show greater potency. Mouse 5E3 and J6M0
were tested as human Alya IgG1 clones conjugated to McMMAF.

| | | Fold-Change at |
|---|---|---|
| IgG | EC₅₀ (nM) | 6 hours (10 nM) |
| Mouse 5E3 | 0.25 | 160 |
| J6M0 | 10.96 | 37 |
| C0120903 | 0.98 | 118 |
| C0120905 | 3.72 | 91 |
| C0120906 | 4.10 | 116 |
| C0120914 | 3.29 | 112 |
| C0120917 | 8.29 | 53 |
| C0120918 | 3.62 | 79 |
| 5Hg1/5Lg2 | 0.75 | 150 |
| 5Hg1/5Lg1 | 0.54 | 121 |
| 5Hg2/5Lg2 | 0.59 | 103 |
| 5Hg2/5Lg1 | 0.62 | 144 |

### 5.5 Determination of Binding Affinities of Lead Panel SEMA4A Specific IgGs (Human Alya IgG1 Conjugated to McMMAF) by Flow Cytometry

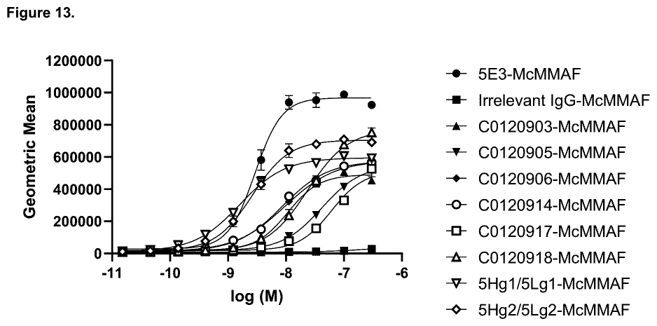

Binding of SEMA4A specific IgGs to membrane-bound SEMA4A was evaluated using flow cytometry in NCI-H929 cells that endogenously express human SEMA4A. Binding assays were performed by incubating the anti-SEMA4A antibodies with 200,000 cells for 3 hours at 4° C. followed by two washes with PBS, 1% (w/v) BSA (FACS buffer). A range of antibody concentrations were evaluated using a 10-point, 3-fold dilution series. Cells were then incubated with Alexa Fluor 647 conjugated AffiniPure Fab Fragment Goat Anti-Human IgG (H+L) (Jackson Immunoresearch Europe Limited, Ely, Cambridgeshire) diluted 1:100, at 4° C. for 1 hour, followed by two washes in FACS buffer. Cells were then stained with LIVE/DEAD™ Fixable Violet Dead Cell Stain (Life Technologies, L34955) for 30 minutes at 4° C., followed by two washes in FACS buffer. Cells were then fixed using BD Cell Fix (BD Biosciences, 340181). Fluorescence of live, single cells was measured using a Novocyte™ 3000 flow cytometer and Novoexpress software (Acea Biosciences, Inc, San Diego, CA). Data were analysed using FlowJo software (FlowJo, LLC, Ashland, OR). Mean fluorescence intensity values were plotted against antibody conjugate concentrations and K_{D} values determined using Prism software (GraphPad Software Inc, La Jolla, CA). The results of this experiment are shown in FIG. 13 and Table 12.

TABLE 12
K_{D} binding affinity values of SEMA4A specific Alya IgGs conjugated
to McMMAF measured by flow cytometry.

| IgG | K_{D} (M) |
|---|---|
| Mouse 5E3-McMMAF | 3.18E−09 |
| C0120906-McMMAF | 9.67E−09 |
| C0120914-McMMAF | 9.04E−09 |
| C0120903-McMMAF | 9.88E−09 |
| C0120905-McMMAF | 3.43E−08 |
| C0120917-McMMAF | 4.05E−08 |
| C0120918-McMMAF | 1.60E−09 |
| 5Hg1/5Lg1-McMMAF | 1.46E−09 |
| 5Hg2/5Lg2-McMMAF | 2.46E−09 |

### Example 6: Germlining Fully Human, Phage Display Derived Leads and Verification of Retained Function

### 6.1 Germlining Fully Human, Phage Display Derived Leads

Fully human leads identified from phage display selections were germlined to help mitigate potential immunogenicity risks. Individual antibody sequences were compared to human germline V, D and J regions accessible via IMGT (ImMunoGeneTics; www.imgt.org) or ImmuneDiscover databases. Amino acid residues differing from the canonical human germline sequence that resided outside of VHCDR1, VHCDR2, VHCDR3, VLCDR1, VLCDR2, VLCDR3 regions and which were not Vernier residues, were reverted. Each fully germlined V_{H} and V_{L} gene sequence was synthesised de novo and cloned into the appropriate IgG expression vector (Persic, et al., 1997) using standard molecular biology techniques.

### 6.2 Confirming the Binding Specificity of Germlined Fully Human, Phage Display Derived, Human Alya IgG1 (Purified, Unconjugated) Using a Direct Binding Assay (Mirrorball)

To confirm the SEMA4A binding specificity of each germlined fully human, phage display derived human Alya IgG1 a dilution series was tested for binding to human SEMA4A, mouse SEMA4A, cyno SEMA4A, human SEMA4B and mock transfected cells (transient transfections set up in expi 293 cells.) The five assays employed methods described in section 2.3, using the appropriate cells and substituting the un-purified scFv periplasmic preparation with the recombinant germlined human IgG1 Alya dilution series. Data was analysed as Count x median mean intensity (x FLU).

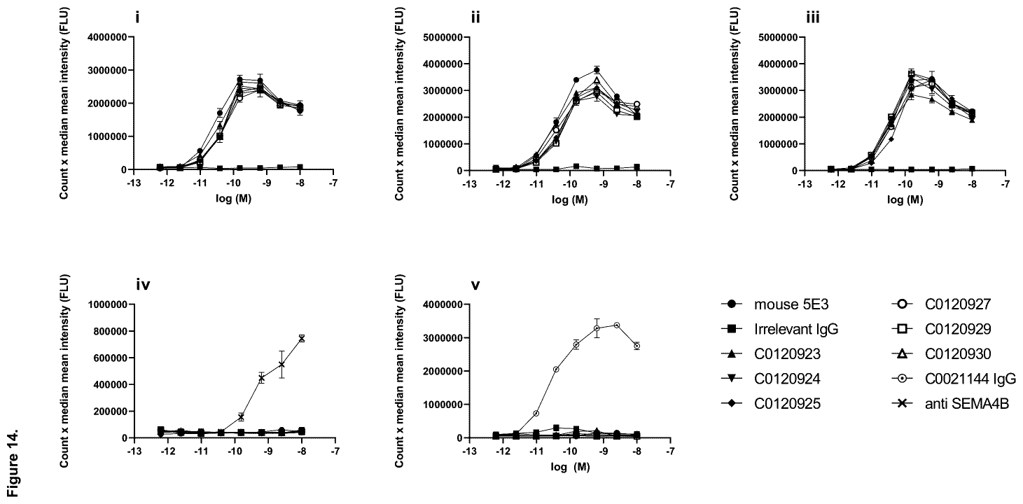

A further modification to the assay was the substitution of mouse anti c-myc antibody and anti-mouse antibody conjugated to Alexa Fluor 647 with anti-human Alexa Fluor 647 detection reagent (ThermoFisher Scientific, UK; cat: A21445) as the recombinant IgG had no c-myc tag. The required binding profile was for binding to human SEMA4A, mouse SEMA4A, cyno SEMA4A but not to human SEMA4B and mock transfected expi293 cells. The results of this experiment are shown in FIG. 14.

### Example 7. Assessing Serum Levels of SEMA4A in MM Patient Samples and Understanding Whether Soluble SEMA4A (sSEMA4A) Impacts on Lead Panel Cell Kill Potency

### 7.1 Assessing Levels of SEMA4A in Patient Samples

Having determined that SEMA4A was a potential ADC target, we further explored the feasibility of targeting SEMA4A in human MM patients by measuring serum concentrations of SEMA4A. Many cell surface proteins are shed into the circulation and this soluble protein can act as a sink, “soaking up” therapeutic antibody. This means that unacceptable blood concentrations of the antibody are required to achieve efficacy. To understand whether this might disrupt therapeutic targeting of SEMA4A, we designed a sandwich ELISA to detect sSEMA4A.

White maxisorp plates (ThermoScientific, UK, 436110) were coated with 50 μl of capture antibody (mouse anti-human SEMA4A clone 5E3, BioLegend®, San Diego, CA, 148402) at 5 μg/mL in PBS and incubated overnight at 4° C. Unbound antibody was removed and plates washed with PBS. Non-specific binding was blocked by incubation with casein (ThermoScientific, UK, 37528) for one hour at room temperature, followed by a PBS wash. 50 μl per well of the unknown sample or the diluted standard (recombinant human SEMA4A (Abcam, UK, ab182683)) were incubated for one to two hours at room temperature. Samples were removed and the plate washed three times with PBS-Tween 20 (0.1% v/v) wash buffer. Captured sSEMA4A was detected by an anti-SEMA4A polyclonal antibody (R&D Systems, AF4694, 50 μl at 5 μg/ml in 1:20 casein) with an hour incubation at room temperature. Plates were washed three times with wash buffer and the secondary detection antibody (donkey anti-sheep IgG conjugated HRP, Abcam, ab97125) added at 1/5000 in 1:20 casein (50 μl) for one hour at room temperature. The secondary detection antibody was removed, and the plate washed five times with wash buffer. 50 μl Pico ELISA substrate (Thermo Scientific, UK, 37070) were added to the plate following manufacturer's guidelines, incubated for five minutes at room temperature and luminescence detected using an EnVision plate reader (PerkinElmer).

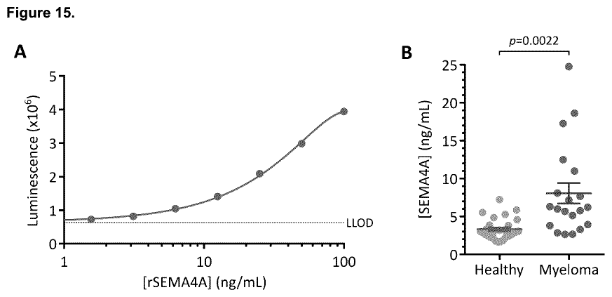

Dilution studies demonstrated that the assay had a limit of detection of no greater than 2 ng/ml (FIG. 15). We assayed sSEMA4A in the serum of healthy controls and in patients with myeloma and found these to be 3.3 ng/ml and 8.1 ng/ml, respectively (FIG. 15). The difference between the two groups was significant (p=0.0022; t-test), but the absolute levels of sSEMA4A are highly unlikely to disrupt the activity of a SEMA4A ADC in myeloma. For example, successful targeting of TNFRSF17 (BCMA) in myeloma by belantamab mafodotin or by CAR-T cells does not seem to have been impaired by median serum expression levels of 176 ng/ml of soluble BCMA. We therefore concluded that shed SEMA4A would be very unlikely to impact on the ability to target this cell surface protein.

7.2 Measuring the Impact of sSEMA4A on the Ability of Fully Human SEMA4A Specific Human Alya IgG1s (McMMAF Conjugated) to Kill NCI-H929 Cells.

The cell kill assay described in section 5.3 was used to assess whether the presence of sSEMA4A has an impact on the potency of fully human SEMA4A specific human Alya IgGs conjugated to McMMAF to kill NCI-H929 cells. sSEMA4A was derived from the cell culture media (CCM) of NCI-H929 cells that had been concentrated 100-fold. sSEMA4A levels in the CCM were quantified using the ELISA described in Example 7.1. CCM containing no sSEMA4A was derived from NCI-H929 SEMA4A knockout cells and was used for the 0 ng/ml SEMA4A concentration. Titrations of fully human, phage display derived, human Alya IgG1s conjugated to McMMAF were tested in the presence of sSEMA4A spiked into the assay at 0, 8, 25 and 100 ng/ml.

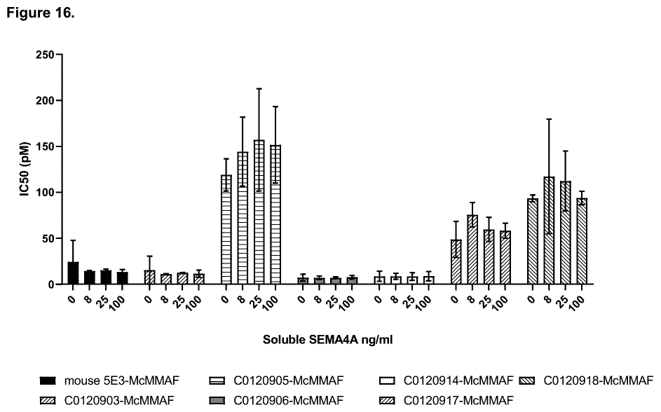

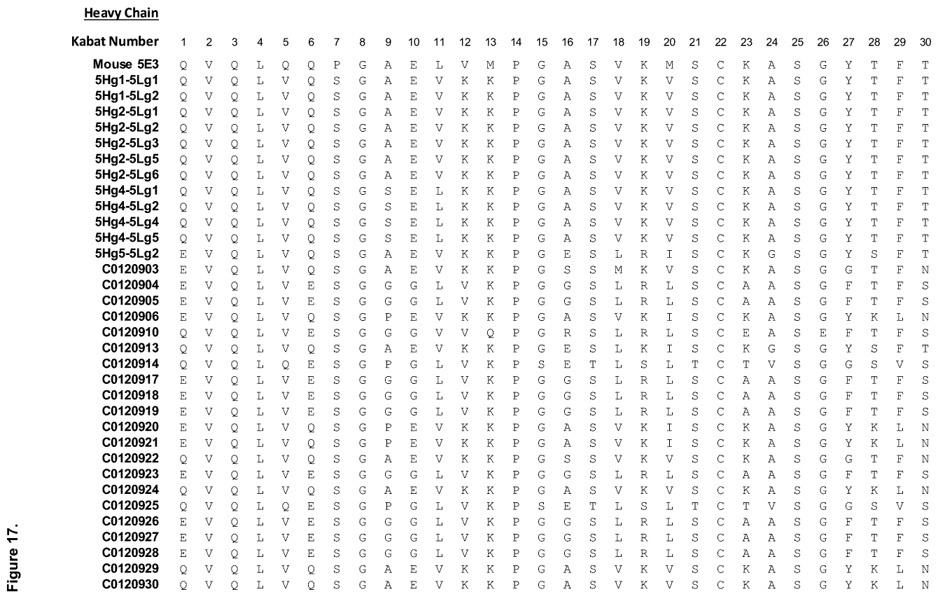

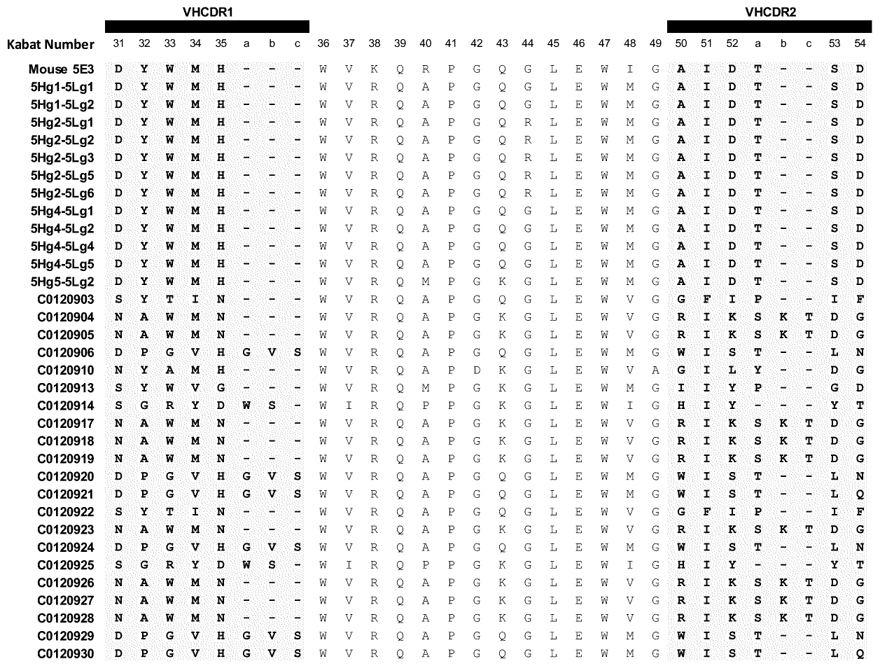

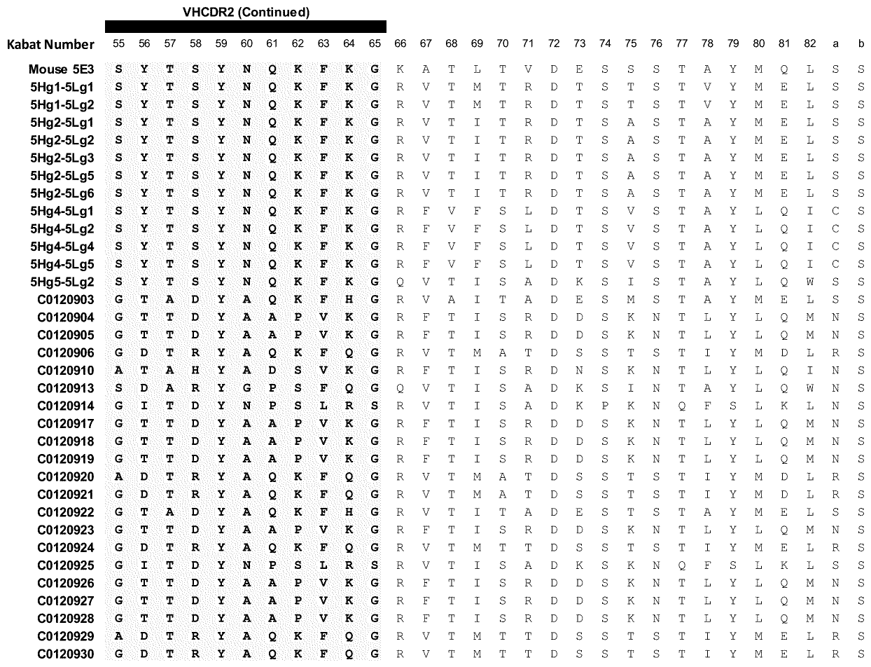

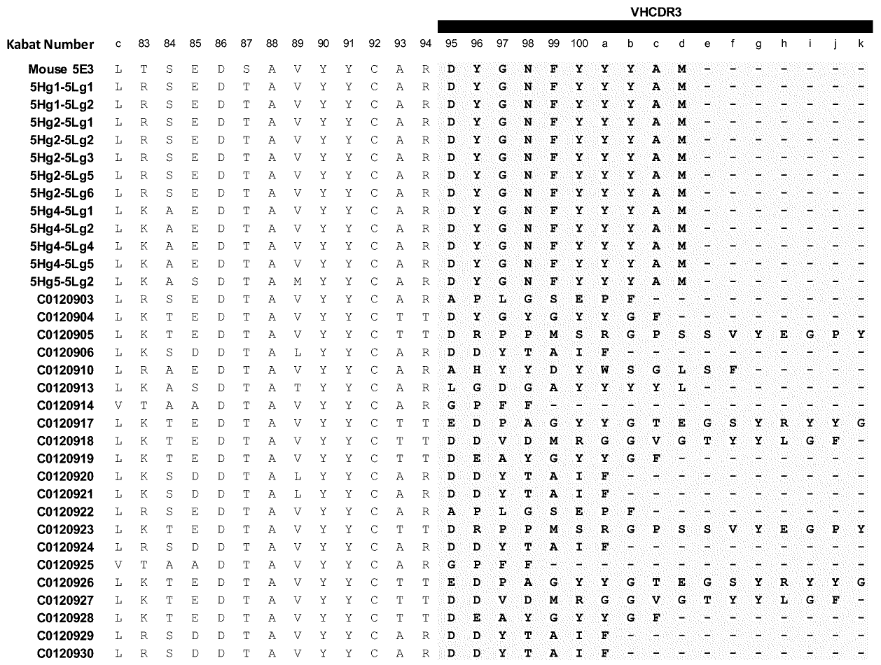

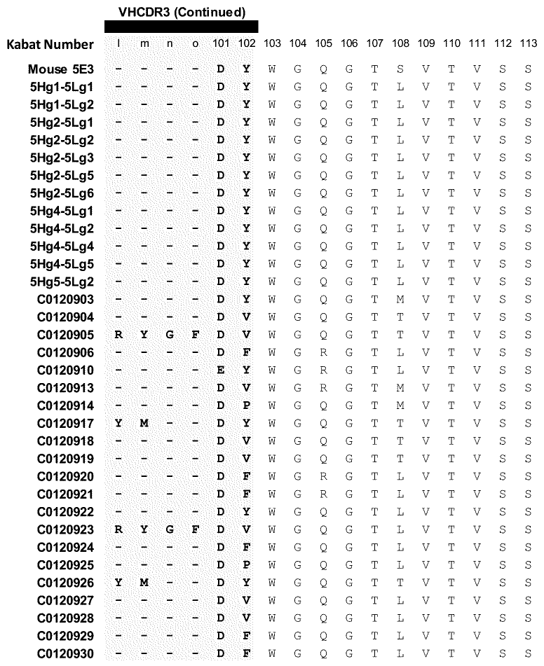

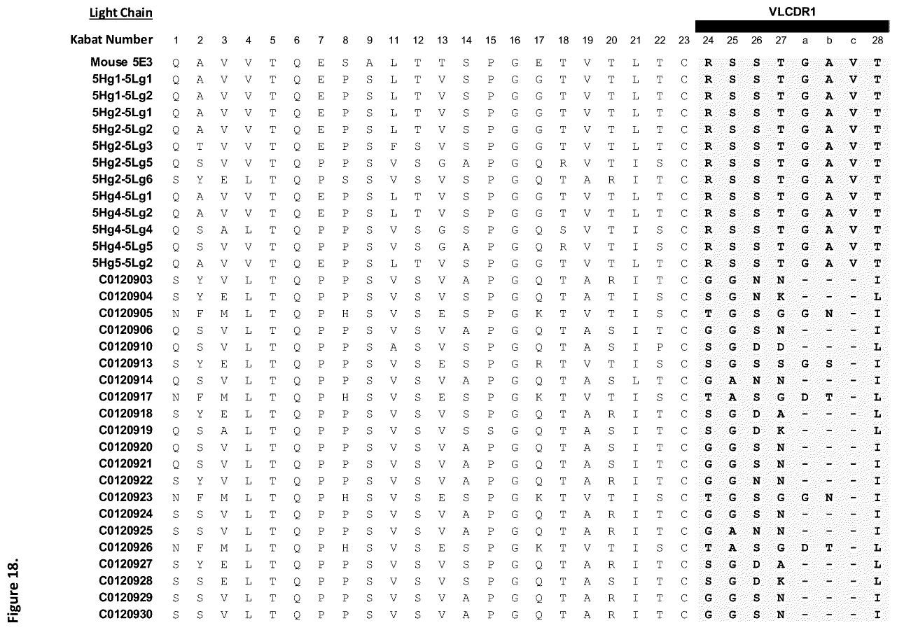

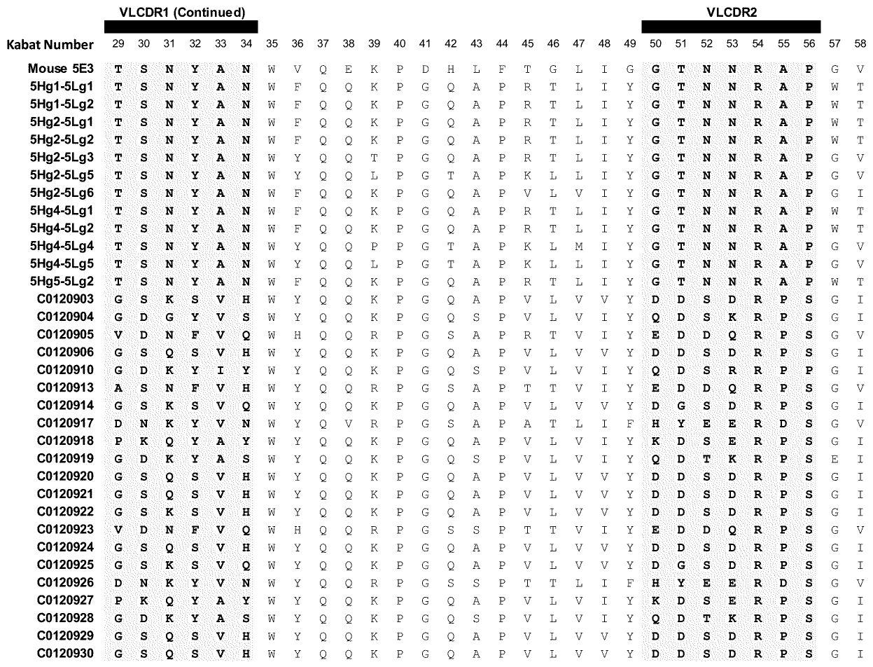

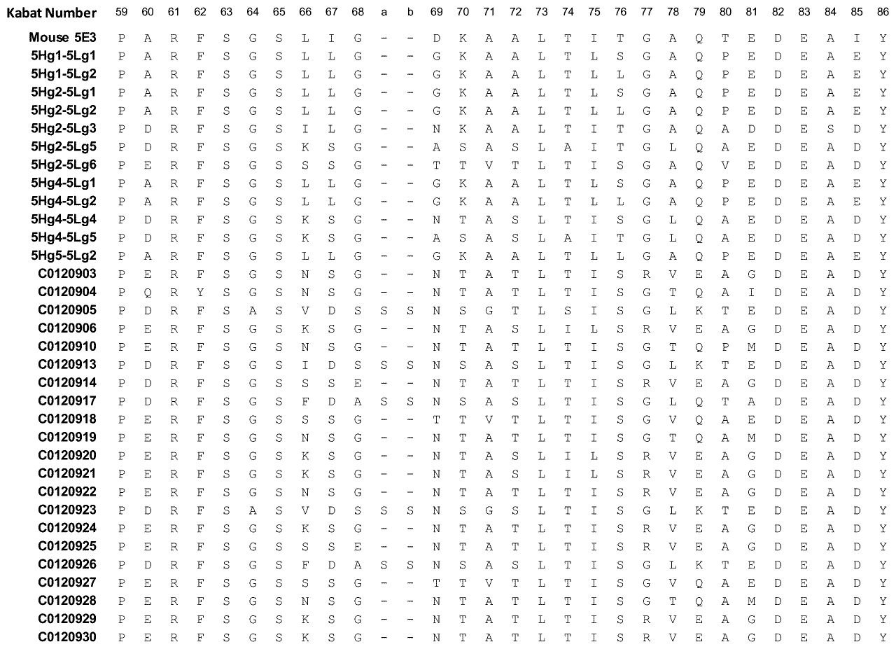

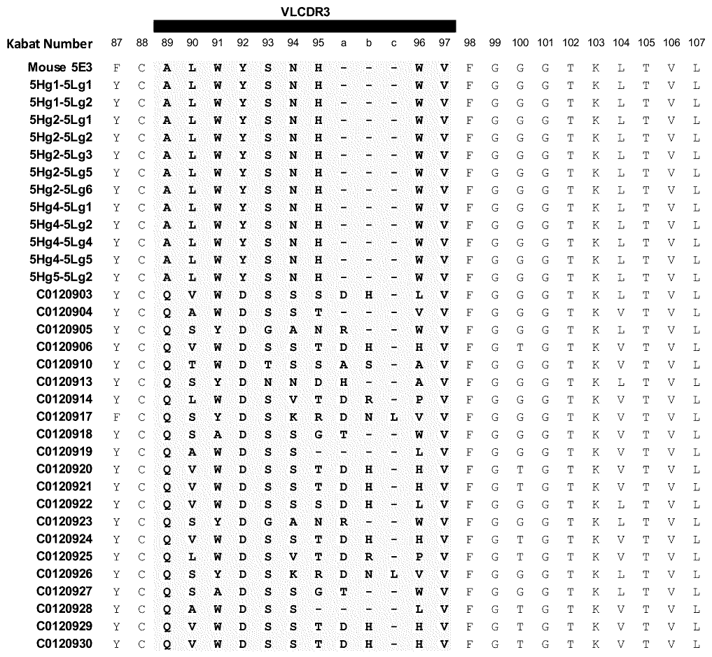

The results of this experiment (n=2) are shown in FIG. 16. Cell kill IC50 values of the fully human, phage display derived, human Alya IgG1s conjugated to McMMAF showed no significant changes compared to IC50 values measured in the absence of sSEMA4A.

### Example 8. Expression Profile of SEMA4A, BCMA and CD22 on Normal and Haematological Tissue

### 8.1 Immunohistochemistry (IHC) Methods

SEMA4A: Sections of formalin-fixed, paraffin-embedded (FFPE) tissues were cut at 4 microns thickness. Using a Ventana Discovery autostainer, sections were deparaffinised, and antigen retrieval was performed in cell conditioning solution or CC1 (low pH) solution at 95° C. for 64 minutes. An antibody against SEMA4A (Atlas antibodies, HPA069136, rabbit polyclonal) was applied at 0.2 μg/ml concentration for 60 minutes. Detection was completed using the Ventana HQ kit, with 3,3′-Diaminobenzidine (DAB) as chromogen, and hematoxylin as counterstain.

A range of FFPE cell pellets comprising cell lines with known SEMA4A expression, including cells overexpressing its closest family member SEMA4B, were used to optimise the staining protocol and confirm its specificity. Normal human tissue with expected SEMA4A expression (tonsil, spleen) was used to confirm the suitability of the assay for FFPE tissue.

BCMA: Sections of formalin-fixed, paraffin-embedded (FFPE) tissues were cut at 4 microns thickness. Using a Ventana Discovery autostainer, sections were deparaffinised, and antigen retrieval was performed in CC2 (high pH) solution at 95° C. for 48 minutes. An antibody against BCMA (Cell Signaling Technology #88183S, rabbit monoclonal) was applied at 1.6 μg/ml concentration for 60 mins. Detection was completed using the Ventana HQ kit, with DAB as chromogen, and hematoxylin as counterstain. A range of FFPE cell pellets comprising cell lines with known BCMA expression were used to optimise the staining protocol and confirm its specificity.

CD22: Sections of formalin-fixed, paraffin-embedded (FFPE) tissues were cut at 4 microns thickness. Using a Leica Bond autostainer, sections were deparaffinised, and antigen retrieval was performed in ER2 (high pH) solution at 95° C. for 20 minutes. An antibody against CD22 (Cell Signaling Technology #98035, rabbit monoclonal) was applied at 0.2 μg/ml concentration for 30 mins. Detection was completed using the Leica Bond Refine kit, with DAB as chromogen, and hematoxylin as counterstain. A range of FFPE cell pellets comprising cell lines with known CD22 expression were used to optimise the staining protocol and confirm its specificity.

### 8.2 SEMA4A Expression Evaluation

The IHC protocol was applied to a collection of normal and tumour tissues, to evaluate prevalence and intensity of expression in different tumour types of interest. The sections were examined by a board-certified pathologist, and staining results were graded for proportion of positive staining tumour cells, as well as intensity of the staining in the majority of cells. Predominant localisation (membrane, cytoplasmic or nuclear) was also noted for each sample. In normal tissues, positive staining for human SEMA4A was observed in immune cells (consistent with B-cells as well as certain cells of myeloid/dendritic lineage), as well as in some cells of the brain.

In tumour tissue, a high prevalence of positive staining was observed in samples of MM (92% of samples with some positive tumour staining), DLBCL (100%), FL (100%) and AML (74%). Expression intensity was strongest overall in MM and DLBCL. The high prevalence of expression in these tumour indications, coupled with absent or low expression level in non-tumour tissues (apart from the brain) suggested that SEMA4A is a potentially valuable target for a specific antibody-based anti-tumour therapeutic approach.

SEMA4A expression in multiple myeloma and DLBCL was compared with that of other potential therapeutic targets for these tumours, namely BCMA for MM and CD22 for DLBCL. The same samples stained for SEMA4A were stained and scored for BCMA or CD22 expression, using the same scoring system. High expression was defined as a score ≥6. Results for MM are shown in Tables 13 and 14, and those for DLBCL in Tables 15 and 16.

All MM samples had some positivity for both SEMA4A and BCMA. Overall expression intensity was considered similar between SEMA4A and BCMA, though expression tended to be more localised to the membrane for SEMA4A, while it was frequently predominant in the cytoplasm for BCMA. There did not appear to be a strong association between the intensity of SEMA4A and BCMA expression in individual samples (Table 13).

TABLE 13
Comparative expression of SEMA4A and BCMA in 34 MM samples.

| | Total score (0-12) | | | High* expressors |
|---|---|---|---|---|
| Marker | Mean | Median | Range | (score ≥6) |
| SEMA4A | 5.1 | 4.5 | 1-10 | 16/34 (47%) |
| BCMA | 5.7 | 6 | 1-12 | 19/34 (56%) |

TABLE 14
Association between SEMA4A and BCMA expression in 34 MM samples.

| | | SEMA4A | |
|---|---|---|---|
| | | High | Low |
| BCMA | High | 12 (35%) | 7 (21%) |
| | Low | 4 (12%) | 11 (32%) |

For DLBCL samples, all cases had some positivity for SEMA4A, while 19/20 (95%) did for CD22. Overall expression intensity tended to be higher for SEMA4A than for CD22, with higher scores and a higher proportion of high expressor cases (Table 15). As for MM cases, SEMA4A expression was predominantly membrane, while CD22 was often more predominantly cytoplasmic. There did not appear to be an association between intensity of SEMA4A and CD22 expression in individual samples (Table 16).

TABLE 15
Comparative expression of SEMA4A and CD22 in 20 DLBCL samples

| | Total score (0-12) | | | High expressors |
|---|---|---|---|---|
| Marker | Mean | Median | Range | (score ≥6) |
| SEMA4A | 7.3 | 8 | 1-12 | 15/20 (75%) |
| CD22 | 5.6 | 6 | 1-12 | 11/20 (55%) |

TABLE 16
Association between SEMA4A and CD22 expression in 20 DLBCL
samples

| | | SEMA4a | |
|---|---|---|---|
| | | High | Low |
| CD22 | High | 9 (45%) | 2 (10%) |
| | Low (or −) | 6 (30%) | 3 (15%) |

### Example 9. In Vivo Models of Efficacy

NCI-H929, MM.1S, and MM.1R xenograft models of human MM (amongst others) are established in 4- to 6-week old female CB-17 mice (or appropriate species) (Envigo, Frederick, MD) by implanting 5×10⁶ cells (for example) subcutaneously in the flank. The JJN-3 xenograft model of human MM is established in female athymic nude mice (Envigo, Frederick, MD) by implanting 10×10⁶ cells (for example) subcutaneously in the flank. Tumour growth can be monitored over the course of the study, and tumour volume calculated as [length (mm)×width (mm)×width (mm)]/2. When the tumours reach 150-200 mm³, mice are assigned randomly into groups (n=5, 6, 8, or 10 animals per group for the NCI-H929, MM.1S, MM.1R, and JJN-3 models, amongst others, respectively) using (for example) the deterministic randomization method built into the Study Director software package (Studylog Systems, South San Francisco, CA) before administering a single intravenous injection of lead antibody or IgG isotype ADC control. The treatment information is not blinded during tumour measurement. Mouse body weight and tumour measurements are determined twice weekly for the duration of the study. Sample size estimates for 100 percent regression in tumour volume (compared to control) are calculated using nQuery version 4.0 (for example) (Statistical Solutions Ltd., Cork, Ireland, 2015). A two-group t-test of equal means is used with 80 percent power at a significance level of 0.05 (1-sided test).

### REFERENCES

1. Altschul S F, Gish W, Miller W, Myers E W, Lipman D J. Basic local alignment search tool. J Mol Biol. 1990 Oct. 5; 215 (3): 403-10. doi: 10.1016/S0022-2836 (05) 80360-2. PMID: 2231712.
2. Altschul S F, Madden T L, Schaffer A A, Zhang J, Zhang Z, Miller W, Lipman D J. Gapped BLAST and PSI-BLAST: a new generation of protein database search programs. Nucleic Acids Res. 1997 Sep. 1; 25 (17): 3389-402. doi: 10.1093/nar/25.17.3389. PMID: 9254694; PMCID: PMC146917.
3. Dimasi N, Fleming R, Zhong H, Bezabeh B, Kinneer K, Christie R J, Fazenbaker C, Wu H, Gao C. Efficient Preparation of Site-Specific Antibody-Drug Conjugates Using Cysteine Insertion. Mol Pharm. 2017 May 1; 14 (5): 1501-1516. doi: 10.1021/acs.molpharmaceut.6b00995. Epub 2017 Mar. 16. PMID: 28245132.
4. Doronina S O, Mendelsohn B A, Bovee T D, Cerveny C G, Alley S C, Meyer D L, Oflazoglu E, Toki B E, Sanderson R J, Zabinski R F, Wahl A F, Senter P D. Enhanced activity of monomethylauristatin F through monoclonal antibody delivery: effects of linker technology on efficacy and toxicity. Bioconjug Chem. 2006 January-February; 17 (1): 114-24. doi: 10.1021/bc0502917. PMID: 16417259.
5. Doronina S O, Toki B E, Torgov M Y, Mendelsohn B A, Cerveny C G, Chace D F, DeBlanc R L, Gearing R P, Bovee T D, Siegall C B, Francisco J A, Wahl A F, Meyer D L, Senter P D. Development of potent monoclonal antibody auristatin conjugates for cancer therapy. Nat Biotechnol. 2003 July; 21 (7): 778-84. doi: 10.1038/nbt832. Epub 2003 Jun. 1. PMID: 12778055.
6. Edgar R C. MUSCLE: multiple sequence alignment with high accuracy and high throughput. Nucleic Acids Res. 2004 Mar. 19; 32 (5): 1792-7. doi: 10.1093/nar/gkh340. PMID: 15034147; PMCID: PMC390337.
7. Gallagher D T, Mccullough C, Brinson R G, Ahn J, Marino J P, Dimasi N. Structure and Dynamics of a Site-Specific Labeled Fc Fragment with Altered Effector Functions. Pharmaceutics. 2019 Oct. 21; 11 (10): 546. doi: 10.3390/pharmaceutics11100546. PMID: 31640157; PMCID: PMC6835914.
8. Hawkins R E, Russell S J, Winter G. Selection of phage antibodies by binding affinity. Mimicking affinity maturation. J Mol Biol. 1992 Aug. 5; 226 (3): 889-96. doi: 10.1016/0022-2836 (92) 90639-2.
9. Hu S, Shively L, Raubitschek A, Sherman M, Williams L E, Wong J Y, Shively J E, Wu A M. Minibody: A novel engineered anti-carcinoembryonic antigen antibody fragment (single-chain Fv-CH3) which exhibits rapid, high-level targeting of xenografts. Cancer Res. 1996 Jul. 1; 56 (13): 3055-61. PMID: 8674062.
10. Janeway C A Jr, Travers P, Walport M, Shlomchik M J. Immunobiology: The Immune System in Health and Disease. 5th edition. New York: Garland Science; 2001.
11. Kabat E A, Wu T T. Identical V region amino acid sequences and segments of sequences in antibodies of different specificities. Relative contributions of VH and VL genes, minigenes, and complementarity-determining regions to binding of antibody-combining sites. J Immunol. 1991 Sep. 1; 147 (5): 1709-19. PMID: 1908882.
12. Kaur G, Hollingshead M, Holbeck S, Schauer-Vukasinović V, Camalier R F, Domling A, Agarwal S. Biological evaluation of tubulysin A: a potential anticancer and antiangiogenic natural product. Biochem J. 2006 Jun. 1; 396 (2): 235-42. doi: 10.1042/BJ20051735. PMID: 16489930; PMCID: PMC1462728.
13. Khalil M W, Sasse F, Lunsdorf H, Elnakady Y A, Reichenbach H. Mechanism of action of tubulysin, an antimitotic peptide from myxobacteria. Chembiochem. 2006 April; 7 (4): 678-83. doi: 10.1002/cbic.200500421. PMID: 16491500.
14. Kontermann R E. Dual targeting strategies with bispecific antibodies. MAbs. 2012 March-April; 4 (2): 182-97. doi: 10.4161/mabs.4.2.19000. Epub 2012 Mar. 1. PMID: 22453100; PMCID: PMC3361654.
15. Kupchan S M, Sneden A T, Branfman A R, Howie G A, Rebhun L I, Mclvor W E, Wang R W, Schnaitman T C. Structural requirements for antileukemic activity among the naturally occurring and semisynthetic maytansinoids. J Med Chem. 1978 January; 21 (1): 31-7. doi: 10.1021/jm00199a006. PMID: 563462.
16. Law C L, Gordon K A, Toki B E, Yamane A K, Hering M A, Cerveny C G, Petroziello J M, Ryan M C, Smith L, Simon R, Sauter G, Oflazoglu E, Doronina S O, Meyer D L, Francisco J A, Carter P, Senter P D, Copland J A, Wood C G, Wahl A F. Lymphocyte activation antigen CD70 expressed by renal cell carcinoma is a potential therapeutic target for anti-CD70 antibody-drug conjugates. Cancer Res. 2006 Feb. 15; 66 (4): 2328-37. doi: 10.1158/0008-5472.CAN-05-2883. PMID: 16489038.
17. Lefranc M P, Pommie C, Kaas Q, Duprat E, Bosc N, Guiraudou D, Jean C, Ruiz M, Da Piedade I, Rouard M, Foulquier E, Thouvenin V, Lefranc G. IMGT unique numbering for immunoglobulin and T cell receptor constant domains and Ig superfamily C-like domains. Dev Comp Immunol. 2005; 29 (3): 185-203. doi: 10.1016/j.dci.2004.07.003. PMID: 15572068.
18. Li J Y, Perry S R, Muniz-Medina V, Wang X, Wetzel L K, Rebelatto M C, Hinrichs M J, Bezabeh B Z, Fleming R L, Dimasi N, Feng H, Toader D, Yuan A Q, Xu L, Lin J, Gao C, Wu H, Dixit R, Osbourn J K, Coats S R. A Biparatopic HER2-Targeting Antibody-Drug Conjugate Induces Tumor Regression in Primary Models Refractory to or Ineligible for Therapy. Cancer Cell. 2016 Jan. 11; 29 (1): 117-29. doi: HER2-Targeted 10.1016/j.ccell.2015.12.008. Erratum in: Cancer Cell. 2019 Jun. 10; 35 (6): 948-949. PMID: 26766593.
19. Lloyd C, Lowe D, Edwards B, Welsh F, Dilks T, Hardman C, Vaughan T. Modelling the human immune response: performance of a 1011 human antibody repertoire against a broad panel of therapeutically relevant antigens. Protein Eng Des Sel. 2009 March; 22 (3): 159-68. doi: 10.1093/protein/gzn058. Epub 2008 Oct. 29.
20. Ma D, Hopf C E, Malewicz A D, Donovan G P, Senter P D, Goeckeler W F, Maddon P J, Olson W C. Potent antitumor activity of an auristatin-conjugated, fully human monoclonal antibody to prostate-specific membrane antigen. Clin Cancer Res. 2006 Apr. 15; 12 (8): 2591-6. doi: 10.1158/1078-0432.CCR-05-2107. PMID: 16638870.
21. Oflazoglu E, Kissler K M, Sievers E L, Grewal I S, Gerber H P. Combination of the anti-CD30-auristatin-E antibody-drug conjugate (SGN-35) with chemotherapy improves antitumour activity in Hodgkin lymphoma. Br J Haematol. 2008 July; 142 (1): 69-73. doi: 10.1111/j.1365-2141.2008.07146.x. Epub 2008 May 8. PMID: 18477046; PMCID: PMC2440525.
22. Oflazoglu E, Stone I J, Gordon K, Wood C G, Repasky E A, Grewal I S, Law C L, Gerber H P. Potent anticarcinoma activity of the humanized anti-CD70 antibody h1F6 conjugated to the tubulin inhibitor auristatin via an uncleavable linker. Clin Cancer Res. 2008 Oct. 1; 14 (19): 6171-80. doi: 10.1158/1078-0432.CCR-08-0916. Epub 2008 Sep. 22. PMID: 18809969.
23. Osbourn J K, Field A, Wilton J, Derbyshire E, Earnshaw J C, Jones P T, Allen D, McCafferty J. Generation of a panel of related human scFv antibodies with high affinities for human CEA. Immunotechnology. 1996 September; 2 (3): 181-96. doi: 10.1016/s1380-2933 (96) 00046-2.
24. Pearson W R, Lipman D J. Improved tools for biological sequence comparison. Proc Natl Acad Sci USA. 1988 April; 85 (8): 2444-8. doi: 10.1073/pnas.85.8.2444. PMID: 3162770; PMCID: PMC280013.
25. Persic L, Roberts A, Wilton J, Cattaneo A, Bradbury A, Hoogenboom H R. An integrated vector system for the eukaryotic expression of antibodies or their fragments after selection from phage display libraries. Gene. 1997 Mar. 10; 187 (1): 9-18. doi: 10.1016/s0378-1119 (96) 00628-2.
26. Remillard S, Rebhun L I, Howie G A, Kupchan S M. Antimitotic activity of the potent tumor inhibitor maytansine. Science. 1975 Sep. 19; 189 (4207): 1002-5. doi: 10.1126/science. 1241159. PMID: 1241159.
27. Sasse F, Steinmetz H, Heil J, Höfle G, Reichenbach H. Tubulysins, new cytostatic peptides from myxobacteria acting on microtubuli. Production, isolation, physico-chemical and biological properties. J Antibiot (Tokyo). 2000 September; 53 (9): 879-85. doi: 10.7164/antibiotics.53.879. PMID: 11099220.
28. Smith T F, Waterman M S. Identification of common molecular subsequences. J Mol Biol. 1981 Mar. 25; 147 (1): 195-7. doi: 10.1016/0022-2836 (81) 90087-5. PMID: 7265238.
29. Spiess C, Zhai Q, Carter P J. Alternative molecular formats and therapeutic applications for bispecific antibodies. Mol Immunol. 2015 October; 67 (2 Pt A): 95-106. doi: 10.1016/j.molimm.2015.01.003. Epub 2015 Jan. 27. PMID: 25637431.
30. Steinmetz H, Glaser N, Herdtweck E, Sasse F, Reichenbach H, Höfle G. Isolation, crystal and solution structure determination, and biosynthesis of tubulysins—powerful inhibitors of tubulin polymerization from myxobacteria. Angew Chem Int Ed Engl. 2004 Sep. 20; 43 (37): 4888-92. doi: 10.1002/anie.200460147. PMID: 15372566.
31. Tai Y T, Mayes P A, Acharya C, Zhong M Y, Cea M, Cagnetta A, Craigen J, Yates J, Gliddon L, Fieles W, Hoang B, Tunstead J, Christie A L, Kung A L, Richardson P, Munshi N C, Anderson K C. Novel anti-B-cell maturation antigen antibody-drug conjugate (GSK2857916) selectively induces killing of multiple myeloma. Blood. 2014 May 15; 123 (20): 3128-38. doi: 10.1182/blood-2013-10-535088. Epub 2014 Feb. 25. PMID: 24569262; PMCID: PMC4023420.
32. Thompson P, Fleming R, Bezabeh B, Huang F, Mao S, Chen C, Harper J, Zhong H, Gao X, Yu X Q, Hinrichs M J, Reed M, Kamal A, Strout P, Cho S, Woods R, Hollingsworth R E, Dixit R, Wu H, Gao C, Dimasi N. Rational design, biophysical and biological characterization of site-specific antibody-tubulysin conjugates with improved stability, efficacy and pharmacokinetics. J Control Release. 2016 Aug. 28; 236:100-16. doi: 10.1016/j.jconrel.2016.06.025. Epub 2016 Jun. 18.
33. Tse K F, Jeffers M, Pollack V A, McCabe D A, Shadish M L, Khramtsov N V, Hackett C S, Shenoy S G, Kuang B, Boldog F L, MacDougall JR, Rastelli L, Herrmann J, Gallo M, Gazit-Bornstein G, Senter P D, Meyer D L, Lichenstein H S, LaRochelle W J. CR011, a fully human monoclonal antibody-auristatin E conjugate, for the treatment of melanoma. Clin Cancer Res. 2006 Feb. 15; 12 (4): 1373-82. doi: 10.1158/1078-0432.CCR-05-2018. PMID: 16489096.
34. Vaughan T J, Williams A J, Pritchard K, Osbourn J K, Pope A R, Earnshaw J C, McCafferty J, Hodits R A, Wilton J, Johnson K S. Human antibodies with sub-nanomolar affinities isolated from a large non-immunized phage display library. Nat Biotechnol. 1996 March; 14 (3): 309-14. doi: 10.1038/nbt0396-309.
35. Xiao X, Douthwaite J A, Chen Y, Kemp B, Kidd S, Percival-Alwyn J, Smith A, Goode K, Swerdlow B, Lowe D, Wu H, Dall'Acqua W F, Chowdhury P S. A high-throughput platform for population reformatting and mammalian expression of phage display libraries to enable functional screening as full-length IgG. MAbs. 2017 August/September; 9 (6): 996-1006. doi: 10.1080/19420862.2017.1337617. Epub 2017 Jun. 14.
36. Anderson G S F, Ballester-Beltran J, Giotopoulos G, Guerrero J A, Surget S, Williamson J C, So T, Bloxham D, Aubareda A, Asby R, Walker I, Jenkinson L, Soilleux E J, Roy J P, Teodósio A, Ficken C, Officer-Jones L, Nasser S, Skerget S, Keats J J, Greaves P, Tai Y T, Anderson K C, MacFarlane M, Thaventhiran J E, Huntly B J P, Lehner P J, Chapman M A. Unbiased cell surface proteomics identifies SEMA4A as an effective immunotherapy target for myeloma. Blood. 2022 Apr. 21; 139 (16): 2471-2482. doi: 10.1182/blood.2021015161. PMID: 35134130.

Sequence Listing Information

| Seq ID NO: | Clone Name | Type |
|---|---|---|
| 1 | 5E3 | VH DNA |
| 2 | 5E3 | VH PRT |
| 3 | 5E3 | CDR1 PRT |
| 4 | 5E3 | CDR2 PRT |
| 5 | 5E3 | CDR3 PRT |
| 6 | 5E3 | FW1 PRT |
| 7 | 5E3 | FW2 PRT |
| 8 | 5E3 | FW3 PRT |
| 9 | 5E3 | FW4 PRT |
| 10 | 5E3 | VL DNA |
| 11 | 5E3 | VL PRT |
| 12 | 5E3 | CDR1 PRT |
| 13 | 5E3 | CDR2 PRT |
| 14 | 5E3 | CDR3 PRT |
| 15 | 5E3 | FW1 PRT |
| 16 | 5E3 | FW2 PRT |
| 17 | 5E3 | FW3 PRT |
| 18 | 5E3 | FW4 PRT |
| 19 | 5Hg1-5Lg1 | VH DNA |
| 20 | 5Hg1-5Lg1 | VH PRT |
| 21 | 5Hg1-5Lg1 | CDR1 PRT |
| 22 | 5Hg1-5Lg1 | CDR2 PRT |
| 23 | 5Hg1-5Lg1 | CDR3 PRT |
| 24 | 5Hg1-5Lg1 | FW1 PRT |
| 25 | 5Hg1-5Lg1 | FW2 PRT |
| 26 | 5Hg1-5Lg1 | FW3 PRT |
| 27 | 5Hg1-5Lg1 | FW4 PRT |
| 28 | 5Hg1-5Lg1 | VL DNA |
| 29 | 5Hg1-5Lg1 | VL PRT |
| 30 | 5Hg1-5Lg1 | CDR1 PRT |
| 31 | 5Hg1-5Lg1 | CDR2 PRT |
| 32 | 5Hg1-5Lg1 | CDR3 PRT |
| 33 | 5Hg1-5Lg1 | FW1 PRT |
| 34 | 5Hg1-5Lg1 | FW2 PRT |
| 35 | 5Hg1-5Lg1 | FW3 PRT |
| 36 | 5Hg1-5Lg1 | FW4 PRT |
| 37 | 5Hg1-5Lg2 | VH DNA |
| 38 | 5Hg1-5Lg2 | VH PRT |
| 39 | 5Hg1-5Lg2 | CDR1 PRT |
| 40 | 5Hg1-5Lg2 | CDR2 PRT |
| 41 | 5Hg1-5Lg2 | CDR3 PRT |
| 42 | 5Hg1-5Lg2 | FW1 PRT |
| 43 | 5Hg1-5Lg2 | FW2 PRT |
| 44 | 5Hg1-5Lg2 | FW3 PRT |
| 45 | 5Hg1-5Lg2 | FW4 PRT |
| 46 | 5Hg1-5Lg2 | VL DNA |
| 47 | 5Hg1-5Lg2 | VL PRT |
| 48 | 5Hg1-5Lg2 | CDR1 PRT |
| 49 | 5Hg1-5Lg2 | CDR2 PRT |
| 50 | 5Hg1-5Lg2 | CDR3 PRT |
| 51 | 5Hg1-5Lg2 | FW1 PRT |
| 52 | 5Hg1-5Lg2 | FW2 PRT |
| 53 | 5Hg1-5Lg2 | FW3 PRT |
| 54 | 5Hg1-5Lg2 | FW4 PRT |
| 55 | 5Hg2-5Lg1 | VH DNA |
| 56 | 5Hg2-5Lg1 | VH PRT |
| 57 | 5Hg2-5Lg1 | CDR1 PRT |
| 58 | 5Hg2-5Lg1 | CDR2 PRT |
| 59 | 5Hg2-5Lg1 | CDR3 PRT |
| 60 | 5Hg2-5Lg1 | FW1 PRT |
| 61 | 5Hg2-5Lg1 | FW2 PRT |
| 62 | 5Hg2-5Lg1 | FW3 PRT |
| 63 | 5Hg2-5Lg1 | FW4 PRT |
| 64 | 5Hg2-5Lg1 | VL DNA |
| 65 | 5Hg2-5Lg1 | VL PRT |
| 66 | 5Hg2-5Lg1 | CDR1 PRT |
| 67 | 5Hg2-5Lg1 | CDR2 PRT |
| 68 | 5Hg2-5Lg1 | CDR3 PRT |
| 69 | 5Hg2-5Lg1 | FW1 PRT |
| 70 | 5Hg2-5Lg1 | FW2 PRT |
| 71 | 5Hg2-5Lg1 | FW3 PRT |
| 72 | 5Hg2-5Lg1 | FW4 PRT |
| 73 | 5Hg2-5Lg2 | VH DNA |
| 74 | 5Hg2-5Lg2 | VH PRT |
| 75 | 5Hg2-5Lg2 | CDR1 PRT |
| 76 | 5Hg2-5Lg2 | CDR2 PRT |
| 77 | 5Hg2-5Lg2 | CDR3 PRT |
| 78 | 5Hg2-5Lg2 | FW1 PRT |
| 79 | 5Hg2-5Lg2 | FW2 PRT |
| 80 | 5Hg2-5Lg2 | FW3 PRT |
| 81 | 5Hg2-5Lg2 | FW4 PRT |
| 82 | 5Hg2-5Lg2 | VL DNA |
| 83 | 5Hg2-5Lg2 | VL PRT |
| 84 | 5Hg2-5Lg2 | CDR1 PRT |
| 85 | 5Hg2-5Lg2 | CDR2 PRT |
| 86 | 5Hg2-5Lg2 | CDR3 PRT |
| 87 | 5Hg2-5Lg2 | FW1 PRT |
| 88 | 5Hg2-5Lg2 | FW2 PRT |
| 89 | 5Hg2-5Lg2 | FW3 PRT |
| 90 | 5Hg2-5Lg2 | FW4 PRT |
| 91 | 5Hg2-5Lg3 | VH DNA |
| 92 | 5Hg2-5Lg3 | VH PRT |
| 93 | 5Hg2-5Lg3 | CDR1 PRT |
| 94 | 5Hg2-5Lg3 | CDR2 PRT |
| 95 | 5Hg2-5Lg3 | CDR3 PRT |
| 96 | 5Hg2-5Lg3 | FW1 PRT |
| 97 | 5Hg2-5Lg3 | FW2 PRT |
| 98 | 5Hg2-5Lg3 | FW3 PRT |
| 99 | 5Hg2-5Lg3 | FW4 PRT |
| 100 | 5Hg2-5Lg3 | VL DNA |
| 101 | 5Hg2-5Lg3 | VL PRT |
| 102 | 5Hg2-5Lg3 | CDR1 PRT |
| 103 | 5Hg2-5Lg3 | CDR2 PRT |
| 104 | 5Hg2-5Lg3 | CDR3 PRT |
| 105 | 5Hg2-5Lg3 | FW1 PRT |
| 106 | 5Hg2-5Lg3 | FW2 PRT |
| 107 | 5Hg2-5Lg3 | FW3 PRT |
| 108 | 5Hg2-5Lg3 | FW4 PRT |
| 109 | 5Hg2-5Lg5 | VH DNA |
| 110 | 5Hg2-5Lg5 | VH PRT |
| 111 | 5Hg2-5Lg5 | CDR1 PRT |
| 112 | 5Hg2-5Lg5 | CDR2 PRT |
| 113 | 5Hg2-5Lg5 | CDR3 PRT |
| 114 | 5Hg2-5Lg5 | FW1 PRT |
| 115 | 5Hg2-5Lg5 | FW2 PRT |
| 116 | 5Hg2-5Lg5 | FW3 PRT |
| 117 | 5Hg2-5Lg5 | FW4 PRT |
| 118 | 5Hg2-5Lg5 | VL DNA |
| 119 | 5Hg2-5Lg5 | VL PRT |
| 120 | 5Hg2-5Lg5 | CDR1 PRT |
| 121 | 5Hg2-5Lg5 | CDR2 PRT |
| 122 | 5Hg2-5Lg5 | CDR3 PRT |
| 123 | 5Hg2-5Lg5 | FW1 PRT |
| 124 | 5Hg2-5Lg5 | FW2 PRT |
| 125 | 5Hg2-5Lg5 | FW3 PRT |
| 126 | 5Hg2-5Lg5 | FW4 PRT |
| 127 | 5Hg2-5Lg6 | VH DNA |
| 128 | 5Hg2-5Lg6 | VH PRT |
| 129 | 5Hg2-5Lg6 | CDR1 PRT |
| 130 | 5Hg2-5Lg6 | CDR2 PRT |
| 131 | 5Hg2-5Lg6 | CDR3 PRT |
| 132 | 5Hg2-5Lg6 | FW1 PRT |
| 133 | 5Hg2-5Lg6 | FW2 PRT |
| 134 | 5Hg2-5Lg6 | FW3 PRT |
| 135 | 5Hg2-5Lg6 | FW4 PRT |
| 136 | 5Hg2-5Lg6 | VL DNA |
| 137 | 5Hg2-5Lg6 | VL PRT |
| 138 | 5Hg2-5Lg6 | CDR1 PRT |
| 139 | 5Hg2-5Lg6 | CDR2 PRT |
| 140 | 5Hg2-5Lg6 | CDR3 PRT |
| 141 | 5Hg2-5Lg6 | FW1 PRT |
| 142 | 5Hg2-5Lg6 | FW2 PRT |
| 143 | 5Hg2-5Lg6 | FW3 PRT |
| 144 | 5Hg2-5Lg6 | FW4 PRT |
| 145 | 5Hg4-5Lg1 | VH DNA |
| 146 | 5Hg4-5Lg1 | VH PRT |
| 147 | 5Hg4-5Lg1 | CDR1 PRT |
| 148 | 5Hg4-5Lg1 | CDR2 PRT |
| 149 | 5Hg4-5Lg1 | CDR3 PRT |
| 150 | 5Hg4-5Lg1 | FW1 PRT |
| 151 | 5Hg4-5Lg1 | FW2 PRT |
| 152 | 5Hg4-5Lg1 | FW3 PRT |
| 153 | 5Hg4-5Lg1 | FW4 PRT |
| 154 | 5Hg4-5Lg1 | VL DNA |
| 155 | 5Hg4-5Lg1 | VL PRT |
| 156 | 5Hg4-5Lg1 | CDR1 PRT |
| 157 | 5Hg4-5Lg1 | CDR2 PRT |
| 158 | 5Hg4-5Lg1 | CDR3 PRT |
| 159 | 5Hg4-5Lg1 | FW1 PRT |
| 160 | 5Hg4-5Lg1 | FW2 PRT |
| 161 | 5Hg4-5Lg1 | FW3 PRT |
| 162 | 5Hg4-5Lg1 | FW4 PRT |
| 163 | 5Hg4-5Lg2 | VH DNA |
| 164 | 5Hg4-5Lg2 | VH PRT |
| 165 | 5Hg4-5Lg2 | CDR1 PRT |
| 166 | 5Hg4-5Lg2 | CDR2 PRT |
| 167 | 5Hg4-5Lg2 | CDR3 PRT |
| 168 | 5Hg4-5Lg2 | FW1 PRT |
| 169 | 5Hg4-5Lg2 | FW2 PRT |
| 170 | 5Hg4-5Lg2 | FW3 PRT |
| 171 | 5Hg4-5Lg2 | FW4 PRT |
| 172 | 5Hg4-5Lg2 | VL DNA |
| 173 | 5Hg4-5Lg2 | VL PRT |
| 174 | 5Hg4-5Lg2 | CDR1 PRT |
| 175 | 5Hg4-5Lg2 | CDR2 PRT |
| 176 | 5Hg4-5Lg2 | CDR3 PRT |
| 177 | 5Hg4-5Lg2 | FW1 PRT |
| 178 | 5Hg4-5Lg2 | FW2 PRT |
| 179 | 5Hg4-5Lg2 | FW3 PRT |
| 180 | 5Hg4-5Lg2 | FW4 PRT |
| 181 | 5Hg4-5Lg4 | VH DNA |
| 182 | 5Hg4-5Lg4 | VH PRT |
| 183 | 5Hg4-5Lg4 | CDR1 PRT |
| 184 | 5Hg4-5Lg4 | CDR2 PRT |
| 185 | 5Hg4-5Lg4 | CDR3 PRT |
| 186 | 5Hg4-5Lg4 | FW1 PRT |
| 187 | 5Hg4-5Lg4 | FW2 PRT |
| 188 | 5Hg4-5Lg4 | FW3 PRT |
| 189 | 5Hg4-5Lg4 | FW4 PRT |
| 190 | 5Hg4-5Lg4 | VL DNA |
| 191 | 5Hg4-5Lg4 | VL PRT |
| 192 | 5Hg4-5Lg4 | CDR1 PRT |
| 193 | 5Hg4-5Lg4 | CDR2 PRT |
| 194 | 5Hg4-5Lg4 | CDR3 PRT |
| 195 | 5Hg4-5Lg4 | FW1 PRT |
| 196 | 5Hg4-5Lg4 | FW2 PRT |
| 197 | 5Hg4-5Lg4 | FW3 PRT |
| 198 | 5Hg4-5Lg4 | FW4 PRT |
| 199 | 5Hg4-5Lg5 | VH DNA |
| 200 | 5Hg4-5Lg5 | VH PRT |
| 201 | 5Hg4-5Lg5 | CDR1 PRT |
| 202 | 5Hg4-5Lg5 | CDR2 PRT |
| 203 | 5Hg4-5Lg5 | CDR3 PRT |
| 204 | 5Hg4-5Lg5 | FW1 PRT |
| 205 | 5Hg4-5Lg5 | FW2 PRT |
| 206 | 5Hg4-5Lg5 | FW3 PRT |
| 207 | 5Hg4-5Lg5 | FW4 PRT |
| 208 | 5Hg4-5Lg5 | VL DNA |
| 209 | 5Hg4-5Lg5 | VL PRT |
| 210 | 5Hg4-5Lg5 | CDR1 PRT |
| 211 | 5Hg4-5Lg5 | CDR2 PRT |
| 212 | 5Hg4-5Lg5 | CDR3 PRT |
| 213 | 5Hg4-5Lg5 | FW1 PRT |
| 214 | 5Hg4-5Lg5 | FW2 PRT |
| 215 | 5Hg4-5Lg5 | FW3 PRT |
| 216 | 5Hg4-5Lg5 | FW4 PRT |
| 217 | 5Hg5-5Lg2 | VH DNA |
| 218 | 5Hg5-5Lg2 | VH PRT |
| 219 | 5Hg5-5Lg2 | CDR1 PRT |
| 220 | 5Hg5-5Lg2 | CDR2 PRT |
| 221 | 5Hg5-5Lg2 | CDR3 PRT |
| 222 | 5Hg5-5Lg2 | FW1 PRT |
| 223 | 5Hg5-5Lg2 | FW2 PRT |
| 224 | 5Hg5-5Lg2 | FW3 PRT |
| 225 | 5Hg5-5Lg2 | FW4 PRT |
| 226 | 5Hg5-5Lg2 | VL DNA |
| 227 | 5Hg5-5Lg2 | VL PRT |
| 228 | 5Hg5-5Lg2 | CDR1 PRT |
| 229 | 5Hg5-5Lg2 | CDR2 PRT |
| 230 | 5Hg5-5Lg2 | CDR3 PRT |
| 231 | 5Hg5-5Lg2 | FW1 PRT |
| 232 | 5Hg5-5Lg2 | FW2 PRT |
| 233 | 5Hg5-5Lg2 | FW3 PRT |
| 234 | 5Hg5-5Lg2 | FW4 PRT |
| 235 | C0120903 | VH DNA |
| 236 | C0120903 | VH PRT |
| 237 | C0120903 | CDR1 PRT |
| 238 | C0120903 | CDR2 PRT |
| 239 | C0120903 | CDR3 PRT |
| 240 | C0120903 | FW1 PRT |
| 241 | C0120903 | FW2 PRT |
| 242 | C0120903 | FW3 PRT |
| 243 | C0120903 | FW4 PRT |
| 244 | C0120903 | VL DNA |
| 245 | C0120903 | VL PRT |
| 246 | C0120903 | CDR1 PRT |
| 247 | C0120903 | CDR2 PRT |
| 248 | C0120903 | CDR3 PRT |
| 249 | C0120903 | FW1 PRT |
| 250 | C0120903 | FW2 PRT |
| 251 | C0120903 | FW3 PRT |
| 252 | C0120903 | FW4 PRT |
| 253 | C0120904 | VH DNA |
| 254 | C0120904 | VH PRT |
| 255 | C0120904 | CDR1 PRT |
| 256 | C0120904 | CDR2 PRT |
| 257 | C0120904 | CDR3 PRT |
| 258 | C0120904 | FW1 PRT |
| 259 | C0120904 | FW2 PRT |
| 260 | C0120904 | FW3 PRT |
| 261 | C0120904 | FW4 PRT |
| 262 | C0120904 | VL DNA |
| 263 | C0120904 | VL PRT |
| 264 | C0120904 | CDR1 PRT |
| 265 | C0120904 | CDR2 PRT |
| 266 | C0120904 | CDR3 PRT |
| 267 | C0120904 | FW1 PRT |
| 268 | C0120904 | FW2 PRT |
| 269 | C0120904 | FW3 PRT |
| 270 | C0120904 | FW4 PRT |
| 271 | C0120905 | VH DNA |
| 272 | C0120905 | VH PRT |
| 273 | C0120905 | CDR1 PRT |
| 274 | C0120905 | CDR2 PRT |
| 275 | C0120905 | CDR3 PRT |
| 276 | C0120905 | FW1 PRT |
| 277 | C0120905 | FW2 PRT |
| 278 | C0120905 | FW3 PRT |
| 279 | C0120905 | FW4 PRT |
| 280 | C0120905 | VL DNA |
| 281 | C0120905 | VL PRT |
| 282 | C0120905 | CDR1 PRT |
| 283 | C0120905 | CDR2 PRT |
| 284 | C0120905 | CDR3 PRT |
| 285 | C0120905 | FW1 PRT |
| 286 | C0120905 | FW2 PRT |
| 287 | C0120905 | FW3 PRT |
| 288 | C0120905 | FW4 PRT |
| 289 | C0120906 | VH DNA |
| 290 | C0120906 | VH PRT |
| 291 | C0120906 | CDR1 PRT |
| 292 | C0120906 | CDR2 PRT |
| 293 | C0120906 | CDR3 PRT |
| 294 | C0120906 | FW1 PRT |
| 295 | C0120906 | FW2 PRT |
| 296 | C0120906 | FW3 PRT |
| 297 | C0120906 | FW4 PRT |
| 298 | C0120906 | VL DNA |
| 299 | C0120906 | VL PRT |
| 300 | C0120906 | CDR1 PRT |
| 301 | C0120906 | CDR2 PRT |
| 302 | C0120906 | CDR3 PRT |
| 303 | C0120906 | FW1 PRT |
| 304 | C0120906 | FW2 PRT |
| 305 | C0120906 | FW3 PRT |
| 306 | C0120906 | FW4 PRT |
| 307 | C0120910 | VH DNA |
| 308 | C0120910 | VH PRT |
| 309 | C0120910 | CDR1 PRT |
| 310 | C0120910 | CDR2 PRT |
| 311 | C0120910 | CDR3 PRT |
| 312 | C0120910 | FW1 PRT |
| 313 | C0120910 | FW2 PRT |
| 314 | C0120910 | FW3 PRT |
| 315 | C0120910 | FW4 PRT |
| 316 | C0120910 | VL DNA |
| 317 | C0120910 | VL PRT |
| 318 | C0120910 | CDR1 PRT |
| 319 | C0120910 | CDR2 PRT |
| 320 | C0120910 | CDR3 PRT |
| 321 | C0120910 | FW1 PRT |
| 322 | C0120910 | FW2 PRT |
| 323 | C0120910 | FW3 PRT |
| 324 | C0120910 | FW4 PRT |
| 325 | C0120913 | VH DNA |
| 326 | C0120913 | VH PRT |
| 327 | C0120913 | CDR1 PRT |
| 328 | C0120913 | CDR2 PRT |
| 329 | C0120913 | CDR3 PRT |
| 330 | C0120913 | FW1 PRT |
| 331 | C0120913 | FW2 PRT |
| 332 | C0120913 | FW3 PRT |
| 333 | C0120913 | FW4 PRT |
| 334 | C0120913 | VL DNA |
| 335 | C0120913 | VL PRT |
| 336 | C0120913 | CDR1 PRT |
| 337 | C0120913 | CDR2 PRT |
| 338 | C0120913 | CDR3 PRT |
| 339 | C0120913 | FW1 PRT |
| 340 | C0120913 | FW2 PRT |
| 341 | C0120913 | FW3 PRT |
| 342 | C0120913 | FW4 PRT |
| 343 | C0120914 | VH DNA |
| 344 | C0120914 | VH PRT |
| 345 | C0120914 | CDR1 PRT |
| 346 | C0120914 | CDR2 PRT |
| 347 | C0120914 | CDR3 PRT |
| 348 | C0120914 | FW1 PRT |
| 349 | C0120914 | FW2 PRT |
| 350 | C0120914 | FW3 PRT |
| 351 | C0120914 | FW4 PRT |
| 352 | C0120914 | VL DNA |
| 353 | C0120914 | VL PRT |
| 354 | C0120914 | CDR1 PRT |
| 355 | C0120914 | CDR2 PRT |
| 356 | C0120914 | CDR3 PRT |
| 357 | C0120914 | FW1 PRT |
| 358 | C0120914 | FW2 PRT |
| 359 | C0120914 | FW3 PRT |
| 360 | C0120914 | FW4 PRT |
| 361 | C0120917 | VH DNA |
| 362 | C0120917 | VH PRT |
| 363 | C0120917 | CDR1 PRT |
| 364 | C0120917 | CDR2 PRT |
| 365 | C0120917 | CDR3 PRT |
| 366 | C0120917 | FW1 PRT |
| 367 | C0120917 | FW2 PRT |
| 368 | C0120917 | FW3 PRT |
| 369 | C0120917 | FW4 PRT |
| 370 | C0120917 | VL DNA |
| 371 | C0120917 | VL PRT |
| 372 | C0120917 | CDR1 PRT |
| 373 | C0120917 | CDR2 PRT |
| 374 | C0120917 | CDR3 PRT |
| 375 | C0120917 | FW1 PRT |
| 376 | C0120917 | FW2 PRT |
| 377 | C0120917 | FW3 PRT |
| 378 | C0120917 | FW4 PRT |
| 379 | C0120918 | VH DNA |
| 380 | C0120918 | VH PRT |
| 381 | C0120918 | CDR1 PRT |
| 382 | C0120918 | CDR2 PRT |
| 383 | C0120918 | CDR3 PRT |
| 384 | C0120918 | FW1 PRT |
| 385 | C0120918 | FW2 PRT |
| 386 | C0120918 | FW3 PRT |
| 387 | C0120918 | FW4 PRT |
| 388 | C0120918 | VL DNA |
| 389 | C0120918 | VL PRT |
| 390 | C0120918 | CDR1 PRT |
| 391 | C0120918 | CDR2 PRT |
| 392 | C0120918 | CDR3 PRT |
| 393 | C0120918 | FW1 PRT |
| 394 | C0120918 | FW2 PRT |
| 395 | C0120918 | FW3 PRT |
| 396 | C0120918 | FW4 PRT |
| 397 | C0120919 | VH DNA |
| 398 | C0120919 | VH PRT |
| 399 | C0120919 | CDR1 PRT |
| 400 | C0120919 | CDR2 PRT |
| 401 | C0120919 | CDR3 PRT |
| 402 | C0120919 | FW1 PRT |
| 403 | C0120919 | FW2 PRT |
| 404 | C0120919 | FW3 PRT |
| 405 | C0120919 | FW4 PRT |
| 406 | C0120919 | VL DNA |
| 407 | C0120919 | VL PRT |
| 408 | C0120919 | CDR1 PRT |
| 409 | C0120919 | CDR2 PRT |
| 410 | C0120919 | CDR3 PRT |
| 411 | C0120919 | FW1 PRT |
| 412 | C0120919 | FW2 PRT |
| 413 | C0120919 | FW3 PRT |
| 414 | C0120919 | FW4 PRT |
| 415 | C0120920 | VH DNA |
| 416 | C0120920 | VH PRT |
| 417 | C0120920 | CDR1 PRT |
| 418 | C0120920 | CDR2 PRT |
| 419 | C0120920 | CDR3 PRT |
| 420 | C0120920 | FW1 PRT |
| 421 | C0120920 | FW2 PRT |
| 422 | C0120920 | FW3 PRT |
| 423 | C0120920 | FW4 PRT |
| 424 | C0120920 | VL DNA |
| 425 | C0120920 | VL PRT |
| 426 | C0120920 | CDR1 PRT |
| 427 | C0120920 | CDR2 PRT |
| 428 | C0120920 | CDR3 PRT |
| 429 | C0120920 | FW1 PRT |
| 430 | C0120920 | FW2 PRT |
| 431 | C0120920 | FW3 PRT |
| 432 | C0120920 | FW4 PRT |
| 433 | C0120921 | VH DNA |
| 434 | C0120921 | VH PRT |
| 435 | C0120921 | CDR1 PRT |
| 436 | C0120921 | CDR2 PRT |
| 437 | C0120921 | CDR3 PRT |
| 438 | C0120921 | FW1 PRT |
| 439 | C0120921 | FW2 PRT |
| 440 | C0120921 | FW3 PRT |
| 441 | C0120921 | FW4 PRT |
| 442 | C0120921 | VL DNA |
| 443 | C0120921 | VL PRT |
| 444 | C0120921 | CDR1 PRT |
| 445 | C0120921 | CDR2 PRT |
| 446 | C0120921 | CDR3 PRT |
| 447 | C0120921 | FW1 PRT |
| 448 | C0120921 | FW2 PRT |
| 449 | C0120921 | FW3 PRT |
| 450 | C0120921 | FW4 PRT |
| 451 | C0120922 | VH DNA |
| 452 | C0120922 | VH PRT |
| 453 | C0120922 | CDR1 PRT |
| 454 | C0120922 | CDR2 PRT |
| 455 | C0120922 | CDR3 PRT |
| 456 | C0120922 | FW1 PRT |
| 457 | C0120922 | FW2 PRT |
| 458 | C0120922 | FW3 PRT |
| 459 | C0120922 | FW4 PRT |
| 460 | C0120922 | VL DNA |
| 461 | C0120922 | VL PRT |
| 462 | C0120922 | CDR1 PRT |
| 463 | C0120922 | CDR2 PRT |
| 464 | C0120922 | CDR3 PRT |
| 465 | C0120922 | FW1 PRT |
| 466 | C0120922 | FW2 PRT |
| 467 | C0120922 | FW3 PRT |
| 468 | C0120922 | FW4 PRT |
| 469 | C0120923 | VH DNA |
| 470 | C0120923 | VH PRT |
| 471 | C0120923 | CDR1 PRT |
| 472 | C0120923 | CDR2 PRT |
| 473 | C0120923 | CDR3 PRT |
| 474 | C0120923 | FW1 PRT |
| 475 | C0120923 | FW2 PRT |
| 476 | C0120923 | FW3 PRT |
| 477 | C0120923 | FW4 PRT |
| 478 | C0120923 | VL DNA |
| 479 | C0120923 | VL PRT |
| 480 | C0120923 | CDR1 PRT |
| 481 | C0120923 | CDR2 PRT |
| 482 | C0120923 | CDR3 PRT |
| 483 | C0120923 | FW1 PRT |
| 484 | C0120923 | FW2 PRT |
| 485 | C0120923 | FW3 PRT |
| 486 | C0120923 | FW4 PRT |
| 487 | C0120924 | VH DNA |
| 488 | C0120924 | VH PRT |
| 489 | C0120924 | CDR1 PRT |
| 490 | C0120924 | CDR2 PRT |
| 491 | C0120924 | CDR3 PRT |
| 492 | C0120924 | FW1 PRT |
| 493 | C0120924 | FW2 PRT |
| 494 | C0120924 | FW3 PRT |
| 495 | C0120924 | FW4 PRT |
| 496 | C0120924 | VL DNA |
| 497 | C0120924 | VL PRT |
| 498 | C0120924 | CDR1 PRT |
| 499 | C0120924 | CDR2 PRT |
| 500 | C0120924 | CDR3 PRT |
| 501 | C0120924 | FW1 PRT |
| 502 | C0120924 | FW2 PRT |
| 503 | C0120924 | FW3 PRT |
| 504 | C0120924 | FW4 PRT |
| 505 | C0120925 | VH DNA |
| 506 | C0120925 | VH PRT |
| 507 | C0120925 | CDR1 PRT |
| 508 | C0120925 | CDR2 PRT |
| 509 | C0120925 | CDR3 PRT |
| 510 | C0120925 | FW1 PRT |
| 511 | C0120925 | FW2 PRT |
| 512 | C0120925 | FW3 PRT |
| 513 | C0120925 | FW4 PRT |
| 514 | C0120925 | VL DNA |
| 515 | C0120925 | VL PRT |
| 516 | C0120925 | CDR1 PRT |
| 517 | C0120925 | CDR2 PRT |
| 518 | C0120925 | CDR3 PRT |
| 519 | C0120925 | FW1 PRT |
| 520 | C0120925 | FW2 PRT |
| 521 | C0120925 | FW3 PRT |
| 522 | C0120925 | FW4 PRT |
| 523 | C0120926 | VH DNA |
| 524 | C0120926 | VH PRT |
| 525 | C0120926 | CDR1 PRT |
| 526 | C0120926 | CDR2 PRT |
| 527 | C0120926 | CDR3 PRT |
| 528 | C0120926 | FW1 PRT |
| 529 | C0120926 | FW2 PRT |
| 530 | C0120926 | FW3 PRT |
| 531 | C0120926 | FW4 PRT |
| 532 | C0120926 | VL DNA |
| 533 | C0120926 | VL PRT |
| 534 | C0120926 | CDR1 PRT |
| 535 | C0120926 | CDR2 PRT |
| 536 | C0120926 | CDR3 PRT |
| 537 | C0120926 | FW1 PRT |
| 538 | C0120926 | FW2 PRT |
| 539 | C0120926 | FW3 PRT |
| 540 | C0120926 | FW4 PRT |
| 541 | C0120927 | VH DNA |
| 542 | C0120927 | VH PRT |
| 543 | C0120927 | CDR1 PRT |
| 544 | C0120927 | CDR2 PRT |
| 545 | C0120927 | CDR3 PRT |
| 546 | C0120927 | FW1 PRT |
| 547 | C0120927 | FW2 PRT |
| 548 | C0120927 | FW3 PRT |
| 549 | C0120927 | FW4 PRT |
| 550 | C0120927 | VL DNA |
| 551 | C0120927 | VL PRT |
| 552 | C0120927 | CDR1 PRT |
| 553 | C0120927 | CDR2 PRT |
| 554 | C0120927 | CDR3 PRT |
| 555 | C0120927 | FW1 PRT |
| 556 | C0120927 | FW2 PRT |
| 557 | C0120927 | FW3 PRT |
| 558 | C0120927 | FW4 PRT |
| 559 | C0120928 | VH DNA |
| 560 | C0120928 | VH PRT |
| 561 | C0120928 | CDR1 PRT |
| 562 | C0120928 | CDR2 PRT |
| 563 | C0120928 | CDR3 PRT |
| 564 | C0120928 | FW1 PRT |
| 565 | C0120928 | FW2 PRT |
| 566 | C0120928 | FW3 PRT |
| 567 | C0120928 | FW4 PRT |
| 568 | C0120928 | VL DNA |
| 569 | C0120928 | VL PRT |
| 570 | C0120928 | CDR1 PRT |
| 571 | C0120928 | CDR2 PRT |
| 572 | C0120928 | CDR3 PRT |
| 573 | C0120928 | FW1 PRT |
| 574 | C0120928 | FW2 PRT |
| 575 | C0120928 | FW3 PRT |
| 576 | C0120928 | FW4 PRT |
| 577 | C0120929 | VH DNA |
| 578 | C0120929 | VH PRT |
| 579 | C0120929 | CDR1 PRT |
| 580 | C0120929 | CDR2 PRT |
| 581 | C0120929 | CDR3 PRT |
| 582 | C0120929 | FW1 PRT |
| 583 | C0120929 | FW2 PRT |
| 584 | C0120929 | FW3 PRT |
| 585 | C0120929 | FW4 PRT |
| 586 | C0120929 | VL DNA |
| 587 | C0120929 | VL PRT |
| 588 | C0120929 | CDR1 PRT |
| 589 | C0120929 | CDR2 PRT |
| 590 | C0120929 | CDR3 PRT |
| 591 | C0120929 | FW1 PRT |
| 592 | C0120929 | FW2 PRT |
| 593 | C0120929 | FW3 PRT |
| 594 | C0120929 | FW4 PRT |
| 595 | C0120930 | VH DNA |
| 596 | C0120930 | VH PRT |
| 597 | C0120930 | CDR1 PRT |
| 598 | C0120930 | CDR2 PRT |
| 599 | C0120930 | CDR3 PRT |
| 600 | C0120930 | FW1 PRT |
| 601 | C0120930 | FW2 PRT |
| 602 | C0120930 | FW3 PRT |
| 603 | C0120930 | FW4 PRT |
| 604 | C0120930 | VL DNA |
| 605 | C0120930 | VL PRT |
| 606 | C0120930 | CDR1 PRT |
| 607 | C0120930 | CDR2 PRT |
| 608 | C0120930 | CDR3 PRT |
| 609 | C0120930 | FW1 PRT |
| 610 | C0120930 | FW2 PRT |
| 611 | C0120930 | FW3 PRT |
| 612 | C0120930 | FW4 PRT |

```
recombinant human SEMA4A (UniProt ID Q9H3S1, isoform 1)
SEQ ID NO: 613
                10 20 30 40 50
MALPALGLDP WSLLGLFLFQ LLQLLLPTTT AGGGGQGPMP RVRYYAGDER
                60 70 80 90 100
RALSFFHQKG LQDFDTLLLS GDGNTLYVGA REAILALDIQ DPGVPRLKNM
              110 120 130 140 150
IPWPASDRKK SECAFKKKSN ETQCFNFIRV LVSYNVTHLY TCGTFAFSPA
              160 170 180 190 200
CTFIELQDSY LLPISEDKVM EGKGQSPFDP AHKHTAVLVD GMLYSGTMNN
              210 220 230 240 250
FLGSEPILMR TLGSQPVLKT DNFLRWLHHD ASFVAAIPST QVVYFFFEET
              260 270 280 290 300
ASEFDFFERL HTSRVARVCK NDVGGEKLLQ KKWTTFLKAQ LLCTQPGQLP
              310 320 330 340 350
FNVIRHAVLL PADSPTAPHI YAVFTSQWQV GGTRSSAVCA FSLLDIERVF
              360 370 380 390 400
KGKYKELNKE TSRWTTYRGP ETNPRPGSCS VGPSSDKALT FMKDHFLMDE
              410 420 430 440 450
QVVGTPLLVK SGVEYTRLAV ETAQGLDGHS HLVMYLGTTT GSLHKAVVSG
              460 470 480 490 500
DSSAHLVEEI QLFPDPEPVR NLQLAPTQGA VFVGFSGGVW RVPRANCSVY
              510 520 530 540 550
ESCVDCVLAR DPHCAWDPES RTCCLLSAPN LNSWKQDMER GNPEWACASG
              560 570 580 590 600
PMSRSLRPQS RPQIIKEVLA VPNSILELPC PHLSALASYY WSHGPAAVPE
              610 620 630 640 650
ASSTVYNGSL LLIVQDGVGG LYQCWATENG FSYPVISYWV DSQDQTLALD
              660 670 680 690 700
PELAGIPREH VKVPLTRVSG GAALAAQQSY WPHFVTVTVL FALVLSGALI
              710 720 730 740 750
ILVASPLRAL RARGKVQGCE TLRPGEKAPL SREQHLQSPK ECRTSASDVD
              760
ADNNCLGTEV A
recombinant cynomolgus SEMA4A (UniProt ID G7NV79)
SEQ ID NO: 614
                10 20 30 40 50
MALPALGLDP WSLLGLFLFQ LLQLLLPTTT AGGGGQGPMP RVRYYAGDER
                60 70 80 90 100
RALSFFHQKG LQDFDTLLLS GNGNTLYVGA REAILALDIQ DPGVPRLKNM
              110 120 130 140 150
IPWPASDRKK SECAFKKKSN ETQCFNFIRV LVSYNVTHLY TCGTFAFSPA
              160 170 180 190 200
CTFIELQDSH LLPILEDKVM EGKGQSPFDP AHKHTAVLVD GMLYSGTMNN
              210 220 230 240 250
FLGSEPILMR TLGSQPVLKT DNFLRWLHPD ASFVAAIPST QVVYFFFEET
              260 270 280 290 300
ASEFDFFERL HTSRVARVCK NDVGGEKLLQ RKWTTFLKAQ LLCTKPGQLP
              310 320 330 340 350
FNVIRHAVLL PADSPTAPHI YAVFTSQWQI GGTRSSAVCA FSLLDIERVF
              360 370 380 390 400
KGKYKELNKE TSRWTTYRGP ETNPRPGSCS VGPSSDKALT FMKDHFLMDE
              410 420 430 440 450
QVVGTPLLVK SGVEYTRLAV ETAQGLDGRS HLVMYLGTTT GSLHKAVVSG
              460 470 480 490 500
DSGAHLVEEI QLFPDPEPVR NLQLAPTQGA VFVGFSGGVW RVPRANCSVY
              510 520 530 540 550
ESCVDCVLAR DPHCAWDPES RTCCFLSAPT LNSWKQDMER GNPEWACASG
              560 570 580 590 600
PMSRSLRPQS RPQIIKEVLA VPNSFLELPC PHLSALASYY WSHGPAAVPE
              610 620 630 640 650
ASSTVYNGSL LLIVQDGVGG LYQCWATENG FSYPVVSYWV DSQDQSLALD
              660 670 680 690 700
PELAGIPREH VEVPLTRVSG GTALAAQRSY WPHFVTVTVL LALVLSGALI
              710 720 730 740 750
ILLASPLGAL RARGKVQGCE TLPPGEKAPL SREQHLQSPK ECRTSASDVD
              760
ADNCLGTEVA
recombinant mouse SEMA4A (UniProt ID Q62178)
SEQ ID NO: 615
                10 20 30 40 50
MALPSLGQDS WSLLRVFFFQ LFLLPSLPPA SGTGGQGPMP RVKYHAGDGH
                60 70 80 90 100
RALSFFQQKG LRDFDTLLLS DDGNTLYVGA REAVLALNIQ NPGIPRLKNM
              110 120 130 140 150
IPWPASERKK TECAFKKKSN ETQCFNFIRV LVSYNATHLY ACGTFAFSPA
              160 170 180 190 200
CTFIELQDSL LLPILIDKVM DGKGQSPFDP VHKHTAVLVD GMLYSGTMNN
              210 220 230 240 250
FLGSEPILMR TLGSQPVLKT DIFLRWLHAD ASFVAAIPST QVVYFFFEET
              260 270 280 290 300
ASEFDFFEEL YISRVAQVCK NDVGGEKLLQ KKWTTFLKAQ LLCAQPGQLP
              310 320 330 340 350
FNIIRHAVLL PADSPSVSRI YAVFTSQWQV GGTRSSAVCA FSLTDIERVF
              360 370 380 390 400
KGKYKELNKE TSRWTTYRGS EVSPRPGSCS MGPSSDKALT FMKDHFLMDE
              410 420 430 440 450
HVVGTPLLVK SGVEYTRLAV ESARGLDGSS HVVMYLGTST GSLHKAVVPQ
              460 470 480 490 500
DSSAYLVEEI QLSPDSEPVR NLQLAPAQGA VFAGFSGGIW RVPRANCSVY
              510 520 530 540 550
ESCVDCVLAR DPHCAWDPES RLCSLLSGST KPWKQDMERG NPEWVCTRGP
              560 570 580 590 600
MARSPRRQSP PQLIKEVLTV PNSILELPCP HLSALASYHW SHGRAKISEA
              610 620 630 640 650
SATVYNGSLL LLPQDGVGGL YQCVATENGY SYPVVSYWVD SQDQPLALDP
              660 670 680 690 700
ELAGVPRERV QVPLTRVGGG ASMAAQRSYW PHFLIVTVLL AIVLLGVLTL
              710 720 730 740 750
LLASPLGALR ARGKVQGCGM LPPREKAPLS RDQHLQPSKD HRTSASDVDA
              760
DNNHLGAEVA
```

| | |
|---|---|
| Human | MALPALGLDPWSLLGLFLFQLLQLLLPTTTAGGGGQGPMPRVRYYAGDERR |
| SEMA4A | ALSFFHQKGLQDFDTLLLSGDGNTLYVGAREAILALDIQDPGVPRLKNMIPWP |
| AviHis10 | ASDRKKSECAFKKKSNETQCFNFIRVLVSYNVTHLYTCGTFAFSPACTFIELQ |
| SEQ ID | DSYLLPISEDKVMEGKGQSPFDPAHKHTAVLVDGMLYSGTMNNFLGSEPILM |
| NO: 616 | RTLGSQPVLKTDNFLRWLHHDASFVAAIPSTQVVYFFFEETASEFDFFERLHT |
| | SRVARVCKNDVGGEKLLQKKWTTFLKAQLLCTQPGQLPFNVIRHAVLLPADS |
| | PTAPHIYAVFTSQWQVGGTRSSAVCAFSLLDIERVFKGKYKELNKETSRWTT |
| | YRGPETNPRPGSCSVGPSSDKALTFMKDHFLMDEQVVGTPLLVKSGVEYTR |
| | LAVETAQGLDGHSHLVMYLGTTTGSLHKAVVSGDSSAHLVEEIQLFPDPEPV |
| | RNLQLAPTQGAVFVGFSGGVWRVPRANCSVYESCVDCVLARDPHCAWDPE |
| | SRTCCLLSAPNLNSWKQDMERGNPEWACASGPMSRSLRPQSRPQIIKEVLA |
| | VPNSILELPCPHLSALASYYWSHGPAAVPEASSTVYNGSLLLIVQDGVGGLY |
| | QCWATENGFSYPVISYWVDSQDQTLALDPELAGIPREHVKVPLTRVSGGAA |
| | LAAQQSYWPHGTGGGSGGGSGLNDIFEAQKIEWHEHHHHHHHHHH |
| Mouse | MTGGQGPMPRVKYHAGDGHRALSFFQQKGLRDFDTLLLSDDGNTLYVGAR |
| SEMA4A | EAVLALNIQNPGIPRLKNMIPWPASERKKTECAFKKKSNETQCFNFIRVLVSY |
| AviHis10 | NATHLYACGTFAFSPACTFIELQDSLLLPILIDKVMDGKGQSPFDPVHKHTAV |
| SEQ ID | LVDGMLYSGTMNNFLGSEPILMRTLGSQPVLKTDIFLRWLHADASFVAAIPST |
| NO: 617 | QVVYFFFEETASEFDFFEELYISRVAQVCKNDVGGEKLLQKKWTTFLKAQLL |
| | CAQPGQLPFNIIRHAVLLPADSPSVSRIYAVFTSQWQVGGTRSSAVCAFSLT |
| | DIERVFKGKYKELNKETSRWTTYRGSEVSPRPGSCSMGPSSDKALTFMKDH |
| | FLMDEHVVGTPLLVKSGVEYTRLAVESARGLDGSSHVVMYLGTSTGSLHKA |
| | VVPQDSSAYLVEEIQLSPDSEPVRNLQLAPAQGAVFAGFSGGIWRVPRANC |
| | SVYESCVDCVLARDPHCAWDPESRLCSLLSGSTKPWKQDMERGNPEWVC |
| | TRGPMARSPRRQSPPQLIKEVLTVPNSILELPCPHLSALASYHWSHGRAKISE |
| | ASATVYNGSLLLLPQDGVGGLYQCVATENGYSYPVVSYWVDSQDQPLALDP |
| | ELAGVPRERVQVPLTRVGGGASMAAQRSYWPHGTGGGSGGGSGLNDIFEA |
| | QKIEWHEHHHHHHHHHH |
| Cyno | MGGGGQGPMPRVRYYAGDERRALSFFHQKGLQDFDTLLLSGNGNTLYVGA |
| SEMA4A | REAILALDIQDPGVPRLKNMIPWPASDRKKSECAFKKKSNETQCFNFIRVLVS |
| AviHis10 | YNVTHLYTCGTFAFSPACTFIELQDSHLLPILEDKVMEGKGQSPFDPAHKHTA |
| SEQ ID | VLVDGMLYSGTMNNFLGSEPILMRTLGSQPVLKTDNFLRWLHPDASFVAAIP |
| NO: 618 | STQVVYFFFEETASEFDFFERLHTSRVARVCKNDVGGEKLLQRKWTTFLKA |
| | QLLCTKPGQLPFNVIRHAVLLPADSPTAPHIYAVFTSQWQIGGTRSSAVCAFS |
| | LLDIERVFKGKYKELNKETSRWTTYRGPETNPRPGSCSVGPSSDKALTFMK |
| | DHFLMDEQVVGTPLLVKSGVEYTRLAVETAQGLDGRSHLVMYLGTTTGSLH |
| | KAVVSGDSGAHLVEEIQLFPDPEPVRNLQLAPTQGAVFVGFSGGVWRVPRA |
| | NCSVYESCVDCVLAQDPHCAWDPESRTCCFLSAPTLNSWKQDMERGNPE |
| | WACASGPMSRSLRPQSRPQIIKEVLAVPNSFLELPCPHLSALASYYWSHGPA |
| | AVPEASSTVYNGSLLLIVQDGVGGLYQCWATENGFSYPVVSYWVDSQDQSL |
| | ALDPELAGIPREHVEVPLTRVSGGTALAAQRSGTGGGSGGGSGLNDIFEAQ |
| | KIEWHEHHHHHHHHHH |

1. An antibody-drug conjugate (ADC) comprising an anti-human-SEMA4A human or humanised antibody or an antigen-binding fragment thereof, a linker and a cytotoxin.

2. An ADC according to claim 1, wherein the antibody comprises a V_{H} comprising HCDR1, HCDR2 and HCDR3 and a V_{L} comprising LCDR1, LCDR2 and LCDR3, wherein the CDRs are selected from the CDRs of:
(a) 5E3 (SEQ ID NOS.: 3, 4, 5, 12, 13, 14);
(b) 5Hg1-5Lg1 (SEQ ID NOS.: 21, 22, 23, 30, 31, 32);
(c) 5Hg1-5Lg2 (SEQ ID NOS.: 39, 40, 41, 48, 49, 50);
(d) 5Hg2-5Lg1 (SEQ ID NOS.: 57, 58, 59, 66, 67, 68);
(e) 5Hg2-5Lg2 (SEQ ID NOS.: 75, 76, 77, 84, 85, 86);
(f) 5Hg2-5Lg3 (SEQ ID NOS.: 93, 94, 95, 102, 103, 104);
(g) 5Hg2-5Lg5 (SEQ ID NOS.: 111, 112, 113, 120, 121, 122);
(h) 5Hg2-5Lg6 (SEQ ID NOS.: 129, 130, 131, 138, 139, 140);
(i) 5Hg4-5Lg1 (SEQ ID NOS.: 147, 148, 149, 156, 157, 158);
(j) 5Hg4-5Lg2 (SEQ ID NOS.: 165, 166, 167, 174, 175, 176);
(k) 5Hg4-5Lg4 (SEQ ID NOS.: 183, 184, 185, 192, 193, 194);
(l) 5Hg4-5Lg5 (SEQ ID NOS.: 201, 202, 203, 210, 211, 212);
(m) 5Hg5-5Lg2 (SEQ ID NOS.: 219, 220, 221, 228, 229, 230);
(n) C0120903 (SEQ ID NOS.: 237, 238, 239, 246, 247, 248);
(o) C0120904 (SEQ ID NOS.: 255, 256, 257, 264, 265, 266);
(p) C0120905 (SEQ ID NOS.: 273, 274, 275, 282, 283, 284);
(q) C0120906 (SEQ ID NOS.: 291, 292, 293, 300, 301, 302);
(r) C0120910 (SEQ ID NOS.: 309, 310, 311, 318, 319, 320);
(s) C0120913 (SEQ ID NOS.: 327, 328, 329, 336, 337, 338);
(t) C0120914 (SEQ ID NOS.: 345, 346, 347, 354, 355, 356);
(u) C0120917 (SEQ ID NOS.: 363, 364, 365, 372, 373, 374);
(v) C0120918 (SEQ ID NOS.: 381, 382, 383, 390, 391, 392);
(w) C0120919 (SEQ ID NOS.: 399, 400, 401, 408, 409, 410);
(x) C0120920 (SEQ ID NOS.: 417, 418, 419, 426, 427, 428);
(y) C0120921 (SEQ ID NOS.: 435, 436, 437, 444, 445, 446);
(z) C0120922 (SEQ ID NOS.: 453, 453, 455, 462, 463, 464);
(aa) C0120923 (SEQ ID NOS.: 471, 472, 473, 480, 481, 482);
(bb) C0120924 (SEQ ID NOS.: 489, 490, 491, 498, 499, 500);
(cc) C0120925 (SEQ ID NOS.: 507, 508, 509, 516, 517, 518);
(dd) C0120926 (SEQ ID NOS.: 525, 526, 527, 534, 535, 536);
(ee) C0120927 (SEQ ID NOS.: 543, 544, 545, 552, 553, 554);
(ff) C0120928 (SEQ ID NOS.: 561, 562, 563, 570, 571, 572);
(gg) C0120929 (SEQ ID NOS.: 579, 580, 581, 588, 589, 590) and
(hh) C0120930 (SEQ ID NOS.: 597, 598, 599, 606, 607, 608)
wherein the CDRs are defined according by Kabat nomenclature.

3. The ADC according to claim 1, wherein the antibody comprises a V_{H} and a V_{L} selected from the V_{H} and V_{L} of:
a. 5Hg1-5Lg1 (SEQ ID NOS.: 20 and 29);
b. 5Hg1-5Lg2 (SEQ ID NOS.: 38 and 47);
c. 5Hg2-5Lg1 (SEQ ID NOS.: 56 and 65);
d. 5Hg2-5Lg2 (SEQ ID NOS.: 74 and 83);
e. 5Hg2-5Lg3 (SEQ ID NOS.: 92 and 101);
f. 5Hg2-5Lg5 (SEQ ID NOS.: 110 and 119);
g. 5Hg2-5Lg6 (SEQ ID NOS.: 128 and 137);
h. 5Hg4-5Lg1 (SEQ ID NOS.: 146 and 155);
i. 5Hg4-5Lg2 (SEQ ID NOS.: 164 and 173);
j. 5Hg4-5Lg4 (SEQ ID NOS.: 182 and 191);
k. 5Hg4-5Lg5 (SEQ ID NOS.: 200 and 209);
l. 5Hg5-5Lg2 (SEQ ID NOS.: 218 and 227);
m. C0120903 (SEQ ID NOS.: 236 and 245);
n. C0120904 (SEQ ID NOS.: 254 and 263);
o. C0120905 (SEQ ID NOS.: 272 and 281);
p. C0120906 (SEQ ID NOS.: 290 and 299);
q. C0120910 (SEQ ID NOS.: 308 and 317);
r. C0120913 (SEQ ID NOS.: 326 and 335);
s. C0120914 (SEQ ID NOS.: 344 and 353);
t. C0120917 (SEQ ID NOS.: 362 and 371);
u. C0120918 (SEQ ID NOS.: 380 and 389);
v. C0120919 (SEQ ID NOS.: 398 and 407);
w. C0120920 (SEQ ID NOS.: 416 and 425);
x. C0120921 (SEQ ID NOS.: 434 and 443);
y. C0120922 (SEQ ID NOS.: 452 and 461);
z. C0120923 (SEQ ID NOS.: 470 and 479);
aa. C0120924 (SEQ ID NOS.: 488 and 497);
bb. C0120925 (SEQ ID NOS.: 506 and 515);
cc. C0120926 (SEQ ID NOS.: 524 and 533);
dd. C0120927 (SEQ ID NOS.: 542 and 551);
ee. C0120928 (SEQ ID NOS.: 560 and 569);
ff. C0120929 (SEQ ID NOS.: 578 and 587); and
gg. C0120930 (SEQ ID NOS.: 596 and 605);
wherein the sequences are defined by Kabat nomenclature.

4. The ADC according to claim 1 wherein the cytotoxin is a microtubule inhibitor (e.g., a maytansinoid or an auristatin).

5. The ADC according to claim 1 wherein the cytotoxin is selected from monomethyl auristatin F (MMAF), monomethyl auristatin E (MMAE, vedotin) and mertansine (DM1 or as emtansine with an SMCC linker).

6. The ADC according to claim 1 wherein the linker is a non-cleavable linker or is a cleavable linker.

7. The ADC according to claim 1 wherein the cytotoxin is monomethyl auristatin E (MMAE) and the linker is a mc-vcPAB linker (malemide-based linker, cysteine linked) or the cytotoxin is mertansine (DM1) and the linker is a SMCC linker (NHS-ester based, lysine).

8. (canceled)

9. (canceled)

10. A human or humanised anti-human SEMA4A antibody comprising a V_{H} comprising HCDR1, HCDR2 and HCDR3 and a V_{L} comprising LCDR1, LCDR2 and LCDR3, wherein the CDRs are selected from the CDRs of:
(a) 5E3 (SEQ ID NOS.: 3, 4, 5, 12, 13, 14);
(b) 5Hg1-5Lg1 (SEQ ID NOS.: 21, 22, 23, 30, 31, 32);
(c) 5Hg1-5Lg2 (SEQ ID NOS.: 39, 40, 41, 48, 49, 50);
(d) 5Hg2-5Lg1 (SEQ ID NOS.: 57, 58, 59, 66, 67, 68);
(e) 5Hg2-5Lg2 (SEQ ID NOS.: 75, 76, 77, 84, 85, 86);
(f) 5Hg2-5Lg3 (SEQ ID NOS.: 93, 94, 95, 102, 103, 104);
(g) 5Hg2-5Lg5 (SEQ ID NOS.: 111, 112, 113, 120, 121, 122);
(h) 5Hg2-5Lg6 (SEQ ID NOS.: 129, 130, 131, 138, 139, 140);
(i) 5Hg4-5Lg1 (SEQ ID NOS.: 147, 148, 149, 156, 157, 158);
(j) 5Hg4-5Lg2 (SEQ ID NOS.: 165, 166, 167, 174, 175, 176);
(k) 5Hg4-5Lg4 (SEQ ID NOS.: 183, 184, 185, 192, 193, 194);
(l) 5Hg4-5Lg5 (SEQ ID NOS.: 201, 202, 203, 210, 211, 212);
(m) 5Hg5-5Lg2 (SEQ ID NOS.: 219, 220, 221, 228, 229, 230);
(n) C0120903 (SEQ ID NOS.: 237, 238, 239, 246, 247, 248);
(o) C0120904 (SEQ ID NOS.: 255, 256, 257, 264, 265, 266);
(p) C0120905 (SEQ ID NOS.: 273, 274, 275, 282, 283, 284);
(q) C0120906 (SEQ ID NOS.: 291, 292, 293, 300, 301, 302);
(r) C0120910 (SEQ ID NOS.: 309, 310, 311, 318, 319, 320);
(s) C0120913 (SEQ ID NOS.: 327, 328, 329, 336, 337, 338);
(t) C0120914 (SEQ ID NOS.: 345, 346, 347, 354, 355, 356);
(u) C0120917 (SEQ ID NOS.: 363, 364, 365, 372, 373, 374);
(v) C0120918 (SEQ ID NOS.: 381, 382, 383, 390, 391, 392);
(w) C0120919 (SEQ ID NOS.: 399, 400, 401, 408, 409, 410);
(x) C0120920 (SEQ ID NOS.: 417, 418, 419, 426, 427, 428);
(y) C0120921 (SEQ ID NOS.: 435, 436, 437, 444, 445, 446);
(z) C0120922 (SEQ ID NOS.: 453, 454, 455, 462, 463, 464);
(aa) C0120923 (SEQ ID NOS.: 471, 472, 473, 480, 481, 482);
(bb) C0120924 (SEQ ID NOS.: 489, 490, 491, 498, 499, 500);
(cc) C0120925 (SEQ ID NOS.: 507, 508, 509, 516, 517, 518);
(dd) C0120926 (SEQ ID NOS.: 525, 526, 527, 534, 535, 536);
(ee) C0120927 (SEQ ID NOS.: 543, 544, 545, 552, 553, 554);
(ff) C0120928 (SEQ ID NOS.: 561, 562, 563, 570, 571, 572);
(gg) C0120929 (SEQ ID NOS.: 579, 580, 581, 588, 589, 590) and
(hh) C0120930 (SEQ ID NOS: 597, 598, 599, 606, 607, 608)
wherein the sequence of the CDRs is defined by Kabat nomenclature.

11. The antibody according to claim 10 wherein the antibody comprises a V_{H} and a V_{L} selected from the V_{H} and V_{L} of:
(a) 5Hg1-5Lg1 (SEQ ID NOS.: 20 and 29);
(b) 5Hg1-5Lg2 (SEQ ID NOS.: 38 and 47);
(c) 5Hg2-5Lg1 (SEQ ID NOS.: 56 and 65);
(d) 5Hg2-5Lg2 (SEQ ID NOS.: 74 and 83);
(e) 5Hg2-5Lg3 (SEQ ID NOS.: 92 and 101);
(f) 5Hg2-5Lg5 (SEQ ID NOS.: 110 and 119);
(g) 5Hg2-5Lg6 (SEQ ID NOS.: 128 and 137);
(h) 5Hg4-5Lg1 (SEQ ID NOS.: 146 and 155);
(i) 5Hg4-5Lg2 (SEQ ID NOS.: 164 and 173);
(j) 5Hg4-5Lg4 (SEQ ID NOS.: 182 and 191);
(k) 5Hg4-5Lg5 (SEQ ID NOS.: 200 and 209);
(l) 5Hg5-5Lg2 (SEQ ID NOS.: 218 and 227);
(m) C0120903 (SEQ ID NOS.: 236 and 245);
(n) C0120904 (SEQ ID NOS.: 254 and 263);
(o) C0120905 (SEQ ID NOS.: 272 and 281);
(p) C0120906 (SEQ ID NOS.: 290 and 299);
(q) C0120910 (SEQ ID NOS.: 308 and 317);
(r) C0120913 (SEQ ID NOS.: 326 and 335);
(s) C0120914 (SEQ ID NOS.: 344 and 353);
(t) C0120917 (SEQ ID NOS.: 362 and 371);
(u) C0120918 (SEQ ID NOS.: 380 and 389);
(v) C0120919 (SEQ ID NOS.: 398 and 407);
(w) C0120920 (SEQ ID NOS.: 416 and 425);
(x) C0120921 (SEQ ID NOS.: 434 and 443);
(y) C0120922 (SEQ ID NOS.: 452 and 461);
(z) C0120923 (SEQ ID NOS.: 470 and 479);
(aa) C0120924 (SEQ ID NOS.: 488 and 497);
(bb) C0120925 (SEQ ID NOS.: 506 and 515);
(cc) C0120926 (SEQ ID NOS.: 524 and 533);
(dd) C0120927 (SEQ ID NOS.: 542 and 551);
(ee) C0120928 (SEQ ID NOS.: 560 and 569);
(ff) C0120929 (SEQ ID NOS.: 578 and 587) and
(gg) C0120930 (SEQ ID NOS.: 596 and 605);
wherein the V_{H} and V_{L} sequences are defined by Kabat nomenclature

12. A chimeric antigen receptor (CAR) comprising an antigen-binding domain of the monoclonal antibody of claim 10 linked to a T-cell activation moiety.

13. A CAR of claim 12, wherein the antigen-binding domain comprises a single chain Fv (scFv) fragment of the anti-human SEMA4A antibody.

14. The ADC according to claim 1 further comprising a diluent.

15. (canceled)

16. A method of treatment of a haematological cancer, comprising administration of an ADC according to claim 1 to a subject in need thereof.

17. A method for manufacture of an ADC according to claim 1 comprising conjugation of the antibody to a cytotoxin via a linker.

18. An isolated recombinant DNA or RNA sequence comprising a sequence encoding an isolated antibody or antigen-binding fragment thereof, according to claim 10.

19. An isolated recombinant DNA sequence of claim 18 which is an expression vector.

20. (canceled)

21. An isolated recombinant DNA sequence of claim 18 encoding an antibody or antigen-binding fragment thereof under control of a promoter.

22. A host cell comprising a DNA or RNA sequence according to claim 18.

23. A host cell of claim 22 capable of expressing an isolated antibody or antigen-binding fragment thereof.

24. A method of making an isolated antibody or antigen-binding fragment thereof, comprising culturing a host cell according to claim 22 in conditions suitable for expression of the isolated antibody or antigen-binding fragment thereof.


SEQUENCE LISTING (618 sequences)

SEQ ID NO: 1 - DNA, 363 bp, Mus musculus
  misc_feature 1..363: note = 5E3
  source 1..363: mol_type = other DNA
caggttcagctgcaacagcctggcgccgaacttgttatgcctggcgctagcgtgaagatgagctgcaaggccagcggctacaccttcaccgactactggatgcactgggtcaagcagaggcctggacagggactcgaatggatcggcgccatcgataccagcgacagctacaccagctacaaccagaagttcaagggcaaagccacactgaccgtggacgagagcagcagcacagcctacatgcagctgagcagcctgaccagcgaagatagcgccgtgtactactgcgccagagactacggcaacttctactactacgccatggattactggggccagggcaccagcgtgacagtctcttct

SEQ ID NO: 2 - protein, 121 aa, Mus musculus
  REGION 1..121: note = 5E3
QVQLQQPGAELVMPGASVKMSCKASGYTFTDYWMHWVKQRPGQGLEWIGAIDTSDSYTSYNQKFKGKATLTVDESSSTAYMQLSSLTSEDSAVYYCARDYGNFYYYAMDYWGQGTSVTVSS

SEQ ID NO: 3 - protein, 5 aa, Mus musculus
  REGION 1..5: note = 5E3
DYWMH

SEQ ID NO: 4 - protein, 17 aa, Mus musculus
  REGION 1..17: note = 5E3
AIDTSDSYTSYNQKFKG

SEQ ID NO: 5 - protein, 12 aa, Mus musculus
  REGION 1..12: note = 5E3
DYGNFYYYAMDY

SEQ ID NO: 6 - protein, 30 aa, Mus musculus
  REGION 1..30: note = 5E3
QVQLQQPGAELVMPGASVKMSCKASGYTFT

SEQ ID NO: 7 - protein, 14 aa, Mus musculus
  REGION 1..14: note = 5E3
WVKQRPGQGLEWIG

SEQ ID NO: 8 - protein, 32 aa, Mus musculus
  REGION 1..32: note = 5E3
KATLTVDESSSTAYMQLSSLTSEDSAVYYCAR

SEQ ID NO: 9 - protein, 11 aa, Mus musculus
  REGION 1..11: note = 5E3
WGQGTSVTVSS

SEQ ID NO: 10 - DNA, 327 bp, Mus musculus
  misc_feature 1..327: note = 5E3
  source 1..327: mol_type = other DNA
caggccgtggtcacacaagaaagcgccctgacaacaagccctggcgagacagtgaccctgacctgcagatcttctacaggcgccgtgaccaccagcaactacgccaattgggtgcaagagaagcccgaccacctgttcacaggactgatcggcggcaccaacaatagagcacctggcgtgccagccagattcagcggatctctgatcggagacaaggccgcactgacaatcacaggcgcccagacagaggacgaggccatctacttttgcgccctgtggtacagcaaccactgggttttcggcggaggcaccaagctgacagttctg

SEQ ID NO: 11 - protein, 109 aa, Mus musculus
  REGION 1..109: note = 5E3
QAVVTQESALTTSPGETVTLTCRSSTGAVTTSNYANWVQEKPDHLFTGLIGGTNNRAPGVPARFSGSLIGDKAALTITGAQTEDEAIYFCALWYSNHWVFGGGTKLTVL

SEQ ID NO: 12 - protein, 14 aa, Mus musculus
  REGION 1..14: note = 5E3
RSSTGAVTTSNYAN

SEQ ID NO: 13 - protein, 7 aa, Mus musculus
  REGION 1..7: note = 5E3
GTNNRAP

SEQ ID NO: 14 - protein, 9 aa, Mus musculus
  REGION 1..9: note = 5E3
ALWYSNHWV

SEQ ID NO: 15 - protein, 22 aa, Mus musculus
  REGION 1..22: note = 5E3
QAVVTQESALTTSPGETVTLTC

SEQ ID NO: 16 - protein, 15 aa, Mus musculus
  REGION 1..15: note = 5E3
WVQEKPDHLFTGLIG

SEQ ID NO: 17 - protein, 32 aa, Mus musculus
  REGION 1..32: note = 5E3
GVPARFSGSLIGDKAALTITGAQTEDEAIYFC

SEQ ID NO: 18 - protein, 10 aa, Mus musculus
  REGION 1..10: note = 5E3
FGGGTKLTVL

SEQ ID NO: 19 - DNA, 363 bp, synthetic construct
  misc_feature 1..363: note = 5Hg1-5Lg1
  source 1..363: mol_type = other DNA
caagtgcagctggtgcagagcggcgccgaggtgaagaagcccggcgctagcgtgaaggtgagctgcaaggctagcggctacaccttcaccgactactggatgcactgggtgagacaagcccccggccaaggcctggagtggatgggcgccatcgacacaagcgacagctacacaagctacaatcagaagttcaagggcagagtgaccatgacaagagacacaagcacaagcaccgtgtacatggagctgagcagcctgagaagcgaggacaccgccgtgtactactgcgctagagactacggcaacttctactactacgccatggactactggggccaaggcaccctggtcaccgtctcctca

SEQ ID NO: 20 - protein, 121 aa, synthetic construct
  REGION 1..121: note = 5Hg1-5Lg1
QVQLVQSGAEVKKPGASVKVSCKASGYTFTDYWMHWVRQAPGQGLEWMGAIDTSDSYTSYNQKFKGRVTMTRDTSTSTVYMELSSLRSEDTAVYYCARDYGNFYYYAMDYWGQGTLVTVSS

SEQ ID NO: 21 - protein, 5 aa, synthetic construct
  REGION 1..5: note = 5Hg1-5Lg1
DYWMH

SEQ ID NO: 22 - protein, 17 aa, synthetic construct
  REGION 1..17: note = 5Hg1-5Lg1
AIDTSDSYTSYNQKFKG

SEQ ID NO: 23 - protein, 12 aa, synthetic construct
  REGION 1..12: note = 5Hg1-5Lg1
DYGNFYYYAMDY

SEQ ID NO: 24 - protein, 30 aa, synthetic construct
  REGION 1..30: note = 5Hg1-5Lg1
QVQLVQSGAEVKKPGASVKVSCKASGYTFT

SEQ ID NO: 25 - protein, 14 aa, synthetic construct
  REGION 1..14: note = 5Hg1-5Lg1
WVRQAPGQGLEWMG

SEQ ID NO: 26 - protein, 32 aa, synthetic construct
  REGION 1..32: note = 5Hg1-5Lg1
RVTMTRDTSTSTVYMELSSLRSEDTAVYYCAR

SEQ ID NO: 27 - protein, 11 aa, synthetic construct
  REGION 1..11: note = 5Hg1-5Lg1
WGQGTLVTVSS

SEQ ID NO: 28 - DNA, 327 bp, synthetic construct
  misc_feature 1..327: note = 5Hg1-5Lg1
  source 1..327: mol_type = other DNA
caagccgtggtgacccaagaaccctctctaaccgtcagccccgggggcacggttaccctgacctgcagaagcagcaccggcgccgtgaccacaagcaactacgccaactggtttcagcagaagcccggccaagcccctagaaccctgatctacggcaccaacaacagagccccctggacccccgcccggttcagtgggagcctgctgggcgggaaagccgccctgactctgtcgggagctcagcccgaggacgaggccgagtactactgcgccctgtggtacagcaaccactgggtgttcggcggcggcaccaagctgaccgtgctg

SEQ ID NO: 29 - protein, 109 aa, synthetic construct
  REGION 1..109: note = 5Hg1-5Lg1
QAVVTQEPSLTVSPGGTVTLTCRSSTGAVTTSNYANWFQQKPGQAPRTLIYGTNNRAPWTPARFSGSLLGGKAALTLSGAQPEDEAEYYCALWYSNHWVFGGGTKLTVL

SEQ ID NO: 30 - protein, 14 aa, synthetic construct
  REGION 1..14: note = 5Hg1-5Lg1
RSSTGAVTTSNYAN

SEQ ID NO: 31 - protein, 7 aa, synthetic construct
  REGION 1..7: note = 5Hg1-5Lg1
GTNNRAP

SEQ ID NO: 32 - protein, 9 aa, synthetic construct
  REGION 1..9: note = 5Hg1-5Lg1
ALWYSNHWV

SEQ ID NO: 33 - protein, 22 aa, synthetic construct
  REGION 1..22: note = 5Hg1-5Lg1
QAVVTQEPSLTVSPGGTVTLTC

SEQ ID NO: 34 - protein, 15 aa, synthetic construct
  REGION 1..15: note = 5Hg1-5Lg1
WFQQKPGQAPRTLIY

SEQ ID NO: 35 - protein, 32 aa, synthetic construct
  REGION 1..32: note = 5Hg1-5Lg1
WTPARFSGSLLGGKAALTLSGAQPEDEAEYYC

SEQ ID NO: 36 - protein, 10 aa, synthetic construct
  REGION 1..10: note = 5Hg1-5Lg1
FGGGTKLTVL

SEQ ID NO: 37 - DNA, 363 bp, synthetic construct; identical to SEQ ID NO: 19
  misc_feature 1..363: note = 5Hg1-5Lg2
  source 1..363: mol_type = other DNA

SEQ ID NO: 38 - protein, 121 aa, synthetic construct; identical to SEQ ID NO: 20
  REGION 1..121: note = 5Hg1-5Lg2

SEQ ID NO: 39 - protein, 5 aa, synthetic construct
  REGION 1..5: note = 5Hg1-5Lg2
DYWMH

SEQ ID NO: 40 - protein, 17 aa, synthetic construct
  REGION 1..17: note = 5Hg1-5Lg2
AIDTSDSYTSYNQKFKG

SEQ ID NO: 41 - protein, 12 aa, synthetic construct
  REGION 1..12: note = 5Hg1-5Lg2
DYGNFYYYAMDY

SEQ ID NO: 42 - protein, 30 aa, synthetic construct; identical to SEQ ID NO: 24
  REGION 1..30: note = 5Hg1-5Lg2

SEQ ID NO: 43 - protein, 14 aa, synthetic construct
  REGION 1..14: note = 5Hg1-5Lg2
WVRQAPGQGLEWMG

SEQ ID NO: 44 - protein, 32 aa, synthetic construct; identical to SEQ ID NO: 26
  REGION 1..32: note = 5Hg1-5Lg2

SEQ ID NO: 45 - protein, 11 aa, synthetic construct
  REGION 1..11: note = 5Hg1-5Lg2
WGQGTLVTVSS

SEQ ID NO: 46 - DNA, 327 bp, synthetic construct
  misc_feature 1..327: note = 5Hg1-5Lg2
  source 1..327: mol_type = other DNA
caagccgtggtgacccaagaaccctctctaaccgtcagccccgggggcacggttaccctgacctgcagaagcagcaccggcgccgtgaccacaagcaactacgccaactggtttcagcagaagcccggccaagcccctagaaccctgatctacggcaccaacaacagagccccctggacccccgcccggttcagtgggagcctgctgggcgggaaagccgccctgactctgctaggagctcagcccgaggacgaggccgagtactactgcgccctgtggtacagcaaccactgggtgttcggcggcggcaccaagctgaccgtgctg

SEQ ID NO: 47 - protein, 109 aa, synthetic construct
  REGION 1..109: note = 5Hg1-5Lg2
QAVVTQEPSLTVSPGGTVTLTCRSSTGAVTTSNYANWFQQKPGQAPRTLIYGTNNRAPWTPARFSGSLLGGKAALTLLGAQPEDEAEYYCALWYSNHWVFGGGTKLTVL

SEQ ID NO: 48 - protein, 14 aa, synthetic construct
  REGION 1..14: note = 5Hg1-5Lg2
RSSTGAVTTSNYAN

SEQ ID NO: 49 - protein, 7 aa, synthetic construct
  REGION 1..7: note = 5Hg1-5Lg2
GTNNRAP

SEQ ID NO: 50 - protein, 9 aa, synthetic construct
  REGION 1..9: note = 5Hg1-5Lg2
ALWYSNHWV

SEQ ID NO: 51 - protein, 22 aa, synthetic construct
  REGION 1..22: note = 5Hg1-5Lg2
QAVVTQEPSLTVSPGGTVTLTC

SEQ ID NO: 52 - protein, 15 aa, synthetic construct
  REGION 1..15: note = 5Hg1-5Lg2
WFQQKPGQAPRTLIY

SEQ ID NO: 53 - protein, 32 aa, synthetic construct
  REGION 1..32: note = 5Hg1-5Lg2
WTPARFSGSLLGGKAALTLLGAQPEDEAEYYC

SEQ ID NO: 54 - protein, 10 aa, synthetic construct
  REGION 1..10: note = 5Hg1-5Lg2
FGGGTKLTVL

SEQ ID NO: 55 - DNA, 363 bp, synthetic construct
  misc_feature 1..363: note = 5Hg2-5Lg1
  source 1..363: mol_type = other DNA
caagtgcagctggtgcagagcggcgccgaggtgaagaagcccggcgctagcgtgaaggtgagctgcaaggctagcggctacaccttcaccgactactggatgcactgggtgagacaagcccccgggcagagactggagtggatgggcgccatcgacacaagcgacagctacacaagctacaatcagaagttcaagggcagagtgaccatcacaagagacacaagcgctagcaccgcctacatggagctgagcagcctgagaagcgaggacaccgccgtgtactactgcgctagagactacggcaacttctactactacgccatggactactggggccaaggcaccctggtcaccgtctcctca

SEQ ID NO: 56 - protein, 121 aa, synthetic construct
  REGION 1..121: note = 5Hg2-5Lg1
QVQLVQSGAEVKKPGASVKVSCKASGYTFTDYWMHWVRQAPGQRLEWMGAIDTSDSYTSYNQKFKGRVTITRDTSASTAYMELSSLRSEDTAVYYCARDYGNFYYYAMDYWGQGTLVTVSS

SEQ ID NO: 57 - protein, 5 aa, synthetic construct
  REGION 1..5: note = 5Hg2-5Lg1
DYWMH

SEQ ID NO: 58 - protein, 17 aa, synthetic construct
  REGION 1..17: note = 5Hg2-5Lg1
AIDTSDSYTSYNQKFKG

SEQ ID NO: 59 - protein, 12 aa, synthetic construct
  REGION 1..12: note = 5Hg2-5Lg1
DYGNFYYYAMDY

SEQ ID NO: 60 - protein, 30 aa, synthetic construct; identical to SEQ ID NO: 24
  REGION 1..30: note = 5Hg2-5Lg1

SEQ ID NO: 61 - protein, 14 aa, synthetic construct
  REGION 1..14: note = 5Hg2-5Lg1
WVRQAPGQRLEWMG

SEQ ID NO: 62 - protein, 32 aa, synthetic construct
  REGION 1..32: note = 5Hg2-5Lg1
RVTITRDTSASTAYMELSSLRSEDTAVYYCAR

SEQ ID NO: 63 - protein, 11 aa, synthetic construct
  REGION 1..11: note = 5Hg2-5Lg1
WGQGTLVTVSS

SEQ ID NO: 64 - DNA, 327 bp, synthetic construct; identical to SEQ ID NO: 28
  misc_feature 1..327: note = 5Hg2-5Lg1
  source 1..327: mol_type = other DNA

SEQ ID NO: 65 - protein, 109 aa, synthetic construct; identical to SEQ ID NO: 29
  REGION 1..109: note = 5Hg2-5Lg1

SEQ ID NO: 66 - protein, 14 aa, synthetic construct
  REGION 1..14: note = 5Hg2-5Lg1
RSSTGAVTTSNYAN

SEQ ID NO: 67 - protein, 7 aa, synthetic construct
  REGION 1..7: note = 5Hg2-5Lg1
GTNNRAP

SEQ ID NO: 68 - protein, 9 aa, synthetic construct
  REGION 1..9: note = 5Hg2-5Lg1
ALWYSNHWV

SEQ ID NO: 69 - protein, 22 aa, synthetic construct
  REGION 1..22: note = 5Hg2-5Lg1
QAVVTQEPSLTVSPGGTVTLTC

SEQ ID NO: 70 - protein, 15 aa, synthetic construct
  REGION 1..15: note = 5Hg2-5Lg1
WFQQKPGQAPRTLIY

SEQ ID NO: 71 - protein, 32 aa, synthetic construct; identical to SEQ ID NO: 35
  REGION 1..32: note = 5Hg2-5Lg1

SEQ ID NO: 72 - protein, 10 aa, synthetic construct
  REGION 1..10: note = 5Hg2-5Lg1
FGGGTKLTVL

SEQ ID NO: 73 - DNA, 363 bp, synthetic construct; identical to SEQ ID NO: 55
  misc_feature 1..363: note = 5Hg2-5Lg2
  source 1..363: mol_type = other DNA

SEQ ID NO: 74 - protein, 121 aa, synthetic construct; identical to SEQ ID NO: 56
  REGION 1..121: note = 5Hg2-5Lg2

SEQ ID NO: 75 - protein, 5 aa, synthetic construct
  REGION 1..5: note = 5Hg2-5Lg2
DYWMH

SEQ ID NO: 76 - protein, 17 aa, synthetic construct
  REGION 1..17: note = 5Hg2-5Lg2
AIDTSDSYTSYNQKFKG

SEQ ID NO: 77 - protein, 12 aa, synthetic construct
  REGION 1..12: note = 5Hg2-5Lg2
DYGNFYYYAMDY

SEQ ID NO: 78 - protein, 30 aa, synthetic construct; identical to SEQ ID NO: 24
  REGION 1..30: note = 5Hg2-5Lg2

SEQ ID NO: 79 - protein, 14 aa, synthetic construct
  REGION 1..14: note = 5Hg2-5Lg2
WVRQAPGQRLEWMG

SEQ ID NO: 80 - protein, 32 aa, synthetic construct; identical to SEQ ID NO: 62
  REGION 1..32: note = 5Hg2-5Lg2

SEQ ID NO: 81 - protein, 11 aa, synthetic construct
  REGION 1..11: note = 5Hg2-5Lg2
WGQGTLVTVSS

SEQ ID NO: 82 - DNA, 327 bp, synthetic construct; identical to SEQ ID NO: 46
  misc_feature 1..327: note = 5Hg2-5Lg2
  source 1..327: mol_type = other DNA

SEQ ID NO: 83 - protein, 109 aa, synthetic construct; identical to SEQ ID NO: 47
  REGION 1..109: note = 5Hg2-5Lg2

SEQ ID NO: 84 - protein, 14 aa, synthetic construct
  REGION 1..14: note = 5Hg2-5Lg2
RSSTGAVTTSNYAN

SEQ ID NO: 85 - protein, 7 aa, synthetic construct
  REGION 1..7: note = 5Hg2-5Lg2
GTNNRAP

SEQ ID NO: 86 - protein, 9 aa, synthetic construct
  REGION 1..9: note = 5Hg2-5Lg2
ALWYSNHWV

SEQ ID NO: 87 - protein, 22 aa, synthetic construct
  REGION 1..22: note = 5Hg2-5Lg2
QAVVTQEPSLTVSPGGTVTLTC

SEQ ID NO: 88 - protein, 15 aa, synthetic construct
  REGION 1..15: note = 5Hg2-5Lg2
WFQQKPGQAPRTLIY

SEQ ID NO: 89 - protein, 32 aa, synthetic construct; identical to SEQ ID NO: 53
  REGION 1..32: note = 5Hg2-5Lg2

SEQ ID NO: 90 - protein, 10 aa, synthetic construct
  REGION 1..10: note = 5Hg2-5Lg2
FGGGTKLTVL

SEQ ID NO: 91 - DNA, 363 bp, synthetic construct; identical to SEQ ID NO: 55
  misc_feature 1..363: note = 5Hg2-5Lg3
  source 1..363: mol_type = other DNA

SEQ ID NO: 92 - protein, 121 aa, synthetic construct; identical to SEQ ID NO: 56
  REGION 1..121: note = 5Hg2-5Lg3

SEQ ID NO: 93 - protein, 5 aa, synthetic construct
  REGION 1..5: note = 5Hg2-5Lg3
DYWMH

SEQ ID NO: 94 - protein, 17 aa, synthetic construct
  REGION 1..17: note = 5Hg2-5Lg3
AIDTSDSYTSYNQKFKG

SEQ ID NO: 95 - protein, 12 aa, synthetic construct
  REGION 1..12: note = 5Hg2-5Lg3
DYGNFYYYAMDY

SEQ ID NO: 96 - protein, 30 aa, synthetic construct; identical to SEQ ID NO: 24
  REGION 1..30: note = 5Hg2-5Lg3

SEQ ID NO: 97 - protein, 14 aa, synthetic construct
  REGION 1..14: note = 5Hg2-5Lg3
WVRQAPGQRLEWMG

SEQ ID NO: 98 - protein, 32 aa, synthetic construct; identical to SEQ ID NO: 62
  REGION 1..32: note = 5Hg2-5Lg3

SEQ ID NO: 99 - protein, 11 aa, synthetic construct
  REGION 1..11: note = 5Hg2-5Lg3
WGQGTLVTVSS

SEQ ID NO: 100 - DNA, 327 bp, synthetic construct
  misc_feature 1..327: note = 5Hg2-5Lg3
  source 1..327: mol_type = other DNA
cagacggtggtgacccaagagcctagcttcagcgtgagccccggtggcaccgtgaccctgacctgcagaagcagcaccggcgccgtgaccacaagcaactacgccaactggtatcagcagacccccggccaagcccctagaaccctgatctacggcaccaacaacagagcccccggcgtgcccgacagattcagcggcagcatcctgggcaacaaggccgccctgaccatcaccggcgcccaagccgacgacgagagcgactactactgcgccctgtggtacagcaaccactgggtgttcggcggcggcaccaagctgaccgtgctg

SEQ ID NO: 101 - protein, 109 aa, synthetic construct
  REGION 1..109: note = 5Hg2-5Lg3
QTVVTQEPSFSVSPGGTVTLTCRSSTGAVTTSNYANWYQQTPGQAPRTLIYGTNNRAPGVPDRFSGSILGNKAALTITGAQADDESDYYCALWYSNHWVFGGGTKLTVL

SEQ ID NO: 102 - protein, 14 aa, synthetic construct
  REGION 1..14: note = 5Hg2-5Lg3
RSSTGAVTTSNYAN

SEQ ID NO: 103 - protein, 7 aa, synthetic construct
  REGION 1..7: note = 5Hg2-5Lg3
GTNNRAP

SEQ ID NO: 104 - protein, 9 aa, synthetic construct
  REGION 1..9: note = 5Hg2-5Lg3
ALWYSNHWV

SEQ ID NO: 105 - protein, 22 aa, synthetic construct
  REGION 1..22: note = 5Hg2-5Lg3
QTVVTQEPSFSVSPGGTVTLTC

SEQ ID NO: 106 - protein, 15 aa, synthetic construct
  REGION 1..15: note = 5Hg2-5Lg3
WYQQTPGQAPRTLIY

SEQ ID NO: 107 - protein, 32 aa, synthetic construct
  REGION 1..32: note = 5Hg2-5Lg3
GVPDRFSGSILGNKAALTITGAQADDESDYYC

SEQ ID NO: 108 - protein, 10 aa, synthetic construct
  REGION 1..10: note = 5Hg2-5Lg3
FGGGTKLTVL

SEQ ID NO: 109 - DNA, 363 bp, synthetic construct; identical to SEQ ID NO: 55
  misc_feature 1..363: note = 5Hg2-5Lg5
  source 1..363: mol_type = other DNA

SEQ ID NO: 110 - protein, 121 aa, synthetic construct; identical to SEQ ID NO: 56
  REGION 1..121: note = 5Hg2-5Lg5

SEQ ID NO: 111 - protein, 5 aa, synthetic construct
  REGION 1..5: note = 5Hg2-5Lg5
DYWMH

SEQ ID NO: 112 - protein, 17 aa, synthetic construct
  REGION 1..17: note = 5Hg2-5Lg5
AIDTSDSYTSYNQKFKG

SEQ ID NO: 113 - protein, 12 aa, synthetic construct
  REGION 1..12: note = 5Hg2-5Lg5
DYGNFYYYAMDY

SEQ ID NO: 114 - protein, 30 aa, synthetic construct; identical to SEQ ID NO: 24
  REGION 1..30: note = 5Hg2-5Lg5

SEQ ID NO: 115 - protein, 14 aa, synthetic construct
  REGION 1..14: note = 5Hg2-5Lg5
WVRQAPGQRLEWMG

SEQ ID NO: 116 - protein, 32 aa, synthetic construct; identical to SEQ ID NO: 62
  REGION 1..32: note = 5Hg2-5Lg5

SEQ ID NO: 117 - protein, 11 aa, synthetic construct
  REGION 1..11: note = 5Hg2-5Lg5
WGQGTLVTVSS

SEQ ID NO: 118 - DNA, 327 bp, synthetic construct
  misc_feature 1..327: note = 5Hg2-5Lg5
  source 1..327: mol_type = other DNA
cagagcgtggtgacacagccccctagcgtgagcggcgcccccgggcagagagtgaccatcagctgcagaagcagcaccggcgccgtgaccacaagcaactacgccaactggtatcagcagctgcccggcaccgcccccaagctgctgatctacggcaccaacaacagagcccccggagtgccggaccgctttagcggcagcaagtctggtgctagcgctagcctggcaataaccggcctgcaagccgaggacgaggccgactactactgcgccctgtggtacagcaaccactgggtgttcggcggcggcaccaagctgaccgtgctg

SEQ ID NO: 119 - protein, 109 aa, synthetic construct
  REGION 1..109: note = 5Hg2-5Lg5
QSVVTQPPSVSGAPGQRVTISCRSSTGAVTTSNYANWYQQLPGTAPKLLIYGTNNRAPGVPDRFSGSKSGASASLAITGLQAEDEADYYCALWYSNHWVFGGGTKLTVL

SEQ ID NO: 120 - protein, 14 aa, synthetic construct
  REGION 1..14: note = 5Hg2-5Lg5
RSSTGAVTTSNYAN

SEQ ID NO: 121 - protein, 7 aa, synthetic construct
  REGION 1..7: note = 5Hg2-5Lg5
GTNNRAP

SEQ ID NO: 122 - protein, 9 aa, synthetic construct
  REGION 1..9: note = 5Hg2-5Lg5
ALWYSNHWV

SEQ ID NO: 123 - protein, 22 aa, synthetic construct
  REGION 1..22: note = 5Hg2-5Lg5
QSVVTQPPSVSGAPGQRVTISC

SEQ ID NO: 124 - protein, 15 aa, synthetic construct
  REGION 1..15: note = 5Hg2-5Lg5
WYQQLPGTAPKLLIY

SEQ ID NO: 125 - protein, 32 aa, synthetic construct
  REGION 1..32: note = 5Hg2-5Lg5
GVPDRFSGSKSGASASLAITGLQAEDEADYYC

SEQ ID NO: 126 - protein, 10 aa, synthetic construct
  REGION 1..10: note = 5Hg2-5Lg5
FGGGTKLTVL

SEQ ID NO: 127 - DNA, 363 bp, synthetic construct; identical to SEQ ID NO: 55
  misc_feature 1..363: note = 5Hg2-5Lg6
  source 1..363: mol_type = other DNA

SEQ ID NO: 128 - protein, 121 aa, synthetic construct; identical to SEQ ID NO: 56
  REGION 1..121: note = 5Hg2-5Lg6

SEQ ID NO: 129 - protein, 5 aa, synthetic construct
  REGION 1..5: note = 5Hg2-5Lg6
DYWMH

SEQ ID NO: 130 - protein, 17 aa, synthetic construct
  REGION 1..17: note = 5Hg2-5Lg6
AIDTSDSYTSYNQKFKG

SEQ ID NO: 131 - protein, 12 aa, synthetic construct
  REGION 1..12: note = 5Hg2-5Lg6
DYGNFYYYAMDY

SEQ ID NO: 132 - protein, 30 aa, synthetic construct; identical to SEQ ID NO: 24
  REGION 1..30: note = 5Hg2-5Lg6

SEQ ID NO: 133 - protein, 14 aa, synthetic construct
  REGION 1..14: note = 5Hg2-5Lg6
WVRQAPGQRLEWMG

SEQ ID NO: 134 - protein, 32 aa, synthetic construct; identical to SEQ ID NO: 62
  REGION 1..32: note = 5Hg2-5Lg6

SEQ ID NO: 135 - protein, 11 aa, synthetic construct
  REGION 1..11: note = 5Hg2-5Lg6
WGQGTLVTVSS

SEQ ID NO: 136 - DNA, 327 bp, synthetic construct
  misc_feature 1..327: note = 5Hg2-5Lg6
  source 1..327: mol_type = other DNA
agctacgagctgacacagcctagcagcgtgagcgtgagccccgggcagaccgctagaatcacctgcagaagcagcaccggcgccgtgaccacaagcaactacgccaactggtttcagcagaagcccggccaagcccccgtgctggtgatctacggcaccaacaacagagcccccggcatccccgagagattcagcggcagcagcagcggcaccaccgtgaccctgaccatcagcggcgcccaagtggaggacgaggccgactactactgcgccctgtggtacagcaaccactgggtgttcggcggcggcaccaagctgaccgtgctg

SEQ ID NO: 137 - protein, 109 aa, synthetic construct
  REGION 1..109: note = 5Hg2-5Lg6
SYELTQPSSVSVSPGQTARITCRSSTGAVTTSNYANWFQQKPGQAPVLVIYGTNNRAPGIPERFSGSSSGTTVTLTISGAQVEDEADYYCALWYSNHWVFGGGTKLTVL

SEQ ID NO: 138 - protein, 14 aa, synthetic construct
  REGION 1..14: note = 5Hg2-5Lg6
RSSTGAVTTSNYAN

SEQ ID NO: 139 - protein, 7 aa, synthetic construct
  REGION 1..7: note = 5Hg2-5Lg6
GTNNRAP

SEQ ID NO: 140 - protein, 9 aa, synthetic construct
  REGION 1..9: note = 5Hg2-5Lg6
ALWYSNHWV

SEQ ID NO: 141 - protein, 22 aa, synthetic construct
  REGION 1..22: note = 5Hg2-5Lg6
SYELTQPSSVSVSPGQTARITC

SEQ ID NO: 142 - protein, 15 aa, synthetic construct
  REGION 1..15: note = 5Hg2-5Lg6
WFQQKPGQAPVLVIY

SEQ ID NO: 143 - protein, 32 aa, synthetic construct
  REGION 1..32: note = 5Hg2-5Lg6
GIPERFSGSSSGTTVTLTISGAQVEDEADYYC

SEQ ID NO: 144 - protein, 10 aa, synthetic construct
  REGION 1..10: note = 5Hg2-5Lg6
FGGGTKLTVL

SEQ ID NO: 145 - DNA, 363 bp, synthetic construct
  misc_feature 1..363: note = 5Hg4-5Lg1
  source 1..363: mol_type = other DNA
caagtgcagctggtgcagagcggcagcgagctgaagaagcccggcgctagcgtgaaggtgagctgcaaggctagcggctacaccttcaccgactactggatgcactgggtgagacaagcccccggccaaggcctggagtggatgggcgccatcgacacaagcgacagctacacaagctacaatcagaagttcaagggcagattcgtgttcagcctggacacaagcgtgagcaccgcctacctgcagatctgcagcctgaaggccgaggacaccgccgtgtactactgcgctagagactacggcaacttctactactacgccatggactactggggccaaggcaccctggtcaccgtctcctca

SEQ ID NO: 146 - protein, 121 aa, synthetic construct
  REGION 1..121: note = 5Hg4-5Lg1
QVQLVQSGSELKKPGASVKVSCKASGYTFTDYWMHWVRQAPGQGLEWMGAIDTSDSYTSYNQKFKGRFVFSLDTSVSTAYLQICSLKAEDTAVYYCARDYGNFYYYAMDYWGQGTLVTVSS

SEQ ID NO: 147 - protein, 5 aa, synthetic construct
  REGION 1..5: note = 5Hg4-5Lg1
DYWMH

SEQ ID NO: 148 - protein, 17 aa, synthetic construct
  REGION 1..17: note = 5Hg4-5Lg1
AIDTSDSYTSYNQKFKG

SEQ ID NO: 149 - protein, 12 aa, synthetic construct
  REGION 1..12: note = 5Hg4-5Lg1
DYGNFYYYAMDY

SEQ ID NO: 150 - protein, 30 aa, synthetic construct
  REGION 1..30: note = 5Hg4-5Lg1
QVQLVQSGSELKKPGASVKVSCKASGYTFT

SEQ ID NO: 151 - protein, 14 aa, synthetic construct
  REGION 1..14: note = 5Hg4-5Lg1
WVRQAPGQGLEWMG

SEQ ID NO: 152 - protein, 32 aa, synthetic construct
  REGION 1..32: note = 5Hg4-5Lg1
RFVFSLDTSVSTAYLQICSLKAEDTAVYYCAR

SEQ ID NO: 153 - protein, 11 aa, synthetic construct
  REGION 1..11: note = 5Hg4-5Lg1
WGQGTLVTVSS

SEQ ID NO: 154 - DNA, 327 bp, synthetic construct; identical to SEQ ID NO: 28
  misc_feature 1..327: note = 5Hg4-5Lg1
  source 1..327: mol_type = other DNA

SEQ ID NO: 155 - protein, 109 aa, synthetic construct; identical to SEQ ID NO: 29
  REGION 1..109: note = 5Hg4-5Lg1

SEQ ID NO: 156 - protein, 14 aa, synthetic construct
  REGION 1..14: note = 5Hg4-5Lg1
RSSTGAVTTSNYAN

SEQ ID NO: 157 - protein, 7 aa, synthetic construct
  REGION 1..7: note = 5Hg4-5Lg1
GTNNRAP

SEQ ID NO: 158 - protein, 9 aa, synthetic construct
  REGION 1..9: note = 5Hg4-5Lg1
ALWYSNHWV

SEQ ID NO: 159 - protein, 22 aa, synthetic construct
  REGION 1..22: note = 5Hg4-5Lg1
QAVVTQEPSLTVSPGGTVTLTC

SEQ ID NO: 160 - protein, 15 aa, synthetic construct
  REGION 1..15: note = 5Hg4-5Lg1
WFQQKPGQAPRTLIY

SEQ ID NO: 161 - protein, 32 aa, synthetic construct; identical to SEQ ID NO: 35
  REGION 1..32: note = 5Hg4-5Lg1

SEQ ID NO: 162 - protein, 10 aa, synthetic construct
  REGION 1..10: note = 5Hg4-5Lg1
FGGGTKLTVL

SEQ ID NO: 163 - DNA, 363 bp, synthetic construct; identical to SEQ ID NO: 145
  misc_feature 1..363: note = 5Hg4-5Lg2
  source 1..363: mol_type = other DNA

SEQ ID NO: 164 - protein, 121 aa, synthetic construct; identical to SEQ ID NO: 146
  REGION 1..121: note = 5Hg4-5Lg2

SEQ ID NO: 165 - protein, 5 aa, synthetic construct
  REGION 1..5: note = 5Hg4-5Lg2
DYWMH

SEQ ID NO: 166 - protein, 17 aa, synthetic construct
  REGION 1..17: note = 5Hg4-5Lg2
AIDTSDSYTSYNQKFKG

SEQ ID NO: 167 - protein, 12 aa, synthetic construct
  REGION 1..12: note = 5Hg4-5Lg2
DYGNFYYYAMDY

SEQ ID NO: 168 - protein, 30 aa, synthetic construct; identical to SEQ ID NO: 150
  REGION 1..30: note = 5Hg4-5Lg2

SEQ ID NO: 169 - protein, 14 aa, synthetic construct
  REGION 1..14: note = 5Hg4-5Lg2
WVRQAPGQGLEWMG

SEQ ID NO: 170 - protein, 32 aa, synthetic construct; identical to SEQ ID NO: 152
  REGION 1..32: note = 5Hg4-5Lg2

SEQ ID NO: 171 - protein, 11 aa, synthetic construct
  REGION 1..11: note = 5Hg4-5Lg2
WGQGTLVTVSS

SEQ ID NO: 172 - DNA, 327 bp, synthetic construct; identical to SEQ ID NO: 46
  misc_feature 1..327: note = 5Hg4-5Lg2
  source 1..327: mol_type = other DNA

SEQ ID NO: 173 - protein, 109 aa, synthetic construct; identical to SEQ ID NO: 47
  REGION 1..109: note = 5Hg4-5Lg2

SEQ ID NO: 174 - protein, 14 aa, synthetic construct
  REGION 1..14: note = 5Hg4-5Lg2
RSSTGAVTTSNYAN

SEQ ID NO: 175 - protein, 7 aa, synthetic construct
  REGION 1..7: note = 5Hg4-5Lg2
GTNNRAP

SEQ ID NO: 176 - protein, 9 aa, synthetic construct
  REGION 1..9: note = 5Hg4-5Lg2
ALWYSNHWV

SEQ ID NO: 177 - protein, 22 aa, synthetic construct
  REGION 1..22: note = 5Hg4-5Lg2
QAVVTQEPSLTVSPGGTVTLTC

SEQ ID NO: 178 - protein, 15 aa, synthetic construct
  REGION 1..15: note = 5Hg4-5Lg2
WFQQKPGQAPRTLIY

SEQ ID NO: 179 - protein, 32 aa, synthetic construct; identical to SEQ ID NO: 53
  REGION 1..32: note = 5Hg4-5Lg2

SEQ ID NO: 180 - protein, 10 aa, synthetic construct
  REGION 1..10: note = 5Hg4-5Lg2
FGGGTKLTVL

SEQ ID NO: 181 - DNA, 363 bp, synthetic construct; identical to SEQ ID NO: 145
  misc_feature 1..363: note = 5Hg4-5Lg4
  source 1..363: mol_type = other DNA

SEQ ID NO: 182 - protein, 121 aa, synthetic construct; identical to SEQ ID NO: 146
  REGION 1..121: note = 5Hg4-5Lg4

SEQ ID NO: 183 - protein, 5 aa, synthetic construct
  REGION 1..5: note = 5Hg4-5Lg4
DYWMH

SEQ ID NO: 184 - protein, 17 aa, synthetic construct
  REGION 1..17: note = 5Hg4-5Lg4
AIDTSDSYTSYNQKFKG

SEQ ID NO: 185 - protein, 12 aa, synthetic construct
  REGION 1..12: note = 5Hg4-5Lg4
DYGNFYYYAMDY

SEQ ID NO: 186 - protein, 30 aa, synthetic construct; identical to SEQ ID NO: 150
  REGION 1..30: note = 5Hg4-5Lg4

SEQ ID NO: 187 - protein, 14 aa, synthetic construct
  REGION 1..14: note = 5Hg4-5Lg4
WVRQAPGQGLEWMG

SEQ ID NO: 188 - protein, 32 aa, synthetic construct; identical to SEQ ID NO: 152
  REGION 1..32: note = 5Hg4-5Lg4

SEQ ID NO: 189 - protein, 11 aa, synthetic construct
  REGION 1..11: note = 5Hg4-5Lg4
WGQGTLVTVSS

SEQ ID NO: 190 - DNA, 327 bp, synthetic construct
  misc_feature 1..327: note = 5Hg4-5Lg4
  source 1..327: mol_type = other DNA
cagagcgccctgacacagccccctagcgtgagcggcagccccgggcagagcgtgaccatcagctgcagaagcagcaccggcgccgtgaccacaagcaactacgccaactggtatcagcagccccccggcaccgcccccaagctgatgatctacggcaccaacaacagagcccccggcgtgcccgacagattcagcggcagcaagagcggcaacaccgctagcctgaccatcagcggcctgcaagccgaggacgaggccgactactactgcgccctgtggtacagcaaccactgggtgttcggcggcggcaccaagctgaccgtgctg

SEQ ID NO: 191 - protein, 109 aa, synthetic construct
  REGION 1..109: note = 5Hg4-5Lg4
QSALTQPPSVSGSPGQSVTISCRSSTGAVTTSNYANWYQQPPGTAPKLMIYGTNNRAPGVPDRFSGSKSGNTASLTISGLQAEDEADYYCALWYSNHWVFGGGTKLTVL

SEQ ID NO: 192 - protein, 14 aa, synthetic construct
  REGION 1..14: note = 5Hg4-5Lg4
RSSTGAVTTSNYAN

SEQ ID NO: 193 - protein, 7 aa, synthetic construct
  REGION 1..7: note = 5Hg4-5Lg4
GTNNRAP

SEQ ID NO: 194 - protein, 9 aa, synthetic construct
  REGION 1..9: note = 5Hg4-5Lg4
ALWYSNHWV

SEQ ID NO: 195 - protein, 22 aa, synthetic construct
  REGION 1..22: note = 5Hg4-5Lg4
QSALTQPPSVSGSPGQSVTISC

SEQ ID NO: 196 - protein, 15 aa, synthetic construct
  REGION 1..15: note = 5Hg4-5Lg4
WYQQPPGTAPKLMIY

SEQ ID NO: 197 - protein, 32 aa, synthetic construct
  REGION 1..32: note = 5Hg4-5Lg4
GVPDRFSGSKSGNTASLTISGLQAEDEADYYC

SEQ ID NO: 198 - protein, 10 aa, synthetic construct
  REGION 1..10: note = 5Hg4-5Lg4
FGGGTKLTVL

SEQ ID NO: 199 - DNA, 363 bp, synthetic construct; identical to SEQ ID NO: 145
  misc_feature 1..363: note = 5Hg4-5Lg5
  source 1..363: mol_type = other DNA

SEQ ID NO: 200 - protein, 121 aa, synthetic construct; identical to SEQ ID NO: 146
  REGION 1..121: note = 5Hg4-5Lg5

SEQ ID NO: 201 - protein, 5 aa, synthetic construct
  REGION 1..5: note = 5Hg4-5Lg5
DYWMH

SEQ ID NO: 202 - protein, 17 aa, synthetic construct
  REGION 1..17: note = 5Hg4-5Lg5
AIDTSDSYTSYNQKFKG

SEQ ID NO: 203 - protein, 12 aa, synthetic construct
  REGION 1..12: note = 5Hg4-5Lg5
DYGNFYYYAMDY

SEQ ID NO: 204 - protein, 30 aa, synthetic construct; identical to SEQ ID NO: 150
  REGION 1..30: note = 5Hg4-5Lg5

SEQ ID NO: 205 - protein, 14 aa, synthetic construct
  REGION 1..14: note = 5Hg4-5Lg5
WVRQAPGQGLEWMG

SEQ ID NO: 206 - protein, 32 aa, synthetic construct; identical to SEQ ID NO: 152
  REGION 1..32: note = 5Hg4-5Lg5

SEQ ID NO: 207 - protein, 11 aa, synthetic construct
  REGION 1..11: note = 5Hg4-5Lg5
WGQGTLVTVSS

SEQ ID NO: 208 - DNA, 327 bp, synthetic construct; identical to SEQ ID NO: 118
  misc_feature 1..327: note = 5Hg4-5Lg5
  source 1..327: mol_type = other DNA

SEQ ID NO: 209 - protein, 109 aa, synthetic construct; identical to SEQ ID NO: 119
  REGION 1..109: note = 5Hg4-5Lg5

SEQ ID NO: 210 - protein, 14 aa, synthetic construct
  REGION 1..14: note = 5Hg4-5Lg5
RSSTGAVTTSNYAN

SEQ ID NO: 211 - protein, 7 aa, synthetic construct
  REGION 1..7: note = 5Hg4-5Lg5
GTNNRAP

SEQ ID NO: 212 - protein, 9 aa, synthetic construct
  REGION 1..9: note = 5Hg4-5Lg5
ALWYSNHWV

SEQ ID NO: 213 - protein, 22 aa, synthetic construct
  REGION 1..22: note = 5Hg4-5Lg5
QSVVTQPPSVSGAPGQRVTISC

SEQ ID NO: 214 - protein, 15 aa, synthetic construct
  REGION 1..15: note = 5Hg4-5Lg5
WYQQLPGTAPKLLIY

SEQ ID NO: 215 - protein, 32 aa, synthetic construct; identical to SEQ ID NO: 125
  REGION 1..32: note = 5Hg4-5Lg5

SEQ ID NO: 216 - protein, 10 aa, synthetic construct
  REGION 1..10: note = 5Hg4-5Lg5
FGGGTKLTVL

SEQ ID NO: 217 - DNA, 363 bp, synthetic construct
  misc_feature 1..363: note = 5Hg5-5Lg2
  source 1..363: mol_type = other DNA
gaggtgcagctggtgcagagcggcgccgaggtgaagaagcccggcgagagcctgagaatcagctgcaagggcagcggctacagcttcaccgactactggatgcactgggtgagacagatgcccggcaagggcctggagtggatgggcgccatcgacacaagcgacagctacacaagctacaatcagaagttcaagggccaagtgaccatcagcgccgacaagagcatcagcaccgcctacctgcagtggagcagcctgaaggctagcgacaccgccatgtactactgcgctagagactacggcaacttctactactacgccatggactactggggccaaggcaccctggtcaccgtctcctca

SEQ ID NO: 218 - protein, 121 aa, synthetic construct
  REGION 1..121: note = 5Hg5-5Lg2
EVQLVQSGAEVKKPGESLRISCKGSGYSFTDYWMHWVRQMPGKGLEWMGAIDTSDSYTSYNQKFKGQVTISADKSISTAYLQWSSLKASDTAMYYCARDYGNFYYYAMDYWGQGTLVTVSS

SEQ ID NO: 219 - protein, 5 aa, synthetic construct
  REGION 1..5: note = 5Hg5-5Lg2
DYWMH

SEQ ID NO: 220 - protein, 17 aa, synthetic construct
  REGION 1..17: note = 5Hg5-5Lg2
AIDTSDSYTSYNQKFKG

SEQ ID NO: 221 - protein, 12 aa, synthetic construct
  REGION 1..12: note = 5Hg5-5Lg2
DYGNFYYYAMDY

SEQ ID NO: 222 - protein, 30 aa, synthetic construct
  REGION 1..30: note = 5Hg5-5Lg2
EVQLVQSGAEVKKPGESLRISCKGSGYSFT

SEQ ID NO: 223 - protein, 14 aa, synthetic construct
  REGION 1..14: note = 5Hg5-5Lg2
WVRQMPGKGLEWMG

SEQ ID NO: 224 - protein, 32 aa, synthetic construct
  REGION 1..32: note = 5Hg5-5Lg2
QVTISADKSISTAYLQWSSLKASDTAMYYCAR

SEQ ID NO: 225 - protein, 11 aa, synthetic construct
  REGION 1..11: note = 5Hg5-5Lg2
WGQGTLVTVSS

SEQ ID NO: 226 - DNA, 327 bp, synthetic construct; identical to SEQ ID NO: 46
  misc_feature 1..327: note = 5Hg5-5Lg2
  source 1..327: mol_type = other DNA

SEQ ID NO: 227 - protein, 109 aa, synthetic construct; identical to SEQ ID NO: 47
  REGION 1..109: note = 5Hg5-5Lg2

SEQ ID NO: 228 - protein, 14 aa, synthetic construct
  REGION 1..14: note = 5Hg5-5Lg2
RSSTGAVTTSNYAN

SEQ ID NO: 229 - protein, 7 aa, synthetic construct
  REGION 1..7: note = 5Hg5-5Lg2
GTNNRAP

SEQ ID NO: 230 - protein, 9 aa, synthetic construct
  REGION 1..9: note = 5Hg5-5Lg2
ALWYSNHWV

SEQ ID NO: 231 - protein, 22 aa, synthetic construct
  REGION 1..22: note = 5Hg5-5Lg2
QAVVTQEPSLTVSPGGTVTLTC

SEQ ID NO: 232 - protein, 15 aa, synthetic construct
  REGION 1..15: note = 5Hg5-5Lg2
WFQQKPGQAPRTLIY

SEQ ID NO: 233 - protein, 32 aa, synthetic construct; identical to SEQ ID NO: 53
  REGION 1..32: note = 5Hg5-5Lg2

SEQ ID NO: 234 - protein, 10 aa, synthetic construct
  REGION 1..10: note = 5Hg5-5Lg2
FGGGTKLTVL

SEQ ID NO: 235 - DNA, 357 bp, Homo sapiens
  misc_feature 1..357: note = C0120903
  source 1..357: mol_type = other DNA
gaggtgcagctggtgcagtctggagctgaggtgaagaagcctgggtcctcgatgaaggtctcctgcaaggcttctggaggcaccttcaacagctatactatcaactgggtgcgacaggcccctggacaagggcttgagtgggtgggagggttcatccctatctttggtacagcagactacgcacagaagttccacggcagagtcgcgattaccgcggacgaatccatgagcacagcctacatggagctgagcagcctgagatctgaggacacggccgtgtattactgtgcgagagctccccttgggtctgaaccatttgactactgggggcaagggacaatggtcaccgtctcctca

SEQ ID NO: 236 - protein, 119 aa, Homo sapiens
  REGION 1..119: note = C0120903
EVQLVQSGAEVKKPGSSMKVSCKASGGTFNSYTINWVRQAPGQGLEWVGGFIPIFGTADYAQKFHGRVAITADESMSTAYMELSSLRSEDTAVYYCARAPLGSEPFDYWGQGTMVTVSS

SEQ ID NO: 237 - protein, 5 aa, Homo sapiens
  REGION 1..5: note = C0120903
SYTIN

SEQ ID NO: 238 - protein, 17 aa, Homo sapiens
  REGION 1..17: note = C0120903
GFIPIFGTADYAQKFHG

SEQ ID NO: 239 - protein, 10 aa, Homo sapiens
  REGION 1..10: note = C0120903
APLGSEPFDY

SEQ ID NO: 240 - protein, 30 aa, Homo sapiens
  REGION 1..30: note = C0120903
EVQLVQSGAEVKKPGSSMKVSCKASGGTFN

SEQ ID NO: 241 - protein, 14 aa, Homo sapiens
  REGION 1..14: note = C0120903
WVRQAPGQGLEWVG

SEQ ID NO: 242 - protein, 32 aa, Homo sapiens
  REGION 1..32: note = C0120903
RVAITADESMSTAYMELSSLRSEDTAVYYCAR

SEQ ID NO: 243 - protein, 11 aa, Homo sapiens
  REGION 1..11: note = C0120903
WGQGTMVTVSS

SEQ ID NO: 244 - DNA, 324 bp, Homo sapiens
  misc_feature 1..324: note = C0120903
  source 1..324: mol_type = other DNA
tcctatgtgctgactcagccaccctcggtgtcagtggccccaggacagacggccaggattacctgtgggggaaacaacattggaagtaaaagtgtccactggtaccagcagaagccaggccaggcccctgtgctggtcgtctatgatgatagcgaccggccctcagggatccctgagcgattctctggctccaactctgggaacacggccaccctgaccatcagcagggtcgaagccggggatgaggccgactattactgtcaggtgtgggatagtagtagtgatcatctggtgttcggcggagggaccaagctgaccgtccta

SEQ ID NO: 245 - protein, 108 aa, Homo sapiens
  REGION 1..108: note = C0120903
SYVLTQPPSVSVAPGQTARITCGGNNIGSKSVHWYQQKPGQAPVLVVYDDSDRPSGIPERFSGSNSGNTATLTISRVEAGDEADYYCQVWDSSSDHLVFGGGTKLTVL

SEQ ID NO: 246 - protein, 11 aa, Homo sapiens
  REGION 1..11: note = C0120903
GGNNIGSKSVH

SEQ ID NO: 247 - protein, 7 aa, Homo sapiens
  REGION 1..7: note = C0120903
DDSDRPS

SEQ ID NO: 248 - protein, 11 aa, Homo sapiens
  REGION 1..11: note = C0120903
QVWDSSSDHLV

SEQ ID NO: 249 - protein, 22 aa, Homo sapiens
  REGION 1..22: note = C0120903
SYVLTQPPSVSVAPGQTARITC

SEQ ID NO: 250 - protein, 15 aa, Homo sapiens
  REGION 1..15: note = C0120903
WYQQKPGQAPVLVVY

SEQ ID NO: 251 - protein, 32 aa, Homo sapiens
  REGION 1..32: note = C0120903
GIPERFSGSNSGNTATLTISRVEAGDEADYYC

SEQ ID NO: 252 - protein, 10 aa, Homo sapiens
  REGION 1..10: note = C0120903
FGGGTKLTVL

SEQ ID NO: 253 - DNA, 366 bp, Homo sapiens
  misc_feature 1..366: note = C0120904
  source 1..366: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagattacggctacggctactacggcttcgatgtgtggggtcaggggaccacggtcaccgtctcctca

SEQ ID NO: 254 - protein, 122 aa, Homo sapiens
  REGION 1..122: note = C0120904
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTDYGYGYYGFDVWGQGTTVTVSS

SEQ ID NO: 255 - protein, 5 aa, Homo sapiens
  REGION 1..5: note = C0120904
NAWMN

SEQ ID NO: 256 - protein, 19 aa, Homo sapiens
  REGION 1..19: note = C0120904
RIKSKTDGGTTDYAAPVKG

SEQ ID NO: 257 - protein, 11 aa, Homo sapiens
  REGION 1..11: note = C0120904
DYGYGYYGFDV

SEQ ID NO: 258 - protein, 30 aa, Homo sapiens
  REGION 1..30: note = C0120904
EVQLVESGGGLVKPGGSLRLSCAASGFTFS

SEQ ID NO: 259 - protein, 14 aa, Homo sapiens
  REGION 1..14: note = C0120904
WVRQAPGKGLEWVG

SEQ ID NO: 260 - protein, 32 aa, Homo sapiens
  REGION 1..32: note = C0120904
RFTISRDDSKNTLYLQMNSLKTEDTAVYYCTT

SEQ ID NO: 261 - protein, 11 aa, Homo sapiens
  REGION 1..11: note = C0120904
WGQGTTVTVSS

SEQ ID NO: 262 - DNA, 318 bp, Homo sapiens
  misc_feature 1..318: note = C0120904
  source 1..318: mol_type = other DNA
tcctatgagctgactcagccaccctcagtgtccgtgtccccaggacagacagccaccatctcctgctctggaaataaattgggggatggatatgtttcctggtatcagcagaagccaggccagtcccctgtgttggtcatctatcaagattccaagcggccctcagggatccctcagcgatactctggctccaactctgggaacaccgccactctgaccatcagcgggacccaggcaatagatgaggctgactattactgccaggcgtgggacagtagtactgtggttttcggcggagggaccaaggtcaccgtccta

SEQ ID NO: 263 - protein, 106 aa, Homo sapiens
  REGION 1..106: note = C0120904
SYELTQPPSVSVSPGQTATISCSGNKLGDGYVSWYQQKPGQSPVLVIYQDSKRPSGIPQRYSGSNSGNTATLTISGTQAIDEADYYCQAWDSSTVVFGGGTKVTVL

SEQ ID NO: 264 - protein, 11 aa, Homo sapiens
  REGION 1..11: note = C0120904
SGNKLGDGYVS

SEQ ID NO: 265 - protein, 7 aa, Homo sapiens
  REGION 1..7: note = C0120904
QDSKRPS

SEQ ID NO: 266 - protein, 9 aa, Homo sapiens
  REGION 1..9: note = C0120904
QAWDSSTVV

SEQ ID NO: 267 - protein, 22 aa, Homo sapiens
  REGION 1..22: note = C0120904
SYELTQPPSVSVSPGQTATISC

SEQ ID NO: 268 - protein, 15 aa, Homo sapiens
  REGION 1..15: note = C0120904
WYQQKPGQSPVLVIY

SEQ ID NO: 269 - protein, 32 aa, Homo sapiens
  REGION 1..32: note = C0120904
GIPQRYSGSNSGNTATLTISGTQAIDEADYYC

SEQ ID NO: 270 - protein, 10 aa, Homo sapiens
  REGION 1..10: note = C0120904
FGGGTKVTVL

SEQ ID NO: 271 - DNA, 402 bp, Homo sapiens
  misc_feature 1..402: note = C0120905
  source 1..402: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagatcgtccgccgatgtctcgtggcccgtcttctgtgtacgaaggcccgtaccgttacggcttcgatgtgtggggtcaggggaccacggtcaccgtctcctca

SEQ ID NO: 272 - protein, 134 aa, Homo sapiens
  REGION 1..134: note = C0120905
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTDRPPMSRGPSSVYEGPYRYGFDVWGQGTTVTVSS

SEQ ID NO: 273 - protein, 5 aa, Homo sapiens
  REGION 1..5: note = C0120905
NAWMN

SEQ ID NO: 274 - protein, 19 aa, Homo sapiens
  REGION 1..19: note = C0120905
RIKSKTDGGTTDYAAPVKG

SEQ ID NO: 275 - protein, 23 aa, Homo sapiens
  REGION 1..23: note = C0120905
DRPPMSRGPSSVYEGPYRYGFDV

SEQ ID NO: 276 - protein, 30 aa, Homo sapiens; identical to SEQ ID NO: 258
  REGION 1..30: note = C0120905

SEQ ID NO: 277 - protein, 14 aa, Homo sapiens
  REGION 1..14: note = C0120905
WVRQAPGKGLEWVG

SEQ ID NO: 278 - protein, 32 aa, Homo sapiens; identical to SEQ ID NO: 260
  REGION 1..32: note = C0120905

SEQ ID NO: 279 - protein, 11 aa, Homo sapiens
  REGION 1..11: note = C0120905
WGQGTTVTVSS

SEQ ID NO: 280 - DNA, 333 bp, Homo sapiens
  misc_feature 1..333: note = C0120905
  source 1..333: mol_type = other DNA
aattttatgctgactcagccccactctgtgtcggagtctccggggaagacggtaaccatctcctgcaccggcagcggcggcaacattgtcgataattttgtacaatggcaccagcagcgcccgggcagtgctccccgcactgtgatctatgaggatgaccaaagaccctctggggtccctgatcggttctctgcctccgtcgacagctcctccaactctggcaccctctccatctctggactgaagactgaggacgaggctgattactactgtcaatcttatgatggcgccaatcgttgggtcttcggcggagggaccaagctgaccgtccta

SEQ ID NO: 281 - protein, 111 aa, Homo sapiens
  REGION 1..111: note = C0120905
NFMLTQPHSVSESPGKTVTISCTGSGGNIVDNFVQWHQQRPGSAPRTVIYEDDQRPSGVPDRFSASVDSSSNSGTLSISGLKTEDEADYYCQSYDGANRWVFGGGTKLTVL

SEQ ID NO: 282 - protein, 13 aa, Homo sapiens
  REGION 1..13: note = C0120905
TGSGGNIVDNFVQ

SEQ ID NO: 283 - protein, 7 aa, Homo sapiens
  REGION 1..7: note = C0120905
EDDQRPS

SEQ ID NO: 284 - protein, 10 aa, Homo sapiens
  REGION 1..10: note = C0120905
QSYDGANRWV

SEQ ID NO: 285 - protein, 22 aa, Homo sapiens
  REGION 1..22: note = C0120905
NFMLTQPHSVSESPGKTVTISC

SEQ ID NO: 286 - protein, 15 aa, Homo sapiens
  REGION 1..15: note = C0120905
WHQQRPGSAPRTVIY

SEQ ID NO: 287 - protein, 34 aa, Homo sapiens
  REGION 1..34: note = C0120905
GVPDRFSASVDSSSNSGTLSISGLKTEDEADYYC

SEQ ID NO: 288 - protein, 10 aa, Homo sapiens
  REGION 1..10: note = C0120905
FGGGTKLTVL

SEQ ID NO: 289 - DNA, 363 bp, Homo sapiens
  misc_feature 1..363: note = C0120906
  source 1..363: mol_type = other DNA
gaggtgcagctggtgcagtctggacctgaggtgaagaagcctggggcctcagtgaaaatctcctgcaaggcctctggttataagttaaatgaccctggtgtccatggtgtcagctgggtgcgacaggcccctggacaagggcttgagtggatgggatggatcagcacactcaatggtgacacaagatatgcgcagaaattccagggcagagtcaccatggccacggactcatccacaagcacaatctacatggacctgaggagcctaaaatctgacgacacggccctttattactgtgcgagagatgactatactgccatttttgacttctggggccggggaaccctggtcaccgtctcctca

SEQ ID NO: 290 - protein, 121 aa, Homo sapiens
  REGION 1..121: note = C0120906
EVQLVQSGPEVKKPGASVKISCKASGYKLNDPGVHGVSWVRQAPGQGLEWMGWISTLNGDTRYAQKFQGRVTMATDSSTSTIYMDLRSLKSDDTALYYCARDDYTAIFDFWGRGTLVTVSS

SEQ ID NO: 291 - protein, 7 aa, Homo sapiens
  REGION 1..7: note = C0120906
DPGVHGV

SEQ ID NO: 292 - protein, 17 aa, Homo sapiens
  REGION 1..17: note = C0120906
WISTLNGDTRYAQKFQG

SEQ ID NO: 293 - protein, 9 aa, Homo sapiens
  REGION 1..9: note = C0120906
DDYTAIFDF

SEQ ID NO: 294 - protein, 30 aa, Homo sapiens
  REGION 1..30: note = C0120906
EVQLVQSGPEVKKPGASVKISCKASGYKLN

SEQ ID NO: 295 - protein, 14 aa, Homo sapiens
  REGION 1..14: note = C0120906
WVRQAPGQGLEWMG

SEQ ID NO: 296 - protein, 32 aa, Homo sapiens
  REGION 1..32: note = C0120906
RVTMATDSSTSTIYMDLRSLKSDDTALYYCAR

SEQ ID NO: 297 - protein, 11 aa, Homo sapiens
  REGION 1..11: note = C0120906
WGRGTLVTVSS

SEQ ID NO: 298 - DNA, 324 bp, Homo sapiens
  misc_feature 1..324: note = C0120906
  source 1..324: mol_type = other DNA
cagtctgtgttgactcagcccccctcggtgtcagtggccccaggacagacggccagcattacctgtgggggaagcaacattggaagtcagagtgttcactggtaccagcagaagccaggccaggcccctgtgctggtcgtctatgatgatagcgaccggccctcagggatccctgagcgattctctggctccaagtctgggaacacggcctccctgatacttagcagggtcgaagccggggatgaggccgactattactgtcaggtgtgggatagtagtactgatcatcatgtcttcggaactgggaccaaggtcaccgtccta

SEQ ID NO: 299 - protein, 108 aa, Homo sapiens
  REGION 1..108: note = C0120906
QSVLTQPPSVSVAPGQTASITCGGSNIGSQSVHWYQQKPGQAPVLVVYDDSDRPSGIPERFSGSKSGNTASLILSRVEAGDEADYYCQVWDSSTDHHVFGTGTKVTVL

SEQ ID NO: 300 - protein, 11 aa, Homo sapiens
  REGION 1..11: note = C0120906
GGSNIGSQSVH

SEQ ID NO: 301 - protein, 7 aa, Homo sapiens
  REGION 1..7: note = C0120906
DDSDRPS

SEQ ID NO: 302 - protein, 11 aa, Homo sapiens
  REGION 1..11: note = C0120906
QVWDSSTDHHV

SEQ ID NO: 303 - protein, 22 aa, Homo sapiens
  REGION 1..22: note = C0120906
QSVLTQPPSVSVAPGQTASITC

SEQ ID NO: 304 - protein, 15 aa, Homo sapiens
  REGION 1..15: note = C0120906
WYQQKPGQAPVLVVY

SEQ ID NO: 305 - protein, 32 aa, Homo sapiens
  REGION 1..32: note = C0120906
GIPERFSGSKSGNTASLILSRVEAGDEADYYC

SEQ ID NO: 306 - protein, 10 aa, Homo sapiens
  REGION 1..10: note = C0120906
FGTGTKVTVL

SEQ ID NO: 307 - DNA, 369 bp, Homo sapiens
  misc_feature 1..369: note = C0120910
  source 1..369: mol_type = other DNA
caggtgcagctggtggagtccgggggaggcgtggtccagcctgggaggtccctgagactctcctgtgaagcctctgaattcaccttcagtaactatgctatgcattgggtccgccaggctccagacaaggggctggagtgggtggcaggtattttgtatgatggagccacagcacactacgcagactccgtgaagggccggttcaccatctccagagacaattccaagaacacgctctatctgcaaattaacagcctgagagccgaggacacggctgtgtattactgtgcgagagctcactattacgattattggagtggtctaagttttgagtactggggcagaggcaccctggtcaccgtctcctca

SEQ ID NO: 308 - protein, 123 aa, Homo sapiens
  REGION 1..123: note = C0120910
QVQLVESGGGVVQPGRSLRLSCEASEFTFSNYAMHWVRQAPDKGLEWVAGILYDGATAHYADSVKGRFTISRDNSKNTLYLQINSLRAEDTAVYYCARAHYYDYWSGLSFEYWGRGTLVTVSS

SEQ ID NO: 309 - protein, 5 aa, Homo sapiens
  REGION 1..5: note = C0120910
NYAMH

SEQ ID NO: 310 - protein, 17 aa, Homo sapiens
  REGION 1..17: note = C0120910
GILYDGATAHYADSVKG

SEQ ID NO: 311 - protein, 14 aa, Homo sapiens
  REGION 1..14: note = C0120910
AHYYDYWSGLSFEY

SEQ ID NO: 312 - protein, 30 aa, Homo sapiens
  REGION 1..30: note = C0120910
QVQLVESGGGVVQPGRSLRLSCEASEFTFS

SEQ ID NO: 313 - protein, 14 aa, Homo sapiens
  REGION 1..14: note = C0120910
WVRQAPDKGLEWVA

SEQ ID NO: 314 - protein, 32 aa, Homo sapiens
  REGION 1..32: note = C0120910
RFTISRDNSKNTLYLQINSLRAEDTAVYYCAR

SEQ ID NO: 315 - protein, 11 aa, Homo sapiens
  REGION 1..11: note = C0120910
WGRGTLVTVSS

SEQ ID NO: 316 - DNA, 324 bp, Homo sapiens
  misc_feature 1..324: note = C0120910
  source 1..324: mol_type = other DNA
cagtctgtgttgactcagccaccctcagcgtccgtctccccaggacagacagccagcatcccctgctctggagatgatttgggggataaatatatctactggtatcagcagaagccaggccagtctcctgtcctggtcatctatcaagatagcaggcggccgccagggatccctgagcgattctctggctccaactctgggaacacagccactctgaccatcagcgggacccagcctatggatgaggctgactattactgtcagacgtgggacaccagttctgcgtctgcggtgttcggcggagggaccaaggtcaccgtccta

SEQ ID NO: 317 - protein, 108 aa, Homo sapiens
  REGION 1..108: note = C0120910
QSVLTQPPSASVSPGQTASIPCSGDDLGDKYIYWYQQKPGQSPVLVIYQDSRRPPGIPERFSGSNSGNTATLTISGTQPMDEADYYCQTWDTSSASAVFGGGTKVTVL

SEQ ID NO: 318 - protein, 11 aa, Homo sapiens
  REGION 1..11: note = C0120910
SGDDLGDKYIY

SEQ ID NO: 319 - protein, 7 aa, Homo sapiens
  REGION 1..7: note = C0120910
QDSRRPP

SEQ ID NO: 320 - protein, 11 aa, Homo sapiens
  REGION 1..11: note = C0120910
QTWDTSSASAV

SEQ ID NO: 321 - protein, 22 aa, Homo sapiens
  REGION 1..22: note = C0120910
QSVLTQPPSASVSPGQTASIPC

SEQ ID NO: 322 - protein, 15 aa, Homo sapiens
  REGION 1..15: note = C0120910
WYQQKPGQSPVLVIY

SEQ ID NO: 323 - protein, 32 aa, Homo sapiens
  REGION 1..32: note = C0120910
GIPERFSGSNSGNTATLTISGTQPMDEADYYC

SEQ ID NO: 324 - protein, 10 aa, Homo sapiens
  REGION 1..10: note = C0120910
FGGGTKVTVL

SEQ ID NO: 325 - DNA, 363 bp, Homo sapiens
  misc_feature 1..363: note = C0120913
  source 1..363: mol_type = other DNA
caggtgcagctggtgcagtctggagcagaggtgaaaaagcccggggagtctctgaagatctcctgtaagggttctggatacagttttaccagctattgggtcggctgggtgcgccagatgcccgggaaaggcctggagtggatggggatcatctatcctggtgactctgatgccagatatggcccgtccttccaaggccaggtcaccatctcagccgacaagtccatcaacaccgcctacctgcagtggaacagcctgaaggcctcggacaccgccacgtattactgtgcgagactaggggatggggcctactactactatttggacgtctggggcagggggacaatggtcaccgtctcctca

SEQ ID NO: 326 - protein, 121 aa, Homo sapiens
  REGION 1..121: note = C0120913
QVQLVQSGAEVKKPGESLKISCKGSGYSFTSYWVGWVRQMPGKGLEWMGIIYPGDSDARYGPSFQGQVTISADKSINTAYLQWNSLKASDTATYYCARLGDGAYYYYLDVWGRGTMVTVSS

SEQ ID NO: 327 - protein, 5 aa, Homo sapiens
  REGION 1..5: note = C0120913
SYWVG

SEQ ID NO: 328 - protein, 17 aa, Homo sapiens
  REGION 1..17: note = C0120913
IIYPGDSDARYGPSFQG

SEQ ID NO: 329 - protein, 12 aa, Homo sapiens
  REGION 1..12: note = C0120913
LGDGAYYYYLDV

SEQ ID NO: 330 - protein, 30 aa, Homo sapiens
  REGION 1..30: note = C0120913
QVQLVQSGAEVKKPGESLKISCKGSGYSFT

SEQ ID NO: 331 - protein, 14 aa, Homo sapiens
  REGION 1..14: note = C0120913
WVRQMPGKGLEWMG

SEQ ID NO: 332 - protein, 32 aa, Homo sapiens
  REGION 1..32: note = C0120913
QVTISADKSINTAYLQWNSLKASDTATYYCAR

SEQ ID NO: 333 - protein, 11 aa, Homo sapiens
  REGION 1..11: note = C0120913
WGRGTMVTVSS

SEQ ID NO: 334 - DNA, 333 bp, Homo sapiens
  misc_feature 1..333: note = C0120913
  source 1..333: mol_type = other DNA
tcctatgagctgactcagccaccctctgtgtcggagtctccggggaggacggtaaccatctcctgctccggcagcagtggcagcattgccagcaactttgtgcattggtaccagcagcgcccgggcagtgcccccaccactgtgatctatgaagatgaccaaagaccctctggggtccctgatcggttctctggctccatcgacagctcctccaactctgcctccctcaccatctctggactgaagactgaggacgaggctgactactactgtcaatcttatgataacaacgatcatgcagtgttcggcggagggaccaagctgaccgtccta

SEQ ID NO: 335 - protein, 111 aa, Homo sapiens
  REGION 1..111: note = C0120913
SYELTQPPSVSESPGRTVTISCSGSSGSIASNFVHWYQQRPGSAPTTVIYEDDQRPSGVPDRFSGSIDSSSNSASLTISGLKTEDEADYYCQSYDNNDHAVFGGGTKLTVL

SEQ ID NO: 336 - protein, 13 aa, Homo sapiens
  REGION 1..13: note = C0120913
SGSSGSIASNFVH

SEQ ID NO: 337 - protein, 7 aa, Homo sapiens
  REGION 1..7: note = C0120913
EDDQRPS

SEQ ID NO: 338 - protein, 10 aa, Homo sapiens
  REGION 1..10: note = C0120913
QSYDNNDHAV

SEQ ID NO: 339 - protein, 22 aa, Homo sapiens
  REGION 1..22: note = C0120913
SYELTQPPSVSESPGRTVTISC

SEQ ID NO: 340 - protein, 15 aa, Homo sapiens
  REGION 1..15: note = C0120913
WYQQRPGSAPTTVIY

SEQ ID NO: 341 - protein, 34 aa, Homo sapiens
  REGION 1..34: note = C0120913
GVPDRFSGSIDSSSNSASLTISGLKTEDEADYYC

SEQ ID NO: 342 - protein, 10 aa, Homo sapiens
  REGION 1..10: note = C0120913
FGGGTKLTVL

SEQ ID NO: 343 - DNA, 348 bp, Homo sapiens
  misc_feature 1..348: note = C0120914
  source 1..348: mol_type = other DNA
caggtgcagctgcaggagtccggcccaggactggtgaagccttcggagaccctgtccctcacctgcactgtgtctggtggctccgtcagcagtgggcgttacgactggagttggatccggcagcccccagggaagggactggaatggattggccatatttattacactgggatcaccgactacaatccctccctcaggagtcgagtcaccatttcagcagacaagcccaagaaccagttctccctgaagttgaattctgtgaccgctgcggacacggccgtctattattgtgcgagaggcccctttttcgacccctggggccaagggacaatggtcaccgtctcctca

SEQ ID NO: 344 - protein, 116 aa, Homo sapiens
  REGION 1..116: note = C0120914
QVQLQESGPGLVKPSETLSLTCTVSGGSVSSGRYDWSWIRQPPGKGLEWIGHIYYTGITDYNPSLRSRVTISADKPKNQFSLKLNSVTAADTAVYYCARGPFFDPWGQGTMVTVSS

SEQ ID NO: 345 - protein, 7 aa, Homo sapiens
  REGION 1..7: note = C0120914
SGRYDWS

SEQ ID NO: 346 - protein, 16 aa, Homo sapiens
  REGION 1..16: note = C0120914
HIYYTGITDYNPSLRS

SEQ ID NO: 347 - protein, 6 aa, Homo sapiens
  REGION 1..6: note = C0120914
GPFFDP

SEQ ID NO: 348 - protein, 30 aa, Homo sapiens
  REGION 1..30: note = C0120914
QVQLQESGPGLVKPSETLSLTCTVSGGSVS

SEQ ID NO: 349 - protein, 14 aa, Homo sapiens
  REGION 1..14: note = C0120914
WIRQPPGKGLEWIG

SEQ ID NO: 350 - protein, 32 aa, Homo sapiens
  REGION 1..32: note = C0120914
RVTISADKPKNQFSLKLNSVTAADTAVYYCAR

SEQ ID NO: 351 - protein, 11 aa, Homo sapiens
  REGION 1..11: note = C0120914
WGQGTMVTVSS

SEQ ID NO: 352 - DNA, 324 bp, Homo sapiens
  misc_feature 1..324: note = C0120914
  source 1..324: mol_type = other DNA
cagtctgtgttgacgcagccaccctcggtgtccgtggccccaggacagacggccagccttacctgtggggcaaacaacattggaagtaaaagtgttcagtggtaccaacagaagccaggccaggcccctgttctggtcgtctatgatggtagcgaccggccctcagggatccctgaccgattctctggctccagttctgagaacacggccaccctgaccatcagcagggtcgaagccggggatgaggccgactattactgtcagctgtgggatagtgttacagatcgtccggtattcggcggagggaccaaggtcaccgtccta

SEQ ID NO: 353 - protein, 108 aa, Homo sapiens
  REGION 1..108: note = C0120914
QSVLTQPPSVSVAPGQTASLTCGANNIGSKSVQWYQQKPGQAPVLVVYDGSDRPSGIPDRFSGSSSENTATLTISRVEAGDEADYYCQLWDSVTDRPVFGGGTKVTVL

SEQ ID NO: 354 - protein, 11 aa, Homo sapiens
  REGION 1..11: note = C0120914
GANNIGSKSVQ

SEQ ID NO: 355 - protein, 7 aa, Homo sapiens
  REGION 1..7: note = C0120914
DGSDRPS

SEQ ID NO: 356 - protein, 11 aa, Homo sapiens
  REGION 1..11: note = C0120914
QLWDSVTDRPV

SEQ ID NO: 357 - protein, 22 aa, Homo sapiens
  REGION 1..22: note = C0120914
QSVLTQPPSVSVAPGQTASLTC

SEQ ID NO: 358 - protein, 15 aa, Homo sapiens
  REGION 1..15: note = C0120914
WYQQKPGQAPVLVVY

SEQ ID NO: 359 - protein, 32 aa, Homo sapiens
  REGION 1..32: note = C0120914
GIPDRFSGSSSENTATLTISRVEAGDEADYYC

SEQ ID NO: 360 - protein, 10 aa, Homo sapiens
  REGION 1..10: note = C0120914
FGGGTKVTVL

SEQ ID NO: 361 - DNA, 396 bp, Homo sapiens
  misc_feature 1..396: note = C0120917
  source 1..396: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagaagatccggctggctactacggcaccgaaggctcttaccgttactacggctacatggattactggggtcaggggaccacggtcaccgtctcctca

SEQ ID NO: 362 - protein, 132 aa, Homo sapiens
  REGION 1..132: note = C0120917
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTEDPAGYYGTEGSYRYYGYMDYWGQGTTVTVSS

SEQ ID NO: 363 - protein, 5 aa, Homo sapiens
  REGION 1..5: note = C0120917
NAWMN

SEQ ID NO: 364 - protein, 19 aa, Homo sapiens
  REGION 1..19: note = C0120917
RIKSKTDGGTTDYAAPVKG

SEQ ID NO: 365 - protein, 21 aa, Homo sapiens
  REGION 1..21: note = C0120917
EDPAGYYGTEGSYRYYGYMDY

SEQ ID NO: 366 - protein, 30 aa, Homo sapiens; identical to SEQ ID NO: 258
  REGION 1..30: note = C0120917

SEQ ID NO: 367 - protein, 14 aa, Homo sapiens
  REGION 1..14: note = C0120917
WVRQAPGKGLEWVG

SEQ ID NO: 368 - protein, 32 aa, Homo sapiens; identical to SEQ ID NO: 260
  REGION 1..32: note = C0120917

SEQ ID NO: 369 - protein, 11 aa, Homo sapiens
  REGION 1..11: note = C0120917
WGQGTTVTVSS

SEQ ID NO: 370 - DNA, 339 bp, Homo sapiens
  misc_feature 1..339: note = C0120917
  source 1..339: mol_type = other DNA
aattttatgctgactcagccccactctgtgtcggaatctccggggaagacggtgaccatctcctgcaccgccagcggtgacacccttgacaacaaatatgtgaactggtatcaagtccgcccgggcagtgcccccgccactttgatcttccattatgaagaaagagactctggggtccctgatcggttctctggctcctttgacgcctcctccaattctgcctccctcaccatctctggactgcagactgcggacgaggctgattatttttgtcagtcttatgactccaagcgcgacaacttagttgttttcggcggagggaccaaggtcaccgtccta

SEQ ID NO: 371 - protein, 113 aa, Homo sapiens
  REGION 1..113: note = C0120917
NFMLTQPHSVSESPGKTVTISCTASGDTLDNKYVNWYQVRPGSAPATLIFHYEERDSGVPDRFSGSFDASSNSASLTISGLQTADEADYFCQSYDSKRDNLVVFGGGTKVTVL

SEQ ID NO: 372 - protein, 13 aa, Homo sapiens
  REGION 1..13: note = C0120917
TASGDTLDNKYVN

SEQ ID NO: 373 - protein, 7 aa, Homo sapiens
  REGION 1..7: note = C0120917
HYEERDS

SEQ ID NO: 374 - protein, 12 aa, Homo sapiens
  REGION 1..12: note = C0120917
QSYDSKRDNLVV

SEQ ID NO: 375 - protein, 22 aa, Homo sapiens
  REGION 1..22: note = C0120917
NFMLTQPHSVSESPGKTVTISC

SEQ ID NO: 376 - protein, 15 aa, Homo sapiens
  REGION 1..15: note = C0120917
WYQVRPGSAPATLIF

SEQ ID NO: 377 - protein, 34 aa, Homo sapiens
  REGION 1..34: note = C0120917
GVPDRFSGSFDASSNSASLTISGLQTADEADYFC

SEQ ID NO: 378 - protein, 10 aa, Homo sapiens
  REGION 1..10: note = C0120917
FGGGTKVTVL

SEQ ID NO: 379 - DNA, 387 bp, Homo sapiens
  misc_feature 1..387: note = C0120918
  source 1..387: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagatgatgtggatatgcgtggcggcgtgggcacctactacctgggcttcgatgtgtggggtcaggggaccacggtcaccgtctcctca

SEQ ID NO: 380 - protein, 129 aa, Homo sapiens
  REGION 1..129: note = C0120918
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTDDVDMRGGVGTYYLGFDVWGQGTTVTVSS

SEQ ID NO: 381 - protein, 5 aa, Homo sapiens
  REGION 1..5: note = C0120918
NAWMN

SEQ ID NO: 382 - protein, 19 aa, Homo sapiens
  REGION 1..19: note = C0120918
RIKSKTDGGTTDYAAPVKG

SEQ ID NO: 383 - protein, 18 aa, Homo sapiens
  REGION 1..18: note = C0120918
DDVDMRGGVGTYYLGFDV

SEQ ID NO: 384 - protein, 30 aa, Homo sapiens; identical to SEQ ID NO: 258
  REGION 1..30: note = C0120918

SEQ ID NO: 385 - protein, 14 aa, Homo sapiens
  REGION 1..14: note = C0120918
WVRQAPGKGLEWVG

SEQ ID NO: 386 - protein, 32 aa, Homo sapiens; identical to SEQ ID NO: 260
  REGION 1..32: note = C0120918

SEQ ID NO: 387 - protein, 11 aa, Homo sapiens
  REGION 1..11: note = C0120918
WGQGTTVTVSS

SEQ ID NO: 388 - DNA, 321 bp, Homo sapiens
  misc_feature 1..321: note = C0120918
  source 1..321: mol_type = other DNA
tcctatgagctgactcagccaccctcggtgtcagtgtccccaggacagacggccaggatcacctgctctggagatgcattgccaaagcaatatgcttattggtaccagcagaagccaggccaggcccctgtgctggtgatatataaagacagtgagaggccctcagggatccctgagcgattctctggctccagctcagggacaacagttacgttgaccatcagtggagtccaggcagaagacgaggctgactattactgtcaatcagcagacagcagtggtacttgggtgttcggcggagggaccaaggtcaccgtccta

SEQ ID NO: 389 - protein, 107 aa, Homo sapiens
  REGION 1..107: note = C0120918
SYELTQPPSVSVSPGQTARITCSGDALPKQYAYWYQQKPGQAPVLVIYKDSERPSGIPERFSGSSSGTTVTLTISGVQAEDEADYYCQSADSSGTWVFGGGTKVTVL

SEQ ID NO: 390 - protein, 11 aa, Homo sapiens
  REGION 1..11: note = C0120918
SGDALPKQYAY

SEQ ID NO: 391 - protein, 7 aa, Homo sapiens
  REGION 1..7: note = C0120918
KDSERPS

SEQ ID NO: 392 - protein, 10 aa, Homo sapiens
  REGION 1..10: note = C0120918
QSADSSGTWV

SEQ ID NO: 393 - protein, 22 aa, Homo sapiens
  REGION 1..22: note = C0120918
SYELTQPPSVSVSPGQTARITC

SEQ ID NO: 394 - protein, 15 aa, Homo sapiens
  REGION 1..15: note = C0120918
WYQQKPGQAPVLVIY

SEQ ID NO: 395 - protein, 32 aa, Homo sapiens
  REGION 1..32: note = C0120918
GIPERFSGSSSGTTVTLTISGVQAEDEADYYC

SEQ ID NO: 396 - protein, 10 aa, Homo sapiens
  REGION 1..10: note = C0120918
FGGGTKVTVL

SEQ ID NO: 397 - DNA, 366 bp, Homo sapiens
  misc_feature 1..366: note = C0120919
  source 1..366: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagatgaagcttacggctactacggcttcgatgtgtggggtcaggggaccacggtcaccgtctcctca

SEQ ID NO: 398 - protein, 122 aa, Homo sapiens
  REGION 1..122: note = C0120919
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTDEAYGYYGFDVWGQGTTVTVSS

SEQ ID NO: 399 - protein, 5 aa, Homo sapiens
  REGION 1..5: note = C0120919
NAWMN

SEQ ID NO: 400 - protein, 19 aa, Homo sapiens
  REGION 1..19: note = C0120919
RIKSKTDGGTTDYAAPVKG

SEQ ID NO: 401 - protein, 11 aa, Homo sapiens
  REGION 1..11: note = C0120919
DEAYGYYGFDV

SEQ ID NO: 402 - protein, 30 aa, Homo sapiens; identical to SEQ ID NO: 258
  REGION 1..30: note = C0120919

SEQ ID NO: 403 - protein, 14 aa, Homo sapiens
  REGION 1..14: note = C0120919
WVRQAPGKGLEWVG

SEQ ID NO: 404 - protein, 32 aa, Homo sapiens; identical to SEQ ID NO: 260
  REGION 1..32: note = C0120919

SEQ ID NO: 405 - protein, 11 aa, Homo sapiens
  REGION 1..11: note = C0120919
WGQGTTVTVSS

SEQ ID NO: 406 - DNA, 315 bp, Homo sapiens
  misc_feature 1..315: note = C0120919
  source 1..315: mol_type = other DNA
cagtctgccctgactcagccaccctcagtgtccgtgtcctcagggcagacagccagcatcacctgctctggagataaattgggtgataaatatgcttcttggtatcagcagaagccaggccagtcccctgttctggtcatctatcaagataccaagcggccctcagagatccctgagcgattctctggctccaactctgggaacacagccactctgaccatcagcgggacccaggctatggatgaggctgactattactgtcaggcgtgggacagcagcttggtattcggcggagggaccaaggtcaccgtccta

SEQ ID NO: 407 - protein, 105 aa, Homo sapiens
  REGION 1..105: note = C0120919
QSALTQPPSVSVSSGQTASITCSGDKLGDKYASWYQQKPGQSPVLVIYQDTKRPSEIPERFSGSNSGNTATLTISGTQAMDEADYYCQAWDSSLVFGGGTKVTVL

SEQ ID NO: 408 - protein, 11 aa, Homo sapiens
  REGION 1..11: note = C0120919
SGDKLGDKYAS

SEQ ID NO: 409 - protein, 7 aa, Homo sapiens
  REGION 1..7: note = C0120919
QDTKRPS

SEQ ID NO: 410 - protein, 8 aa, Homo sapiens
  REGION 1..8: note = C0120919
QAWDSSLV

SEQ ID NO: 411 - protein, 22 aa, Homo sapiens
  REGION 1..22: note = C0120919
QSALTQPPSVSVSSGQTASITC

SEQ ID NO: 412 - protein, 15 aa, Homo sapiens
  REGION 1..15: note = C0120919
WYQQKPGQSPVLVIY

SEQ ID NO: 413 - protein, 32 aa, Homo sapiens
  REGION 1..32: note = C0120919
EIPERFSGSNSGNTATLTISGTQAMDEADYYC

SEQ ID NO: 414 - protein, 10 aa, Homo sapiens
  REGION 1..10: note = C0120919
FGGGTKVTVL

SEQ ID NO: 415 - DNA, 363 bp, Homo sapiens
  misc_feature 1..363: note = C0120920
  source 1..363: mol_type = other DNA
gaggtgcagctggtgcagtctggacctgaggtgaagaagcctggggcctcagtgaaaatctcctgcaaggcctctggttataagttaaatgaccctggtgtccatggtgtcagctgggtgcgacaggcccctggacaagggcttgagtggatgggatggatcagcacactcaatgctgacacaagatatgcgcagaaattccagggcagagtcaccatggccacggactcatccacaagcacaatctacatggacctgaggagcctaaaatctgacgacacggccctttattactgtgcgagagatgactatactgccatttttgacttctggggccggggaaccctggtcaccgtctcctca

SEQ ID NO: 416 - protein, 121 aa, Homo sapiens
  REGION 1..121: note = C0120920
EVQLVQSGPEVKKPGASVKISCKASGYKLNDPGVHGVSWVRQAPGQGLEWMGWISTLNADTRYAQKFQGRVTMATDSSTSTIYMDLRSLKSDDTALYYCARDDYTAIFDFWGRGTLVTVSS

SEQ ID NO: 417 - protein, 7 aa, Homo sapiens
  REGION 1..7: note = C0120920
DPGVHGV

SEQ ID NO: 418 - protein, 17 aa, Homo sapiens
  REGION 1..17: note = C0120920
WISTLNADTRYAQKFQG

SEQ ID NO: 419 - protein, 9 aa, Homo sapiens
  REGION 1..9: note = C0120920
DDYTAIFDF

SEQ ID NO: 420 - protein, 30 aa, Homo sapiens; identical to SEQ ID NO: 294
  REGION 1..30: note = C0120920

SEQ ID NO: 421 - protein, 14 aa, Homo sapiens
  REGION 1..14: note = C0120920
WVRQAPGQGLEWMG

SEQ ID NO: 422 - protein, 32 aa, Homo sapiens; identical to SEQ ID NO: 296
  REGION 1..32: note = C0120920

SEQ ID NO: 423 - protein, 11 aa, Homo sapiens
  REGION 1..11: note = C0120920
WGRGTLVTVSS

SEQ ID NO: 424 - DNA, 324 bp, Homo sapiens; identical to SEQ ID NO: 298
  misc_feature 1..324: note = C0120920
  source 1..324: mol_type = other DNA

SEQ ID NO: 425 - protein, 108 aa, Homo sapiens; identical to SEQ ID NO: 299
  REGION 1..108: note = C0120920

SEQ ID NO: 426 - protein, 11 aa, Homo sapiens
  REGION 1..11: note = C0120920
GGSNIGSQSVH

SEQ ID NO: 427 - protein, 7 aa, Homo sapiens
  REGION 1..7: note = C0120920
DDSDRPS

SEQ ID NO: 428 - protein, 11 aa, Homo sapiens
  REGION 1..11: note = C0120920
QVWDSSTDHHV

SEQ ID NO: 429 - protein, 22 aa, Homo sapiens
  REGION 1..22: note = C0120920
QSVLTQPPSVSVAPGQTASITC

SEQ ID NO: 430 - protein, 15 aa, Homo sapiens
  REGION 1..15: note = C0120920
WYQQKPGQAPVLVVY

SEQ ID NO: 431 - protein, 32 aa, Homo sapiens; identical to SEQ ID NO: 305
  REGION 1..32: note = C0120920

SEQ ID NO: 432 - protein, 10 aa, Homo sapiens
  REGION 1..10: note = C0120920
FGTGTKVTVL

SEQ ID NO: 433 - DNA, 363 bp, Homo sapiens
  misc_feature 1..363: note = C0120921
  source 1..363: mol_type = other DNA
gaggtgcagctggtgcagtctggacctgaggtgaagaagcctggggcctcagtgaaaatctcctgcaaggcctctggttataagttaaatgaccctggtgtccatggtgtcagctgggtgcgacaggcccctggacaagggcttgagtggatgggatggatcagcacactccagggtgacacaagatatgcgcagaaattccagggcagagtcaccatggccacggactcatccacaagcacaatctacatggacctgaggagcctaaaatctgacgacacggccctttattactgtgcgagagatgactatactgccatttttgacttctggggccggggaaccctggtcaccgtctcctca

SEQ ID NO: 434 - protein, 121 aa, Homo sapiens
  REGION 1..121: note = C0120921
EVQLVQSGPEVKKPGASVKISCKASGYKLNDPGVHGVSWVRQAPGQGLEWMGWISTLQGDTRYAQKFQGRVTMATDSSTSTIYMDLRSLKSDDTALYYCARDDYTAIFDFWGRGTLVTVSS

SEQ ID NO: 435 - protein, 7 aa, Homo sapiens
  REGION 1..7: note = C0120921
DPGVHGV

SEQ ID NO: 436 - protein, 17 aa, Homo sapiens
  REGION 1..17: note = C0120921
WISTLQGDTRYAQKFQG

SEQ ID NO: 437 - protein, 9 aa, Homo sapiens
  REGION 1..9: note = C0120921
DDYTAIFDF

SEQ ID NO: 438 - protein, 30 aa, Homo sapiens; identical to SEQ ID NO: 294
  REGION 1..30: note = C0120921

SEQ ID NO: 439 - protein, 14 aa, Homo sapiens
  REGION 1..14: note = C0120921
WVRQAPGQGLEWMG

SEQ ID NO: 440 - protein, 32 aa, Homo sapiens; identical to SEQ ID NO: 296
  REGION 1..32: note = C0120921

SEQ ID NO: 441 - protein, 11 aa, Homo sapiens
  REGION 1..11: note = C0120921
WGRGTLVTVSS

SEQ ID NO: 442 - DNA, 324 bp, Homo sapiens; identical to SEQ ID NO: 298
  misc_feature 1..324: note = C0120921
  source 1..324: mol_type = other DNA

SEQ ID NO: 443 - protein, 108 aa, Homo sapiens; identical to SEQ ID NO: 299
  REGION 1..108: note = C0120921

SEQ ID NO: 444 - protein, 11 aa, Homo sapiens
  REGION 1..11: note = C0120921
GGSNIGSQSVH

SEQ ID NO: 445 - protein, 7 aa, Homo sapiens
  REGION 1..7: note = C0120921
DDSDRPS

SEQ ID NO: 446 - protein, 11 aa, Homo sapiens
  REGION 1..11: note = C0120921
QVWDSSTDHHV

SEQ ID NO: 447 - protein, 22 aa, Homo sapiens
  REGION 1..22: note = C0120921
QSVLTQPPSVSVAPGQTASITC

SEQ ID NO: 448 - protein, 15 aa, Homo sapiens
  REGION 1..15: note = C0120921
WYQQKPGQAPVLVVY

SEQ ID NO: 449 - protein, 32 aa, Homo sapiens; identical to SEQ ID NO: 305
  REGION 1..32: note = C0120921

SEQ ID NO: 450 - protein, 10 aa, Homo sapiens
  REGION 1..10: note = C0120921
FGTGTKVTVL

SEQ ID NO: 451 - DNA, 357 bp, Homo sapiens
  misc_feature 1..357: note = C0120922
  source 1..357: mol_type = other DNA
caggtgcagctggtgcagtctggagctgaggtgaagaagcctgggtcctcggtgaaggtctcctgcaaggcttctggaggcaccttcaacagctatactatcaactgggtgcgacaggcccctggacaagggcttgagtgggtgggagggttcatccctatctttggtacagcagactacgcacagaagttccacggcagagtcaccattaccgcggacgaatccaccagcacagcctacatggagctgagcagcctgagatctgaggacacggccgtgtattactgtgcgagagctccccttgggtctgaaccatttgactactgggggcaagggacactggtcaccgtctcctca

SEQ ID NO: 452 - protein, 119 aa, Homo sapiens
  REGION 1..119: note = C0120922
QVQLVQSGAEVKKPGSSVKVSCKASGGTFNSYTINWVRQAPGQGLEWVGGFIPIFGTADYAQKFHGRVTITADESTSTAYMELSSLRSEDTAVYYCARAPLGSEPFDYWGQGTLVTVSS

SEQ ID NO: 453 - protein, 5 aa, Homo sapiens
  REGION 1..5: note = C0120922
SYTIN

SEQ ID NO: 454 - protein, 17 aa, Homo sapiens
  REGION 1..17: note = C0120922
GFIPIFGTADYAQKFHG

SEQ ID NO: 455 - protein, 10 aa, Homo sapiens
  REGION 1..10: note = C0120922
APLGSEPFDY

SEQ ID NO: 456 - protein, 30 aa, Homo sapiens
  REGION 1..30: note = C0120922
QVQLVQSGAEVKKPGSSVKVSCKASGGTFN

SEQ ID NO: 457 - protein, 14 aa, Homo sapiens
  REGION 1..14: note = C0120922
WVRQAPGQGLEWVG

SEQ ID NO: 458 - protein, 32 aa, Homo sapiens
  REGION 1..32: note = C0120922
RVTITADESTSTAYMELSSLRSEDTAVYYCAR

SEQ ID NO: 459 - protein, 11 aa, Homo sapiens
  REGION 1..11: note = C0120922
WGQGTLVTVSS

SEQ ID NO: 460 - DNA, 324 bp, Homo sapiens; identical to SEQ ID NO: 244
  misc_feature 1..324: note = C0120922
  source 1..324: mol_type = other DNA

SEQ ID NO: 461 - protein, 108 aa, Homo sapiens; identical to SEQ ID NO: 245
  REGION 1..108: note = C0120922

SEQ ID NO: 462 - protein, 11 aa, Homo sapiens
  REGION 1..11: note = C0120922
GGNNIGSKSVH

SEQ ID NO: 463 - protein, 7 aa, Homo sapiens
  REGION 1..7: note = C0120922
DDSDRPS

SEQ ID NO: 464 - protein, 11 aa, Homo sapiens
  REGION 1..11: note = C0120922
QVWDSSSDHLV

SEQ ID NO: 465 - protein, 22 aa, Homo sapiens
  REGION 1..22: note = C0120922
SYVLTQPPSVSVAPGQTARITC

SEQ ID NO: 466 - protein, 15 aa, Homo sapiens
  REGION 1..15: note = C0120922
WYQQKPGQAPVLVVY

SEQ ID NO: 467 - protein, 32 aa, Homo sapiens; identical to SEQ ID NO: 251
  REGION 1..32: note = C0120922

SEQ ID NO: 468 - protein, 10 aa, Homo sapiens
  REGION 1..10: note = C0120922
FGGGTKLTVL

SEQ ID NO: 469 - DNA, 402 bp, Homo sapiens
  misc_feature 1..402: note = C0120923
  source 1..402: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagatcgtccgccgatgtctcgtggcccgtcttctgtgtacgaaggcccgtaccgttacggcttcgatgtgtggggtcaggggaccctggtcaccgtctcctca

SEQ ID NO: 470 - protein, 134 aa, Homo sapiens
  REGION 1..134: note = C0120923
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTDRPPMSRGPSSVYEGPYRYGFDVWGQGTLVTVSS

SEQ ID NO: 471 - protein, 5 aa, Homo sapiens
  REGION 1..5: note = C0120923
NAWMN

SEQ ID NO: 472 - protein, 19 aa, Homo sapiens
  REGION 1..19: note = C0120923
RIKSKTDGGTTDYAAPVKG

SEQ ID NO: 473 - protein, 23 aa, Homo sapiens
  REGION 1..23: note = C0120923
DRPPMSRGPSSVYEGPYRYGFDV

SEQ ID NO: 474 - protein, 30 aa, Homo sapiens; identical to SEQ ID NO: 258
  REGION 1..30: note = C0120923

SEQ ID NO: 475 - protein, 14 aa, Homo sapiens
  REGION 1..14: note = C0120923
WVRQAPGKGLEWVG

SEQ ID NO: 476 - protein, 32 aa, Homo sapiens; identical to SEQ ID NO: 260
  REGION 1..32: note = C0120923

SEQ ID NO: 477 - protein, 11 aa, Homo sapiens
  REGION 1..11: note = C0120923
WGQGTLVTVSS

SEQ ID NO: 478 - DNA, 333 bp, Homo sapiens
  misc_feature 1..333: note = C0120923
  source 1..333: mol_type = other DNA
aattttatgctgactcagccccactctgtgtcggagtctccggggaagacggtaaccatctcctgcaccggcagcggcggcaacattgtcgataattttgtacaatggcaccagcagcgcccgggcagtagccccaccactgtgatctatgaggatgaccaaagaccctctggggtccctgatcggttctctgcctccgtcgacagctcctccaactctggcagcctcaccatctctggactgaagactgaggacgaggctgattactactgtcaatcttatgatggcgccaatcgttgggtcttcggcggagggaccaagctgaccgtccta

SEQ ID NO: 479 - protein, 111 aa, Homo sapiens
  REGION 1..111: note = C0120923
NFMLTQPHSVSESPGKTVTISCTGSGGNIVDNFVQWHQQRPGSSPTTVIYEDDQRPSGVPDRFSASVDSSSNSGSLTISGLKTEDEADYYCQSYDGANRWVFGGGTKLTVL

SEQ ID NO: 480 - protein, 13 aa, Homo sapiens
  REGION 1..13: note = C0120923
TGSGGNIVDNFVQ

SEQ ID NO: 481 - protein, 7 aa, Homo sapiens
  REGION 1..7: note = C0120923
EDDQRPS

SEQ ID NO: 482 - protein, 10 aa, Homo sapiens
  REGION 1..10: note = C0120923
QSYDGANRWV

SEQ ID NO: 483 - protein, 22 aa, Homo sapiens
  REGION 1..22: note = C0120923
NFMLTQPHSVSESPGKTVTISC

SEQ ID NO: 484 - protein, 15 aa, Homo sapiens
  REGION 1..15: note = C0120923
WHQQRPGSSPTTVIY

SEQ ID NO: 485 - protein, 34 aa, Homo sapiens
  REGION 1..34: note = C0120923
GVPDRFSASVDSSSNSGSLTISGLKTEDEADYYC

SEQ ID NO: 486 - protein, 10 aa, Homo sapiens
  REGION 1..10: note = C0120923
FGGGTKLTVL

SEQ ID NO: 487 - DNA, 363 bp, Homo sapiens
  misc_feature 1..363: note = C0120924
  source 1..363: mol_type = other DNA
caggtgcagctggtgcagtctggagccgaggtgaagaagcctggggcctcagtgaaagtgtcctgcaaggcctctggttataagttaaatgaccctggtgtccatggtgtcagctgggtgcgacaggcccctggacaagggcttgagtggatgggatggatcagcacactcaatggtgacacaagatatgcgcagaaattccagggcagagtcaccatgaccacggactcatccacaagcacaatctacatggagctgaggagcctacggtctgacgacacggccgtgtattactgtgcgagagatgactatactgccatttttgacttctggggccagggaaccctggtcaccgtctcctca

SEQ ID NO: 488 - protein, 121 aa, Homo sapiens
  REGION 1..121: note = C0120924
QVQLVQSGAEVKKPGASVKVSCKASGYKLNDPGVHGVSWVRQAPGQGLEWMGWISTLNGDTRYAQKFQGRVTMTTDSSTSTIYMELRSLRSDDTAVYYCARDDYTAIFDFWGQGTLVTVSS

SEQ ID NO: 489 - protein, 7 aa, Homo sapiens
  REGION 1..7: note = C0120924
DPGVHGV

SEQ ID NO: 490 - protein, 17 aa, Homo sapiens
  REGION 1..17: note = C0120924
WISTLNGDTRYAQKFQG

SEQ ID NO: 491 - protein, 9 aa, Homo sapiens
  REGION 1..9: note = C0120924
DDYTAIFDF

SEQ ID NO: 492 - protein, 30 aa, Homo sapiens
  REGION 1..30: note = C0120924
QVQLVQSGAEVKKPGASVKVSCKASGYKLN

SEQ ID NO: 493 - protein, 14 aa, Homo sapiens
  REGION 1..14: note = C0120924
WVRQAPGQGLEWMG

SEQ ID NO: 494 - protein, 32 aa, Homo sapiens
  REGION 1..32: note = C0120924
RVTMTTDSSTSTIYMELRSLRSDDTAVYYCAR

SEQ ID NO: 495 - protein, 11 aa, Homo sapiens
  REGION 1..11: note = C0120924
WGQGTLVTVSS

SEQ ID NO: 496 - DNA, 324 bp, Homo sapiens
  misc_feature 1..324: note = C0120924
  source 1..324: mol_type = other DNA
agctctgtgttgactcagcccccctcggtgtcagtggccccaggacagacggcccggattacctgtgggggaagcaacattggaagtcagagtgttcactggtaccagcagaagccaggccaggcccctgtgctggtcgtctatgatgatagcgaccggccctcagggatccctgagcgattctctggctccaagtctgggaacacggccaccctgaccatcagcagggtcgaagccggggatgaggccgactattactgtcaggtgtgggatagtagtactgatcatcatgtcttcggaactgggaccaaggtcaccgtccta

SEQ ID NO: 497 - protein, 108 aa, Homo sapiens
  REGION 1..108: note = C0120924
SSVLTQPPSVSVAPGQTARITCGGSNIGSQSVHWYQQKPGQAPVLVVYDDSDRPSGIPERFSGSKSGNTATLTISRVEAGDEADYYCQVWDSSTDHHVFGTGTKVTVL

SEQ ID NO: 498 - protein, 11 aa, Homo sapiens
  REGION 1..11: note = C0120924
GGSNIGSQSVH

SEQ ID NO: 499 - protein, 7 aa, Homo sapiens
  REGION 1..7: note = C0120924
DDSDRPS

SEQ ID NO: 500 - protein, 11 aa, Homo sapiens
  REGION 1..11: note = C0120924
QVWDSSTDHHV

SEQ ID NO: 501 - protein, 22 aa, Homo sapiens
  REGION 1..22: note = C0120924
SSVLTQPPSVSVAPGQTARITC

SEQ ID NO: 502 - protein, 15 aa, Homo sapiens
  REGION 1..15: note = C0120924
WYQQKPGQAPVLVVY

SEQ ID NO: 503 - protein, 32 aa, Homo sapiens
  REGION 1..32: note = C0120924
GIPERFSGSKSGNTATLTISRVEAGDEADYYC

SEQ ID NO: 504 - protein, 10 aa, Homo sapiens
  REGION 1..10: note = C0120924
FGTGTKVTVL

SEQ ID NO: 505 - DNA, 348 bp, Homo sapiens
  misc_feature 1..348: note = C0120925
  source 1..348: mol_type = other DNA
caggtgcagctgcaggagtccggcccaggactggtgaagccttcggagaccctgtccctcacctgcactgtgtctggtggctccgtcagcagtgggcgttacgactggagttggatccggcagcccccagggaagggactggaatggattggccatatttattacactgggatcaccgactacaatccctccctcaggagtcgagtcaccatttcagcagacaagagcaagaaccagttctccctgaagttgagctctgtgaccgctgcggacacggccgtctattattgtgcgagaggcccctttttcgacccctggggccaagggacactggtcaccgtctcctca

SEQ ID NO: 506 - protein, 116 aa, Homo sapiens
  REGION 1..116: note = C0120925
QVQLQESGPGLVKPSETLSLTCTVSGGSVSSGRYDWSWIRQPPGKGLEWIGHIYYTGITDYNPSLRSRVTISADKSKNQFSLKLSSVTAADTAVYYCARGPFFDPWGQGTLVTVSS

SEQ ID NO: 507 - protein, 7 aa, Homo sapiens
  REGION 1..7: note = C0120925
SGRYDWS

SEQ ID NO: 508 - protein, 16 aa, Homo sapiens
  REGION 1..16: note = C0120925
HIYYTGITDYNPSLRS

SEQ ID NO: 509 - protein, 6 aa, Homo sapiens
  REGION 1..6: note = C0120925
GPFFDP

SEQ ID NO: 510 - protein, 30 aa, Homo sapiens; identical to SEQ ID NO: 348
  REGION 1..30: note = C0120925

SEQ ID NO: 511 - protein, 14 aa, Homo sapiens
  REGION 1..14: note = C0120925
WIRQPPGKGLEWIG

SEQ ID NO: 512 - protein, 32 aa, Homo sapiens
  REGION 1..32: note = C0120925
RVTISADKSKNQFSLKLSSVTAADTAVYYCAR

SEQ ID NO: 513 - protein, 11 aa, Homo sapiens
  REGION 1..11: note = C0120925
WGQGTLVTVSS

SEQ ID NO: 514 - DNA, 324 bp, Homo sapiens
  misc_feature 1..324: note = C0120925
  source 1..324: mol_type = other DNA
agctctgtgttgacgcagccaccctcggtgtccgtggccccaggacagacggcccggatcacctgtggggcaaacaacattggaagtaaaagtgttcagtggtaccaacagaagccaggccaggcccctgttctggtcgtctatgatggtagcgaccggccctcagggatccctgagcgattctctggctccagttctgagaacacggccaccctgaccatcagcagggtcgaagccggggatgaggccgactattactgtcagctgtgggatagtgttacagatcgtccggtattcggcaccgggaccaaggtcaccgtccta

SEQ ID NO: 515 - protein, 108 aa, Homo sapiens
  REGION 1..108: note = C0120925
SSVLTQPPSVSVAPGQTARITCGANNIGSKSVQWYQQKPGQAPVLVVYDGSDRPSGIPERFSGSSSENTATLTISRVEAGDEADYYCQLWDSVTDRPVFGTGTKVTVL

SEQ ID NO: 516 - protein, 11 aa, Homo sapiens
  REGION 1..11: note = C0120925
GANNIGSKSVQ

SEQ ID NO: 517 - protein, 7 aa, Homo sapiens
  REGION 1..7: note = C0120925
DGSDRPS

SEQ ID NO: 518 - protein, 11 aa, Homo sapiens
  REGION 1..11: note = C0120925
QLWDSVTDRPV

SEQ ID NO: 519 - protein, 22 aa, Homo sapiens
  REGION 1..22: note = C0120925
SSVLTQPPSVSVAPGQTARITC

SEQ ID NO: 520 - protein, 15 aa, Homo sapiens
  REGION 1..15: note = C0120925
WYQQKPGQAPVLVVY

SEQ ID NO: 521 - protein, 32 aa, Homo sapiens
  REGION 1..32: note = C0120925
GIPERFSGSSSENTATLTISRVEAGDEADYYC

SEQ ID NO: 522 - protein, 10 aa, Homo sapiens
  REGION 1..10: note = C0120925
FGTGTKVTVL

SEQ ID NO: 523 - DNA, 396 bp, Homo sapiens; identical to SEQ ID NO: 361
  misc_feature 1..396: note = C0120926
  source 1..396: mol_type = other DNA

SEQ ID NO: 524 - protein, 132 aa, Homo sapiens; identical to SEQ ID NO: 362
  REGION 1..132: note = C0120926

SEQ ID NO: 525 - protein, 5 aa, Homo sapiens
  REGION 1..5: note = C0120926
NAWMN

SEQ ID NO: 526 - protein, 19 aa, Homo sapiens
  REGION 1..19: note = C0120926
RIKSKTDGGTTDYAAPVKG

SEQ ID NO: 527 - protein, 21 aa, Homo sapiens
  REGION 1..21: note = C0120926
EDPAGYYGTEGSYRYYGYMDY

SEQ ID NO: 528 - protein, 30 aa, Homo sapiens; identical to SEQ ID NO: 258
  REGION 1..30: note = C0120926

SEQ ID NO: 529 - protein, 14 aa, Homo sapiens
  REGION 1..14: note = C0120926
WVRQAPGKGLEWVG

SEQ ID NO: 530 - protein, 32 aa, Homo sapiens; identical to SEQ ID NO: 260
  REGION 1..32: note = C0120926

SEQ ID NO: 531 - protein, 11 aa, Homo sapiens
  REGION 1..11: note = C0120926
WGQGTTVTVSS

SEQ ID NO: 532 - DNA, 339 bp, Homo sapiens
  misc_feature 1..339: note = C0120926
  source 1..339: mol_type = other DNA
aattttatgctgactcagccccactctgtgtcggaatctccggggaagacggtgaccatctcctgcaccgccagcggtgacacccttgacaacaaatatgtgaactggtatcaacagcgcccgggcagtagccccaccactttgatcttccattatgaagaaagagactctggggtccctgatcggttctctggctcctttgacgcctcctccaattctgcctccctcaccatctctggactgaagactgaggacgaggctgattattactgtcagtcttatgactccaagcgcgacaacttagttgttttcggcggagggaccaagctgaccgtccta

SEQ ID NO: 533 - protein, 113 aa, Homo sapiens
  REGION 1..113: note = C0120926
NFMLTQPHSVSESPGKTVTISCTASGDTLDNKYVNWYQQRPGSSPTTLIFHYEERDSGVPDRFSGSFDASSNSASLTISGLKTEDEADYYCQSYDSKRDNLVVFGGGTKLTVL

SEQ ID NO: 534 - protein, 13 aa, Homo sapiens
  REGION 1..13: note = C0120926
TASGDTLDNKYVN

SEQ ID NO: 535 - protein, 7 aa, Homo sapiens
  REGION 1..7: note = C0120926
HYEERDS

SEQ ID NO: 536 - protein, 12 aa, Homo sapiens
  REGION 1..12: note = C0120926
QSYDSKRDNLVV

SEQ ID NO: 537 - protein, 22 aa, Homo sapiens
  REGION 1..22: note = C0120926
NFMLTQPHSVSESPGKTVTISC

SEQ ID NO: 538 - protein, 15 aa, Homo sapiens
  REGION 1..15: note = C0120926
WYQQRPGSSPTTLIF

SEQ ID NO: 539 - protein, 34 aa, Homo sapiens
  REGION 1..34: note = C0120926
GVPDRFSGSFDASSNSASLTISGLKTEDEADYYC

SEQ ID NO: 540 - protein, 10 aa, Homo sapiens
  REGION 1..10: note = C0120926
FGGGTKLTVL

SEQ ID NO: 541 - DNA, 387 bp, Homo sapiens
  misc_feature 1..387: note = C0120927
  source 1..387: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagatgatgtggatatgcgtggcggcgtgggcacctactacctgggcttcgatgtgtggggtcaggggaccctggtcaccgtctcctca

SEQ ID NO: 542 - protein, 129 aa, Homo sapiens
  REGION 1..129: note = C0120927
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTDDVDMRGGVGTYYLGFDVWGQGTLVTVSS

SEQ ID NO: 543 - protein, 5 aa, Homo sapiens
  REGION 1..5: note = C0120927
NAWMN

SEQ ID NO: 544 - protein, 19 aa, Homo sapiens
  REGION 1..19: note = C0120927
RIKSKTDGGTTDYAAPVKG

SEQ ID NO: 545 - protein, 18 aa, Homo sapiens
  REGION 1..18: note = C0120927
DDVDMRGGVGTYYLGFDV

SEQ ID NO: 546 - protein, 30 aa, Homo sapiens; identical to SEQ ID NO: 258
  REGION 1..30: note = C0120927

SEQ ID NO: 547 - protein, 14 aa, Homo sapiens
  REGION 1..14: note = C0120927
WVRQAPGKGLEWVG

SEQ ID NO: 548 - protein, 32 aa, Homo sapiens; identical to SEQ ID NO: 260
  REGION 1..32: note = C0120927

SEQ ID NO: 549 - protein, 11 aa, Homo sapiens
  REGION 1..11: note = C0120927
WGQGTLVTVSS

SEQ ID NO: 550 - DNA, 321 bp, Homo sapiens
  misc_feature 1..321: note = C0120927
  source 1..321: mol_type = other DNA
tcctatgagctgactcagccaccctcggtgtcagtgtccccaggacagacggccaggatcacctgctctggagatgcattgccaaagcaatatgcttattggtaccagcagaagccaggccaggcccctgtgctggtgatatataaagacagtgagaggccctcagggatccctgagcgattctctggctccagctcagggacaacagttacgttgaccatcagtggagtccaggcagaagacgaggctgactattactgtcaatcagcagacagcagtggtacttgggtgttcggcggagggaccaagctcaccgtccta

SEQ ID NO: 551 - protein, 107 aa, Homo sapiens
  REGION 1..107: note = C0120927
SYELTQPPSVSVSPGQTARITCSGDALPKQYAYWYQQKPGQAPVLVIYKDSERPSGIPERFSGSSSGTTVTLTISGVQAEDEADYYCQSADSSGTWVFGGGTKLTVL

SEQ ID NO: 552 - protein, 11 aa, Homo sapiens
  REGION 1..11: note = C0120927
SGDALPKQYAY

SEQ ID NO: 553 - protein, 7 aa, Homo sapiens
  REGION 1..7: note = C0120927
KDSERPS

SEQ ID NO: 554 - protein, 10 aa, Homo sapiens
  REGION 1..10: note = C0120927
QSADSSGTWV

SEQ ID NO: 555 - protein, 22 aa, Homo sapiens
  REGION 1..22: note = C0120927
SYELTQPPSVSVSPGQTARITC

SEQ ID NO: 556 - protein, 15 aa, Homo sapiens
  REGION 1..15: note = C0120927
WYQQKPGQAPVLVIY

SEQ ID NO: 557 - protein, 32 aa, Homo sapiens; identical to SEQ ID NO: 395
  REGION 1..32: note = C0120927

SEQ ID NO: 558 - protein, 10 aa, Homo sapiens
  REGION 1..10: note = C0120927
FGGGTKLTVL

SEQ ID NO: 559 - DNA, 366 bp, Homo sapiens
  misc_feature 1..366: note = C0120928
  source 1..366: mol_type = other DNA
gaggtgcagctggtggagtctgggggaggcttggtaaagcctggggggtcccttagactctcctgtgcagcctctggtttcactttcagtaacgcctggatgaactgggtccgccaggctccagggaaggggctggagtgggtcggccgtattaaaagcaaaactgatggtgggacaacagactacgctgcacccgtgaaaggcagattcaccatctcaagagatgattcaaaaaacacgctgtatctgcaaatgaacagcctgaaaaccgaggacacagccgtgtattactgtaccacagatgaagcttacggctactacggcttcgatgtgtggggtcaggggaccctggtcaccgtctcctca

SEQ ID NO: 560 - protein, 122 aa, Homo sapiens
  REGION 1..122: note = C0120928
EVQLVESGGGLVKPGGSLRLSCAASGFTFSNAWMNWVRQAPGKGLEWVGRIKSKTDGGTTDYAAPVKGRFTISRDDSKNTLYLQMNSLKTEDTAVYYCTTDEAYGYYGFDVWGQGTLVTVSS

SEQ ID NO: 561 - protein, 5 aa, Homo sapiens
  REGION 1..5: note = C0120928
NAWMN

SEQ ID NO: 562 - protein, 19 aa, Homo sapiens
  REGION 1..19: note = C0120928
RIKSKTDGGTTDYAAPVKG

SEQ ID NO: 563 - protein, 11 aa, Homo sapiens
  REGION 1..11: note = C0120928
DEAYGYYGFDV

SEQ ID NO: 564 - protein, 30 aa, Homo sapiens; identical to SEQ ID NO: 258
  REGION 1..30: note = C0120928

SEQ ID NO: 565 - protein, 14 aa, Homo sapiens
  REGION 1..14: note = C0120928
WVRQAPGKGLEWVG

SEQ ID NO: 566 - protein, 32 aa, Homo sapiens; identical to SEQ ID NO: 260
  REGION 1..32: note = C0120928

SEQ ID NO: 567 - protein, 11 aa, Homo sapiens
  REGION 1..11: note = C0120928
WGQGTLVTVSS

SEQ ID NO: 568 - DNA, 315 bp, Homo sapiens
  misc_feature 1..315: note = C0120928
  source 1..315: mol_type = other DNA
agctctgagctgactcagccaccctcagtgtccgtgtcccccgggcagacagccagcatcacctgctctggagataaattgggtgataaatatgcttcttggtatcagcagaagccaggccagtcccctgttctggtcatctatcaagataccaagcggccctcaggcatccctgagcgattctctggctccaactctgggaacacagccactctgaccatcagcgggacccaggctatggatgaggctgactattactgtcaggcgtgggacagcagcttggtattcggcaccgggaccaaggtcaccgtccta

SEQ ID NO: 569 - protein, 105 aa, Homo sapiens
  REGION 1..105: note = C0120928
SSELTQPPSVSVSPGQTASITCSGDKLGDKYASWYQQKPGQSPVLVIYQDTKRPSGIPERFSGSNSGNTATLTISGTQAMDEADYYCQAWDSSLVFGTGTKVTVL

SEQ ID NO: 570 - protein, 11 aa, Homo sapiens
  REGION 1..11: note = C0120928
SGDKLGDKYAS

SEQ ID NO: 571 - protein, 7 aa, Homo sapiens
  REGION 1..7: note = C0120928
QDTKRPS

SEQ ID NO: 572 - protein, 8 aa, Homo sapiens
  REGION 1..8: note = C0120928
QAWDSSLV

SEQ ID NO: 573 - protein, 22 aa, Homo sapiens
  REGION 1..22: note = C0120928
SSELTQPPSVSVSPGQTASITC

SEQ ID NO: 574 - protein, 15 aa, Homo sapiens
  REGION 1..15: note = C0120928
WYQQKPGQSPVLVIY

SEQ ID NO: 575 - protein, 32 aa, Homo sapiens
  REGION 1..32: note = C0120928
GIPERFSGSNSGNTATLTISGTQAMDEADYYC

SEQ ID NO: 576 - protein, 10 aa, Homo sapiens
  REGION 1..10: note = C0120928
FGTGTKVTVL

SEQ ID NO: 577 - DNA, 363 bp, Homo sapiens
  misc_feature 1..363: note = C0120929
  source 1..363: mol_type = other DNA
caggtgcagctggtgcagtctggagccgaggtgaagaagcctggggcctcagtgaaagtgtcctgcaaggcctctggttataagttaaatgaccctggtgtccatggtgtcagctgggtgcgacaggcccctggacaagggcttgagtggatgggatggatcagcacactcaatgctgacacaagatatgcgcagaaattccagggcagagtcaccatgaccacggactcatccacaagcacaatctacatggagctgaggagcctacggtctgacgacacggccgtgtattactgtgcgagagatgactatactgccatttttgacttctggggccagggaaccctggtcaccgtctcctca

SEQ ID NO: 578 - protein, 121 aa, Homo sapiens
  REGION 1..121: note = C0120929
QVQLVQSGAEVKKPGASVKVSCKASGYKLNDPGVHGVSWVRQAPGQGLEWMGWISTLNADTRYAQKFQGRVTMTTDSSTSTIYMELRSLRSDDTAVYYCARDDYTAIFDFWGQGTLVTVSS

SEQ ID NO: 579 - protein, 7 aa, Homo sapiens
  REGION 1..7: note = C0120929
DPGVHGV

SEQ ID NO: 580 - protein, 17 aa, Homo sapiens
  REGION 1..17: note = C0120929
WISTLNADTRYAQKFQG

SEQ ID NO: 581 - protein, 9 aa, Homo sapiens
  REGION 1..9: note = C0120929
DDYTAIFDF

SEQ ID NO: 582 - protein, 30 aa, Homo sapiens; identical to SEQ ID NO: 492
  REGION 1..30: note = C0120929

SEQ ID NO: 583 - protein, 14 aa, Homo sapiens
  REGION 1..14: note = C0120929
WVRQAPGQGLEWMG

SEQ ID NO: 584 - protein, 32 aa, Homo sapiens; identical to SEQ ID NO: 494
  REGION 1..32: note = C0120929

SEQ ID NO: 585 - protein, 11 aa, Homo sapiens
  REGION 1..11: note = C0120929
WGQGTLVTVSS

SEQ ID NO: 586 - DNA, 324 bp, Homo sapiens; identical to SEQ ID NO: 496
  misc_feature 1..324: note = C0120929
  source 1..324: mol_type = other DNA

SEQ ID NO: 587 - protein, 108 aa, Homo sapiens; identical to SEQ ID NO: 497
  REGION 1..108: note = C0120929

SEQ ID NO: 588 - protein, 11 aa, Homo sapiens
  REGION 1..11: note = C0120929
GGSNIGSQSVH

SEQ ID NO: 589 - protein, 7 aa, Homo sapiens
  REGION 1..7: note = C0120929
DDSDRPS

SEQ ID NO: 590 - protein, 11 aa, Homo sapiens
  REGION 1..11: note = C0120929
QVWDSSTDHHV

SEQ ID NO: 591 - protein, 22 aa, Homo sapiens
  REGION 1..22: note = C0120929
SSVLTQPPSVSVAPGQTARITC

SEQ ID NO: 592 - protein, 15 aa, Homo sapiens
  REGION 1..15: note = C0120929
WYQQKPGQAPVLVVY

SEQ ID NO: 593 - protein, 32 aa, Homo sapiens; identical to SEQ ID NO: 503
  REGION 1..32: note = C0120929

SEQ ID NO: 594 - protein, 10 aa, Homo sapiens
  REGION 1..10: note = C0120929
FGTGTKVTVL

SEQ ID NO: 595 - DNA, 363 bp, Homo sapiens
  misc_feature 1..363: note = C0120930
  source 1..363: mol_type = other DNA
caggtgcagctggtgcagtctggagccgaggtgaagaagcctggggcctcagtgaaagtgtcctgcaaggcctctggttataagttaaatgaccctggtgtccatggtgtcagctgggtgcgacaggcccctggacaagggcttgagtggatgggatggatcagcacactccagggtgacacaagatatgcgcagaaattccagggcagagtcaccatgaccacggactcatccacaagcacaatctacatggagctgaggagcctacggtctgacgacacggccgtgtattactgtgcgagagatgactatactgccatttttgacttctggggccagggaaccctggtcaccgtctcctca

SEQ ID NO: 596 - protein, 121 aa, Homo sapiens
  REGION 1..121: note = C0120930
QVQLVQSGAEVKKPGASVKVSCKASGYKLNDPGVHGVSWVRQAPGQGLEWMGWISTLQGDTRYAQKFQGRVTMTTDSSTSTIYMELRSLRSDDTAVYYCARDDYTAIFDFWGQGTLVTVSS

SEQ ID NO: 597 - protein, 7 aa, Homo sapiens
  REGION 1..7: note = C0120930
DPGVHGV

SEQ ID NO: 598 - protein, 17 aa, Homo sapiens
  REGION 1..17: note = C0120930
WISTLQGDTRYAQKFQG

SEQ ID NO: 599 - protein, 9 aa, Homo sapiens
  REGION 1..9: note = C0120930
DDYTAIFDF

SEQ ID NO: 600 - protein, 30 aa, Homo sapiens; identical to SEQ ID NO: 492
  REGION 1..30: note = C0120930

SEQ ID NO: 601 - protein, 14 aa, Homo sapiens
  REGION 1..14: note = C0120930
WVRQAPGQGLEWMG

SEQ ID NO: 602 - protein, 32 aa, Homo sapiens; identical to SEQ ID NO: 494
  REGION 1..32: note = C0120930

SEQ ID NO: 603 - protein, 11 aa, Homo sapiens
  REGION 1..11: note = C0120930
WGQGTLVTVSS

SEQ ID NO: 604 - DNA, 324 bp, Homo sapiens; identical to SEQ ID NO: 496
  misc_feature 1..324: note = C0120930
  source 1..324: mol_type = other DNA

SEQ ID NO: 605 - protein, 108 aa, Homo sapiens; identical to SEQ ID NO: 497
  REGION 1..108: note = C0120930

SEQ ID NO: 606 - protein, 11 aa, Homo sapiens
  REGION 1..11: note = C0120930
GGSNIGSQSVH

SEQ ID NO: 607 - protein, 7 aa, Homo sapiens
  REGION 1..7: note = C0120930
DDSDRPS

SEQ ID NO: 608 - protein, 11 aa, Homo sapiens
  REGION 1..11: note = C0120930
QVWDSSTDHHV

SEQ ID NO: 609 - protein, 22 aa, Homo sapiens
  REGION 1..22: note = C0120930
SSVLTQPPSVSVAPGQTARITC

SEQ ID NO: 610 - protein, 15 aa, Homo sapiens
  REGION 1..15: note = C0120930
WYQQKPGQAPVLVVY

SEQ ID NO: 611 - protein, 32 aa, Homo sapiens; identical to SEQ ID NO: 503
  REGION 1..32: note = C0120930

SEQ ID NO: 612 - protein, 10 aa, Homo sapiens
  REGION 1..10: note = C0120930
FGTGTKVTVL

SEQ ID NO: 613 - protein, 761 aa, Homo sapiens
MALPALGLDPWSLLGLFLFQLLQLLLPTTTAGGGGQGPMPRVRYYAGDERRALSFFHQKGLQDFDTLLLSGDGNTLYVGAREAILALDIQDPGVPRLKNMIPWPASDRKKSECAFKKKSNETQCFNFIRVLVSYNVTHLYTCGTFAFSPACTFIELQDSYLLPISEDKVMEGKGQSPFDPAHKHTAVLVDGMLYSGTMNNFLGSEPILMRTLGSQPVLKTDNFLRWLHHDASFVAAIPSTQVVYFFFEETASEFDFFERLHTSRVARVCKNDVGGEKLLQKKWTTFLKAQLLCTQPGQLPFNVIRHAVLLPADSPTAPHIYAVFTSQWQVGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGHSHLVMYLGTTTGSLHKAVVSGDSSAHLVEEIQLFPDPEPVRNLQLAPTQGAVFVGFSGGVWRVPRANCSVYESCVDCVLARDPHCAWDPESRTCCLLSAPNLNSWKQDMERGNPEWACASGPMSRSLRPQSRPQIIKEVLAVPNSILELPCPHLSALASYYWSHGPAAVPEASSTVYNGSLLLIVQDGVGGLYQCWATENGFSYPVISYWVDSQDQTLALDPELAGIPREHVKVPLTRVSGGAALAAQQSYWPHFVTVTVLFALVLSGALIILVASPLRALRARGKVQGCETLRPGEKAPLSREQHLQSPKECRTSASDVDADNNCLGTEVA

SEQ ID NO: 614 - protein, 760 aa, Macaca fascicularis
MALPALGLDPWSLLGLFLFQLLQLLLPTTTAGGGGQGPMPRVRYYAGDERRALSFFHQKGLQDFDTLLLSGNGNTLYVGAREAILALDIQDPGVPRLKNMIPWPASDRKKSECAFKKKSNETQCFNFIRVLVSYNVTHLYTCGTFAFSPACTFIELQDSHLLPILEDKVMEGKGQSPFDPAHKHTAVLVDGMLYSGTMNNFLGSEPILMRTLGSQPVLKTDNFLRWLHPDASFVAAIPSTQVVYFFFEETASEFDFFERLHTSRVARVCKNDVGGEKLLQRKWTTFLKAQLLCTKPGQLPFNVIRHAVLLPADSPTAPHIYAVFTSQWQIGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGRSHLVMYLGTTTGSLHKAVVSGDSGAHLVEEIQLFPDPEPVRNLQLAPTQGAVFVGFSGGVWRVPRANCSVYESCVDCVLARDPHCAWDPESRTCCFLSAPTLNSWKQDMERGNPEWACASGPMSRSLRPQSRPQIIKEVLAVPNSFLELPCPHLSALASYYWSHGPAAVPEASSTVYNGSLLLIVQDGVGGLYQCWATENGFSYPVVSYWVDSQDQSLALDPELAGIPREHVEVPLTRVSGGTALAAQRSYWPHFVTVTVLLALVLSGALIILLASPLGALRARGKVQGCETLPPGEKAPLSREQHLQSPKECRTSASDVDADNCLGTEVA

SEQ ID NO: 615 - protein, 760 aa, Mus musculus
MALPSLGQDSWSLLRVFFFQLFLLPSLPPASGTGGQGPMPRVKYHAGDGHRALSFFQQKGLRDFDTLLLSDDGNTLYVGAREAVLALNIQNPGIPRLKNMIPWPASERKKTECAFKKKSNETQCFNFIRVLVSYNATHLYACGTFAFSPACTFIELQDSLLLPILIDKVMDGKGQSPFDPVHKHTAVLVDGMLYSGTMNNFLGSEPILMRTLGSQPVLKTDIFLRWLHADASFVAAIPSTQVVYFFFEETASEFDFFEELYISRVAQVCKNDVGGEKLLQKKWTTFLKAQLLCAQPGQLPFNIIRHAVLLPADSPSVSRIYAVFTSQWQVGGTRSSAVCAFSLTDIERVFKGKYKELNKETSRWTTYRGSEVSPRPGSCSMGPSSDKALTFMKDHFLMDEHVVGTPLLVKSGVEYTRLAVESARGLDGSSHVVMYLGTSTGSLHKAVVPQDSSAYLVEEIQLSPDSEPVRNLQLAPAQGAVFAGFSGGIWRVPRANCSVYESCVDCVLARDPHCAWDPESRLCSLLSGSTKPWKQDMERGNPEWVCTRGPMARSPRRQSPPQLIKEVLTVPNSILELPCPHLSALASYHWSHGRAKISEASATVYNGSLLLLPQDGVGGLYQCVATENGYSYPVVSYWVDSQDQPLALDPELAGVPRERVQVPLTRVGGGASMAAQRSYWPHFLIVTVLLAIVLLGVLTLLLASPLGALRARGKVQGCGMLPPREKAPLSRDQHLQPSKDHRTSASDVDADNNHLGAEVA

SEQ ID NO: 616 - protein, 718 aa, synthetic construct
  REGION 1..718: note = Human SEMA4A AviHis10 Recombinant Human SEMA4A UniProt ID Q9H3S1 amino acids33 - 683 with C-terminal Avi tag and His10 tag.
MALPALGLDPWSLLGLFLFQLLQLLLPTTTAGGGGQGPMPRVRYYAGDERRALSFFHQKGLQDFDTLLLSGDGNTLYVGAREAILALDIQDPGVPRLKNMIPWPASDRKKSECAFKKKSNETQCFNFIRVLVSYNVTHLYTCGTFAFSPACTFIELQDSYLLPISEDKVMEGKGQSPFDPAHKHTAVLVDGMLYSGTMNNFLGSEPILMRTLGSQPVLKTDNFLRWLHHDASFVAAIPSTQVVYFFFEETASEFDFFERLHTSRVARVCKNDVGGEKLLQKKWTTFLKAQLLCTQPGQLPFNVIRHAVLLPADSPTAPHIYAVFTSQWQVGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGHSHLVMYLGTTTGSLHKAVVSGDSSAHLVEEIQLFPDPEPVRNLQLAPTQGAVFVGFSGGVWRVPRANCSVYESCVDCVLARDPHCAWDPESRTCCLLSAPNLNSWKQDMERGNPEWACASGPMSRSLRPQSRPQIIKEVLAVPNSILELPCPHLSALASYYWSHGPAAVPEASSTVYNGSLLLIVQDGVGGLYQCWATENGFSYPVISYWVDSQDQTLALDPELAGIPREHVKVPLTRVSGGAALAAQQSYWPHGTGGGSGGGSGLNDIFEAQKIEWHEHHHHHHHHHH

SEQ ID NO: 617 - protein, 686 aa, synthetic construct
  REGION 1..686: note = Mouse SEMA4A AviHis10 Recombinant mouse SEMA4A (UniProt ID Q62178, amino acids 33-682)with C-terminal Avi tag and His10 tag
MTGGQGPMPRVKYHAGDGHRALSFFQQKGLRDFDTLLLSDDGNTLYVGAREAVLALNIQNPGIPRLKNMIPWPASERKKTECAFKKKSNETQCFNFIRVLVSYNATHLYACGTFAFSPACTFIELQDSLLLPILIDKVMDGKGQSPFDPVHKHTAVLVDGMLYSGTMNNFLGSEPILMRTLGSQPVLKTDIFLRWLHADASFVAAIPSTQVVYFFFEETASEFDFFEELYISRVAQVCKNDVGGEKLLQKKWTTFLKAQLLCAQPGQLPFNIIRHAVLLPADSPSVSRIYAVFTSQWQVGGTRSSAVCAFSLTDIERVFKGKYKELNKETSRWTTYRGSEVSPRPGSCSMGPSSDKALTFMKDHFLMDEHVVGTPLLVKSGVEYTRLAVESARGLDGSSHVVMYLGTSTGSLHKAVVPQDSSAYLVEEIQLSPDSEPVRNLQLAPAQGAVFAGFSGGIWRVPRANCSVYESCVDCVLARDPHCAWDPESRLCSLLSGSTKPWKQDMERGNPEWVCTRGPMARSPRRQSPPQLIKEVLTVPNSILELPCPHLSALASYHWSHGRAKISEASATVYNGSLLLLPQDGVGGLYQCVATENGYSYPVVSYWVDSQDQPLALDPELAGVPRERVQVPLTRVGGGASMAAQRSYWPHGTGGGSGGGSGLNDIFEAQKIEWHEHHHHHHHHHH

SEQ ID NO: 618 - protein, 684 aa, synthetic construct
  REGION 1..684: note = Cyno SEMA4A AviHis10 Recombinant cynomolgus SEMA4A (UniProt ID G7NV79, amino acids 32- 679) with C-terminal Avi tag and His10 tag.
MGGGGQGPMPRVRYYAGDERRALSFFHQKGLQDFDTLLLSGNGNTLYVGAREAILALDIQDPGVPRLKNMIPWPASDRKKSECAFKKKSNETQCFNFIRVLVSYNVTHLYTCGTFAFSPACTFIELQDSHLLPILEDKVMEGKGQSPFDPAHKHTAVLVDGMLYSGTMNNFLGSEPILMRTLGSQPVLKTDNFLRWLHPDASFVAAIPSTQVVYFFFEETASEFDFFERLHTSRVARVCKNDVGGEKLLQRKWTTFLKAQLLCTKPGQLPFNVIRHAVLLPADSPTAPHIYAVFTSQWQIGGTRSSAVCAFSLLDIERVFKGKYKELNKETSRWTTYRGPETNPRPGSCSVGPSSDKALTFMKDHFLMDEQVVGTPLLVKSGVEYTRLAVETAQGLDGRSHLVMYLGTTTGSLHKAVVSGDSGAHLVEEIQLFPDPEPVRNLQLAPTQGAVFVGFSGGVWRVPRANCSVYESCVDCVLAQDPHCAWDPESRTCCFLSAPTLNSWKQDMERGNPEWACASGPMSRSLRPQSRPQIIKEVLAVPNSFLELPCPHLSALASYYWSHGPAAVPEASSTVYNGSLLLIVQDGVGGLYQCWATENGFSYPVVSYWVDSQDQSLALDPELAGIPREHVEVPLTRVSGGTALAAQRSGTGGGSGGGSGLNDIFEAQKIEWHEHHHHHHHHHH